(19) United States
(12) Patent Application Publication
(10) Pub. No.: US 2007/0105114 A1
(43) Pub. Date: May 10, 2007

(54) Biomarkers of cyclin-dependent kinase modulation

(76) Inventors: Martha Li, Short Hills, NJ (US); Brent A. Rupnow, Ewing, NJ (US); Kevin R. Webster, Hopkinton, MA (US); Donald G. Jackson, Lawrenceville, NJ (US); Tai W. Wong, Belle Mead, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 10/567,867

(22) PCT Filed: Jul. 29, 2004
(86) PCT No.: PCT/US04/24424

§ 371 Date: Aug. 18, 2006

Related U.S. Application Data
(60) Provisional application No. 60/490,890, filed on Jul. 29, 2003.

(51) Int. Cl. C12Q 1/68; G01N 33/574

(52) U.S. Cl. 435/6; 435/7.23

(57) ABSTRACT

Biomarkers having expression patterns that correlate with a response of cells to treatment with one or more cdk modulating agents, and uses thereof. Also provided are methods for testing or predicting whether a mammal will respond therapeutically to a method of treating cancer that comprises administering an agent that modulates cdk activity.

### SEQUENCE LISTING

The present application includes a Sequence Listing. A compact disc labeled “COPY 1—SEQUENCE LISTING PART” contains the Sequence Listing as D0310 PCT.sequence listing.ST25.txt. The Sequence Listing is 13394 KB in size and was recorded on Jul. 28, 2004. The compact disc is 1 of 3 compact discs. Duplicate copies of the compact disc are labeled “COPY 2—SEQUENCE LISTING PART” and “COPY 3—SEQUENCE LISTING PART.” Also included is a computer readable form of the Sequence Listing.

The compact disc and duplicate copies are identical and are hereby incorporated by reference into the present application.

### BACKGROUND OF THE INVENTION

The present invention relates generally to the field of pharmacogenomics and, more specifically, to pharmacodynamic biomarkers whose expression patterns correlate with a response of cells to treatment with one or more cdk modulating agents.

Uncontrolled proliferation is a hallmark of cancer cells. Over the past two decades, it has become increasingly clear that the molecules, which directly control cell cycle progression, accumulate defects during tumorigenesis. These defects can result in the loss of checkpoint control and/or the inappropriate activation of the drivers of cell cycle progression, the cyclin-dependent kinases (referred to as “cdks” or “CDKs”). Misregulation of cdk function occurs with high frequency in major solid tumor types (including breast, colon, ovarian, prostate, and NSCL carcinomas). Therefore, inhibitors of cdks and cell cycle progression have the potential to fill a large therapeutic need.

The cdks are serine/threonine protein kinases that are the driving force behind the cell cycle and cell proliferation. Cdks are multisubunit enzymes composed of at least a catalytic subunit and a regulatory (cyclin) subunit Morgan, D. O., Nature 1995; 374:131-134. To date, nine cdks (cdk1 through cdk9) and eleven cyclin subunits have been identified which can form in excess of thirteen active kinase complexes. Gould, K. L. (1994) in Protein Kinases (Woodgett, J. R., ed), pp. 149-166, Oxford University Press, Oxford. In normal cells, many of these enzymes can be categorized as G1, S, or G2/M phase enzymes which perform distinct roles in cell cycle progression. van den Heuvel, S., and Harlow, E., Science 1993; 262: 2050-2054. Cdks phosphorylate and modulate the activity of a variety of cellular proteins that include tumor suppressors (e.g., RB, p53), transcription factors (e.g., E2F-DP1, RNA pol II), replication factors (e.g., DNA pol α, replication protein A), and organizational factors which influence cellular and chromatin structures (e.g., Histone HI, lamin A, MAP4). Nigg, E. A., Trends in Cell Biology 1993; 3:296-301; Rickert, P. et al., Oncogene 1996; 12:2631-2640; Dynlacht, B. D. et al., Mol Cell Biol 1997; 17:3867-3875; Ookata, K. et al., Biochemistry 1997; 36:15873-15883.

Cdk activity is regulated through a variety of co-ordinated mechanisms, which include cell cycle dependent transcription and translation, cell cycle dependent proteolysis, subcellular localization, post-translational modifications, and interaction with cdk inhibitor proteins (referred to as “CKIs”). Pines, J., and Hunter, T., Cell 1989; 58:833-846; King, R. W. et al., Science 1996; 274:1652-1659; Li, J. et al., Proc Natl Acad Sci USA 1997; 94:502-507; Draetta, G., and Beach, D., Cell 1988; 54:17-26; Harper, J. W., Cancer Surv 1997; 29:91-107. It is through these mechanisms that cell cycle checkpoints are constructed. This realization that checkpoint control is implemented through the regulation of cdk function has made the cdks and their regulatory pathways compelling targets for the development of chemotherapeutic agents. The p27/cdk2/cyclinE/RB checkpoint pathway has been clearly implicated in tumorigenesis.

Numerous reports have demonstrated that both the co-activator, cyclin E, and inhibitor, p27, of cdk2 are either over-expressed or under-expressed respectively in solid tumors. Porter, P. L. et al., Nat Med 1997; 3:222-225; Kitahara, K et al., Int J Cancer 1995; 62:25-28; Wang, A. et al., J Cancer Res Clin Oncol 1996; 122:122-126; Keyomarsi, K et al., Cancer Res 1994; 54:380-385; Coinjal, F. et al. Int J Cancer 1996; 69:247-253; Akama, Y. et al., Jpn J Cancer Res 1995; 86:617-621; Tan, P. et al., Cancer Res 1997; 57:1259-1263; Catzavelos, C. et al., Nat Med 1997; 3:227-230; Fredersdorf, S. et al., Proc Natl Acad Sci USA 1997; 94:6380-6385. Their altered expression has been shown to correlate with increased cdk2 activity levels and poor prognosis.

In the early clinical development of anti-cancer agents, clinical trials are typically designed to evaluate the safety, tolerability, and pharmacokinetics, as well as to identify a suitable dose and schedule for further clinical evaluation. Increasingly, there is a need to also evaluate the pharmacologic effects of novel agents in early clinical trials, particularly in cases where dosing to maximum tolerated doses may not be appropriate. As a result, there is considerable interest in identifying pharmacodynamic (PD) biomarkers that correlate with the pharmacologic modulation of a tumor target. These PD biomarkers may be tumor-specific, but ideally should also be expressed in accessible surrogate tissues such as skin or peripheral blood cells. The identification of these PD biomarkers may be carried out by analyzing changes in specific polypeptides or mRNA, as predicted by the known biology associated with the molecule targeted by the agent of interest. Alternatively, PD biomarkers can be identified by analyzing global changes in polypeptides or mRNA in cells or tissues exposed to efficacious doses of the agent. Once identified, these PD biomarkers can be used to demonstrate the desired pharmacologic modulation (e.g., inhibition) of a tumor target upon the achievement of an appropriate level of agent in the patient.

There remains a need to identify biomarkers whose expression patterns correlate with a response of cells to treatment with one or more cdk modulating agents.

The development of microarray technologies for large scale characterization of mRNA expression pattern has made it possible to systematically search for molecular biomarkers whose expression is modulated by drug treatment Such technologies and molecular tools have made it possible to monitor the expression level of a large number of transcripts within a cell population at any given time (see, e.g., Schena et al., 1995, Science, 270:467-470; Lockhart et al., 1996, Nature Biotechnology, 14:1675-1680; Blanchard et al., 1996, Nature Biotechnology, 14:1649; U.S. Pat. No. 5,569,588).

### SUMMARY OF THE INVENTION

The invention provides methods and procedures for determining patient sensitivity to one or more agents that modulate cyclin-dependent kinase (cdk) activity. The invention also provides methods for determining or predicting whether an individual requiring therapy for a disease state or disorder such as cancer will or will not respond to treatment, prior to administration of the treatment, wherein the treatment comprises of one or more agents that modulate cdk activity. The one or more agents that modulate cdk activity can be small molecules or biological molecules. In one aspect, the agent is a small molecule that inhibits cyclin-dependent kinase 2 (cdk2)/cyclin E.

The invention also provides a method for testing or predicting whether a mammal will respond therapeutically to a method of treating cancer comprising administering an agent that modulates cdk activity, wherein the method comprises: (a) measuring in the mammal the level of at least one biomarker selected from the biomarkers of Table 1; (b) exposing the mammal to the agent that modulates cdk activity; (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides a method for determining whether a mammal is responding to an agent that modulates cdk activity, comprising: (a) exposing the mammal to the agent; and (b) following the exposing of step (a), measuring in the mammal the level of at least one biomarker selected from the biomarkers of Table 1, wherein a difference in the level of the at least one biomarker measured in step (b), compared to the level of the at least one biomarker in a mammal that has not been exposed to said agent, indicates that the mammal is responding to the agent that modulates cdk activity.

As used herein, responding includes, for example, a biological response (e.g., a cellular response) or a clinical response (e.g., improved symptoms, a therapeutic effect, or an adverse event) in the mammal.

The invention also provides a method for determining whether a mammal is responding to an agent that modulates cdk activity, comprising: (a) obtaining a biological sample from the mammal; (b) measuring in said biological sample the level of at least one biomarker selected from the biomarkers of Table 1; (c) correlating said level of at least one biomarker with a baseline level; and (d) determining whether the mammal is responding to an agent that modulates cdk activity based on said correlation.

As used herein, the baseline level used for the correlation can be determined by one of skill in the art. In one aspect, the baseline level is the level of the at least one biomarker selected from the biomarkers of Table 1 in a mammal that has not been exposed to the agent. In another aspect, the baseline level is the level of the at least one biomarker selected from the biomarkers of Table 1 in the mammal that will be treated with a cdk modulating agent but has not yet been exposed to the agent. In yet another aspect, the baseline level is the level of the at least one biomarker selected from the biomarkers of Table 1 in the mammal that has been treated with a cdk modulating agent, and wherein the baseline level is selected at a point during the treatment with the cdk modulating agent. The point can be, for example, an established time period or measurement of a criteria (e.g., a biological or clinical response) set prior to initiation of the treatment.

A difference between the level of at least one biomarker from the mammal and the baseline level that is statistically significant can be used in the methods of the invention. A statistically significant difference between the level of at least one biomarker from the mammal and the baseline level is readily determined by one of skill in the art and can be, for example, at least a two-fold difference, at least a three-fold difference, or at least a four-fold difference in the level of the at least one biomarker.

The invention also provides a method for identifying a mammal that will respond therapeutically to a method of treating cancer comprising administering an agent that modulates cdk activity, wherein the method comprises: (a) measuring in the mammal the level of at least one biomarker selected from the biomarkers of Table 1; (b) exposing a biological sample from the mammal to the agent; (c) following the exposing in step (b), measuring in said biological sample the level of the at least one biomarker, wherein a difference in the level of the at least one biomarker measured in step (c) compared to the level of the at least one biomarker measured in step (a) indicates that the mammal will respond therapeutically to the said method of treating cancer.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The invention also provides a method for identifying a mammal that will respond therapeutically to a method of treating cancer comprising administering an agent that modulates cdk activity, wherein the method comprises: (a) exposing a biological sample from the mammal to the agent that modulates cdk activity; (b) following the exposing of step (a), measuring in said biological sample the level of at least one biomarker selected from the biomarkers of Table 1, wherein a difference in the level of the at least one biomarker measured in step (b), compared to the level of the at least one biomarker in a mammal that has not been exposed to said agent that modulates cdk activity, indicates that the mammal will respond therapeutically to said method of treating cancer.

The invention also provides a method for determining whether an agent modulates cdk activity in a mammal, comprising: (a) exposing the mammal to the agent; and (b) following the exposing of step (a), measuring in the mammal the level of at least one biomarker selected from the biomarkers of Table 1, wherein a difference in the level of said biomarker measured in step (b), compared to the level of the biomarker in a mammal that has not been exposed to said agent, indicates that the agent modulates cdk activity in the mammal.

The invention also provides a method for determining whether a mammal has been exposed to an agent that modulates cdk activity, comprising (a) exposing a biological sample from the mammal to the agent; and (b) following the exposing of step (a), measuring in the biological sample the level of at least one biomarker selected from the biomarkers of Table 1, wherein a difference in the level of said biomarker measured in step (b), compared to the level of the biomarker in a mammal that has not been exposed to said agent, indicates that the mammal has been exposed to an agent that modulates cdk activity.

The mammal can be, for example, a human, rat, mouse, dog, rabbit, pig sheep, cow, horse, cat, primate, or monkey.

The method of the invention can be an in vivo or an in vitro method. In one aspect, the step of measuring in the mammal the level of at least one biomarker is in vitro and comprises taking a biological sample from the mammal and then measuring the level of the at least one biomarker in the biological sample. The biological sample can comprise, for example, at least one of whole fresh blood, peripheral blood mononuclear cells, frozen whole blood, fresh plasma, frozen plasma, urine, saliva, skin, hair follicle, bone marrow, or tumor tissue.

In one aspect of the invention, the method of the invention comprises use of the biomarker W28729 (SEQ ID NO:1246).

The level of the at least one biomarker can be, for example, the level of protein and/or mRNA transcript of the at least one biomarker.

The invention also provides an isolated biomarker selected from the biomarkers of Table 1. The biomarkers of the invention comprise sequences selected from the nucleotide and amino acid sequences provided in Table 1 and the Sequence Listing, including fragments and variants thereof.

The invention also provides one or more biomarkers that can serve as targets for the development of therapies for disease treatment. Such targets may be particularly applicable for treatment of cancers or tumors.

The invention also provides a biomarker set comprising two or more biomarkers selected from the biomarkers of Table 1.

The invention also provides kits for determining or predicting whether a patient would be susceptible or resistant to a treatment that comprises one or more agents that modulate cdk activity. In one aspect, the patient has a cancer.

In one aspect, the kit comprises a suitable container that comprises one or more specialized microarrays of the invention, one or more agents that modulate cdk activity for use in testing cells from patient tissue specimens or patient samples, and instructions for use. The kit may further comprise reagents or materials for monitoring the expression of a biomarker set at the level of mRNA or protein.

The invention also provides a kit that comprises two or more biomarkers selected from the biomarkers of Table 1.

The invention also provides a kit that comprises at least one of an antibody and a nucleic acid for detecting the presence of at least one of the biomarkers selected from the biomarkers of Table 1. In one aspect, the kit further comprises instructions for determining whether or not a mammal will respond therapeutically to a method of treating cancer comprising administering an agent that modulates cdk activity. In another aspect, the instructions comprise the steps of (a) measuring in the mammal the level of at least one biomarker selected from the biomarkers of Table 1, (b) exposing the mammal to the agent, (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides screening assays for determining if a patient will be susceptible or resistant to treatment with one or more agents that modulate cdk activity.

The invention also provides a method of monitoring the treatment of a patient having a disease, wherein said disease is treated by a method comprising administering one or more agents that modulate cdk activity.

The invention also provides individualized genetic profiles which are necessary to treat diseases and disorders based on patient response at a molecular level.

The invention also provides specialized microarrays, e.g., oligonucleotide microarrays or cDNA microarrays, comprising one or more biomarkers having expression profiles that correlate with either sensitivity or resistance to one or more agents that modulate cdk activity.

The invention also provides antibodies, including polyclonal and monoclonal, directed against one or more of the biomarker polypeptides. Such antibodies can be used in a variety of ways, for example, to purify, detect, and target the biomarker polypeptides of the invention, including both in vitro and in vivo diagnostic, detection, screening, and/or therapeutic methods.

The invention also provides a cell culture model to identity biomarkers whose expression levels correlate with cdk modulation.

The invention will be better understood upon a reading of the detailed description of the invention when considered in connection with the accompanying figures.

### BRIEF DESCRIPTION OF THE FIGURES

FIG. 1 illustrates a cdk biomarker identification strategy.

FIGS. 2A and 2B illustrate the reduction of cdk2 protein levels by cdk2 antisense oligonucleotides.

FIGS. 3A, 3B, and 3C illustrate the expression changes of the biomarker W28729 (SEQ ID NO:1246) in A2780s, PBMC, and xenograft A2780s tumors following treatment with a cdk inhibitor.

FIGS. 4A and 4B illustrate the regulation of W28729 expression in A2780 xenograft (FIG. 4A) and the mouse ortholog of W28729 in mouse PBMC (FIG. 4B).

FIGS. 5A and 5B illustrate W28729 gene expression in patients treated with N-5-[[5-(1,1-Dimethylethyl)-2-oxazolyl]methyl]thio]-2-thiazolyl-4-piperidinecarboxamide, 0.5-L-tartaric acid salt.

FIGS. 6A and 6B illustrate W28729 induction and its relation to baseline expression.

FIGS. 7A and 7B illustrate W28729 induction as a function of dose (FIG. 7A) and AUC (FIG. 7B).

FIG. 8 illustrates the prediction of W28729 changes by baseline expression of W28729 and the cdk2 inhibitor exposure.

FIG. 9 illustrates disease outcome, time to tumor progression (TTP) and W28729 changes.

### DETAILED DESCRIPTION OF THE INVENTION

As used herein, the term “agent that modulates cdk activity,” also referred to herein as “cdk modulating agent,” is intended to mean a substance that is a biological molecule or a small molecule, and formulations thereof, that is directly or indirectly involved in cdk activity and/or one or more pathways in which cdk is involved. The cdk modulating agent can be a cdk antagonist or inhibitor. The cdk modulating agent can also be a cdk agonist or activator.

In one aspect, the cdk modulating agent is directly or indirectly involved in cdk2 activity and/or one or more pathways in which cdk2 is involved. In another aspect, the cdk modulating agent is directly or indirectly involved in cdk1 activity and/or one or more pathways in which cdk1 is involved. In yet another aspect, the cdk modulating agent is directly or indirectly involved in cdk4 activity and/or one or more pathways in which cdk4 is involved.

Biological molecules include all lipids and polymers of monosaccharides, amino acids, and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA. Biological molecules further include derivatives of any of the molecules described above. For example, derivatives of biological molecules include lipid and glycosylation derivatives of oligopeptides, polypeptides, peptides, and proteins.

In addition to the biological molecules discussed above, the cdk modulating agents may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the cdk modulating agent is a small molecule that inhibits cdk or a pathway in which cdk is involved.

Numerous small molecules have been described as being useful to inhibit cdk including, for example, flavopiridol (Aventis Pharmaceuticals Inc., Bridgewater, N.J., U.S.A.) and CYC202 (Cyclacel Limited, Dundee, United Kingdom). Cdk inhibitors also include, for example, the small molecules disclosed in U.S. Pat. Nos. 6,040,321, 6,214,852, 6,262,096, 6,515,004, and 6,521,759.

In one aspect, the cdk modulating agent is a small molecule cdk inhibitor. In another aspect, the cdk modulating agent is a small molecule cdk2 inhibitor. In another aspect, the cdk modulating agent is a small molecule cdk1 inhibitor. In yet another aspect, the cdk modulating agent is a small molecule cdk4 inhibitor. In a further aspect, the cdk modulating agent is N-5-[[5-(1,1-Dimethylethyl)-2-oxazolyl]methyl]thio]-2-thiazolyl-4-piperidinecarboxamide, 0.5-L-tartaric acid salt.

The invention provides methods to monitor the response of patients to treatment with a cdk modulating agent. These methods are useful: (i) to follow the response of a patient over a course of treatment with a cdk modulating agent; (ii) to determine whether the specific cdk modulating agent selected for treatment is appropriate to the patient; (iii) to determine whether the dose of the cdk modulating agent being administered is appropriate to the patient; (iv) to determine whether the type and/or amount of cdk modulating agent being administered needs to be changed over the course of the treatment period; (v) to determine when treatment is complete; and (vi) to determine whether treatment that has been terminated needs to be restarted. These methods are also useful to identify whether a patient will benefit from treatment with a cdk modulating agent.

In one aspect, the invention provides a method of determining whether a patient receiving a treatment that comprises a cdk modulating agent has received sufficient treatment to inhibit cdk in the patient's tumors. In accordance with the invention, tumor or surrogate biopsies are obtained from a patient before and after treatment with a cdk modulating agent. The surrogate biopsies can be, for example, skin or peripheral blood. The cells are then assayed to determine the changes in the expression pattern of one or more biomarkers of the invention upon treatment with the cdk modulating agent, to determine whether cdk inhibition has been achieved by the treatment. Success or failure of the treatment can be determined based on the expression pattern of the test cells from the test tissue, e.g., tumor or cancer biopsy, as being relatively the same as or different from the expression pattern of one or more biomarkers. If the test cells show an expression profile which corresponds to that of the biomarker or biomarker set, it is predicted that the individual's cancer or tumor has been exposed to a concentration of the modulating agent that is sufficient to, in one aspect, inhibit cdk. By contrast, if the test cells show a gene expression pattern that does not correspond to the biomarker or biomarker set, it is predicted that the modulating agent exposure has not been sufficient to, in one aspect, inhibit cdk.

In another aspect, the invention provides a method of monitoring the treatment of a patient having a disease treatable by a cdk modulating agent by comparing the expression profile of cells from a patient tissue sample, e.g., a tumor or cancer biopsy, following treatment to a biomarker or biomarker set. The isolated cells from the patient are assayed to determine their expression pattern to determine if a change of the expression profile has occurred so as to warrant a different treatment, such as treatment with a different cdk modulating agent, or to discontinue current treatment. The resulting expression profile of the cells following treatment with a cdk modulating agent is compared with the expression pattern of the biomarker or biomarker set.

Such a monitoring process can indicate success or failure of a patient's treatment with a cdk modulating agent based on the expression pattern of the cells isolated from the patient's sample as being relatively the same as or different from the expression pattern of the biomarker or biomarker set Thus, if, after treatment with a cdk modulating agent, the test cells show a change in their expression profile from the biomarker or biomarker set, it can serve as an indicator that the current treatment should be modified, changed, or even discontinued. Such monitoring processes can be repeated as necessary or desired. The monitoring of a patient's response to a given treatment can also involve testing the patient's cells in the assay as described only after treatment with a cdk modulating agent, rather than before and after treatment with a cdk modulating agent.

The invention is based on the identification of specific pharmacodynamic biomarkers of cdk modulation. In accordance with the invention, oligonucleotide microarrays were used to measure the expression levels of a large number of genes in a panel of treated cell lines for which sensitivity to a cdk modulating agent was determined. The determination of the gene expression profiles in the treated cells allowed the identification of biomarkers whose expression levels highly correlate with the modulation of cdk or a pathway in which cdk is involved. The biomarkers are thus useful for inferring the level of cdk modulation in a patient.

The biomarkers of the invention include polynucleotides, including full-length genes, open reading frames (ORFs), and partial sequences such as expressed sequence tags (ESTs) and structural RNA. In one aspect, the invention is directed to an isolated polynucleotide comprising a nucleotide sequence selected from the nucleotide sequences of Table 1 such as, for example, an isolated polynucleotide comprising the nucleotide sequence of SEQ ID NO:1264. The biomarkers further include polypeptides comprising the amino acid sequences encoded by these polynucleotides. The biomarkers of the invention include those provided below in Table 1. In one aspect, these polynucleotides and polypeptides are in isolated form.

TABLE 1

| SEQ ID | Sequence | Genbank Accession | | |
|---|---|---|---|---|
| NO: | type | No. | Symbol | Description |
| 1 | DNA | NM_005340 | HINT1 | histidine triad nucleotide |
| | | | | binding protein 1 |
| 2 | Protein | NP_005331 | HINT1 | histidine triad nucleotide |
| | | | | binding protein 1 |
| 3 | DNA | NM_003137 | SRPK1 | SFRS protein kinase 1 |
| 4 | Protein | NP_003128 | SRPK1 | SFRS protein kinase 1 |
| 5 | DNA | NM_001951 | E2F5 | E2F transcription factor 5, |
| | | | | p130-binding |
| 6 | Protein | NP_001942 | E2F5 | E2F transcription factor 5, |
| | | | | p130-binding |
| 7 | DNA | U33838 | | NF-kappa-B p65delta3, mRNA |
| | | | | sequence |
| 8 | Protein | U33838 (Translation) | | NF-kappa-B p65delta3, mRNA |
| | | | | sequence |
| 9 | DNA | NM_005195 | CEBPD | CCAAT/enhancer binding |
| | | | | protein (C/EBP), delta |
| 10 | Protein | NP_005186 | CEBPD | CCAAT/enhancer binding |
| | | | | protein (C/EBP), delta |
| 11 | DNA | NM_002916 | RFC4 | replication factor C (activator |
| | | | | 1) 4, 37 kDa |
| 12 | Protein | NP_002907 | RFC4 | replication factor C (activator |
| | | | | 1) 4, 37 kDa |
| 13 | DNA | NM_002050 | MGC2306 | hypothetical protein MGC2306 |
| 14 | Protein | NP_002041 | MGC2306 | hypothetical protein MGC2306 |
| 15 | DNA | NM_032638 | MGC2306 | hypothetical protein MGC2306 |
| 16 | Protein | NP_116027 | MGC2306 | hypothetical protein MGC2306 |
| 17 | DNA | NM_001709 | BDNF | brain-derived neurotrophic |
| | | | | factor |
| 18 | Protein | NP_001700 | BDNF | brain-derived neurotrophic |
| | | | | factor |
| 19 | DNA | NM_170731 | BDNF | brain-derived neurotrophic |
| | | | | factor |
| 20 | Protein | NP_733927 | BDNF | brain-derived neurotrophic |
| | | | | factor |
| 21 | DNA | NM_170732 | BDNF | brain-derived neurotrophic |
| | | | | factor |
| 22 | DNA | NM_170733 | BDNF | brain-derived neurotrophic |
| | | | | factor |
| 23 | DNA | NM_006749 | SLC20A2 | solute carrier family 20 |
| | | | | (phosphate transporter), |
| | | | | member 2 |
| 24 | Protein | NP_006740 | SLC20A2 | solute carrier family 20 |
| | | | | (phosphate transporter), |
| | | | | member 2 |
| 25 | DNA | NM_005415 | SLC20A1 | solute carrier family 20 |
| | | | | (phosphate transporter), |
| | | | | member 1 |
| 26 | Protein | NP_005406 | SLC20A1 | solute carrier family 20 |
| | | | | (phosphate transporter), |
| | | | | member 1 |
| 27 | DNA | HG3510-HT3704 | | V-Erba Related Ear-3 Protein |
| 28 | DNA | HG1471-HT3923 | | Transcription Factor Oct-1a/1b, |
| | | | | Alt. Splice 2, Oct-1b |
| 29 | DNA | NM_002816 | PSMD12 | proteasome (prosome, |
| | | | | macropain) 26S subunit, non- |
| | | | | ATPase, 12 |
| 30 | Protein | NP_002807 | PSMD12 | proteasome (prosome, |
| | | | | macropain) 26S subunit, non- |
| | | | | ATPase, 12 |
| 31 | DNA | NM_003138 | SRPK2 | SFRS protein kinase 2 |
| 32 | Protein | NP_003129 | SRPK2 | SFRS protein kinase 2 |
| 33 | DNA | NM_005930 | MGEA6 | meningioma expressed antigen |
| | | | | 6 (coiled-coil proline-rich) |
| 34 | Protein | NP_005921 | MGEA6 | meningioma expressed antigen |
| | | | | 6 (coiled-coil proline-rich) |
| 35 | DNA | NM_003337 | UBE2B | ubiquitin-conjugating enzyme |
| | | | | E2B (RAD6 homolog) |
| 36 | Protein | NP_003328 | UBE2B | ubiquitin-conjugating enzyme |
| | | | | E2B (RAD6 homolog) |
| 37 | DNA | NM_003406 | YWHAZ | tyrosine 3- |
| | | | | monooxygenase/tryptophan 5- |
| | | | | monooxygenase activation |
| | | | | protein, zeta polypeptide |
| 38 | Protein | NP_003397 | YWHAZ | tyrosine 3- |
| | | | | monooxygenase/tryptophan 5- |
| | | | | monooxygenase activation |
| | | | | protein, zeta polypeptide |
| 39 | DNA | NM_145690 | YWHAZ | tyrosine 3- |
| | | | | monooxygenase/tryptophan 5- |
| | | | | monooxygenase activation |
| | | | | protein, zeta polypeptide |
| 40 | DNA | NM_006494 | ERF | Ets2 repressor factor |
| 41 | Protein | NP_006485 | ERF | Ets2 repressor factor |
| 42 | DNA | NM_006904 | PRKDC | protein kinase, DNA-activated, |
| | | | | catalytic polypeptide |
| 43 | Protein | NP_008835 | PRKDC | protein kinase, DNA-activated, |
| | | | | catalytic polypeptide |
| 44 | DNA | NM_021975 | RELA | v-rel reticuloendotheliosis viral |
| | | | | oncogene homolog A, nuclear |
| | | | | factor of kappa light |
| | | | | polypeptide gene enhancer in |
| | | | | B-cells 3, p65 (avian) |
| 45 | Protein | NP_068810 | RELA | v-rel reticuloendotheliosis viral |
| | | | | oncogene homolog A, nuclear |
| | | | | factor of kappa light |
| | | | | polypeptide gene enhancer in |
| | | | | B-cells 3, p65 (avian) |
| 46 | DNA | NM_004359 | CDC34 | cell division cycle 34 |
| 47 | Protein | NP_004350 | CDC34 | cell division cycle 34 |
| 48 | DNA | NM_000380 | XPA | xeroderma pigmentosum, |
| | | | | complementation group A |
| 49 | Protein | NP_000371 | XPA | xeroderma pigmentosum, |
| | | | | complementation group A |
| 50 | DNA | NM_004152 | OAZ1 | ornithine decarboxylase |
| | | | | antizyme 1 |
| 51 | Protein | NP_004143 | OAZ1 | ornithine decarboxylase |
| | | | | antizyme 1 |
| 52 | DNA | NM_003250 | THRA | thyroid hormone receptor, |
| | | | | alpha (erythroblastic leukemia |
| | | | | viral (v-erb-a) oncogene |
| | | | | homolog, avian) |
| 53 | Protein | NP_003241 | THRA | thyroid hormone receptor, |
| | | | | alpha (erythroblastic leukemia |
| | | | | viral (v-erb-a) oncogene |
| | | | | homolog, avian) |
| 54 | DNA | NM_005900 | MADH1 | MAD, mothers against |
| | | | | decapentaplegic homolog 1 |
| | | | | (Drosophila) |
| 55 | Protein | NP_005891 | MADH1 | MAD, mothers against |
| | | | | decapentaplegic homolog 1 |
| | | | | (Drosophila) |
| 56 | DNA | NM_004444 | EPHB4 | EphB4 |
| 57 | Protein | NP_004435 | EPHB4 | EphB4 |
| 58 | DNA | NM_021009 | UBC | ubiquitin C |
| 59 | Protein | NP_066289 | UBC | ubiquitin C |
| 60 | DNA | NM_003200 | TCF3 | transcription factor 3 (E2A |
| | | | | immunoglobulin enhancer |
| | | | | binding factors E12/E47) |
| 61 | Protein | NP_003191 | TCF3 | transcription factor 3 (E2A |
| | | | | immunoglobulin enhancer |
| | | | | binding factors E12/E47) |
| 62 | DNA | NM_002717 | PPP2R2A | protein phosphatase 2 (formerly |
| | | | | 2A), regulatory subunit B (PR |
| | | | | 52), alpha isoform |
| 63 | Protein | NP_002708 | PPP2R2A | protein phosphatase 2 (formerly |
| | | | | 2A), regulatory subunit B (PR |
| | | | | 52), alpha isoform |
| 64 | DNA | NM_000358 | TGFBI | transforming growth factor, |
| | | | | beta-induced, 68 kDa |
| 65 | Protein | NP_000349 | TGFBI | transforming growth factor, |
| | | | | beta-induced, 68 kDa |
| 66 | DNA | NM_001664 | ARHA | ras homolog gene family, |
| | | | | member A |
| 67 | Protein | NP_001655 | ARHA | ras homolog gene family, |
| | | | | member A |
| 68 | DNA | NM_002419 | MAP3K11 | mitogen-activated protein |
| | | | | kinase kinase kinase 11 |
| 69 | Protein | NP_002410 | MAP3K11 | mitogen-activated protein |
| | | | | kinase kinase kinase 11 |
| 70 | DNA | NM_004593 | SFRS10 | splicing factor, arginine/serine- |
| | | | | rich 10 (transformer 2 homolog, |
| | | | | Drosophila) |
| 71 | Protein | NP_004584 | SFRS10 | splicing factor, arginine/serine- |
| | | | | rich 10 (transformer 2 homolog, |
| | | | | Drosophila) |
| 72 | DNA | NM_003131 | SRF | serum response factor (c-fos |
| | | | | serum response element- |
| | | | | binding transcription factor) |
| 73 | Protein | NP_003122 | SRF | serum response factor (c-fos |
| | | | | serum response element- |
| | | | | binding transcription factor) |
| 74 | DNA | NM_000376 | VDR | vitamin D (1,25- |
| | | | | dihydroxyvitamin D3) receptor |
| 75 | Protein | NP_000367 | VDR | vitamin D (1,25- |
| | | | | dihydroxyvitamin D3) receptor |
| 76 | DNA | D26561 | | D26561 /FEATURE = cds#2 |
| | | | | /DEFINITION = D26561 Homo |
| | | | | sapiens cellular DNA |
| | | | | containing a segment of Human |
| | | | | papilloma virus type 5b, partial |
| | | | | and complete cds |
| 77 | Protein | D26561 (Translation) | | D26561 /FEATURE = cds#2 |
| | | | | /DEFINITION = D26561 Homo |
| | | | | sapiens cellular DNA |
| | | | | containing a segment of Human |
| | | | | papilloma virus type 5b, partial |
| | | | | and complete cds |
| 78 | DNA | NM_002651 | PIK4CB | phosphatidylinositol 4-kinase, |
| | | | | catalytic, beta polypeptide |
| 79 | Protein | NP_002642 | PIK4CB | phosphatidylinositol 4-kinase, |
| | | | | catalytic, beta polypeptide |
| 80 | DNA | NM_002830 | PTPN4 | protein tyrosine phosphatase, |
| | | | | non-receptor type 4 |
| | | | | (megakaryocyte) |
| 81 | Protein | NP_002821 | PTPN4 | protein tyrosine phosphatase, |
| | | | | non-receptor type 4 |
| | | | | (megakaryocyte) |
| 82 | DNA | NM_020529 | NFKBIA | nuclear factor of kappa light |
| | | | | polypeptide gene enhancer in |
| | | | | B-cells inhibitor, alpha |
| 83 | Protein | NP_065390 | NFKBIA | nuclear factor of kappa light |
| | | | | polypeptide gene enhancer in |
| | | | | B-cells inhibitor, alpha |
| 84 | DNA | NM_006292 | TSG101 | tumor susceptibility gene 101 |
| 85 | Protein | NP_006283 | TSG101 | tumor susceptibility gene 101 |
| 86 | DNA | NM_005375 | MYB | v-myb myeloblastosis viral |
| | | | | oncogene homolog (avian) |
| 87 | Protein | NP_005366 | MYB | v-myb myeloblastosis viral |
| | | | | oncogene homolog (avian) |
| 88 | DNA | NM_002836 | PTPRA | protein tyrosine phosphatase, |
| | | | | receptor type, A |
| 89 | Protein | NP_002827 | PTPRA | protein tyrosine phosphatase, |
| | | | | receptor type, A |
| 90 | DNA | NM_080840 | PTPRA | protein tyrosine phosphatase, |
| | | | | receptor type, A |
| 91 | Protein | NP_543030 | PTPRA | protein tyrosine phosphatase, |
| | | | | receptor type, A |
| 92 | DNA | NM_080841 | PTPRA | protein tyrosine phosphatase, |
| | | | | receptor type, A |
| 93 | DNA | NM_002027 | FNTA | farnesyltransferase, CAAX |
| | | | | box, alpha |
| 94 | Protein | NP_002018 | FNTA | farnesyltransferase, CAAX |
| | | | | box, alpha |
| 95 | DNA | X95152 | | X95152 /FEATURE = mRNA |
| | | | | /DEFINITION = HSBRCA22 |
| | | | | H. sapiens brca2 gene exon 2 |
| | | | | (and joined coding region) |
| 96 | Protein | X95152 (Translation) | | X95152 /FEATURE = mRNA |
| | | | | /DEFINITION = HSBRCA22 |
| | | | | H. sapiens brca2 gene exon 2 |
| | | | | (and joined coding region) |
| 97 | DNA | NM_016848 | SHC3 | neuronal Shc |
| 98 | Protein | NP_058544 | SHC3 | neuronal Shc |
| 99 | DNA | HG4074-HT4344 | | Rad2 |
| 100 | DNA | NM_006119 | FGF8 | fibroblast growth factor 8 |
| | | | | (androgen-induced) |
| 101 | Protein | NP_006110 | FGF8 | fibroblast growth factor 8 |
| | | | | (androgen-induced) |
| 102 | DNA | NM_033163 | FGF8 | fibroblast growth factor 8 |
| | | | | (androgen-induced) |
| 103 | Protein | NP_149353 | FGF8 | fibroblast growth factor 8 |
| | | | | (androgen-induced) |
| 104 | DNA | NM_033164 | FGF8 | fibroblast growth factor 8 |
| | | | | (androgen-induced) |
| 105 | Protein | NP_149354 | FGF8 | fibroblast growth factor 8 |
| | | | | (androgen-induced) |
| 106 | DNA | NM_033165 | FGF8 | fibroblast growth factor 8 |
| | | | | (androgen-induced) |
| 107 | Protein | NP_149355 | FGF8 | fibroblast growth factor 8 |
| | | | | (androgen-induced) |
| 108 | DNA | NM_000057 | BLM | Bloom syndrome |
| 109 | Protein | NP_000048 | BLM | Bloom syndrome |
| 110 | DNA | NM_005778 | RBM5 | RNA binding motif protein 5 |
| 111 | Protein | NP_005769 | RBM5 | RNA binding motif protein 5 |
| 112 | DNA | NM_001067 | TOP2A | topoisomerase (DNA) II alpha |
| | | | | 170 kDa |
| 113 | Protein | NP_001058 | TOP2A | topoisomerase (DNA) II alpha |
| | | | | 170 kDa |
| 114 | DNA | NM_003473 | STAM | signal transducing adaptor |
| | | | | molecule (SH3 domain and |
| | | | | ITAM motif) 1 |
| 115 | Protein | NP_003464 | STAM | signal transducing adaptor |
| | | | | molecule (SH3 domain and |
| | | | | ITAM motif) 1 |
| 116 | DNA | NM_005354 | JUND | jun D proto-oncogene |
| 117 | Protein | NP_005345 | JUND | jun D proto-oncogene |
| 118 | DNA | HG3187-HT3366 | | Tyrosine Phosphatase 1, Non- |
| | | | | Receptor, Alt. Splice 3 |
| 119 | DNA | NM_006875 | PIM2 | pim-2 oncogene |
| 120 | Protein | NP_006866 | PIM2 | pim-2 oncogene |
| 121 | DNA | NM_004327 | BCR | breakpoint cluster region |
| 122 | Protein | NP_004318 | BCR | breakpoint cluster region |
| 123 | DNA | NM_021574 | BCR | breakpoint cluster region |
| 124 | Protein | NP_067585 | BCR | breakpoint cluster region |
| 125 | DNA | NM_001969 | EIF5 | eukaryotic translation initiation |
| | | | | factor 5 |
| 126 | Protein | NP_001960 | EIF5 | eukaryotic translation initiation |
| | | | | factor 5 |
| 127 | DNA | NM_002890 | RASA1 | RAS p21 protein activator |
| | | | | (GTPase activating protein) 1 |
| 128 | Protein | NP_002881 | RASA1 | RAS p21 protein activator |
| | | | | (GTPase activating protein) 1 |
| 129 | DNA | NM_022650 | RASA1 | RAS p21 protein activator |
| | | | | (GTPase activating protein) 1 |
| 130 | Protein | NP_072179 | RASA1 | RAS p21 protein activator |
| | | | | (GTPase activating protein) 1 |
| 131 | DNA | NM_001404 | EEF1G | eukaryotic translation |
| | | | | elongation factor 1 gamma |
| 132 | Protein | NP_001395 | EEF1G | eukaryotic translation |
| | | | | elongation factor 1 gamma |
| 133 | DNA | NM_006156 | NEDD8 | neural precursor cell expressed, |
| | | | | developmentally down- |
| | | | | regulated 8 |
| 134 | Protein | NP_006147 | NEDD8 | neural precursor cell expressed, |
| | | | | developmentally down- |
| | | | | regulated 8 |
| 135 | DNA | NM_003010 | MAP2K4 | mitogen-activated protein |
| | | | | kinase kinase 4 |
| 136 | Protein | NP_003001 | MAP2K4 | mitogen-activated protein |
| | | | | kinase kinase 4 |
| 137 | DNA | HG884-HT884 | | Oncogene E6-Ap, |
| | | | | Papillomavirus |
| 138 | DNA | NM_001789 | CDC25A | cell division cycle 25A |
| 139 | Protein | NP_001780 | CDC25A | cell division cycle 25A |
| 140 | DNA | NM_001350 | DAXX | death-associated protein 6 |
| 141 | Protein | NP_001341 | DAXX | death-associated protein 6 |
| 142 | DNA | NM_002719 | PPP2R5C | protein phosphatase 2, |
| | | | | regulatory subunit B (B56), |
| | | | | gamma isoform |
| 143 | Protein | NP_002710 | PPP2R5C | protein phosphatase 2, |
| | | | | regulatory subunit B (B56), |
| | | | | gamma isoform |
| 144 | DNA | NM_002689 | POLA2 | polymerase (DNA-directed), |
| | | | | alpha (70 kD) |
| 145 | Protein | NP_002680 | POLA2 | polymerase (DNA-directed), |
| | | | | alpha (70 kD) |
| 146 | DNA | NM_005056 | RBBP2 | retinoblastoma binding protein 2 |
| 147 | Protein | NP_005047 | RBBP2 | retinoblastoma binding protein 2 |
| 148 | DNA | NM_001800 | CDKN2D | cyclin-dependent kinase |
| | | | | inhibitor 2D (p19, inhibits |
| | | | | CDK4) |
| 149 | Protein | NP_001791 | CDKN2D | cyclin-dependent kinase |
| | | | | inhibitor 2D (p19, inhibits |
| | | | | CDK4) |
| 150 | DNA | NM_079421 | CDKN2D | cyclin-dependent kinase |
| | | | | inhibitor 2D (p19, inhibits |
| | | | | CDK4) |
| 151 | DNA | NM_000465 | BARD1 | BRCA1 associated RING |
| | | | | domain 1 |
| 152 | Protein | NP_000456 | BARD1 | BRCA1 associated RING |
| | | | | domain 1 |
| 153 | DNA | NM_001786 | CDC2 | cell division cycle 2, G1 to S |
| | | | | and G2 to M |
| 154 | Protein | NP_001777 | CDC2 | cell division cycle 2, G1 to S |
| | | | | and G2 to M |
| 155 | DNA | NM_033379 | CDC2 | cell division cycle 2, G1 to S |
| | | | | and G2 to M |
| 156 | Protein | NP_203698 | CDC2 | cell division cycle 2, G1 to S |
| | | | | and G2 to M |
| 157 | DNA | NM_003503 | CDC7L1 | CDC7 cell division cycle 7-like |
| | | | | 1 (S. cerevisiae) |
| 158 | Protein | NP_003494 | CDC7L1 | CDC7 cell division cycle 7-like |
| | | | | 1 (S. cerevisiae) |
| 159 | DNA | NM_006254 | PRKCD | protein kinase C, delta |
| 160 | Protein | NP_006245 | PRKCD | protein kinase C, delta |
| 161 | DNA | NM_003242 | TGFBR2 | transforming growth factor, |
| | | | | beta receptor II (70/80 kDa) |
| 162 | Protein | NP_003233 | TGFBR2 | transforming growth factor, |
| | | | | beta receptor II (70/80 kDa) |
| 163 | DNA | HG1996-HT2044 | | Guanine Nucleotide-Binding |
| | | | | Protein Rap2, Ras-Oncogene |
| | | | | Related |
| 164 | DNA | NM_005904 | MADH7 | MAD, mothers against |
| | | | | decapentaplegic homolog 7 |
| | | | | (Drosophila) |
| 165 | Protein | NP_005895 | MADH7 | MAD, mothers against |
| | | | | decapentaplegic homolog 7 |
| | | | | (Drosophila) |
| 166 | DNA | NM_005426 | TP53BP2 | tumor protein p53 binding |
| | | | | protein, 2 |
| 167 | Protein | NP_005417 | TP53BP2 | tumor protein p53 binding |
| | | | | protein, 2 |
| 168 | DNA | NM_004322 | BAD | BCL2-antagonist of cell death |
| 169 | Protein | NP_004313 | BAD | BCL2-antagonist of cell death |
| 170 | DNA | NM_032989 | BAD | BCL2-antagonist of cell death |
| 171 | DNA | NM_004579 | MAP4K2 | mitogen-activated protein |
| | | | | kinase kinase kinase kinase 2 |
| 172 | Protein | NP_004570 | MAP4K2 | mitogen-activated protein |
| | | | | kinase kinase kinase kinase 2 |
| 173 | DNA | HG1103-HT1103 | | Guanine Nucleotide-Binding |
| | | | | Protein Ral, Ras-Oncogene |
| | | | | Related |
| 174 | DNA | NM_006270 | RRAS | related RAS viral (r-ras) |
| | | | | oncogene homolog |
| 175 | Protein | NP_006261 | RRAS | related RAS viral (r-ras) |
| | | | | oncogene homolog |
| 176 | DNA | NM_002592 | PCNA | proliferating cell nuclear |
| | | | | antigen |
| 177 | Protein | NP_002583 | PCNA | proliferating cell nuclear |
| | | | | antigen |
| 178 | DNA | NM_000038 | APC | adenomatosis polyposis coli |
| 179 | Protein | NP_000029 | APC | adenomatosis polyposis coli |
| 180 | DNA | NM_002880 | RAF1 | v-raf-1 murine leukemia viral |
| | | | | oncogene homolog 1 |
| 181 | Protein | NP_002871 | RAF1 | v-raf-1 murine leukemia viral |
| | | | | oncogene homolog 1 |
| 182 | DNA | NM_005642 | TAF7 | TAF7 RNA polymerase II, |
| | | | | TATA box binding protein |
| | | | | (TBP)-associated factor, 55 kDa |
| 183 | Protein | NP_005633 | TAF7 | TAF7 RNA polymerase II, |
| | | | | TATA box binding protein |
| | | | | (TBP)-associated factor, 55 kDa |
| 184 | DNA | NM_001761 | CCNF | cyclin F |
| 185 | Protein | NP_001752 | CCNF | cyclin F |
| 186 | DNA | NM_004985 | KRAS2 | v-Ki-ras2 Kirsten rat sarcoma 2 |
| | | | | viral oncogene homolog |
| 187 | Protein | NP_004976 | KRAS2 | v-Ki-ras2 Kirsten rat sarcoma 2 |
| | | | | viral oncogene homolog |
| 188 | DNA | NM_033360 | KRAS2 | v-Ki-ras2 Kirsten rat sarcoma 2 |
| | | | | viral oncogene homolog |
| 189 | Protein | NP_203524 | KRAS2 | v-Ki-ras2 Kirsten rat sarcoma 2 |
| | | | | viral oncogene homolog |
| 190 | DNA | NM_000075 | CDK4 | cyclin-dependent kinase 4 |
| 191 | Protein | NP_000066 | CDK4 | cyclin-dependent kinase 4 |
| 192 | DNA | NM_032913 | CDK4 | cyclin-dependent kinase 4 |
| 193 | Protein | NP_116302 | CDK4 | cyclin-dependent kinase 4 |
| 194 | DNA | NM_052984 | CDK4 | cyclin-dependent kinase 4 |
| 195 | Protein | NP_443710 | CDK4 | cyclin-dependent kinase 4 |
| 196 | DNA | NM_001237 | CCNA2 | cyclin A2 |
| 197 | Protein | NP_001228 | CCNA2 | cyclin A2 |
| 198 | DNA | NM_031966 | CCNB1 | cyclin B1 |
| 199 | Protein | NP_114172 | CCNB1 | cyclin B1 |
| 200 | DNA | NM_005903 | MADH5 | MAD, mothers against |
| | | | | decapentaplegic homolog 5 |
| | | | | (Drosophila) |
| 201 | Protein | NP_005894 | MADH5 | MAD, mothers against |
| | | | | decapentaplegic homolog 5 |
| | | | | (Drosophila) |
| 202 | DNA | NM_001799 | CDK7 | cyclin-dependent kinase 7 |
| | | | | (MO15 homolog, Xenopus |
| | | | | laevis, cdk-activating kinase) |
| 203 | Protein | NP_001790 | CDK7 | cyclin-dependent kinase 7 |
| | | | | (MO15 homolog, Xenopus |
| | | | | laevis, cdk-activating kinase) |
| 204 | DNA | NM_002512 | NME2 | non-metastatic cells 2, protein |
| | | | | (NM23B) expressed in |
| 205 | Protein | NP_002503 | NME2 | non-metastatic cells 2, protein |
| | | | | (NM23B) expressed in |
| 206 | DNA | NM_000269 | NME1 | non-metastatic cells 1, protein |
| | | | | (NM23A) expressed in |
| 207 | Protein | NP_000260 | NME1 | non-metastatic cells 1, protein |
| | | | | (NM23A) expressed in |
| 208 | DNA | NM_006256 | PRKCL2 | protein kinase C-like 2 |
| 209 | Protein | NP_006247 | PRKCL2 | protein kinase C-like 2 |
| 210 | DNA | NM_000179 | MSH6 | mutS homolog 6 (E. coli) |
| 211 | Protein | NP_000170 | MSH6 | mutS homolog 6 (E. coli) |
| 212 | DNA | NM_004048 | B2M | beta-2-microglobulin |
| 213 | Protein | NP_004039 | B2M | beta-2-microglobulin |
| 214 | DNA | NM_006013 | RPL10 | ribosomal protein L10 |
| 215 | Protein | NP_006004 | RPL10 | ribosomal protein L10 |
| 216 | DNA | NM_004506 | HSF2 | heat shock transcription factor 2 |
| 217 | Protein | NP_004497 | HSF2 | heat shock transcription factor 2 |
| 218 | DNA | NM_001238 | CCNE1 | cyclin E1 |
| 219 | Protein | NP_001229 | CCNE1 | cyclin E1 |
| 220 | DNA | NM_057182 | CCNE1 | cyclin E1 |
| 221 | Protein | NP_476530 | CCNE1 | cyclin E1 |
| 222 | DNA | NM_001641 | APEX1 | APEX nuclease |
| | | | | (multifunctional DNA repair |
| | | | | enzyme) 1 |
| 223 | Protein | NP_001632 | APEX1 | APEX nuclease |
| | | | | (multifunctional DNA repair |
| | | | | enzyme) 1 |
| 224 | DNA | NM_080648 | APEX1 | APEX nuclease |
| | | | | (multifunctional DNA repair |
| | | | | enzyme) 1 |
| 225 | DNA | NM_080649 | APEX1 | APEX nuclease |
| | | | | (multifunctional DNA repair |
| | | | | enzyme) 1 |
| 226 | DNA | NM_001982 | ERBB3 | v-erb-b2 erythroblastic |
| | | | | leukemia viral oncogene |
| | | | | homolog 3 (avian) |
| 227 | Protein | NP_001973 | ERBB3 | v-erb-b2 erythroblastic |
| | | | | leukemia viral oncogene |
| | | | | homolog 3 (avian) |
| 228 | DNA | NM_001938 | DR1 | down-regulator of transcription |
| | | | | 1, TBP-binding (negative |
| | | | | cofactor 2) |
| 229 | Protein | NP_001929 | DR1 | down-regulator of transcription |
| | | | | 1, TBP-binding (negative |
| | | | | cofactor 2) |
| 230 | DNA | NM_002448 | MSX1 | msh homeo box homolog 1 |
| | | | | (Drosophila) |
| 231 | Protein | NP_002439 | MSX1 | msh homeo box homolog 1 |
| | | | | (Drosophila) |
| 232 | DNA | NM_000127 | EXT1 | exostoses (multiple) 1 |
| 233 | Protein | NP_000118 | EXT1 | exostoses (multiple) 1 |
| 234 | DNA | NM_005760 | CBF2 | CCAAT-box-binding |
| | | | | transcription factor |
| 235 | Protein | NP_005751 | CBF2 | CCAAT-box-binding |
| | | | | transcription factor |
| 236 | DNA | NM_002825 | PTN | pleiotrophin (heparin binding |
| | | | | growth factor 8, neurite growth- |
| | | | | promoting factor 1) |
| 237 | Protein | NP_002816 | PTN | pleiotrophin (heparin binding |
| | | | | growth factor 8, neurite growth- |
| | | | | promoting factor 1) |
| 238 | DNA | NM_002715 | PPP2CA | protein phosphatase 2 (formerly |
| | | | | 2A), catalytic subunit, alpha |
| | | | | isoform |
| 239 | Protein | NP_002706 | PPP2CA | protein phosphatase 2 (formerly |
| | | | | 2A), catalytic subunit, alpha |
| | | | | isoform |
| 240 | DNA | NM_004555 | NFATC3 | nuclear factor of activated T- |
| | | | | cells, cytoplasmic, calcineurin- |
| | | | | dependent 3 |
| 241 | Protein | NP_004546 | NFATC3 | nuclear factor of activated T- |
| | | | | cells, cytoplasmic, calcineurin- |
| | | | | dependent 3 |
| 242 | DNA | NM_173163 | NFATC3 | nuclear factor of activated T- |
| | | | | cells, cytoplasmic, calcineurin- |
| | | | | dependent 3 |
| 243 | Protein | NP_775186 | NFATC3 | nuclear factor of activated T- |
| | | | | cells, cytoplasmic, calcineurin- |
| | | | | dependent 3 |
| 244 | DNA | NM_173164 | NFATC3 | nuclear factor of activated T- |
| | | | | cells, cytoplasmic, calcineurin- |
| | | | | dependent 3 |
| 245 | Protein | NP_775187 | NFATC3 | nuclear factor of activated T- |
| | | | | cells, cytoplasmic, calcineurin- |
| | | | | dependent 3 |
| 246 | DNA | NM_173165 | NFATC3 | nuclear factor of activated T- |
| | | | | cells, cytoplasmic, calcineurin- |
| | | | | dependent 3 |
| 247 | Protein | NP_775188 | NFATC3 | nuclear factor of activated T- |
| | | | | cells, cytoplasmic, calcineurin- |
| | | | | dependent 3 |
| 248 | DNA | NM_002295 | LAMR1 | laminin receptor 1 (ribosomal |
| | | | | protein SA, 67 kDa) |
| 249 | Protein | NP_002286 | LAMR1 | laminin receptor 1 (ribosomal |
| | | | | protein SA, 67 kDa) |
| 250 | DNA | NM_001634 | AMD1 | S-adenosylmethionine |
| | | | | decarboxylase 1 |
| 251 | Protein | NP_001625 | AMD1 | S-adenosylmethionine |
| | | | | decarboxylase 1 |
| 252 | DNA | NM_021960 | MCL1 | myeloid cell leukemia sequence |
| | | | | 1 (BCL2-related) |
| 253 | Protein | NP_068779 | MCL1 | myeloid cell leukemia sequence |
| | | | | 1 (BCL2-related) |
| 254 | DNA | HG4322-HT4592 | | Tubulin, Beta |
| 255 | DNA | NM_001022 | RPS19 | ribosomal protein S19 |
| 256 | Protein | NP_001013 | RPS19 | ribosomal protein S19 |
| 257 | DNA | NM_012185 | FOXE2 | forkhead box E2 |
| 258 | Protein | NP_036317 | FOXE2 | forkhead box E2 |
| 259 | DNA | M20812 | | Cluster Incl. M20812: Human |
| | | | | kappa-immunoglobulin |
| | | | | germline pseudogene (cos118) |
| | | | | variable region (subgroup V |
| | | | | kappa I) /cds = (6.326) |
| | | | | /gb = M20812 /gi = 185958 |
| | | | | /ug = Hs.150224 /len = 351 |
| 260 | Protein | AAA36095 | | Cluster Incl. M20812: Human |
| | | | | kappa-immunoglobulin |
| | | | | germline pseudogene (cos118) |
| | | | | variable region (subgroup V |
| | | | | kappa I) /cds = (6.326) |
| | | | | /gb = M20812 /gi = 185958 |
| | | | | /ug = Hs.150224 /len = 351 |
| 261 | DNA | AF068744 | | Cluster Incl. AF068744: Homo |
| | | | | sapiens double homeodomain |
| | | | | protein (DUX2) mRNA, |
| | | | | complete cds /cds = (211.453) |
| | | | | /gb = AF068744 /gi = 3414864 |
| | | | | /ug = Hs.157425 /len = 556 |
| 262 | Protein | AF068744 | | Cluster Incl. AF068744: Homo |
| | | (Translation) | | sapiens double homeodomain |
| | | | | protein (DUX2) mRNA, |
| | | | | complete cds /cds = (211.453) |
| | | | | /gb = AF068744 /gi = 3414864 |
| | | | | /ug = Hs.157425 /len = 556 |
| 263 | DNA | NM_012369 | | Cluster Incl. AC004853: Homo |
| | | | | sapiens PAC clone DJ0669B10 |
| | | | | from 7q33-q35 /cds = (0.953) |
| | | | | /gb = AC004853 /gi = 3766130 |
| | | | | /ug = Hs.159899 /len = 954 |
| 264 | Protein | NP_036501 | | Cluster Incl. AC004853: Homo |
| | | | | sapiens PAC clone DJ0669B10 |
| | | | | from 7q33-q35 /cds = (0.953) |
| | | | | /gb = AC004853 /gi = 3766130 |
| | | | | /ug = Hs.159899 /len = 954 |
| 265 | DNA | W28732 | | Cluster Incl. W28732: 50h7 |
| | | | | Homo sapiens cDNA |
| | | | | /gb = W28732 /gi = 1308680 |
| | | | | /ug = Hs.177496 /len = 818 |
| 266 | DNA | NM_005336 | HDLBP | high density lipoprotein binding |
| | | | | protein (vigilin) |
| 267 | Protein | NP_005327 | HDLBP | high density lipoprotein binding |
| | | | | protein (vigilin) |
| 268 | DNA | NM_000973 | RPL8 | ribosomal protein L8 |
| 269 | Protein | NP_000964 | RPL8 | ribosomal protein L8 |
| 270 | DNA | NM_033301 | RPL8 | ribosomal protein L8 |
| 271 | DNA | NM_001013 | RPS9 | ribosomal protein S9 |
| 272 | Protein | NP_001004 | RPS9 | ribosomal protein S9 |
| 273 | DNA | M90356 | | Cluster Incl. M90356: Human |
| | | | | BTF3 protein homologue gene, |
| | | | | complete cds /cds = (0.644) |
| | | | | /gb = M90356 /gi = 179575 |
| | | | | /ug = Hs.181967 /len = 645 |
| 274 | Protein | M90356 | | Cluster Incl. M90356: Human |
| | | (Translation) | | BTF3 protein homologue gene, |
| | | | | complete cds /cds = (0.644) |
| | | | | /gb = M90356 /gi = 179575 |
| | | | | /ug = Hs.181967 /len = 645 |
| 275 | DNA | NM_002952 | RPS2 | ribosomal protein S2 |
| 276 | Protein | NP_002943 | RPS2 | ribosomal protein S2 |
| 277 | DNA | NM_001002 | RPLP0 | ribosomal protein, large, P0 |
| 278 | Protein | NP_000993 | RPLP0 | ribosomal protein, large, P0 |
| 279 | DNA | NM_053275 | RPLP0 | ribosomal protein, large, P0 |
| 280 | DNA | NM_022551 | | Homo sapiens ribosomal |
| | | | | protein S18 (RPS18), mRNA |
| 281 | Protein | NP_072045 | | Homo sapiens ribosomal |
| | | | | protein S18 (RPS18) |
| 282 | DNA | NM_021109 | TMSB4X | thymosin, beta 4, X |
| | | | | chromosome |
| 283 | Protein | NP_066932 | TMSB4X | thymosin, beta 4, X |
| | | | | chromosome |
| 284 | DNA | NM_001014 | RPS10 | ribosomal protein S10 |
| 285 | Protein | NP_001005 | RPS10 | ribosomal protein S10 |
| 286 | DNA | NM_004095 | EIF4EBP1 | eukaryotic translation initiation |
| | | | | factor 4E binding protein 1 |
| 287 | Protein | NP_004086 | EIF4EBP1 | eukaryotic translation initiation |
| | | | | factor 4E binding protein 1 |
| 288 | DNA | NM_012231 | PRDM2 | PR domain containing 2, with |
| | | | | ZNF domain |
| 289 | Protein | NP_036363 | PRDM2 | PR domain containing 2, with |
| | | | | ZNF domain |
| 290 | DNA | NM_015866 | PRDM2 | PR domain containing 2, with |
| | | | | ZNF domain |
| 291 | Protein | NP_056950 | PRDM2 | PR domain containing 2, with |
| | | | | ZNF domain |
| 292 | DNA | AF047485 | LOC90586 | amine oxidase pseudogene |
| 293 | Protein | AF047485 | LOC90586 | amine oxidase pseudogene |
| | | (Translation) | | |
| 294 | DNA | NM_024407 | NDUFS7 | NADH dehydrogenase |
| | | | | (ubiquinone) Fe—S protein 7, |
| | | | | 20 kDa (NADH-coenzyme Q |
| | | | | reductase) |
| 295 | Protein | NP_077718 | NDUFS7 | NADH dehydrogenase |
| | | | | (ubiquinone) Fe—S protein 7, |
| | | | | 20 kDa (NADH-coenzyme Q |
| | | | | reductase) |
| 296 | DNA | NM_005271 | | Unknown (protein for |
| | | | | MGC: 13241) [Homo sapiens], |
| | | | | mRNA sequence |
| 297 | Protein | NP_005262 | | Unknown (protein for |
| | | | | MGC: 13241) [Homo sapiens], |
| | | | | mRNA sequence |
| 298 | DNA | NM_012084 | | Unknown (protein for |
| | | | | MGC: 13241) [Homo sapiens], |
| | | | | mRNA sequence |
| 299 | Protein | NP_036216 | | Unknown (protein for |
| | | | | MGC: 13241) [Homo sapiens], |
| | | | | mRNA sequence |
| 300 | DNA | U08997 | | Unknown (protein for |
| | | | | MGC: 13241) [Homo sapiens], |
| | | | | mRNA sequence |
| 301 | DNA | J04755 | | Cluster Incl. J04755: Human |
| | | | | ferritin H processed |
| | | | | pseudogene, complete cds |
| | | | | /cds = UNKNOWN /gb = J04755 |
| | | | | /gi = 182512 /ug = Hs.239542 |
| | | | | /len = 2083 |
| 302 | DNA | NM_003655 | CBX4 | chromobox homolog 4 (Pc |
| | | | | class homolog, Drosophila) |
| 303 | Protein | NP_003646 | CBX4 | chromobox homolog 4 (Pc |
| | | | | class homolog, Drosophila) |
| 304 | DNA | NM_014212 | HOXC11 | homeo box C11 |
| 305 | Protein | NP_055027 | HOXC11 | homeo box C11 |
| 306 | DNA | W28912 | | ESTs |
| 307 | DNA | NM_005160 | ADRBK2 | adrenergic, beta, receptor |
| | | | | kinase 2 |
| 308 | Protein | NP_005151 | ADRBK2 | adrenergic, beta, receptor |
| | | | | kinase 2 |
| 309 | DNA | NM_006026 | H1FX | H1 histone family, member X |
| 310 | Protein | NP_006017 | H1FX | H1 histone family, member X |
| 311 | DNA | NM_015062 | KIAA0595 | KIAA0595 protein |
| 312 | Protein | NP_055877 | KIAA0595 | KIAA0595 protein |
| 313 | DNA | NM_001498 | GCLC | glutamate-cysteine ligase, |
| | | | | catalytic subunit |
| 314 | Protein | NP_001489 | GCLC | glutamate-cysteine ligase, |
| | | | | catalytic subunit |
| 315 | DNA | AL050390 | DKFZP564O043 | hypothetical protein |
| | | | | DKFZp564O043 |
| 316 | DNA | NM_003797 | EED | embryonic ectoderm |
| | | | | development |
| 317 | Protein | NP_003788 | EED | embryonic ectoderm |
| | | | | development |
| 318 | DNA | NM_152991 | EED | embryonic ectoderm |
| | | | | development |
| 319 | Protein | NP_694536 | EED | embryonic ectoderm |
| | | | | development |
| 320 | DNA | NM_005796 | NUTF2 | nuclear transport factor 2 |
| 321 | Protein | NP_005787 | NUTF2 | nuclear transport factor 2 |
| 322 | DNA | NM_003876 | PMI | putative receptor protein |
| 323 | Protein | NP_003867 | PMI | putative receptor protein |
| 324 | DNA | D80001 | KIAA0179 | KIAA0179 protein |
| 325 | Protein | D80001 (Translation) | KIAA0179 | KIAA0179 protein |
| 326 | DNA | NM_005792 | MPHOSPH6 | M-phase phosphoprotein 6 |
| 327 | Protein | NP_005783 | MPHOSPH6 | M-phase phosphoprotein 6 |
| 328 | DNA | NM_006716 | ASK | activator of S phase kinase |
| 329 | Protein | NP_006707 | ASK | activator of S phase kinase |
| 330 | DNA | NM_001812 | CENPC1 | centromere protein C 1 |
| 331 | Protein | NP_001803 | CENPC1 | centromere protein C 1 |
| 332 | DNA | NM_001186 | BACH1 | BTB and CNC homology 1, |
| | | | | basic leucine zipper |
| | | | | transcription factor 1 |
| 333 | Protein | NP_001177 | BACH1 | BTB and CNC homology 1, |
| | | | | basic leucine zipper |
| | | | | transcription factor 1 |
| 334 | DNA | NM_014673 | KIAA0103 | KIAA0103 gene product |
| 335 | Protein | NP_055488 | KIAA0103 | KIAA0103 gene product |
| 336 | DNA | NM_001537 | HSBP1 | heat shock factor binding |
| | | | | protein 1 |
| 337 | Protein | NP_001528 | HSBP1 | heat shock factor binding |
| | | | | protein 1 |
| 338 | DNA | NM_001024 | RPS21 | ribosomal protein S21 |
| 339 | Protein | NP_001015 | RPS21 | ribosomal protein S21 |
| 340 | DNA | NM_001003 | RPLP1 | ribosomal protein, large, P1 |
| 341 | Protein | NP_000994 | RPLP1 | ribosomal protein, large, P1 |
| 342 | DNA | NM_000998 | RPL37A | ribosomal protein L37a |
| 343 | Protein | NP_000989 | RPL37A | ribosomal protein L37a |
| 344 | DNA | AL049430 | | Homo sapiens mRNA; cDNA |
| | | | | DKFZp586H201 (from clone |
| | | | | DKFZp586H201), mRNA |
| | | | | sequence |
| 345 | DNA | NM_030756 | TCF7L2 | transcription factor 7-like 2 (T- |
| | | | | cell specific, HMG-box) |
| 346 | Protein | NP_110383 | TCF7L2 | transcription factor 7-like 2 (T- |
| | | | | cell specific, HMG-box) |
| 347 | DNA | NM_014247 | PDZ-GEF1 | PDZ domain containing |
| | | | | guanine nucleotide exchange |
| | | | | factor(GEF)1 |
| 348 | Protein | NP_055062 | PDZ-GEF1 | PDZ domain containing |
| | | | | guanine nucleotide exchange |
| | | | | factor(GEF)1 |
| 349 | DNA | NM_000303 | PMM2 | phosphomannomutase 2 |
| 350 | Protein | NP_000294 | PMM2 | phosphomannomutase 2 |
| 351 | DNA | NM_022719 | DGCR14 | DiGeorge syndrome critical |
| | | | | region gene 14 |
| 352 | Protein | NP_073210 | DGCR14 | DiGeorge syndrome critical |
| | | | | region gene 14 |
| 353 | DNA | NM_007042 | RPP14 | ribonuclease P (14 kD) |
| 354 | Protein | NP_008973 | RPP14 | ribonuclease P (14 kD) |
| 355 | DNA | NM_014671 | KIAA0010 | ubiquitin-protein isopeptide |
| | | | | ligase (E3) |
| 356 | Protein | NP_055486 | KIAA0010 | ubiquitin-protein isopeptide |
| | | | | ligase (E3) |
| 357 | DNA | NM_004854 | HNK-1ST | HNK-1 sulfotransferase |
| 358 | Protein | NP_004845 | HNK-1ST | HNK-1 sulfotransferase |
| 359 | DNA | NM_004330 | BNIP2 | BCL2/adenovirus E1B 19 kDa |
| | | | | interacting protein 2 |
| 360 | Protein | NP_004321 | BNIP2 | BCL2/adenovirus E1B 19 kDa |
| | | | | interacting protein 2 |
| 361 | DNA | AB002293 | KIAA0295 | KIAA0295 protein |
| 362 | Protein | AB002293 | KIAA0295 | KIAA0295 protein |
| | | (Translation) | | |
| 363 | DNA | AB023198 | KIAA0981 | KIAA0981 protein |
| 364 | Protein | AB023198 | KIAA0981 | KIAA0981 protein |
| | | (Translation) | | |
| 365 | DNA | AB007915 | KIAA0446 | KIAA0446 gene product |
| 366 | Protein | AB007915 | KIAA0446 | KIAA0446 gene product |
| | | (Translation) | | |
| 367 | DNA | NM_004273 | CHST3 | carbohydrate (chondroitin 6) |
| | | | | sulfotransferase 3 |
| 368 | Protein | NP_004264 | CHST3 | carbohydrate (chondroitin 6) |
| | | | | sulfotransferase 3 |
| 369 | DNA | NM_014363 | SACS | spastic ataxia of Charlevoix- |
| | | | | Saguenay (sacsin) |
| 370 | Protein | NP_055178 | SACS | spastic ataxia of Charlevoix- |
| | | | | Saguenay (sacsin) |
| 371 | DNA | NM_000094 | COL7A1 | collagen, type VII, alpha 1 |
| | | | | (epidermolysis bullosa, |
| | | | | dystrophic, dominant and |
| | | | | recessive) |
| 372 | Protein | NP_000085 | COL7A1 | collagen, type VII, alpha 1 |
| | | | | (epidermolysis bullosa, |
| | | | | dystrophic, dominant and |
| | | | | recessive) |
| 373 | DNA | AA928996 | THOC2 | THO complex 2 |
| 374 | DNA | AL079314 | ZNF364 | zinc finger protein 364 |
| 375 | Protein | AL079314 | ZNF364 | zinc finger protein 364 |
| | | (Translation) | | |
| 376 | DNA | NM_015641 | TES | testis derived transcript (3 LIM |
| | | | | domains) |
| 377 | Protein | NP_056456 | TES | testis derived transcript (3 LIM |
| | | | | domains) |
| 378 | DNA | NM_152829 | TES | testis derived transcript (3 LIM |
| | | | | domains) |
| 379 | Protein | NP_690042 | TES | testis derived transcript (3 LIM |
| | | | | domains) |
| 380 | DNA | NM_002856 | PVRL2 | poliovirus receptor-related 2 |
| | | | | (herpesvirus entry mediator B) |
| 381 | Protein | NP_002847 | PVRL2 | poliovirus receptor-related 2 |
| | | | | (herpesvirus entry mediator B) |
| 382 | DNA | AI817548 | | Cluster Incl. |
| | | | | AI817548: wk24e08.x1 Homo |
| | | | | sapiens cDNA, 3′ end |
| | | | | /clone = IMAGE-2413286 |
| | | | | /clone_end = 3′ /gb = AI817548 |
| | | | | /gi = 5436627 /ug = Hs.184093 |
| | | | | /len = 570 |
| 383 | DNA | NM_015002 | FBXO21 | F-box only protein 21 |
| 384 | Protein | NP_055817 | FBXO21 | F-box only protein 21 |
| 385 | DNA | NM_033624 | FBXO21 | F-box only protein 21 |
| 386 | Protein | NP_296373 | FBXO21 | F-box only protein 21 |
| 387 | DNA | NM_001788 | CDC10 | CDC10 cell division cycle 10 |
| | | | | homolog (S. cerevisiae) |
| 388 | Protein | NP_001779 | CDC10 | CDC10 cell division cycle 10 |
| | | | | homolog (S. cerevisiae) |
| 389 | DNA | NM_006989 | CAPRI | Ca2+-promoted Ras inactivator |
| 390 | Protein | NP_008920 | CAPRI | Ca2+-promoted Ras inactivator |
| 391 | DNA | NM_003704 | RES4-22 | gene with multiple splice |
| | | | | variants near HD locus on |
| | | | | 4p16.3 |
| 392 | Protein | NP_003695 | RES4-22 | gene with multiple splice |
| | | | | variants near HD locus on |
| | | | | 4p16.3 |
| 393 | DNA | NM_007144 | ZNF144 | zinc finger protein 144 (Mel- |
| | | | | 18 |
| 394 | Protein | NP_009075 | ZNF144 | zinc finger protein 144 (Mel- |
| | | | | 18) |
| 395 | DNA | AL049450 | | Homo sapiens mRNA; cDNA |
| | | | | DKFZp586B1922 (from clone |
| | | | | DKFZp586B1922), mRNA |
| | | | | sequence |
| 396 | DNA | NM_014686 | KIAA0355 | KIAA0355 gene product |
| 397 | Protein | NP_055501 | KIAA0355 | KIAA0355 gene product |
| 398 | DNA | NM_005837 | RPP20 | POP7 (processing of precursor, |
| | | | | S. cerevisiae) homolog |
| 399 | Protein | NP_005828 | RPP20 | POP7 (processing of precursor, |
| | | | | S. cerevisiae) homolog |
| 400 | DNA | NM_004786 | TXNL | thioredoxin-like, 32 kDa |
| 401 | Protein | NP_004777 | TXNL | thioredoxin-like, 32 kDa |
| 402 | DNA | NM_030809 | C12orf22 | chromosome 12 open reading |
| | | | | frame 22 |
| 403 | Protein | NP_110436 | C12orf22 | chromosome 12 open reading |
| | | | | frame 22 |
| 404 | DNA | NM_012290 | TLK1 | tousled-like kinase 1 |
| 405 | Protein | NP_036422 | TLK1 | tousled-like kinase 1 |
| 406 | DNA | NM_005047 | PSMD5 | proteasome (prosome, |
| | | | | macropain) 26S subunit, non- |
| | | | | ATPase, 5 |
| 407 | Protein | NP_005038 | PSMD5 | proteasome (prosome, |
| | | | | macropain) 26S subunit, non- |
| | | | | ATPase, 5 |
| 408 | DNA | NM_003218 | TERF1 | telomeric repeat binding factor |
| | | | | (NIMA-interacting) 1 |
| 409 | Protein | NP_003209 | TERF1 | telomeric repeat binding factor |
| | | | | (NIMA-interacting) 1 |
| 410 | DNA | NM_017489 | TERF1 | telomeric repeat binding factor |
| | | | | (NIMA-interacting) 1 |
| 411 | Protein | NP_059523 | TERF1 | telomeric repeat binding factor |
| | | | | (NIMA-interacting) 1 |
| 412 | DNA | NM_001991 | EZH1 | enhancer of zeste homolog 1 |
| | | | | (Drosophila) |
| 413 | Protein | NP_001982 | EZH1 | enhancer of zeste homolog 1 |
| | | | | (Drosophila) |
| 414 | DNA | NM_003768 | PEA15 | phosphoprotein enriched in |
| | | | | astrocytes 15 |
| 415 | Protein | NP_003759 | PEA15 | phosphoprotein enriched in |
| | | | | astrocytes 15 |
| 416 | DNA | NM_013287 | PEA15 | phosphoprotein enriched in |
| | | | | astrocytes 15 |
| 417 | DNA | NM_023005 | BAZ1B | bromodomain adjacent to zinc |
| | | | | finger domain, 1B |
| 418 | Protein | NP_075381 | BAZ1B | bromodomain adjacent to zinc |
| | | | | finger domain, 1B |
| 419 | DNA | NM_032408 | BAZ1B | bromodomain adjacent to zinc |
| | | | | finger domain, 1B |
| 420 | Protein | NP_115784 | BAZ1B | bromodomain adjacent to zinc |
| | | | | finger domain, 1B |
| 421 | DNA | NM_015935 | CGI-01 | CGI-01 protein |
| 422 | Protein | NP_057019 | CGI-01 | CGI-01 protein |
| 423 | DNA | AF052148 | | Homo sapiens clone 24507 |
| | | | | mRNA sequence |
| 424 | DNA | NM_000994 | RPL32 | ribosomal protein L32 |
| 425 | Protein | NP_000985 | RPL32 | ribosomal protein L32 |
| 426 | DNA | NM_005395 | PMS2L9 | postmeiotic segregation |
| | | | | increased 2-like 9 |
| 427 | Protein | NP_005386 | PMS2L9 | postmeiotic segregation |
| | | | | increased 2-like 9 |
| 428 | DNA | NM_003289 | TPM2 | tropomyosin 2 (beta) |
| 429 | Protein | NP_003280 | TPM2 | tropomyosin 2 (beta) |
| 430 | DNA | NM_001026 | RPS24 | ribosomal protein S24 |
| 431 | Protein | NP_001017 | RPS24 | ribosomal protein S24 |
| 432 | DNA | NM_033022 | RPS24 | ribosomal protein S24 |
| 433 | Protein | NP_148982 | RPS24 | ribosomal protein S24 |
| 434 | DNA | NM_001101 | ACTB | actin, beta |
| 435 | Protein | NP_001092 | ACTB | actin, beta |
| 436 | DNA | NM_001015 | RPS11 | ribosomal protein S11 |
| 437 | Protein | NP_001006 | RPS11 | ribosomal protein S11 |
| 438 | DNA | NM_013410 | AK3 | adenylate kinase 3 |
| 439 | Protein | NP_037542 | AK3 | adenylate kinase 3 |
| 440 | DNA | NM_000034 | ALDOA | aldolase A, fructose- |
| | | | | bisphosphate |
| 441 | Protein | NP_000025 | ALDOA | aldolase A, fructose- |
| | | | | bisphosphate |
| 442 | DNA | NM_000982 | RPL21 | ribosomal protein L21 |
| 443 | Protein | NP_000973 | RPL21 | ribosomal protein L21 |
| 444 | DNA | NM_004559 | NSEP1 | nuclease sensitive element |
| | | | | binding protein 1 |
| 445 | Protein | NP_004550 | NSEP1 | nuclease sensitive element |
| | | | | binding protein 1 |
| 446 | DNA | NM_000984 | RPL23A | ribosomal protein L23a |
| 447 | Protein | NP_000975 | RPL23A | ribosomal protein L23a |
| 448 | DNA | NM_000498 | CYP11B2 | cytochrome P450, subfamily |
| | | | | XIB (steroid 11-beta- |
| | | | | hydroxylase), polypeptide 2 |
| 449 | Protein | NP_000489 | CYP11B2 | cytochrome P450, subfamily |
| | | | | XIB (steroid 11-beta- |
| | | | | hydroxylase), polypeptide 2 |
| 450 | DNA | NM_002654 | PKM2 | pyruvate kinase, muscle |
| 451 | Protein | NP_002645 | PKM2 | pyruvate kinase, muscle |
| 452 | DNA | W25892 | EST | EST |
| 453 | DNA | NM_000990 | RPL27A | ribosomal protein L27a |
| 454 | Protein | NP_000981 | RPL27A | ribosomal protein L27a |
| 455 | DNA | NM_001009 | RPS5 | ribosomal protein S5 |
| 456 | Protein | NP_001000 | RPS5 | ribosomal protein S5 |
| 457 | DNA | NM_001023 | RPS20 | ribosomal protein S20 |
| 458 | Protein | NP_001014 | RPS20 | ribosomal protein S20 |
| 459 | DNA | NM_001905 | CTPS | CTP synthase |
| 460 | Protein | NP_001896 | CTPS | CTP synthase |
| 461 | DNA | NM_021104 | RPL41 | ribosomal protein L41 |
| 462 | Protein | NP_066927 | RPL41 | ribosomal protein L41 |
| 463 | DNA | NM_002235 | KCNA6 | potassium voltage-gated |
| | | | | channel, shaker-related |
| | | | | subfamily, member 6 |
| 464 | Protein | NP_002226 | KCNA6 | potassium voltage-gated |
| | | | | channel, shaker-related |
| | | | | subfamily, member 6 |
| 465 | DNA | NM_001004 | RPLP2 | ribosomal protein, large P2 |
| 466 | Protein | NP_000995 | RPLP2 | ribosomal protein, large P2 |
| 467 | DNA | NM_002268 | RPLP2 | ribosomal protein, large P2 |
| 468 | Protein | NP_002259 | RPLP2 | ribosomal protein, large P2 |
| 469 | DNA | NM_032771 | RPLP2 | ribosomal protein, large P2 |
| 470 | Protein | NP_116160 | RPLP2 | ribosomal protein, large P2 |
| 471 | DNA | AL096857 | KIAA1096 | KIAA1096 protein |
| 472 | Protein | AL096857 | KIAA1096 | KIAA1096 protein |
| | | (Translation) | | |
| 473 | DNA | AI498132 | | Homo sapiens cDNA FLJ37094 |
| | | | | fis, clone BRACE2018337, |
| | | | | mRNA sequence |
| 474 | DNA | NM_005382 | NEF3 | neurofilament 3 (150 kDa |
| | | | | medium) |
| 475 | Protein | NP_005373 | NEF3 | neurofilament 3 (150 kDa |
| | | | | medium) |
| 476 | DNA | NM_014296 | CAPN7 | calpain 7 |
| 477 | Protein | NP_055111 | CAPN7 | calpain 7 |
| 478 | DNA | NM_006012 | CLPP | ClpP caseinolytic protease, |
| | | | | ATP-dependent, proteolytic |
| | | | | subunit homolog (E. coli) |
| 479 | Protein | NP_006003 | CLPP | ClpP caseinolytic protease, |
| | | | | ATP-dependent, proteolytic |
| | | | | subunit homolog (E. coli) |
| 480 | DNA | NM_000138 | FBN1 | fibrillin 1 (Marfan syndrome) |
| 481 | Protein | NP_000129 | FBN1 | fibrillin 1 (Marfan syndrome) |
| 482 | DNA | NM_006710 | COP9 | COP9 homolog |
| 483 | Protein | NP_006701 | COP9 | COP9 homolog |
| 484 | DNA | NM_012425 | RSU1 | Ras suppressor protein 1 |
| 485 | Protein | NP_036557 | RSU1 | Ras suppressor protein 1 |
| 486 | DNA | NM_012321 | LSM4 | U6 snRNA-associated Sm-like |
| | | | | protein |
| 487 | Protein | NP_036453 | LSM4 | U6 snRNA-associated Sm-like |
| | | | | protein |
| 488 | DNA | NM_000430 | PAFAH1B1 | platelet-activating factor |
| | | | | acetylhydrolase, isoform Ib, |
| | | | | alpha subunit 45 kDa |
| 489 | Protein | NP_000421 | PAFAH1B1 | platelet-activating factor |
| | | | | acetylhydrolase, isoform Ib, |
| | | | | alpha subunit 45 kDa |
| 490 | DNA | D86971 | KIAA0217 | KIAA0217 protein |
| 491 | Protein | D86971 (Translation) | KIAA0217 | KIAA0217 protein |
| 492 | DNA | NM_006887 | ZFP36L2 | zinc finger protein 36, C3H |
| | | | | type-like 2 |
| 493 | Protein | NP_008818 | ZFP36L2 | zinc finger protein 36, C3H |
| | | | | type-like 2 |
| 494 | DNA | NM_005483 | CHAF1A | chromatin assembly factor 1, |
| | | | | subunit A (p150) |
| 495 | Protein | NP_005474 | CHAF1A | chromatin assembly factor 1, |
| | | | | subunit A (p150) |
| 496 | DNA | AF000560 | | Homo sapiens, clone |
| | | | | IMAGE: 4477095, mRNA, |
| | | | | mRNA sequence |
| 497 | Protein | AAB58413 | | Homo sapiens, clone |
| | | | | IMAGE: 4477095, mRNA, |
| | | | | mRNA sequence |
| 498 | DNA | NM_002567 | PBP | prostatic binding protein |
| 499 | Protein | NP_002558 | PBP | prostatic binding protein |
| 500 | DNA | NM_015906 | TRIM33 | tripartite motif-containing 33 |
| 501 | Protein | NP_056990 | TRIM33 | tripartite motif-containing 33 |
| 502 | DNA | NM_033020 | TRIM33 | tripartite motif-containing 33 |
| 503 | Protein | NP_148980 | TRIM33 | tripartite motif-containing 33 |
| 504 | DNA | NM_006696 | SMAP | skeletal muscle abundant |
| | | | | protein |
| 505 | Protein | NP_006687 | SMAP | skeletal muscle abundant |
| | | | | protein |
| 506 | DNA | NM_015636 | EIF2B4 | eukaryotic translation initiation |
| | | | | factor 2B, subunit 4 delta, |
| | | | | 67 kDa |
| 507 | Protein | NP_056451 | EIF2B4 | eukaryotic translation initiation |
| | | | | factor 2B, subunit 4 delta, |
| | | | | 67 kDa |
| 508 | DNA | NM_006195 | PBX3 | pre-B-cell leukemia |
| | | | | transcription factor 3 |
| 509 | Protein | NP_006186 | PBX3 | pre-B-cell leukemia |
| | | | | transcription factor 3 |
| 510 | DNA | NM_003325 | HIRA | HIR histone cell cycle |
| | | | | regulation defective homolog A |
| | | | | (S. cerevisiae) |
| 511 | Protein | NP_003316 | HIRA | HIR histone cell cycle |
| | | | | regulation defective homolog A |
| | | | | (S. cerevisiae) |
| 512 | DNA | NM_001324 | CSTF1 | cleavage stimulation factor, 3′ |
| | | | | pre-RNA, subunit 1, 50 kDa |
| 513 | Protein | NP_001315 | CSTF1 | cleavage stimulation factor, 3′ |
| | | | | pre-RNA, subunit 1, 50 kDa |
| 514 | DNA | NM_006246 | PPP2R5E | protein phosphatase 2, |
| | | | | regulatory subunit B (B56), |
| | | | | epsilon isoform |
| 515 | Protein | NP_006237 | PPP2R5E | protein phosphatase 2, |
| | | | | regulatory subunit B (B56), |
| | | | | epsilon isoform |
| 516 | DNA | AB023148 | KIAA0931 | KIAA0931 protein |
| 517 | Protein | AB023148 | KIAA0931 | KIAA0931 protein |
| | | (Translation) | | |
| 518 | DNA | NM_003610 | RAE1 | RAE1 RNA export 1 homolog |
| | | | | (S. pombe) |
| 519 | Protein | NP_003601 | RAE1 | RAE1 RNA export 1 homolog |
| | | | | (S. pombe) |
| 520 | DNA | NM_001469 | G22P1 | thyroid autoantigen 70 kDa (Ku |
| | | | | antigen) |
| 521 | Protein | NP_001460 | G22P1 | thyroid autoantigen 70 kDa (Ku |
| | | | | antigen) |
| 522 | DNA | NM_003035 | SIL | TAL1 (SCL) interrupting locus |
| 523 | Protein | NP_003026 | SIL | TAL1 (SCL) interrupting locus |
| 524 | DNA | NM_030794 | FLJ21007 | hypothetical protein FLJ21007 |
| 525 | Protein | NP_110421 | FLJ21007 | hypothetical protein FLJ21007 |
| 526 | DNA | NM_006267 | RANBP2 | RAN binding protein 2 |
| 527 | Protein | NP_006258 | RANBP2 | RAN binding protein 2 |
| 528 | DNA | L19183 | MAC30 | hypothetical protein MAC30 |
| 529 | Protein | L19183 (Translation) | MAC30 | hypothetical protein MAC30 |
| 530 | DNA | AF004292 | DKFZP566C134 | DKFZP566C134 protein |
| 531 | DNA | AL118582 | | OVN6-2 [Homo sapiens], |
| | | | | mRNA sequence |
| 532 | DNA | NM_003021 | SGT | small glutamine-rich |
| | | | | tetratricopeptide repeat (TPR)- |
| | | | | containing |
| 533 | Protein | NP_003012 | SGT | small glutamine-rich |
| | | | | tetratricopeptide repeat (TPR)- |
| | | | | containing |
| 534 | DNA | NM_005882 | MAEA | macrophage erythroblast |
| | | | | attacher |
| 535 | Protein | NP_005873 | MAEA | macrophage erythroblast |
| | | | | attacher |
| 536 | DNA | NM_006411 | AGPAT1 | 1-acylglycerol-3-phosphate O- |
| | | | | acyltransferase 1 |
| | | | | (lysophosphatidic acid |
| | | | | acyltransferase, alpha) |
| 537 | Protein | NP_006402 | AGPAT1 | 1-acylglycerol-3-phosphate O- |
| | | | | acyltransferase 1 |
| | | | | (lysophosphatidic acid |
| | | | | acyltransferase, alpha) |
| 538 | DNA | NM_032741 | AGPAT1 | 1-acylglycerol-3-phosphate O- |
| | | | | acyltransferase 1 |
| | | | | (lysophosphatidic acid |
| | | | | acyltransferase, alpha) |
| 539 | DNA | NM_014820 | TOMM70A | translocase of outer |
| | | | | mitochondrial membrane 70 |
| | | | | homolog A (yeast) |
| 540 | Protein | NP_055635 | TOMM70A | translocase of outer |
| | | | | mitochondrial membrane 70 |
| | | | | homolog A (yeast) |
| 541 | DNA | NM_012300 | FBXW1B | F-box and WD-40 domain |
| | | | | protein 1B |
| 542 | Protein | NP_036432 | FBXW1B | F-box and WD-40 domain |
| | | | | protein 1B |
| 543 | DNA | NM_033644 | FBXW1B | F-box and WD-40 domain |
| | | | | protein 1B |
| 544 | Protein | NP_387448 | FBXW1B | F-box and WD-40 domain |
| | | | | protein 1B |
| 545 | DNA | NM_033645 | FBXW1B | F-box and WD-40 domain |
| | | | | protein 1B |
| 546 | Protein | NP_387449 | FBXW1B | F-box and WD-40 domain |
| | | | | protein 1B |
| 547 | DNA | NM_016936 | UBN1 | ubinuclein 1 |
| 548 | Protein | NP_058632 | UBN1 | ubinuclein 1 |
| 549 | DNA | NM_006950 | SYN1 | synapsin I |
| 550 | Protein | NP_008881 | SYN1 | synapsin I |
| 551 | DNA | NM_133499 | SYN1 | synapsin I |
| 552 | Protein | NP_598006 | SYN1 | synapsin I |
| 553 | DNA | NM_153208 | MGC35048 | hypothetical protein |
| | | | | MGC35048 |
| 554 | Protein | NP_694940 | MGC35048 | hypothetical protein |
| | | | | MGC35048 |
| 555 | DNA | NM_014282 | HABP4 | hyaluronan binding protein 4 |
| 556 | Protein | NP_055097 | HABP4 | hyaluronan binding protein 4 |
| 557 | DNA | AF035314 | | Homo sapiens clone 23651 |
| | | | | mRNA sequence |
| 558 | DNA | NM_003637 | ITGA10 | integrin, alpha 10 |
| 559 | Protein | NP_003628 | ITGA10 | integrin, alpha 10 |
| 560 | DNA | NM_001016 | RPS12 | ribosomal protein S12 |
| 561 | Protein | NP_001007 | RPS12 | ribosomal protein S12 |
| 562 | DNA | L10379 | HRIHFB2206 | HRIHFB2206 protein |
| 563 | DNA | NM_003107 | SOX4 | SRY (sex determining region |
| | | | | Y)-box 4 |
| 564 | Protein | NP_003098 | SOX4 | SRY (sex determining region |
| | | | | Y)-box 4 |
| 565 | DNA | NM_003056 | SLC19A1 | solute carrier family 19 (folate |
| | | | | transporter), member 1 |
| 566 | Protein | NP_003047 | SLC19A1 | solute carrier family 19 (folate |
| | | | | transporter), member 1 |
| 567 | DNA | NM_006831 | HEAB | ATP/GTP-binding protein |
| 568 | Protein | NP_006822 | HEAB | ATP/GTP-binding protein |
| 569 | DNA | NM_020368 | SAS10 | disrupter of silencing 10 |
| 570 | Protein | NP_065101 | SAS10 | disrupter of silencing 10 |
| 571 | DNA | NM_002061 | GCLM | glutamate-cysteine ligase, |
| | | | | modifier subunit |
| 572 | Protein | NP_002052 | GCLM | glutamate-cysteine ligase, |
| | | | | modifier subunit |
| 573 | DNA | NM_018121 | C10ORF6 | hypothetical protein FLJ10512 |
| 574 | Protein | NP_060591 | C10ORF6 | hypothetical protein FLJ10512 |
| 575 | DNA | NM_144592 | C10ORF6 | hypothetical protein FLJ10512 |
| 576 | Protein | NP_653193 | C10ORF6 | hypothetical protein FLJ10512 |
| 577 | DNA | NM_006165 | NFRKB | nuclear factor related to kappa |
| | | | | B binding protein |
| 578 | Protein | NP_006156 | NFRKB | nuclear factor related to kappa |
| | | | | B binding protein |
| 579 | DNA | NM_004587 | RRBP1 | ribosome binding protein 1 |
| | | | | homolog 180 kDa (dog) |
| 580 | Protein | NP_004578 | RRBP1 | ribosome binding protein 1 |
| | | | | homolog 180 kDa (dog) |
| 581 | DNA | AA887480 | KIAA0117 | KIAA0117 protein |
| 582 | DNA | NM_014788 | TRIM14 | tripartite motif-containing 14 |
| 583 | Protein | NP_055603 | TRIM14 | tripartite motif-containing 14 |
| 584 | DNA | NM_033219 | TRIM14 | tripartite motif-containing 14 |
| 585 | DNA | NM_033220 | TRIM14 | tripartite motif-containing 14 |
| 586 | DNA | NM_033221 | TRIM14 | tripartite motif-containing 14 |
| 587 | Protein | NP_150090 | TRIM14 | tripartite motif-containing 14 |
| 588 | DNA | NM_003705 | SLC25A12 | solute carrier family 25 |
| | | | | (mitochondrial carrier, Aralar), |
| | | | | member 12 |
| 589 | Protein | NP_003696 | SLC25A12 | solute carrier family 25 |
| | | | | (mitochondrial carrier, Aralar), |
| | | | | member 12 |
| 590 | DNA | NM_021983 | HLA-DRB4 | major histocompatibility |
| | | | | complex, class II, DR beta 4 |
| 591 | Protein | NP_068818 | HLA-DRB4 | major histocompatibility |
| | | | | complex, class II, DR beta 4 |
| 592 | DNA | NM_015004 | KIAA0116 | KIAA0116 protein |
| 593 | Protein | NP_055819 | KIAA0116 | KIAA0116 protein |
| 594 | DNA | NM_015703 | CGI-96 | CGI-96 protein |
| 595 | Protein | NP_056518 | CGI-96 | CGI-96 protein |
| 596 | DNA | NM_000181 | GUSB | glucuronidase, beta |
| 597 | Protein | NP_000172 | GUSB | glucuronidase, beta |
| 598 | DNA | NM_014509 | | Homo sapiens kraken-like |
| | | | | (dJ222E13.1), mRNA |
| 599 | Protein | NP_055324 | | Homo sapiens kraken-like |
| | | | | (dJ222E13.1) |
| 600 | DNA | NM_004290 | RNF14 | ring finger protein 14 |
| 601 | Protein | NP_004281 | RNF14 | ring finger protein 14 |
| 602 | DNA | NM_002254 | KIF3C | kinesin family member 3C |
| 603 | Protein | NP_002245 | KIF3C | kinesin family member 3C |
| 604 | DNA | NM_003205 | TCF12 | transcription factor 12 (HTF4, |
| | | | | helix-loop-helix transcription |
| | | | | factors 4) |
| 605 | Protein | NP_003196 | TCF12 | transcription factor 12 (HTF4, |
| | | | | helix-loop-helix transcription |
| | | | | factors 4) |
| 606 | DNA | NM_005875 | GC20 | translation factor suil homolog |
| 607 | Protein | NP_005866 | GC20 | translation factor suil homolog |
| 608 | DNA | NM_022739 | SMURF2 | E3 ubiquitin ligase SMURF2 |
| 609 | Protein | NP_073576 | SMURF2 | E3 ubiquitin ligase SMURF2 |
| 610 | DNA | NM_012308 | FBXL11 | F-box and leucine-rich repeat |
| | | | | protein 11 |
| 611 | Protein | NP_036440 | FBXL11 | F-box and leucine-rich repeat |
| | | | | protein 11 |
| 612 | DNA | NM_014952 | KIAA0945 | KIAA0945 protein |
| 613 | Protein | NP_055767 | KIAA0945 | KIAA0945 protein |
| 614 | DNA | NM_004793 | PRSS15 | protease, serine, 15 |
| 615 | Protein | NP_004784 | PRSS15 | protease, serine, 15 |
| 616 | DNA | NM_015384 | IDN3 | IDN3 protein |
| 617 | Protein | NP_056199 | IDN3 | IDN3 protein |
| 618 | DNA | NM_133433 | IDN3 | IDN3 protein |
| 619 | Protein | NP_597677 | IDN3 | IDN3 protein |
| 620 | DNA | NM_006999 | POLS | polymerase (DNA directed) |
| | | | | sigma |
| 621 | Protein | NP_008930 | POLS | polymerase (DNA directed) |
| | | | | sigma |
| 622 | DNA | NM_005318 | | Cluster Incl. Z97630: Human |
| | | | | DNA sequence from clone |
| | | | | 466N1 on chromosome 22q12-13 |
| | | | | Contains H1F0(H1 histone |
| | | | | family, member 0) gene, 2- |
| | | | | amino-3-ketobutyrate-CoA |
| | | | | ligase(nuclear gene encoding |
| | | | | mitochondrial protein), GALR3 |
| | | | | (galanin receptor) gene, ESTs, |
| | | | | GSSs an |
| 623 | Protein | NP_005309 | | Cluster Incl. Z97630: Human |
| | | | | DNA sequence from clone |
| | | | | 466N1 on chromosome 22q12-13 |
| | | | | Contains H1F0(H1 histone |
| | | | | family, member 0) gene, 2- |
| | | | | amino-3-ketobutyrate-CoA |
| | | | | ligase(nuclear gene encoding |
| | | | | mitochondrial protein), GALR3 |
| | | | | (galanin receptor) gene, ESTs, |
| | | | | GSSs an |
| 624 | DNA | NM_000852 | GSTP1 | glutathione S-transferase pi |
| 625 | Protein | NP_000843 | GSTP1 | glutathione S-transferase pi |
| 626 | DNA | NM_015607 | DKFZP547E1010 | DKFZP547E1010 protein |
| 627 | Protein | NP_056422 | DKFZP547E1010 | DKFZP547E1010 protein |
| 628 | DNA | AL096752 | | Homo sapiens mRNA; cDNA |
| | | | | DKFZp434A012 (from clone |
| | | | | DKFZp434A012), mRNA |
| | | | | sequence |
| 629 | DNA | NM_000983 | RPL22 | ribosomal protein L22 |
| 630 | Protein | NP_000974 | RPL22 | ribosomal protein L22 |
| 631 | DNA | NM_005269 | GLI | glioma-associated oncogene |
| | | | | homolog (zinc finger protein) |
| 632 | Protein | NP_005260 | GLI | glioma-associated oncogene |
| | | | | homolog (zinc finger protein) |
| 633 | DNA | NM_000968 | RPL4 | ribosomal protein L4 |
| 634 | Protein | NP_000959 | RPL4 | ribosomal protein L4 |
| 635 | DNA | NM_000838 | GRM1 | glutamate receptor, |
| | | | | metabotropic 1 |
| 636 | Protein | NP_000829 | GRM1 | glutamate receptor, |
| | | | | metabotropic 1 |
| 637 | DNA | NM_000704 | ATP4A | ATPase, H+/K+ exchanging, |
| | | | | alpha polypeptide |
| 638 | Protein | NP_000695 | ATP4A | ATPase, H+/K+ exchanging, |
| | | | | alpha polypeptide |
| 639 | DNA | NM_006213 | PHKG1 | phosphorylase kinase, gamma 1 |
| | | | | (muscle) |
| 640 | Protein | NP_006204 | PHKG1 | phosphorylase kinase, gamma 1 |
| | | | | (muscle) |
| 641 | DNA | NM_001060 | TBXA2R | thromboxane A2 receptor |
| 642 | Protein | NP_001051 | TBXA2R | thromboxane A2 receptor |
| 643 | DNA | NM_000980 | RPL18A | ribosomal protein L18a |
| 644 | Protein | NP_000971 | RPL18A | ribosomal protein L18a |
| 645 | DNA | NM_000405 | GM2A | GM2 ganglioside activator |
| | | | | protein |
| 646 | Protein | NP_000396 | GM2A | GM2 ganglioside activator |
| | | | | protein |
| 647 | DNA | NM_000997 | RPL37 | ribosomal protein L37 |
| 648 | Protein | NP_000988 | RPL37 | ribosomal protein L37 |
| 649 | DNA | NM_003431 | ZNF124 | zinc finger protein 124 (HZF- |
| | | | | 16) |
| 650 | Protein | NP_003422 | ZNF124 | zinc finger protein 124 (HZF- |
| | | | | 16) |
| 651 | DNA | NM_005507 | CFL1 | cofilin 1 (non-muscle) |
| 652 | Protein | NP_005498 | CFL1 | cofilin 1 (non-muscle) |
| 653 | DNA | NM_021130 | PPIA | peptidylprolyl isomerase A |
| | | | | (cyclophilin A) |
| 654 | Protein | NP_066953 | PPIA | peptidylprolyl isomerase A |
| | | | | (cyclophilin A) |
| 655 | DNA | NM_000976 | RPL12 | ribosomal protein L12 |
| 656 | Protein | NP_000967 | RPL12 | ribosomal protein L12 |
| 657 | DNA | NM_000992 | RPL29 | ribosomal protein L29 |
| 658 | Protein | NP_000983 | RPL29 | ribosomal protein L29 |
| 659 | DNA | NM_000993 | RPL31 | ribosomal protein L31 |
| 660 | Protein | NP_000984 | RPL31 | ribosomal protein L31 |
| 661 | DNA | D50525 | | Cluster Incl. D50525: Human |
| | | | | mRNA for TI-227H |
| | | | | /cds = UNKNOWN/gb = D50525 |
| | | | | /gi = 1167502 /ug = Hs.184914 |
| | | | | /len = 3911 |
| 662 | DNA | NM_001355 | DDT | D-dopachrome tautomerase |
| 663 | Protein | NP_001346 | DDT | D-dopachrome tautomerase |
| 664 | DNA | NM_005834 | TIMM17B | translocase of inner |
| | | | | mitochondrial membrane 17 |
| | | | | homolog B (yeast) |
| 665 | Protein | NP_005825 | TIMM17B | translocase of inner |
| | | | | mitochondrial membrane 17 |
| | | | | homolog B (yeast) |
| 666 | DNA | NM_007294 | BRCA1 | breast cancer 1, early onset |
| 667 | Protein | NP_009225 | BRCA1 | breast cancer 1, early onset |
| 668 | DNA | NM_007295 | BRCA1 | breast cancer 1, early onset |
| 669 | DNA | NM_007296 | BRCA1 | breast cancer 1, early onset |
| 670 | DNA | NM_007297 | BRCA1 | breast cancer 1, early onset |
| 671 | Protein | NP_009228 | BRCA1 | breast cancer 1, early onset |
| 672 | DNA | NM_007298 | BRCA1 | breast cancer 1, early onset |
| 673 | Protein | NP_009229 | BRCA1 | breast cancer 1, early onset |
| 674 | DNA | NM_004805 | POLR2D | polymerase (RNA) II (DNA |
| | | | | directed) polypeptide D |
| 675 | Protein | NP_004796 | POLR2D | polymerase (RNA) II (DNA |
| | | | | directed) polypeptide D |
| 676 | DNA | NM_015487 | GEMIN4 | gem (nuclear organelle) |
| | | | | associated protein 4 |
| 677 | Protein | NP_056302 | GEMIN4 | gem (nuclear organelle) |
| | | | | associated protein 4 |
| 678 | DNA | NM_015721 | GEMIN4 | gem (nuclear organelle) |
| | | | | associated protein 4 |
| 679 | DNA | AJ006835 | RNU17D | RNA, U17D small nucleolar |
| 680 | DNA | NM_031246 | PSG2 | pregnancy specific beta-1- |
| | | | | glycoprotein 2 |
| 681 | Protein | NP_112536 | PSG2 | pregnancy specific beta-1- |
| | | | | glycoprotein 2 |
| 682 | DNA | NM_004565 | PEX14 | peroxisomal biogenesis factor |
| | | | | 14 |
| 683 | Protein | NP_004556 | PEX14 | peroxisomal biogenesis factor |
| | | | | 14 |
| 684 | DNA | NM_001228 | CASP8 | caspase 8, apoptosis-related |
| | | | | cysteine protease |
| 685 | Protein | NP_001219 | CASP8 | caspase 8, apoptosis-related |
| | | | | cysteine protease |
| 686 | DNA | NM_033355 | CASP8 | caspase 8, apoptosis-related |
| | | | | cysteine protease |
| 687 | Protein | NP_203519 | CASP8 | caspase 8, apoptosis-related |
| | | | | cysteine protease |
| 688 | DNA | NM_033356 | CASP8 | caspase 8, apoptosis-related |
| | | | | cysteine protease |
| 689 | Protein | NP_203520 | CASP8 | caspase 8, apoptosis-related |
| | | | | cysteine protease |
| 690 | DNA | NM_033357 | CASP8 | caspase 8, apoptosis-related |
| | | | | cysteine protease |
| 691 | Protein | NP_203521 | CASP8 | caspase 8, apoptosis-related |
| | | | | cysteine protease |
| 692 | DNA | NM_033358 | CASP8 | caspase 8, apoptosis-related |
| | | | | cysteine protease |
| 693 | Protein | NP_203522 | CASP8 | caspase 8, apoptosis-related |
| | | | | cysteine protease |
| 694 | DNA | NM_001061 | TBXAS1 | thromboxane A synthase 1 |
| | | | | (platelet, cytochrome P450, |
| | | | | subfamily V) |
| 695 | Protein | NP_001052 | TBXAS1 | thromboxane A synthase 1 |
| | | | | (platelet, cytochrome P450, |
| | | | | subfamily V) |
| 696 | DNA | NM_030984 | TBXAS1 | thromboxane A synthase 1 |
| | | | | (platelet, cytochrome P450, |
| | | | | subfamily V) |
| 697 | Protein | NP_112246 | TBXAS1 | thromboxane A synthase 1 |
| | | | | (platelet, cytochrome P450, |
| | | | | subfamily V) |
| 698 | DNA | NM_004901 | LYSAL1 | lysosomal apyrase-like 1 |
| 699 | Protein | NP_004892 | LYSAL1 | lysosomal apyrase-like 1 |
| 700 | DNA | X98494 | MPHOSPH10 | M-phase phosphoprotein 10 |
| | | | | (U3 small nucleolar |
| | | | | ribonucleoprotein) |
| 701 | Protein | X98494 (Translation) | MPHOSPH10 | M-phase phosphoprotein 10 |
| | | | | (U3 small nucleolar |
| | | | | ribonucleoprotein) |
| 702 | DNA | NM_017575 | C17orf31 | chromosome 17 open reading |
| | | | | frame 31 |
| 703 | Protein | NP_060045 | C17orf31 | chromosome 17 open reading |
| | | | | frame 31 |
| 704 | DNA | NM_001116 | ADCY9 | adenylate cyclase 9 |
| 705 | Protein | NP_001107 | ADCY9 | adenylate cyclase 9 |
| 706 | DNA | NM_014810 | CAP350 | centrosome-associated protein |
| | | | | 350 |
| 707 | Protein | NP_055625 | CAP350 | centrosome-associated protein |
| | | | | 350 |
| 708 | DNA | NM_005884 | PAK4 | p21(CDKN1A)-activated |
| | | | | kinase 4 |
| 709 | Protein | NP_005875 | PAK4 | p21(CDKN1A)-activated |
| | | | | kinase 4 |
| 710 | DNA | NM_000373 | UMPS | uridine monophosphate |
| | | | | synthetase (orotate |
| | | | | phosphoribosyl transferase and |
| | | | | orotidine-5′-decarboxylase) |
| 711 | Protein | NP_000364 | UMPS | uridine monophosphate |
| | | | | synthetase (orotate |
| | | | | phosphoribosyl transferase and |
| | | | | orotidine-5′-decarboxylase) |
| 712 | DNA | NM_002273 | KRT8 | keratin 8 |
| 713 | Protein | NP_002264 | KRT8 | keratin 8 |
| 714 | DNA | NM_006985 | NPIP | nuclear pore complex |
| | | | | interacting protein |
| 715 | Protein | NP_008916 | NPIP | nuclear pore complex |
| | | | | interacting protein |
| 716 | DNA | NM_004064 | CDKN1B | cyclin-dependent kinase |
| | | | | inhibitor 1B (p27, Kip1) |
| 717 | Protein | NP_004055 | CDKN1B | cyclin-dependent kinase |
| | | | | inhibitor 1B (p27, Kip1) |
| 718 | DNA | NM_020765 | RBAF600 | retinoblastoma-associated |
| | | | | factor 600 |
| 719 | Protein | NP_065816 | RBAF600 | retinoblastoma-associated |
| | | | | factor 600 |
| 720 | DNA | AI123426 | | EST |
| 721 | DNA | NM_005997 | TCFL1 | transcription factor-like 1 |
| 722 | Protein | NP_005988 | TCFL1 | transcription factor-like 1 |
| 723 | DNA | NM_005866 | SR-BP1 | type I sigma receptor |
| 724 | Protein | NP_005857 | SR-BP1 | type I sigma receptor |
| 725 | DNA | NM_147157 | SR-BP1 | type I sigma receptor |
| 726 | Protein | NP_671513 | SR-BP1 | type I sigma receptor |
| 727 | DNA | NM_147158 | SR-BP1 | type I sigma receptor |
| 728 | Protein | NP_671514 | SR-BP1 | type I sigma receptor |
| 729 | DNA | NM_147159 | SR-BP1 | type I sigma receptor |
| 730 | Protein | NP_671515 | SR-BP1 | type I sigma receptor |
| 731 | DNA | NM_147160 | SR-BP1 | type I sigma receptor |
| 732 | Protein | NP_671516 | SR-BP1 | type I sigma receptor |
| 733 | DNA | NM_004457 | FACL3 | fatty-acid-Coenzyme A ligase, |
| | | | | long-chain 3 |
| 734 | Protein | NP_004448 | FACL3 | fatty-acid-Coenzyme A ligase, |
| | | | | long-chain 3 |
| 735 | DNA | NM_005137 | DGCR2 | DiGeorge syndrome critical |
| | | | | region gene 2 |
| 736 | Protein | NP_005128 | DGCR2 | DiGeorge syndrome critical |
| | | | | region gene 2 |
| 737 | DNA | NM_014812 | KIAA0470 | KIAA0470 gene product |
| 738 | Protein | NP_055627 | KIAA0470 | KIAA0470 gene product |
| 739 | DNA | NM_001348 | DAPK3 | death-associated protein kinase 3 |
| 740 | Protein | NP_001339 | DAPK3 | death-associated protein kinase 3 |
| 741 | DNA | NM_003927 | MBD2 | methyl-CpG binding domain |
| | | | | protein 2 |
| 742 | Protein | NP_003918 | MBD2 | methyl-CpG binding domain |
| | | | | protein 2 |
| 743 | DNA | NM_015832 | MBD2 | methyl-CpG binding domain |
| | | | | protein 2 |
| 744 | Protein | NP_056647 | MBD2 | methyl-CpG binding domain |
| | | | | protein 2 |
| 745 | DNA | NM_004638 | BAT2 | HLA-B associated transcript 2 |
| 746 | Protein | NP_004629 | BAT2 | HLA-B associated transcript 2 |
| 747 | DNA | NM_080686 | BAT2 | HLA-B associated transcript 2 |
| 748 | Protein | NP_542417 | BAT2 | HLA-B associated transcript 2 |
| 749 | DNA | NM_002032 | FTH1 | ferritin, heavy polypeptide 1 |
| 750 | Protein | NP_002023 | FTH1 | ferritin, heavy polypeptide 1 |
| 751 | DNA | NM_000477 | ALB | albumin |
| 752 | Protein | NP_000468 | ALB | albumin |
| 753 | DNA | NM_021019 | MYL6 | myosin, light polypeptide 6, |
| | | | | alkali, smooth muscle and non- |
| | | | | muscle |
| 754 | Protein | NP_066299 | MYL6 | myosin, light polypeptide 6, |
| | | | | alkali, smooth muscle and non- |
| | | | | muscle |
| 755 | DNA | NM_079423 | MYL6 | myosin, light polypeptide 6, |
| | | | | alkali, smooth muscle and non- |
| | | | | muscle |
| 756 | Protein | NP_524147 | MYL6 | myosin, light polypeptide 6, |
| | | | | alkali, smooth muscle and non- |
| | | | | muscle |
| 757 | DNA | NM_079424 | MYL6 | myosin, light polypeptide 6, |
| | | | | alkali, smooth muscle and non- |
| | | | | muscle |
| 758 | Protein | NP_524148 | MYL6 | myosin, light polypeptide 6, |
| | | | | alkali, smooth muscle and non- |
| | | | | muscle |
| 759 | DNA | NM_079425 | MYL6 | myosin, light polypeptide 6, |
| | | | | alkali, smooth muscle and non- |
| | | | | muscle |
| 760 | Protein | NP_524149 | MYL6 | myosin, light polypeptide 6, |
| | | | | alkali, smooth muscle and non- |
| | | | | muscle |
| 761 | DNA | AL049449 | | Homo sapiens mRNA; cDNA |
| | | | | DKFZp586B1722 (from clone |
| | | | | DKFZp586B1722), mRNA |
| | | | | sequence |
| 762 | DNA | NM_002381 | MATN3 | matrilin 3 |
| 763 | Protein | NP_002372 | MATN3 | matrilin 3 |
| 764 | DNA | NM_000365 | TPI1 | triosephosphate isomerase 1 |
| 765 | Protein | NP_000356 | TPI1 | triosephosphate isomerase 1 |
| 766 | DNA | NM_004996 | ABCC1 | ATP-binding cassette, sub- |
| | | | | family C (CFTR/MRP), |
| | | | | member 1 |
| 767 | Protein | NP_004987 | ABCC1 | ATP-binding cassette, sub- |
| | | | | family C (CFTR/MRP), |
| | | | | member 1 |
| 768 | DNA | NM_019862 | ABCC1 | ATP-binding cassette, sub- |
| | | | | family C (CFTR/MRP), |
| | | | | member 1 |
| 769 | Protein | NP_063915 | ABCC1 | ATP-binding cassette, sub- |
| | | | | family C (CFTR/MRP), |
| | | | | member 1 |
| 770 | DNA | NM_019898 | ABCC1 | ATP-binding cassette, sub- |
| | | | | family C (CFTR/MRP), |
| | | | | member 1 |
| 771 | Protein | NP_063953 | ABCC1 | ATP-binding cassette, sub- |
| | | | | family C (CFTR/MRP), |
| | | | | member 1 |
| 772 | DNA | NM_019899 | ABCC1 | ATP-binding cassette, sub- |
| | | | | family C (CFTR/MRP), |
| | | | | member 1 |
| 773 | Protein | NP_063954 | ABCC1 | ATP-binding cassette, sub- |
| | | | | family C (CFTR/MRP), |
| | | | | member 1 |
| 774 | DNA | NM_000490 | AVP | arginine vasopressin |
| | | | | (neurophysin II, antidiuretic |
| | | | | hormone, diabetes insipidus, |
| | | | | neurohypophyseal) |
| 775 | Protein | NP_000481 | AVP | arginine vasopressin |
| | | | | (neurophysin II, antidiuretic |
| | | | | hormone, diabetes insipidus, |
| | | | | neurohypophyseal) |
| 776 | DNA | NM_000999 | RPL38 | ribosomal protein L38 |
| 777 | Protein | NP_000990 | RPL38 | ribosomal protein L38 |
| 778 | DNA | NM_002297 | LCN1 | lipocalin 1 (protein migrating |
| | | | | faster than albumin, tear |
| | | | | prealbumin) |
| 779 | Protein | NP_002288 | LCN1 | lipocalin 1 (protein migrating |
| | | | | faster than albumin, tear |
| | | | | prealbumin) |
| 780 | DNA | NM_006068 | TLR6 | toll-like receptor 6 |
| 781 | Protein | NP_006059 | TLR6 | toll-like receptor 6 |
| 782 | DNA | NM_012302 | LPHH1 | latrophilin 1 |
| 783 | Protein | NP_036434 | LPHH1 | latrophilin 1 |
| 784 | DNA | NM_005453 | ZNF297 | zinc finger protein 297 |
| 785 | Protein | NP_005444 | ZNF297 | zinc finger protein 297 |
| 786 | DNA | AB020676 | KIAA0869 | KIAA0869 protein |
| 787 | Protein | AB020676 | KIAA0869 | KIAA0869 protein |
| | | (Translation) | | |
| 788 | DNA | D83781 | NUP160 | nucleoporin 160 kDa |
| 789 | Protein | D83781 (Translation) | NUP160 | nucleoporin 160 kDa |
| 790 | DNA | NM_015229 | KIAA0664 | KIAA0664 protein |
| 791 | Protein | NP_056044 | KIAA0664 | KIAA0664 protein |
| 792 | DNA | NM_005873 | RGS19 | regulator of G-protein |
| | | | | signalling 19 |
| 793 | Protein | NP_005864 | RGS19 | regulator of G-protein |
| | | | | signalling 19 |
| 794 | DNA | NM_015608 | DKFZp586F1019 | DKFZp586F1019 protein |
| 795 | Protein | NP_056423 | DKFZp586F1019 | DKFZp586F1019 protein |
| 796 | DNA | NM_014892 | KIAA1116 | KIAA1116 protein |
| 797 | Protein | NP_055707 | KIAA1116 | KIAA1116 protein |
| 798 | DNA | NM_025176 | KIAA0980 | KIAA0980 protein |
| 799 | Protein | NP_079452 | KIAA0980 | KIAA0980 protein |
| 800 | DNA | NM_001217 | CA11 | carbonic anhydrase XI |
| 801 | Protein | NP_001208 | CA11 | carbonic anhydrase XI |
| 802 | DNA | NM_014323 | ZNF278 | zinc finger protein 278 |
| 803 | Protein | NP_055138 | ZNF278 | zinc finger protein 278 |
| 804 | DNA | NM_032050 | ZNF278 | zinc finger protein 278 |
| 805 | Protein | NP_114439 | ZNF278 | zinc finger protein 278 |
| 806 | DNA | NM_032051 | ZNF278 | zinc finger protein 278 |
| 807 | Protein | NP_114440 | ZNF278 | zinc finger protein 278 |
| 808 | DNA | NM_032052 | ZNF278 | zinc finger protein 278 |
| 809 | Protein | NP_114441 | ZNF278 | zinc finger protein 278 |
| 810 | DNA | NM_006196 | PCBP1 | poly(rC) binding protein 1 |
| 811 | Protein | NP_006187 | PCBP1 | poly(rC) binding protein 1 |
| 812 | DNA | NM_021038 | MBNL | muscleblind-like (Drosophila) |
| 813 | Protein | NP_066368 | MBNL | muscleblind-like (Drosophila) |
| 814 | DNA | NM_000485 | APRT | adenine |
| | | | | phosphoribosyltransferase |
| 815 | Protein | NP_000476 | APRT | adenine |
| | | | | phosphoribosyltransferase |
| 816 | DNA | AI040324 | | ESTs, Weakly similar to |
| | | | | A56429 I-kappa-B-related |
| | | | | protein - human [H. sapiens] |
| 817 | DNA | NM_006796 | AFG3L2 | AFG3 ATPase family gene 3- |
| | | | | like 2 (yeast) |
| 818 | Protein | NP_006787 | AFG3L2 | AFG3 ATPase family gene 3- |
| | | | | like 2 (yeast) |
| 819 | DNA | NM_014876 | KIAA0063 | KIAA0063 gene product |
| 820 | Protein | NP_055691 | KIAA0063 | KIAA0063 gene product |
| 821 | DNA | NM_007358 | M96 | likely ortholog of mouse metal |
| | | | | response element binding |
| | | | | transcription factor 2 |
| 822 | Protein | NP_031384 | M96 | likely ortholog of mouse metal |
| | | | | response element binding |
| | | | | transcription factor 2 |
| 823 | DNA | NM_002956 | RSN | restin (Reed-Steinberg cell- |
| | | | | expressed intermediate |
| | | | | filament-associated protein) |
| 824 | Protein | NP_002947 | RSN | restin (Reed-Steinberg cell- |
| | | | | expressed intermediate |
| | | | | filament-associated protein) |
| 825 | DNA | NM_000281 | PCBD | 6-pyruvoyl-tetrahydropterin |
| | | | | synthase/dimerization cofactor |
| | | | | of hepatocyte nuclear factor 1 |
| | | | | alpha (TCF1) |
| 826 | Protein | NP_000272 | PCBD | 6-pyruvoyl-tetrahydropterin |
| | | | | synthase/dimerization cofactor |
| | | | | of hepatocyte nuclear factor 1 |
| | | | | alpha (TCF1) |
| 827 | DNA | NM_015200 | KIAA0648 | KIAA0648 protein |
| 828 | Protein | NP_056015 | KIAA0648 | KIAA0648 protein |
| 829 | DNA | NM_004992 | MECP2 | methyl CpG binding protein 2 |
| | | | | (Rett syndrome) |
| 830 | Protein | NP_004983 | MECP2 | methyl CpG binding protein 2 |
| | | | | (Rett syndrome) |
| 831 | DNA | NM_021134 | MRPL23 | mitochondrial ribosomal |
| | | | | protein L23 |
| 832 | Protein | NP_066957 | MRPL23 | mitochondrial ribosomal |
| | | | | protein L23 |
| 833 | DNA | NM_005134 | PPP4R1 | protein phosphatase 4, |
| | | | | regulatory subunit 1 |
| 834 | Protein | NP_005125 | PPP4R1 | protein phosphatase 4, |
| | | | | regulatory subunit 1 |
| 835 | DNA | NM_001122 | ADFP | adipose differentiation-related |
| | | | | protein |
| 836 | Protein | NP_001113 | ADFP | adipose differentiation-related |
| | | | | protein |
| 837 | DNA | NM_003368 | USP1 | ubiquitin specific protease 1 |
| 838 | Protein | NP_003359 | USP1 | ubiquitin specific protease 1 |
| 839 | DNA | NM_003925 | MBD4 | methyl-CpG binding domain |
| | | | | protein 4 |
| 840 | Protein | NP_003916 | MBD4 | methyl-CpG binding domain |
| | | | | protein 4 |
| 841 | DNA | NM_015339 | ADNP | activity-dependent |
| | | | | neuroprotector |
| 842 | Protein | NP_056154 | ADNP | activity-dependent |
| | | | | neuroprotector |
| 843 | DNA | NM_015338 | KIAA0978 | KIAA0978 protein |
| 844 | Protein | NP_056153 | KIAA0978 | KIAA0978 protein |
| 845 | DNA | NM_006107 | OA48-18 | acid-inducible phosphoprotein |
| 846 | Protein | NP_006098 | OA48-18 | acid-inducible phosphoprotein |
| 847 | DNA | NM_014402 | QP-C | low molecular mass |
| | | | | ubiquinone-binding protein |
| | | | | (9.5 kD) |
| 848 | Protein | NP_055217 | QP-C | low molecular mass |
| | | | | ubiquinone-binding protein |
| | | | | (9.5 kD) |
| 849 | DNA | NM_005928 | MFGE8 | milk fat globule-EGF factor 8 |
| | | | | protein |
| 850 | Protein | NP_005919 | MFGE8 | milk fat globule-EGF factor 8 |
| | | | | protein |
| 851 | DNA | NM_003356 | UCP3 | uncoupling protein 3 |
| | | | | (mitochondrial, proton carrier) |
| 852 | Protein | NP_003347 | UCP3 | uncoupling protein 3 |
| | | | | (mitochondrial, proton carrier) |
| 853 | DNA | NM_022803 | UCP3 | uncoupling protein 3 |
| | | | | (mitochondrial, proton carrier) |
| 854 | Protein | NP_073714 | UCP3 | uncoupling protein 3 |
| | | | | (mitochondrial, proton carrier) |
| 855 | DNA | R61362 | | Unknown protein [Homo |
| | | | | sapiens], mRNA sequence |
| 856 | DNA | NM_003176 | SYCP1 | synaptonemal complex protein 1 |
| 857 | Protein | NP_003167 | SYCP1 | synaptonemal complex protein 1 |
| 858 | DNA | NM_005680 | TAF1B | TATA box binding protein |
| | | | | (TBP)-associated factor, RNA |
| | | | | polymerase I, B, 63 kDa |
| 859 | Protein | NP_005671 | TAF1B | TATA box binding protein |
| | | | | (TBP)-associated factor, RNA |
| | | | | polymerase I, B, 63 kDa |
| 860 | DNA | NM_030928 | CDT1 | DNA replication factor |
| 861 | Protein | NP_112190 | CDT1 | DNA replication factor |
| 862 | DNA | AF052108 | | Homo sapiens clone 23687 |
| | | | | mRNA sequence |
| 863 | DNA | NM_021012 | KCNJ12 | potassium inwardly-rectifying |
| | | | | channel, subfamily J, member |
| | | | | 12 |
| 864 | Protein | NP_066292 | KCNJ12 | potassium inwardly-rectifying |
| | | | | channel, subfamily J, member |
| | | | | 12 |
| 865 | DNA | NM_014875 | KIF14 | kinesin family member 14 |
| 866 | Protein | NP_055690 | KIF14 | kinesin family member 14 |
| 867 | DNA | NM_002954 | RPS27A | ribosomal protein S27a |
| 868 | Protein | NP_002945 | RPS27A | ribosomal protein S27a |
| 869 | DNA | NM_001021 | RPS17 | ribosomal protein S17 |
| 870 | Protein | NP_001012 | RPS17 | ribosomal protein S17 |
| 871 | DNA | NM_004983 | KCNJ9 | potassium inwardly-rectifying |
| | | | | channel, subfamily J, member 9 |
| 872 | Protein | NP_004974 | KCNJ9 | potassium inwardly-rectifying |
| | | | | channel, subfamily J, member 9 |
| 873 | DNA | NM_001926 | DEFA6 | defensin, alpha 6, Paneth cell- |
| | | | | specific |
| 874 | Protein | NP_001917 | DEFA6 | defensin, alpha 6, Paneth cell- |
| | | | | specific |
| 875 | DNA | NM_001005 | RPS3 | ribosomal protein S3 |
| 876 | Protein | NP_000996 | RPS3 | ribosomal protein S3 |
| 877 | DNA | NM_001011 | RPS7 | ribosomal protein S7 |
| 878 | Protein | NP_001002 | RPS7 | ribosomal protein S7 |
| 879 | DNA | NM_004396 | DDX5 | DEAD/H (Asp-Glu-Ala- |
| | | | | Asp/His) box polypeptide 5 |
| | | | | (RNA helicase, 68 kDa) |
| 880 | Protein | NP_004387 | DDX5 | DEAD/H (Asp-Glu-Ala- |
| | | | | Asp/His) box polypeptide 5 |
| | | | | (RNA helicase, 68 kDa) |
| 881 | DNA | NM_145809 | LOC220594 | TL132 protein |
| 882 | Protein | NP_665808 | LOC220594 | TL132 protein |
| 883 | DNA | NM_005718 | ARPC4 | actin related protein 2/3 |
| | | | | complex, subunit 4, 20 kDa |
| 884 | Protein | NP_005709 | ARPC4 | actin related protein 2/3 |
| | | | | complex, subunit 4, 20 kDa |
| 885 | DNA | NM_002336 | LRP6 | low density lipoprotein |
| | | | | receptor-related protein 6 |
| 886 | Protein | NP_002327 | LRP6 | low density lipoprotein |
| | | | | receptor-related protein 6 |
| 887 | DNA | NM_012120 | CD2AP | CD2-associated protein |
| 888 | Protein | NP_036252 | CD2AP | CD2-associated protein |
| 889 | DNA | AB011090 | MGA | MAX gene associated |
| 890 | Protein | AB011090 | MGA | MAX gene associated |
| | | (Translation) | | |
| 891 | DNA | NM_000875 | IGF1R | insulin-like growth factor 1 |
| | | | | receptor |
| 892 | Protein | NP_000866 | IGF1R | insulin-like growth factor 1 |
| | | | | receptor |
| 893 | DNA | U44385 | | Cluster Incl. U44385: Human |
| | | | | tissue inhibitor of |
| | | | | metalloproteinases-2 (TIMP-2) |
| | | | | gene /cds = (302.958) |
| | | | | /gb = U44385 /gi = 1517892 |
| | | | | /ug = Hs.239409 /len = 1069 |
| 894 | Protein | U44385 (Translation) | | Cluster Incl. U44385: Human |
| | | | | tissue inhibitor of |
| | | | | metalloproteinases-2 (TIMP-2) |
| | | | | gene /cds = (302.958) |
| | | | | /gb = U44385 /gi = 1517892 |
| | | | | /ug = Hs.239409 /len = 1069 |
| 895 | DNA | NM_004491 | GRLF1 | glucocorticoid receptor DNA |
| | | | | binding factor 1 |
| 896 | Protein | NP_004482 | GRLF1 | glucocorticoid receptor DNA |
| | | | | binding factor 1 |
| 897 | DNA | NM_024342 | GRLF1 | glucocorticoid receptor DNA |
| | | | | binding factor 1 |
| 898 | Protein | NP_077318 | GRLF1 | glucocorticoid receptor DNA |
| | | | | binding factor 1 |
| 899 | DNA | NM_017737 | FLJ20275 | hypothetical protein FLJ20275 |
| 900 | Protein | NP_060207 | FLJ20275 | hypothetical protein FLJ20275 |
| 901 | DNA | NM_005484 | ADPRTL2 | ADP-ribosyltransferase |
| | | | | (NAD+; poly(ADP-ribose) |
| | | | | polymerase)-like 2 |
| 902 | Protein | NP_005475 | ADPRTL2 | ADP-ribosyltransferase |
| | | | | (NAD+; poly(ADP-ribose) |
| | | | | polymerase)-like 2 |
| 903 | DNA | NM_005445 | CSPG6 | chondroitin sulfate |
| | | | | proteoglycan 6 (bamacan) |
| 904 | Protein | NP_005436 | CSPG6 | chondroitin sulfate |
| | | | | proteoglycan 6 (bamacan) |
| 905 | DNA | NM_012121 | CDC42EP4 | CDC42 effector protein (Rho |
| | | | | GTPase binding) 4 |
| 906 | Protein | NP_036253 | CDC42EP4 | CDC42 effector protein (Rho |
| | | | | GTPase binding) 4 |
| 907 | DNA | AB028948 | KIAA1025 | KIAA1025 protein |
| 908 | Protein | AB028948 | KIAA1025 | KIAA1025 protein |
| | | (Translation) | | |
| 909 | DNA | NM_018433 | TSGA | zinc finger protein |
| 910 | Protein | NP_060903 | TSGA | zinc finger protein |
| 911 | DNA | D14678 | KNSL2 | kinesin-like 2 |
| 912 | Protein | D14678 (Translation) | KNSL2 | kinesin-like 2 |
| 913 | DNA | AF022789 | USP12 | ubiquitin specific protease 12 |
| 914 | Protein | AF022789 | USP12 | ubiquitin specific protease 12 |
| | | (Translation) | | |
| 915 | DNA | NM_018155 | FLJ10618 | hypothetical protein FLJ10618 |
| 916 | Protein | NP_060625 | FLJ10618 | hypothetical protein FLJ10618 |
| 917 | DNA | AB023216 | | KIAA0999 protein [Homo |
| | | | | sapiens], mRNA sequence |
| 918 | Protein | AB023216 | | KIAA0999 protein [Homo |
| | | (Translation) | | sapiens], mRNA sequence |
| 919 | DNA | NM_004454 | ETV5 | ets variant gene 5 (ets-related |
| | | | | molecule) |
| 920 | Protein | NP_004445 | ETV5 | ets variant gene 5 (ets-related |
| | | | | molecule) |
| 921 | DNA | NM_016614 | TTRAP | TRAF and TNF receptor- |
| | | | | associated protein |
| 922 | Protein | NP_057698 | TTRAP | TRAF and TNF receptor- |
| | | | | associated protein |
| 923 | DNA | AB002374 | KIAA0376 | KIAA0376 protein |
| 924 | Protein | AB002374 | KIAA0376 | KIAA0376 protein |
| | | (Translation) | | |
| 925 | DNA | NM_014889 | PITRM1 | pitrilysin metalloproteinase 1 |
| 926 | Protein | NP_055704 | PITRM1 | pitrilysin metalloproteinase 1 |
| 927 | DNA | NM_014968 | PITRM1 | pitrilysin metalloproteinase 1 |
| 928 | Protein | NP_055783 | PITRM1 | pitrilysin metalloproteinase 1 |
| 929 | DNA | NM_014643 | KIAA0222 | KIAA0222 gene product |
| 930 | Protein | NP_055458 | KIAA0222 | KIAA0222 gene product |
| 931 | DNA | NM_003158 | STK6 | serine/threonine kinase 6 |
| 932 | Protein | NP_003149 | STK6 | serine/threonine kinase 6 |
| 933 | DNA | NM_003600 | STK6 | serine/threonine kinase 6 |
| 934 | Protein | NP_003591 | STK6 | serine/threonine kinase 6 |
| 935 | DNA | NM_006392 | NOL5A | nucleolar protein 5A (56 kDa |
| | | | | with KKE/D repeat) |
| 936 | Protein | NP_006383 | NOL5A | nucleolar protein 5A (56 kDa |
| | | | | with KKE/D repeat) |
| 937 | DNA | NM_021074 | NDUFV2 | NADH dehydrogenase |
| | | | | (ubiquinone) flavoprotein 2, |
| | | | | 24 kDa |
| 938 | Protein | NP_066552 | NDUFV2 | NADH dehydrogenase |
| | | | | (ubiquinone) flavoprotein 2, |
| | | | | 24 kDa |
| 939 | DNA | U51704 | KIAA1971 | similar to junction-mediating |
| | | | | and regulatory protein p300 |
| | | | | JMY |
| 940 | DNA | AI655458 | OPLAH | 5-oxoprolinase (ATP- |
| | | | | hydrolysing) |
| 941 | DNA | NM_002136 | HNRPA1 | heterogeneous nuclear |
| | | | | ribonucleoprotein A1 |
| 942 | Protein | NP_002127 | HNRPA1 | heterogeneous nuclear |
| | | | | ribonucleoprotein A1 |
| 943 | DNA | NM_031157 | HNRPA1 | heterogeneous nuclear |
| | | | | ribonucleoprotein A1 |
| 944 | Protein | NP_112420 | HNRPA1 | heterogeneous nuclear |
| | | | | ribonucleoprotein A1 |
| 945 | DNA | NM_000337 | SGCD | sarcoglycan, delta (35 kDa |
| | | | | dystrophin-associated |
| | | | | glycoprotein) |
| 946 | Protein | NP_000328 | SGCD | sarcoglycan, delta (35 kDa |
| | | | | dystrophin-associated |
| | | | | glycoprotein) |
| 947 | DNA | NM_172244 | SGCD | sarcoglycan, delta (35 kDa |
| | | | | dystrophin-associated |
| | | | | glycoprotein) |
| 948 | Protein | NP_758447 | SGCD | sarcoglycan, delta (35 kDa |
| | | | | dystrophin-associated |
| | | | | glycoprotein) |
| 949 | DNA | NM_004876 | ZNF254 | zinc finger protein 254 |
| 950 | Protein | NP_004867 | ZNF254 | zinc finger protein 254 |
| 951 | DNA | D87466 | KIAA0276 | KIAA0276 protein |
| 952 | Protein | D87466 (Translation) | KIAA0276 | KIAA0276 protein |
| 953 | DNA | NM_000828 | GRIA3 | glutamate receptor, ionotrophic, |
| | | | | AMPA 3 |
| 954 | Protein | NP_000819 | GRIA3 | glutamate receptor, ionotrophic, |
| | | | | AMPA 3 |
| 955 | DNA | NM_007325 | GRIA3 | glutamate receptor, ionotrophic, |
| | | | | AMPA 3 |
| 956 | Protein | NP_015564 | GRIA3 | glutamate receptor, ionotrophic, |
| | | | | AMPA 3 |
| 957 | DNA | NM_001207 | BTF3 | basic transcription factor 3 |
| 958 | Protein | NP_001198 | BTF3 | basic transcription factor 3 |
| 959 | DNA | NM_152260 | C18B11 | C18B11 homolog (44.9 kD) |
| 960 | Protein | NP_689473 | C18B11 | C18B11 homolog (44.9 kD) |
| 961 | DNA | NM_000146 | FTL | ferritin, light polypeptide |
| 962 | Protein | NP_000137 | FTL | ferritin, light polypeptide |
| 963 | DNA | W27417 | HSMPP8 | M-phase phosphoprotein, mpp8 |
| 964 | DNA | NM_012423 | RPL13A | ribosomal protein L13a |
| 965 | Protein | NP_036555 | RPL13A | ribosomal protein L13a |
| 966 | DNA | NM_005858 | AKAP8 | A kinase (PRKA) anchor |
| | | | | protein 8 |
| 967 | Protein | NP_005849 | AKAP8 | A kinase (PRKA) anchor |
| | | | | protein 8 |
| 968 | DNA | R59697 | | Homo sapiens mRNA |
| | | | | fragment, mRNA sequence |
| 969 | DNA | NM_002485 | NBS1 | Nijmegen breakage syndrome 1 |
| | | | | (nibrin) |
| 970 | Protein | NP_002476 | NBS1 | Nijmegen breakage syndrome 1 |
| | | | | (nibrin) |
| 971 | DNA | NM_003893 | LDB1 | LIM domain binding 1 |
| 972 | Protein | NP_003884 | LDB1 | LIM domain binding 1 |
| 973 | DNA | NM_014947 | KIAA1041 | KIAA1041 protein |
| 974 | Protein | NP_055762 | KIAA1041 | KIAA1041 protein |
| 975 | DNA | NM_006052 | DSCR3 | Down syndrome critical region |
| | | | | gene 3 |
| 976 | Protein | NP_006043 | DSCR3 | Down syndrome critical region |
| | | | | gene 3 |
| 977 | DNA | NM_138350 | LOC90326 | Homo sapiens hypothetical |
| | | | | protein MGC33488 |
| 978 | Protein | NP_612359 | LOC90326 | Homo sapiens hypothetical |
| | | | | protein MGC33488 |
| 979 | DNA | NM_012330 | MORF | monocytic leukemia zinc finger |
| | | | | protein-related factor |
| 980 | Protein | NP_036462 | MORF | monocytic leukemia zinc finger |
| | | | | protein-related factor |
| 981 | DNA | NM_007218 | TRC8 | patched related protein |
| | | | | translocated in renal cancer |
| 982 | Protein | NP_009149 | TRC8 | patched related protein |
| | | | | translocated in renal cancer |
| 983 | DNA | NM_003135 | SRP19 | signal recognition particle |
| | | | | 19 kDa |
| 984 | Protein | NP_003126 | SRP19 | signal recognition particle |
| | | | | 19 kDa |
| 985 | DNA | AA535884 | PCTK3 | PCTAIRE protein kinase 3 |
| 986 | DNA | NM_004860 | FXR2 | fragile X mental retardation, |
| | | | | autosomal homolog 2 |
| 987 | Protein | NP_004851 | FXR2 | fragile X mental retardation, |
| | | | | autosomal homolog 2 |
| 988 | DNA | NM_006698 | BLCAP | bladder cancer associated |
| | | | | protein |
| 989 | Protein | NP_006689 | BLCAP | bladder cancer associated |
| | | | | protein |
| 990 | DNA | NM_022826 | AXOT | axotrophin |
| 991 | Protein | NP_073737 | AXOT | axotrophin |
| 992 | DNA | NM_004597 | SNRPD2 | small nuclear ribonucleoprotein |
| | | | | D2 polypeptide 16.5 kDa |
| 993 | Protein | NP_004588 | SNRPD2 | small nuclear ribonucleoprotein |
| | | | | D2 polypeptide 16.5 kDa |
| 994 | DNA | NM_001032 | | Cluster Incl. |
| | | | | AI541542: libtest16.A02.r |
| | | | | Homo sapiens cDNA, 5′ end |
| | | | | /clone_end = 5′ /gb = AI541542 |
| | | | | /gi = 4458915 /ug = Hs.539 |
| | | | | /len = 639 |
| 995 | Protein | NP_001023 | | Cluster Incl. |
| | | | | AI541542: libtest16.A02.r |
| | | | | Homo sapiens cDNA, 5′ end |
| | | | | /clone_end = 5′ /gb = AI541542 |
| | | | | /gi = 4458915 /ug = Hs.539 |
| | | | | /len = 639 |
| 996 | DNA | NM_004356 | CD81 | CD81 antigen (target of |
| | | | | antiproliferative antibody 1) |
| 997 | Protein | NP_004347 | CD81 | CD81 antigen (target of |
| | | | | antiproliferative antibody 1) |
| 998 | DNA | NM_152758 | FLJ31657 | hypothetical protein FLJ31657 |
| 999 | Protein | NP_689971 | FLJ31657 | hypothetical protein FLJ31657 |
| 1000 | DNA | NM_012399 | PITPNB | phosphotidylinositol transfer |
| | | | | protein, beta |
| 1001 | Protein | NP_036531 | PITPNB | phosphotidylinositol transfer |
| | | | | protein, beta |
| 1002 | DNA | AL049941 | | Homo sapiens mRNA; cDNA |
| | | | | DKFZp564E2222 (from clone |
| | | | | DKFZp564E2222), mRNA |
| | | | | sequence |
| 1003 | DNA | NM_006362 | NXF1 | nuclear RNA export factor 1 |
| 1004 | Protein | NP_006353 | NXF1 | nuclear RNA export factor 1 |
| 1005 | DNA | NM_001358 | DDX15 | DEAD/H (Asp-Glu-Ala- |
| | | | | Asp/His) box polypeptide 15 |
| 1006 | Protein | NP_001349 | DDX15 | DEAD/H (Asp-Glu-Ala- |
| | | | | Asp/His) box polypeptide 15 |
| 1007 | DNA | NM_006570 | RAGA | Ras-related GTP-binding |
| | | | | protein |
| 1008 | Protein | NP_006561 | RAGA | Ras-related GTP-binding |
| | | | | protein |
| 1009 | DNA | NM_006565 | CTCF | CCCTC-binding factor (zinc |
| | | | | finger protein) |
| 1010 | Protein | NP_006556 | CTCF | CCCTC-binding factor (zinc |
| | | | | finger protein) |
| 1011 | DNA | NM_006852 | TLK2 | tousled-like kinase 2 |
| 1012 | Protein | NP_006843 | TLK2 | tousled-like kinase 2 |
| 1013 | DNA | NM_012289 | KEAP1 | Kelch-like ECH-associated |
| | | | | protein 1 |
| 1014 | Protein | NP_036421 | KEAP1 | Kelch-like ECH-associated |
| | | | | protein 1 |
| 1015 | DNA | NM_016322 | RAB14 | RAB14, member RAS |
| | | | | oncogene family |
| 1016 | Protein | NP_057406 | RAB14 | RAB14, member RAS |
| | | | | oncogene family |
| 1017 | DNA | NM_003756 | EIF3S3 | eukaryotic translation initiation |
| | | | | factor 3, subunit 3 gamma, |
| | | | | 40 kDa |
| 1018 | Protein | NP_003747 | EIF3S3 | eukaryotic translation initiation |
| | | | | factor 3, subunit 3 gamma, |
| | | | | 40 kDa |
| 1019 | DNA | NM_002569 | FURIN | furin (paired basic amino acid |
| | | | | cleaving enzyme) |
| 1020 | Protein | NP_002560 | FURIN | furin (paired basic amino acid |
| | | | | cleaving enzyme) |
| 1021 | DNA | NM_014862 | ARNT2 | aryl-hydrocarbon receptor |
| | | | | nuclear translocator 2 |
| 1022 | Protein | NP_055677 | ARNT2 | aryl-hydrocarbon receptor |
| | | | | nuclear translocator 2 |
| 1023 | DNA | NM_014966 | DDX30 | DEAD/H (Asp-Glu-Ala- |
| | | | | Asp/His) box polypeptide 30 |
| 1024 | Protein | NP_055781 | DDX30 | DEAD/H (Asp-Glu-Ala- |
| | | | | Asp/His) box polypeptide 30 |
| 1025 | DNA | NM_138614 | DDX30 | DEAD/H (Asp-Glu-Ala- |
| | | | | Asp/His) box polypeptide 30 |
| 1026 | Protein | NP_619519 | DDX30 | DEAD/H (Asp-Glu-Ala- |
| | | | | Asp/His) box polypeptide 30 |
| 1027 | DNA | NM_138615 | DDX30 | DEAD/H (Asp-Glu-Ala- |
| | | | | Asp/His) box polypeptide 30 |
| 1028 | Protein | NP_619520 | DDX30 | DEAD/H (Asp-Glu-Ala- |
| | | | | Asp/His) box polypeptide 30 |
| 1029 | DNA | NM_152301 | MGC9651 | hypothetical protein MGC9651 |
| 1030 | Protein | NP_689514 | MGC9651 | hypothetical protein MGC9651 |
| 1031 | DNA | NM_015317 | PUM2 | pumilio homolog 2 |
| | | | | (Drosophila) |
| 1032 | Protein | NP_056132 | PUM2 | pumilio homolog 2 |
| | | | | (Drosophila) |
| 1033 | DNA | NM_003457 | ZNF207 | zinc finger protein 207 |
| 1034 | Protein | NP_003448 | ZNF207 | zinc finger protein 207 |
| 1035 | DNA | M61906 | PIK3R1 | phosphoinositide-3-kinase, |
| | | | | regulatory subunit, polypeptide |
| | | | | 1 (p85 alpha) |
| 1036 | DNA | NM_015649 | DKFZP434M154 | DKFZP434M154 protein |
| 1037 | Protein | NP_056464 | DKFZP434M154 | DKFZP434M154 protein |
| 1038 | DNA | NM_004194 | ADAM22 | a disintegrin and |
| | | | | metalloproteinase domain 22 |
| 1039 | Protein | NP_004185 | ADAM22 | a disintegrin and |
| | | | | metalloproteinase domain 22 |
| 1040 | DNA | NM_016351 | ADAM22 | a disintegrin and |
| | | | | metalloproteinase domain 22 |
| 1041 | Protein | NP_057435 | ADAM22 | a disintegrin and |
| | | | | metalloproteinase domain 22 |
| 1042 | DNA | NM_021721 | ADAM22 | a disintegrin and |
| | | | | metalloproteinase domain 22 |
| 1043 | Protein | NP_068367 | ADAM22 | a disintegrin and |
| | | | | metalloproteinase domain 22 |
| 1044 | DNA | NM_005466 | MED6 | mediator of RNA polymerase II |
| | | | | transcription, subunit 6 |
| | | | | homolog (yeast) |
| 1045 | Protein | NP_005457 | MED6 | mediator of RNA polymerase II |
| | | | | transcription, subunit 6 |
| | | | | homolog (yeast) |
| 1046 | DNA | NM_004486 | GOLGA2 | golgi autoantigen, golgin |
| | | | | subfamily a, 2 |
| 1047 | Protein | NP_004477 | GOLGA2 | golgi autoantigen, golgin |
| | | | | subfamily a, 2 |
| 1048 | DNA | NM_021047 | ZNF253 | zinc finger protein 253 |
| 1049 | Protein | NP_066385 | ZNF253 | zinc finger protein 253 |
| 1050 | DNA | NM_017523 | HSXIAPAF1 | XIAP associated factor-1 |
| 1051 | Protein | NP_059993 | HSXIAPAF1 | XIAP associated factor-1 |
| 1052 | DNA | NM_014010 | ASTN2 | astrotactin 2 |
| 1053 | Protein | NP_054729 | ASTN2 | astrotactin 2 |
| 1054 | DNA | NM_006114 | TOMM40 | translocase of outer |
| | | | | mitochondrial membrane 40 |
| | | | | homolog (yeast) |
| 1055 | Protein | NP_006105 | TOMM40 | translocase of outer |
| | | | | mitochondrial membrane 40 |
| | | | | homolog (yeast) |
| 1056 | DNA | NM_006556 | PMVK | phosphomevalonate kinase |
| 1057 | Protein | NP_006547 | PMVK | phosphomevalonate kinase |
| 1058 | DNA | NM_020831 | MKL1 | megakaryoblastic leukemia |
| | | | | (translocation) 1 |
| 1059 | Protein | NP_065882 | MKL1 | megakaryoblastic leukemia |
| | | | | (translocation) 1 |
| 1060 | DNA | NM_003172 | SURF1 | surfeit 1 |
| 1061 | Protein | NP_003163 | SURF1 | surfeit 1 |
| 1062 | DNA | NM_005922 | MAP3K4 | mitogen-activated protein |
| | | | | kinase kinase kinase 4 |
| 1063 | Protein | NP_005913 | MAP3K4 | mitogen-activated protein |
| | | | | kinase kinase kinase 4 |
| 1064 | DNA | NM_006724 | MAP3K4 | mitogen-activated protein |
| | | | | kinase kinase kinase 4 |
| 1065 | Protein | NP_006715 | MAP3K4 | mitogen-activated protein |
| | | | | kinase kinase kinase 4 |
| 1066 | DNA | NM_015446 | ELYS | ELYS transcription factor-like |
| | | | | protein TMBS62 |
| 1067 | Protein | NP_056261 | ELYS | ELYS transcription factor-like |
| | | | | protein TMBS62 |
| 1068 | DNA | NM_002589 | PCDH7 | BH-protocadherin (brain-heart) |
| 1069 | Protein | NP_002580 | PCDH7 | BH-protocadherin (brain-heart) |
| 1070 | DNA | NM_032456 | PCDH7 | BH-protocadherin (brain-heart) |
| 1071 | Protein | NP_115832 | PCDH7 | BH-protocadherin (brain-heart) |
| 1072 | DNA | NM_032457 | PCDH7 | BH-protocadherin (brain-heart) |
| 1073 | Protein | NP_115833 | PCDH7 | BH-protocadherin (brain-heart) |
| 1074 | DNA | NM_020119 | ZAP | zinc finger antiviral protein |
| 1075 | Protein | NP_064504 | ZAP | zinc finger antiviral protein |
| 1076 | DNA | NM_024625 | ZAP | zinc finger antiviral protein |
| 1077 | Protein | NP_078901 | ZAP | zinc finger antiviral protein |
| 1078 | DNA | NM_001211 | BUB1B | BUB1 budding uninhibited by |
| | | | | benzimidazoles 1 homolog beta |
| | | | | (yeast) |
| 1079 | Protein | NP_001202 | BUB1B | BUB1 budding uninhibited by |
| | | | | benzimidazoles 1 homolog beta |
| | | | | (yeast) |
| 1080 | DNA | NM_014042 | DKFZP564M082 | DKFZP564M082 protein |
| 1081 | Protein | NP_054761 | DKFZP564M082 | DKFZP564M082 protein |
| 1082 | DNA | AB011178 | SCOP | SCN Circadian Oscillatory |
| | | | | Protein (SCOP) |
| 1083 | Protein | AB011178 | SCOP | SCN Circadian Oscillatory |
| | | (Translation) | | Protein (SCOP) |
| 1084 | DNA | NM_015542 | RENT2 | regulator of nonsense |
| | | | | transcripts 2 |
| 1085 | Protein | NP_056357 | RENT2 | regulator of nonsense |
| | | | | transcripts 2 |
| 1086 | DNA | NM_080599 | RENT2 | regulator of nonsense |
| | | | | transcripts 2 |
| 1087 | DNA | NM_005722 | ACTR2 | ARP2 actin-related protein 2 |
| | | | | homolog (yeast) |
| 1088 | Protein | NP_005713 | ACTR2 | ARP2 actin-related protein 2 |
| | | | | homolog (yeast) |
| 1089 | DNA | NM_021090 | MTMR3 | myotubularin related protein 3 |
| 1090 | Protein | NP_066576 | MTMR3 | myotubularin related protein 3 |
| 1091 | DNA | NM_153050 | MTMR3 | myotubularin related protein 3 |
| 1092 | Protein | NP_694690 | MTMR3 | myotubularin related protein 3 |
| 1093 | DNA | NM_153051 | MTMR3 | myotubularin related protein 3 |
| 1094 | Protein | NP_694691 | MTMR3 | myotubularin related protein 3 |
| 1095 | DNA | NM_003559 | PIP5K2B | phosphatidylinositol-4- |
| | | | | phosphate 5-kinase, type II, |
| | | | | beta |
| 1096 | Protein | NP_003550 | PIP5K2B | phosphatidylinositol-4- |
| | | | | phosphate 5-kinase, type II, |
| | | | | beta |
| 1097 | DNA | NM_138687 | PIP5K2B | phosphatidylinositol-4- |
| | | | | phosphate 5-kinase, type II, |
| | | | | beta |
| 1098 | Protein | NP_619632 | PIP5K2B | phosphatidylinositol-4- |
| | | | | phosphate 5-kinase, type II, |
| | | | | beta |
| 1099 | DNA | NM_006356 | ATP5H | ATP synthase, H+ transporting, |
| | | | | mitochondrial F0 complex, |
| | | | | subunit d |
| 1100 | Protein | NP_006347 | ATP5H | ATP synthase, H+ transporting, |
| | | | | mitochondrial F0 complex, |
| | | | | subunit d |
| 1101 | DNA | NM_015176 | KIAA0483 | KIAA0483 protein |
| 1102 | Protein | NP_055991 | KIAA0483 | KIAA0483 protein |
| 1103 | DNA | NM_003611 | OFD1 | oral-facial-digital syndrome 1 |
| 1104 | Protein | NP_003602 | OFD1 | oral-facial-digital syndrome 1 |
| 1105 | DNA | NM_002938 | RNF4 | ring finger protein 4 |
| 1106 | Protein | NP_002929 | RNF4 | ring finger protein 4 |
| 1107 | DNA | NM_015310 | EFA6R | ADP-ribosylation factor |
| | | | | guanine nucleotide factor 6 |
| 1108 | Protein | NP_056125 | EFA6R | ADP-ribosylation factor |
| | | | | guanine nucleotide factor 6 |
| 1109 | DNA | NM_015530 | GORASP2 | golgi reassembly stacking |
| | | | | protein 2, 55 kDa |
| 1110 | Protein | NP_056345 | GORASP2 | golgi reassembly stacking |
| | | | | protein 2, 55 kDa |
| 1111 | DNA | NM_006275 | Homo sapiens | Homo sapiens mRNA; cDNA |
| | | | splicing factor, | DKFZp564J223 (from clone |
| | | | arginine/serine- | DKFZp564J223), mRNA |
| | | | rich 6 | sequence |
| | | | (SFRS6), | |
| | | | mRNA | |
| 1112 | Protein | NP_006266 | Homo sapiens | Homo sapiens mRNA; cDNA |
| | | | splicing factor, | DKFZp564J223 (from clone |
| | | | arginine/serine- | DKFZp564J223), mRNA |
| | | | rich 6 | sequence |
| | | | (SFRS6) | |
| 1113 | DNA | NM_012470 | TRN-SR | transportin-SR |
| 1114 | Protein | NP_036602 | TRN-SR | transportin-SR |
| 1115 | DNA | NM_006360 | GA17 | dendritic cell protein |
| 1116 | Protein | NP_006351 | GA17 | dendritic cell protein |
| 1117 | DNA | NM_014159 | HIF1 | huntingtin interacting protein 1 |
| 1118 | Protein | NP_054878 | HIF1 | huntingtin interacting protein 1 |
| 1119 | DNA | NM_000100 | CSTB | cystatin B (stefin B) |
| 1120 | Protein | NP_000091 | CSTB | cystatin B (stefin B) |
| 1121 | DNA | NM_018947 | CYCS | cytochrome c, somatic |
| 1122 | Protein | NP_061820 | CYCS | cytochrome c, somatic |
| 1123 | DNA | NM_001312 | CRIP2 | cysteine-rich protein 2 |
| 1124 | Protein | NP_001303 | CRIP2 | cysteine-rich protein 2 |
| 1125 | DNA | AB002368 | RANBP20 | RAN binding protein 20 |
| 1126 | Protein | AB002368 | RANBP20 | RAN binding protein 20 |
| | | (Translation) | | |
| 1127 | DNA | NM_021188 | APA1 | likely ortholog of mouse |
| | | | | another partner for ARF 1 |
| 1128 | Protein | NP_067011 | APA1 | likely ortholog of mouse |
| | | | | another partner for ARF 1 |
| 1129 | DNA | NM_003129 | SQLE | squalene epoxidase |
| 1130 | Protein | NP_003120 | SQLE | squalene epoxidase |
| 1131 | DNA | NM_020357 | PCNP | PEST-containing nuclear |
| | | | | protein |
| 1132 | Protein | NP_065090 | PCNP | PEST-containing nuclear |
| | | | | protein |
| 1133 | DNA | NM_006323 | SEC24B | SEC24 related gene family, |
| | | | | member B (S. cerevisiae) |
| 1134 | Protein | NP_006314 | SEC24B | SEC24 related gene family, |
| | | | | member B (S. cerevisiae) |
| 1135 | DNA | AB028980 | USP24 | ubiquitin specific protease 24 |
| 1136 | Protein | AB028980 | USP24 | ubiquitin specific protease 24 |
| | | (Translation) | | |
| 1137 | DNA | AL049432 | RAI17 | retinoic acid induced 17 |
| 1138 | DNA | NM_015167 | PTDSR | phosphatidylserine receptor |
| 1139 | Protein | NP_055982 | PTDSR | phosphatidylserine receptor |
| 1140 | DNA | NM_000753 | PDE3B | phosphodiesterase 3B, cGMP- |
| | | | | inhibited |
| 1141 | Protein | NP_000744 | PDE3B | phosphodiesterase 3B, cGMP- |
| | | | | inhibited |
| 1142 | DNA | NM_000922 | PDE3B | phosphodiesterase 3B, cGMP- |
| | | | | inhibited |
| 1143 | Protein | NP_000913 | PDE3B | phosphodiesterase 3B, cGMP- |
| | | | | inhibited |
| 1144 | DNA | NM_005224 | DRIL1 | dead ringer-like 1 (Drosophila) |
| 1145 | Protein | NP_005215 | DRIL1 | dead ringer-like 1 (Drosophila) |
| 1146 | DNA | NM_002155 | HSPA6 | heat shock 70 kDa protein 6 |
| | | | | (HSP70B′) |
| 1147 | Protein | NP_002146 | HSPA6 | heat shock 70 kDa protein 6 |
| | | | | (HSP70B′) |
| 1148 | DNA | NM_012242 | DKK1 | dickkopf homolog 1 (Xenopus |
| | | | | laevis) |
| 1149 | Protein | NP_036374 | DKK1 | dickkopf homolog 1 (Xenopus |
| | | | | laevis) |
| 1150 | DNA | NM_004715 | CTDP1 | CTD (carboxy-terminal |
| | | | | domain, RNA polymerase II, |
| | | | | polypeptide A) phosphatase, |
| | | | | subunit 1 |
| 1151 | Protein | NP_004706 | CTDP1 | CTD (carboxy-terminal |
| | | | | domain, RNA polymerase II, |
| | | | | polypeptide A) phosphatase, |
| | | | | subunit 1 |
| 1152 | DNA | NM_048368 | CTDP1 | CTD (carboxy-terminal |
| | | | | domain, RNA polymerase II, |
| | | | | polypeptide A) phosphatase, |
| | | | | subunit 1 |
| 1153 | Protein | NP_430255 | CTDP1 | CTD (carboxy-terminal |
| | | | | domain, RNA polymerase II, |
| | | | | polypeptide A) phosphatase, |
| | | | | subunit 1 |
| 1154 | DNA | NM_001952 | E2F6 | E2F transcription factor 6 |
| 1155 | Protein | NP_001943 | E2F6 | E2F transcription factor 6 |
| 1156 | DNA | NM_014939 | KIAA1012 | KIAA1012 protein |
| 1157 | Protein | NP_055754 | KIAA1012 | KIAA1012 protein |
| 1158 | DNA | NM_006250 | PRH1 | proline-rich protein HaeIII |
| | | | | subfamily 1 |
| 1159 | Protein | NP_006241 | PRH1 | proline-rich protein HaeIII |
| | | | | subfamily 1 |
| 1160 | DNA | NM_021974 | POLR2F | polymerase (RNA) II (DNA |
| | | | | directed) polypeptide F |
| 1161 | Protein | NP_068809 | POLR2F | polymerase (RNA) II (DNA |
| | | | | directed) polypeptide F |
| 1162 | DNA | NM_001584 | C11orf8 | chromosome 11 open reading |
| | | | | frame 8 |
| 1163 | Protein | NP_001575 | C11orf8 | chromosome 11 open reading |
| | | | | frame 8 |
| 1164 | DNA | NM_015438 | DKFZP586I2223 | intermediate filament-like |
| | | | | MGC: 2625 |
| 1165 | Protein | NP_056253 | DKFZP586I2223 | intermediate filament-like |
| | | | | MGC: 2625 |
| 1166 | DNA | NM_080730 | DKFZP586I2223 | intermediate filament-like |
| | | | | MGC: 2625 |
| 1167 | Protein | NP_542768 | DKFZP586I2223 | intermediate filament-like |
| | | | | MGC: 2625 |
| 1168 | DNA | NM_080731 | DKFZP586I2223 | intermediate filament-like |
| | | | | MGC: 2625 |
| 1169 | Protein | NP_542769 | DKFZP586I2223 | intermediate filament-like |
| | | | | MGC: 2625 |
| 1170 | DNA | NM_003801 | GPAA1 | GPAA1P anchor attachment |
| | | | | protein 1 homolog (yeast) |
| 1171 | Protein | NP_003792 | GPAA1 | GPAA1P anchor attachment |
| | | | | protein 1 homolog (yeast) |
| 1172 | DNA | NM_000347 | SPTB | spectrin, beta, erythrocytic |
| | | | | (includes spherocytosis, clinical |
| | | | | type I) |
| 1173 | Protein | NP_000338 | SPTB | spectrin, beta, erythrocytic |
| | | | | (includes spherocytosis, clinical |
| | | | | type I) |
| 1174 | DNA | NM_003686 | EXO1 | exonuclease 1 |
| 1175 | Protein | NP_003677 | EXO1 | exonuclease 1 |
| 1176 | DNA | NM_006027 | EXO1 | exonuclease 1 |
| 1177 | Protein | NP_006018 | EXO1 | exonuclease 1 |
| 1178 | DNA | NM_130398 | EXO1 | exonuclease 1 |
| 1179 | Protein | NP_569082 | EXO1 | exonuclease 1 |
| 1180 | DNA | NM_014345 | ZFP318 | endocrine regulator |
| 1181 | Protein | NP_055160 | ZFP318 | endocrine regulator |
| 1182 | DNA | NM_001262 | CDKN2C | cyclin-dependent kinase |
| | | | | inhibitor 2C (p18, inhibits |
| | | | | CDK4) |
| 1183 | Protein | NP_001253 | CDKN2C | cyclin-dependent kinase |
| | | | | inhibitor 2C (p18, inhibits |
| | | | | CDK4) |
| 1184 | DNA | NM_078626 | CDKN2C | cyclin-dependent kinase |
| | | | | inhibitor 2C (p18, inhibits |
| | | | | CDK4) |
| 1185 | DNA | AB020699 | KIAA0892 | KIAA0892 protein |
| 1186 | Protein | AB020699 | KIAA0892 | KIAA0892 protein |
| | | (Translation) | | |
| 1187 | DNA | AB007925 | FNBP2 | formin binding protein 2 |
| 1188 | Protein | AB007925 | FNBP2 | formin binding protein 2 |
| | | (Translation) | | |
| 1189 | DNA | NM_004898 | CLOCK | clock homolog (mouse) |
| 1190 | Protein | NP_004889 | CLOCK | clock homolog (mouse) |
| 1191 | DNA | NM_003720 | DSCR2 | Down syndrome critical region |
| | | | | gene 2 |
| 1192 | Protein | NP_003711 | DSCR2 | Down syndrome critical region |
| | | | | gene 2 |
| 1193 | DNA | NM_006924 | SFRS1 | splicing factor, arginine/serine- |
| | | | | rich 1 (splicing factor 2, |
| | | | | alternate splicing factor) |
| 1194 | Protein | NP_008855 | SFRS1 | splicing factor, arginine/serine- |
| | | | | rich 1 (splicing factor 2, |
| | | | | alternate splicing factor) |
| 1195 | DNA | NM_004326 | BCL9 | B-cell CLL/lymphoma 9 |
| 1196 | Protein | NP_004317 | BCL9 | B-cell CLL/lymphoma 9 |
| 1197 | DNA | NM_003283 | TNNT1 | troponin T1, skeletal, slow |
| 1198 | Protein | NP_003274 | TNNT1 | troponin T1, skeletal, slow |
| 1199 | DNA | NM_021126 | MPST | mercaptopyruvate |
| | | | | sulfurtransferase |
| 1200 | Protein | NP_066949 | MPST | mercaptopyruvate |
| | | | | sulfurtransferase |
| 1201 | DNA | NM_001182 | ALDH7A1 | aldehyde dehydrogenase 7 |
| | | | | family, member A1 |
| 1202 | Protein | NP_001173 | ALDH7A1 | aldehyde dehydrogenase 7 |
| | | | | family, member A1 |
| 1203 | DNA | NM_001749 | CAPNS1 | calpain, small subunit 1 |
| 1204 | Protein | NP_001740 | CAPNS1 | calpain, small subunit 1 |
| 1205 | DNA | NM_004346 | CASP3 | caspase 3, apoptosis-related |
| | | | | cysteine protease |
| 1206 | Protein | NP_004337 | CASP3 | caspase 3, apoptosis-related |
| | | | | cysteine protease |
| 1207 | DNA | NM_032991 | CASP3 | caspase 3, apoptosis-related |
| | | | | cysteine protease |
| 1208 | DNA | NM_003145 | SSR2 | signal sequence receptor, beta |
| | | | | (translocon-associated protein |
| | | | | beta) |
| 1209 | Protein | NP_003136 | SSR2 | signal sequence receptor, beta |
| | | | | (translocon-associated protein |
| | | | | beta) |
| 1210 | DNA | NM_153273 | IHPK1 | inositol hexaphosphate kinase 1 |
| 1211 | Protein | NP_695005 | IHPK1 | inositol hexaphosphate kinase 1 |
| 1212 | DNA | NM_001728 | BSG | basigin (OK blood group) |
| 1213 | Protein | NP_001719 | BSG | basigin (OK blood group) |
| 1214 | DNA | NM_004374 | COX6C | cytochrome c oxidase subunit |
| | | | | VIc |
| 1215 | Protein | NP_004365 | COX6C | cytochrome c oxidase subunit |
| | | | | VIc |
| 1216 | DNA | NM_004047 | ATP6V0B | ATPase, H+ transporting, |
| | | | | lysosomal 21 kDa, V0 subunit |
| | | | | c″ |
| 1217 | Protein | NP_004038 | ATP6V0B | ATPase, H+ transporting, |
| | | | | lysosomal 21 kDa, V0 subunit |
| | | | | c″ |
| 1218 | DNA | NM_004541 | NDUFA1 | NADH dehydrogenase |
| | | | | (ubiquinone) 1 alpha |
| | | | | subcomplex, 1, 7.5 kDa |
| 1219 | Protein | NP_004532 | NDUFA1 | NADH dehydrogenase |
| | | | | (ubiquinone) 1 alpha |
| | | | | subcomplex, 1, 7.5 kDa |
| 1220 | DNA | NM_014297 | YF13H12 | protein expressed in thyroid |
| 1221 | Protein | NP_055112 | YF13H12 | protein expressed in thyroid |
| 1222 | DNA | NM_004759 | MAPKAPK2 | mitogen-activated protein |
| | | | | kinase-activated protein kinase 2 |
| 1223 | Protein | NP_004750 | MAPKAPK2 | mitogen-activated protein |
| | | | | kinase-activated protein kinase 2 |
| 1224 | DNA | NM_032960 | MAPKAPK2 | mitogen-activated protein |
| | | | | kinase-activated protein kinase 2 |
| 1225 | Protein | NP_116584 | MAPKAPK2 | mitogen-activated protein |
| | | | | kinase-activated protein kinase 2 |
| 1226 | DNA | NM_000289 | PFKM | phosphofructokinase, muscle |
| 1227 | Protein | NP_000280 | PFKM | phosphofructokinase, muscle |
| 1228 | DNA | NM_005104 | BRD2 | bromodomain containing 2 |
| 1229 | Protein | NP_005095 | BRD2 | bromodomain containing 2 |
| 1230 | DNA | NM_004235 | KLF4 | Kruppel-like factor 4 (gut) |
| 1231 | Protein | NP_004226 | KLF4 | Kruppel-like factor 4 (gut) |
| 1232 | DNA | NM_007271 | STK38 | serine/threonine kinase 38 |
| 1233 | Protein | NP_009202 | STK38 | serine/threonine kinase 38 |
| 1234 | DNA | NM_138448 | ACYP2 | acylphosphatase 2, muscle type |
| 1235 | Protein | NP_612457 | ACYP2 | acylphosphatase 2, muscle type |
| 1236 | DNA | NM_003045 | SLC7A1 | solute carrier family 7 (cationic |
| | | | | amino acid transporter, y+ |
| | | | | system), member 1 |
| 1237 | Protein | NP_003036 | SLC7A1 | solute carrier family 7 (cationic |
| | | | | amino acid transporter, y+ |
| | | | | system), member 1 |
| 1238 | DNA | NM_002446 | MAP3K10 | mitogen-activated protein |
| | | | | kinase kinase kinase 10 |
| 1239 | Protein | NP_002437 | MAP3K10 | mitogen-activated protein |
| | | | | kinase kinase kinase 10 |
| 1240 | DNA | NM_003429 | ZNF85 | zinc finger protein 85 (HPF4, |
| | | | | HTF1) |
| 1241 | Protein | NP_003420 | ZNF85 | zinc finger protein 85 (HPF4, |
| | | | | HTF1) |
| 1242 | DNA | NM_005547 | IVL | involucrin |
| 1243 | Protein | NP_005538 | IVL | involucrin |
| 1244 | DNA | NM_000661 | RPL9 | ribosomal protein L9 |
| 1245 | Protein | NP_000652 | RPL9 | ribosomal protein L9 |
| 1246 | DNA | W28729 | EST | EST |
| 1247 | DNA | NM_052855 | MGC15396 | hypothetical protein |
| | | | | MGC15396 |
| 1248 | Protein | NP_443087 | MGC15396 | hypothetical protein |
| | | | | MGC15396 |
| 1249 | DNA | NM_004160 | PYY | peptide YY |
| 1250 | Protein | NP_004151 | PYY | peptide YY |
| 1251 | DNA | NM_004875 | RPA40 | RNA polymerase I subunit |
| 1252 | Protein | NP_004866 | RPA40 | RNA polymerase I subunit |
| 1253 | DNA | NM_014291 | GCAT | glycine C-acetyltransferase (2- |
| | | | | amino-3-ketobutyrate |
| | | | | coenzyme A ligase) |
| 1254 | Protein | NP_055106 | GCAT | glycine C-acetyltransferase (2- |
| | | | | amino-3-ketobutyrate |
| | | | | coenzyme A ligase) |
| 1255 | DNA | NM_007344 | TTF1 | transcription termination factor, |
| | | | | RNA polymerase I |
| 1256 | Protein | NP_031370 | TTF1 | transcription termination factor, |
| | | | | RNA polymerase I |
| 1257 | DNA | NM_005632 | SOLH | small optic lobes homolog |
| | | | | (Drosophila) |
| 1258 | Protein | NP_005623 | SOLH | small optic lobes homolog |
| | | | | (Drosophila) |
| 1259 | DNA | AB011542 | EGFL5 | EGF-like-domain, multiple 5 |
| 1260 | Protein | AB011542 | EGFL5 | EGF-like-domain, multiple 5 |
| | | (Translation) | | |
| 1261 | DNA | NM_021003 | PPM1A | protein phosphatase 1A |
| | | | | (formerly 2C), magnesium- |
| | | | | dependent, alpha isoform |
| 1262 | Protein | NP_066283 | PPM1A | protein phosphatase 1A |
| | | | | (formerly 2C), magnesium- |
| | | | | dependent, alpha isoform |
| 1263 | DNA | D30612 | ZNF282 | zinc finger protein 282 |
| 1264 | Protein | D30612 (Translation) | ZNF282 | zinc finger protein 282 |
| 1265 | DNA | NM_005476 | GNE | UDP-N-acetylglucosamine-2- |
| | | | | epimerase/N- |
| | | | | acetylmannosamine kinase |
| 1266 | Protein | NP_005467 | GNE | UDP-N-acetylglucosamine-2- |
| | | | | epimerase/N- |
| | | | | acetylmannosamine kinase |
| 1267 | DNA | NM_005926 | MFAP1 | microfibrillar-associated |
| | | | | protein 1 |
| 1268 | Protein | NP_005917 | MFAP1 | microfibrillar-associated |
| | | | | protein 1 |
| 1269 | DNA | NM_006359 | SLC9A6 | solute carrier family 9 |
| | | | | (sodium/hydrogen exchanger), |
| | | | | isoform 6 |
| 1270 | Protein | NP_006350 | SLC9A6 | solute carrier family 9 |
| | | | | (sodium/hydrogen exchanger), |
| | | | | isoform 6 |
| 1271 | DNA | NM_003087 | SNCG | synuclein, gamma (breast |
| | | | | cancer-specific protein 1) |
| 1272 | Protein | NP_003078 | SNCG | synuclein, gamma (breast |
| | | | | cancer-specific protein 1) |
| 1273 | DNA | NM_153341 | FLJ90005 | hypothetical protein FLJ90005 |
| 1274 | Protein | NP_699172 | FLJ90005 | hypothetical protein FLJ90005 |
| 1275 | DNA | NM_006978 | ZNF183 | zinc finger protein 183 (RING |
| | | | | finger, C3HC4 type) |
| 1276 | Protein | NP_008909 | ZNF183 | zinc finger protein 183 (RING |
| | | | | finger, C3HC4 type) |
| 1277 | DNA | NM_004135 | IDH3G | isocitrate dehydrogenase 3 |
| | | | | (NAD+) gamma |
| 1278 | Protein | NP_004126 | IDH3G | isocitrate dehydrogenase 3 |
| | | | | (NAD+) gamma |
| 1279 | DNA | NM_174869 | IDH3G | isocitrate dehydrogenase 3 |
| | | | | (NAD+) gamma |
| 1280 | Protein | NP_777358 | IDH3G | isocitrate dehydrogenase 3 |
| | | | | (NAD+) gamma |
| 1281 | DNA | NM_001166 | BIRC2 | baculoviral IAP repeat- |
| | | | | containing 2 |
| 1282 | Protein | NP_001157 | BIRC2 | baculoviral IAP repeat- |
| | | | | containing 2 |
| 1283 | DNA | NM_004788 | UBE4A | ubiquitination factor E4A |
| | | | | (UFD2 homolog, yeast) |
| 1284 | Protein | NP_004779 | UBE4A | ubiquitination factor E4A |
| | | | | (UFD2 homolog, yeast) |
| 1285 | DNA | D87470 | KIAA0280 | KIAA0280 protein |
| 1286 | Protein | D87470 (Translation) | KIAA0280 | KIAA0280 protein |
| 1287 | DNA | NM_006010 | ARMET | arginine-rich, mutated in early |
| | | | | stage tumors |
| 1288 | Protein | NP_006001 | ARMET | arginine-rich, mutated in early |
| | | | | stage tumors |
| 1289 | DNA | NM_002165 | ID1 | inhibitor of DNA binding 1, |
| | | | | dominant negative helix-loop- |
| | | | | helix protein |
| 1290 | Protein | NP_002156 | ID1 | inhibitor of DNA binding 1, |
| | | | | dominant negative helix-loop- |
| | | | | helix protein |
| 1291 | DNA | NM_000454 | SOD1 | superoxide dismutase 1, soluble |
| | | | | (amyotrophic lateral sclerosis 1 |
| | | | | (adult)) |
| 1292 | Protein | NP_000445 | SOD1 | superoxide dismutase 1, soluble |
| | | | | (amyotrophic lateral sclerosis 1 |
| | | | | (adult)) |
| 1293 | DNA | NM_007202 | AKAP10 | A kinase (PRKA) anchor |
| | | | | protein 10 |
| 1294 | Protein | NP_009133 | AKAP10 | A kinase (PRKA) anchor |
| | | | | protein 10 |
| 1295 | DNA | J00287 | | Cluster Incl. J00287: Human |
| | | | | pepsinogen gene |
| | | | | /cds = (55.1221) /gb = J00287 |
| | | | | /gi = 189798 /ug = Hs.75558 |
| | | | | /len = 1381 |
| 1296 | Protein | J00287 (Translation) | | Cluster Incl. J00287: Human |
| | | | | pepsinogen gene |
| | | | | /cds = (55.1221) /gb = J00287 |
| | | | | /gi = 189798 /ug = Hs.75558 |
| | | | | /len = 1381 |
| 1297 | DNA | NM_004357 | CD151 | CD151 antigen |
| 1298 | Protein | NP_004348 | CD151 | CD151 antigen |
| 1299 | DNA | NM_139030 | CD151 | CD151 antigen |
| 1300 | Protein | NP_620599 | CD151 | CD151 antigen |
| 1301 | DNA | NM_139031 | CD151 | CD151 antigen |
| 1302 | DNA | NM_004270 | CRSP9 | cofactor required for Sp1 |
| | | | | transcriptional activation, |
| | | | | subunit 9, 33 kDa |
| 1303 | Protein | NP_004261 | CRSP9 | cofactor required for Sp1 |
| | | | | transcriptional activation, |
| | | | | subunit 9, 33 kDa |
| 1304 | DNA | NM_000375 | UROS | uroporphyrinogen III synthase |
| | | | | (congenital erythropoietic |
| | | | | porphyria) |
| 1305 | Protein | NP_000366 | UROS | uroporphyrinogen III synthase |
| | | | | (congenital erythropoietic |
| | | | | porphyria) |
| 1306 | DNA | NM_000155 | GALT | galactose-1-phosphate |
| | | | | uridylyltransferase |
| 1307 | Protein | NP_000146 | GALT | galactose-1-phosphate |
| | | | | uridylyltransferase |
| 1308 | DNA | NM_147131 | GALT | galactose-1-phosphate |
| | | | | uridylyltransferase |
| 1309 | Protein | NP_667342 | GALT | galactose-1-phosphate |
| | | | | uridylyltransferase |
| 1310 | DNA | NM_147132 | GALT | galactose-1-phosphate |
| | | | | uridylyltransferase |
| 1311 | Protein | NP_667343 | GALT | galactose-1-phosphate |
| | | | | uridylyltransferase |
| 1312 | DNA | NM_000918 | P4HB | procollagen-proline, 2- |
| | | | | oxoglutarate 4-dioxygenase |
| | | | | (proline 4-hydroxylase), beta |
| | | | | polypeptide (protein disulfide |
| | | | | isomerase; thyroid hormone |
| | | | | binding protein p55) |
| 1313 | Protein | NP_000909 | P4HB | procollagen-proline, 2- |
| | | | | oxoglutarate 4-dioxygenase |
| | | | | (proline 4-hydroxylase), beta |
| | | | | polypeptide (protein disulfide |
| | | | | isomerase; thyroid hormone |
| | | | | binding protein p55) |
| 1314 | DNA | NM_005022 | PFN1 | profilin 1 |
| 1315 | Protein | NP_005013 | PFN1 | profilin 1 |
| 1316 | DNA | NM_001647 | APOD | apolipoprotein D |
| 1317 | Protein | NP_001638 | APOD | apolipoprotein D |
| 1318 | DNA | NM_153747 | | Cluster Incl. AB000359: Homo |
| | | | | sapiens PIGCP1 pseudogene |
| | | | | /cds = (0.416) /gb = AB000359 |
| | | | | /gi = 2547040 /ug = Hs.47974 |
| | | | | /len = 417 |
| 1319 | Protein | NP_714969 | | Cluster Incl. AB000359: Homo |
| | | | | sapiens PIGCP1 pseudogene |
| | | | | /cds = (0.416) /gb = AB000359 |
| | | | | /gi = 2547040 /ug = Hs.47974 |
| | | | | /len = 417 |
| 1320 | DNA | NM_002642 | | Cluster Incl. AB000359: Homo |
| | | | | sapiens PIGCP1 pseudogene |
| | | | | /cds = (0.416) /gb = AB000359 |
| | | | | /gi = 2547040 /ug = Hs.47974 |
| | | | | /len = 417 |
| 1321 | DNA | AL080093 | | Homo sapiens mRNA; cDNA |
| | | | | DKFZp564N1662 (from clone |
| | | | | DKFZp564N1662), mRNA |
| | | | | sequence |
| 1322 | DNA | NM_001831 | CLU | clusterin (complement lysis |
| | | | | inhibitor, SP-40,40, sulfated |
| | | | | glycoprotein 2, testosterone- |
| | | | | repressed prostate message 2, |
| | | | | apolipoprotein J) |
| 1323 | Protein | NP_001822 | CLU | clusterin (complement lysis |
| | | | | inhibitor, SP-40,40, sulfated |
| | | | | glycoprotein 2, testosterone- |
| | | | | repressed prostate message 2, |
| | | | | apolipoprotein J) |
| 1324 | DNA | NM_015852 | H-plk | Krueppel-related zinc finger |
| | | | | protein |
| 1325 | Protein | NP_056936 | H-plk | Krueppel-related zinc finger |
| | | | | protein |
| 1326 | DNA | NM_001318 | CSHL1 | chorionic somatomammotropin |
| | | | | hormone-like 1 |
| 1327 | Protein | NP_001309 | CSHL1 | chorionic somatomammotropin |
| | | | | hormone-like 1 |
| 1328 | DNA | NM_022578 | CSHL1 | chorionic somatomammotropin |
| | | | | hormone-like 1 |
| 1329 | Protein | NP_072100 | CSHL1 | chorionic somatomammotropin |
| | | | | hormone-like 1 |
| 1330 | DNA | NM_022579 | CSHL1 | chorionic somatomammotropin |
| | | | | hormone-like 1 |
| 1331 | Protein | NP_072101 | CSHL1 | chorionic somatomammotropin |
| | | | | hormone-like 1 |
| 1332 | DNA | NM_022580 | CSHL1 | chorionic somatomammotropin |
| | | | | hormone-like 1 |
| 1333 | Protein | NP_072102 | CSHL1 | chorionic somatomammotropin |
| | | | | hormone-like 1 |
| 1334 | DNA | NM_001540 | | 28 kDa heat shock protein |
| | | | | [Homo sapiens], mRNA |
| | | | | sequence |
| 1335 | Protein | NP_001531 | | 28 kDa heat shock protein |
| | | | | [Homo sapiens], mRNA |
| | | | | sequence |
| 1336 | DNA | NM_007104 | RPL10A | ribosomal protein L10a |
| 1337 | Protein | NP_009035 | RPL10A | ribosomal protein L10a |
| 1338 | DNA | NM_002778 | PSAP | prosaposin (variant Gaucher |
| | | | | disease and variant |
| | | | | metachromatic leukodystrophy) |
| 1339 | Protein | NP_002769 | PSAP | prosaposin (variant Gaucher |
| | | | | disease and variant |
| | | | | metachromatic leukodystrophy) |
| 1340 | DNA | NM_001466 | FZD2 | frizzled homolog 2 |
| | | | | (Drosophila) |
| 1341 | Protein | NP_001457 | FZD2 | frizzled homolog 2 |
| | | | | (Drosophila) |
| 1342 | DNA | NM_022735 | GOCAP1 | golgi complex associated |
| | | | | protein 1, 60 kDa |
| 1343 | Protein | NP_073572 | GOCAP1 | golgi complex associated |
| | | | | protein 1, 60 kDa |
| 1344 | DNA | AB002324 | KIAA0326 | KIAA0326 protein |
| 1345 | Protein | AB002324 | KIAA0326 | KIAA0326 protein |
| | | (Translation) | | |
| 1346 | DNA | NM_006845 | KNSL6 | kinesin-like 6 (mitotic |
| | | | | centromere-associated kinesin) |
| 1347 | Protein | NP_006836 | KNSL6 | kinesin-like 6 (mitotic |
| | | | | centromere-associated kinesin) |
| 1348 | DNA | NM_001254 | CDC6 | CDC6 cell division cycle 6 |
| | | | | homolog (S. cerevisiae) |
| 1349 | Protein | NP_001245 | CDC6 | CDC6 cell division cycle 6 |
| | | | | homolog (S. cerevisiae) |
| 1350 | DNA | D50926 | NXP-2 | nuclear matrix protein NXP-2 |
| 1351 | Protein | D50926 (Translation) | NXP-2 | nuclear matrix protein NXP-2 |
| 1352 | DNA | NM_016199 | LSM7 | U6 snRNA-associated Sm-like |
| | | | | protein LSm7 |
| 1353 | Protein | NP_057283 | LSM7 | U6 snRNA-associated Sm-like |
| | | | | protein LSm7 |
| 1354 | DNA | NM_002853 | RAD1 | RAD1 homolog (S. pombe) |
| 1355 | Protein | NP_002844 | RAD1 | RAD1 homolog (S. pombe) |
| 1356 | DNA | NM_133282 | RAD1 | RAD1 homolog (S. pombe) |
| 1357 | Protein | NP_579816 | RAD1 | RAD1 homolog (S. pombe) |
| 1358 | DNA | NM_133377 | RAD1 | RAD1 homolog (S. pombe) |
| 1359 | DNA | NM_015169 | RRS1 | homolog of yeast ribosome |
| | | | | biogenesis regulatory protein |
| | | | | RRS1 |
| 1360 | Protein | NP_055984 | RRS1 | homolog of yeast ribosome |
| | | | | biogenesis regulatory protein |
| | | | | RRS1 |
| 1361 | DNA | AB028987 | C19orf7 | chromosome 19 open reading |
| | | | | frame 7 |
| 1362 | Protein | AB028987 | C19orf7 | chromosome 19 open reading |
| | | (Translation) | | frame 7 |
| 1363 | DNA | NM_014213 | HOXD9 | homeo box D9 |
| 1364 | Protein | NP_055028 | HOXD9 | homeo box D9 |
| 1365 | DNA | NM_003344 | UBE2H | ubiquitin-conjugating enzyme |
| | | | | E2H (UBC8 homolog, yeast) |
| 1366 | Protein | NP_003335 | UBE2H | ubiquitin-conjugating enzyme |
| | | | | E2H (UBC8 homolog, yeast) |
| 1367 | DNA | NM_001665 | ARHG | ras homolog gene family, |
| | | | | member G (rho G) |
| 1368 | Protein | NP_001656 | ARHG | ras homolog gene family, |
| | | | | member G (rho G) |
| 1369 | DNA | NM_003188 | MAP3K7 | mitogen-activated protein |
| | | | | kinase kinase kinase 7 |
| 1370 | Protein | NP_003179 | MAP3K7 | mitogen-activated protein |
| | | | | kinase kinase kinase 7 |
| 1371 | DNA | NM_145331 | MAP3K7 | mitogen-activated protein |
| | | | | kinase kinase kinase 7 |
| 1372 | Protein | NP_663304 | MAP3K7 | mitogen-activated protein |
| | | | | kinase kinase kinase 7 |
| 1373 | DNA | NM_145332 | MAP3K7 | mitogen-activated protein |
| | | | | kinase kinase kinase 7 |
| 1374 | Protein | NP_663305 | MAP3K7 | mitogen-activated protein |
| | | | | kinase kinase kinase 7 |
| 1375 | DNA | NM_145333 | MAP3K7 | mitogen-activated protein |
| | | | | kinase kinase kinase 7 |
| 1376 | Protein | NP_663306 | MAP3K7 | mitogen-activated protein |
| | | | | kinase kinase kinase 7 |
| 1377 | DNA | NM_003390 | WEE1 | WEE1 homolog (S. pombe) |
| 1378 | Protein | NP_003381 | WEE1 | WEE1 homolog (S. pombe) |
| 1379 | DNA | NM_006527 | SLBP | stem-loop (histone) binding |
| | | | | protein |
| 1380 | Protein | NP_006518 | SLBP | stem-loop (histone) binding |
| | | | | protein |
| 1381 | DNA | NM_000856 | GUCY1A3 | guanylate cyclase 1, soluble, |
| | | | | alpha 3 |
| 1382 | Protein | NP_000847 | GUCY1A3 | guanylate cyclase 1, soluble, |
| | | | | alpha 3 |
| 1383 | DNA | NM_002748 | MAPK6 | mitogen-activated protein |
| | | | | kinase 6 |
| 1384 | Protein | NP_002739 | MAPK6 | mitogen-activated protein |
| | | | | kinase 6 |
| 1385 | DNA | NM_007145 | ZNF146 | zinc finger protein 146 |
| 1386 | Protein | NP_009076 | ZNF146 | zinc finger protein 146 |
| 1387 | DNA | NM_003186 | TAGLN | transgelin |
| 1388 | Protein | NP_003177 | TAGLN | transgelin |
| 1389 | DNA | NM_014761 | KIAA0174 | KIAA0174 gene product |
| 1390 | Protein | NP_055576 | KIAA0174 | KIAA0174 gene product |
| 1391 | DNA | NM_001396 | DYRK1A | dual-specificity tyrosine-(Y)- |
| | | | | phosphorylation regulated |
| | | | | kinase 1A |
| 1392 | Protein | NP_001387 | DYRK1A | dual-specificity tyrosine-(Y)- |
| | | | | phosphorylation regulated |
| | | | | kinase 1A |
| 1393 | DNA | NM_101395 | DYRK1A | dual-specificity tyrosine-(Y)- |
| | | | | phosphorylation regulated |
| | | | | kinase 1A |
| 1394 | Protein | NP_567824 | DYRK1A | dual-specificity tyrosine-(Y)- |
| | | | | phosphorylation regulated |
| | | | | kinase 1A |
| 1395 | DNA | NM_130436 | DYRK1A | dual-specificity tyrosine-(Y)- |
| | | | | phosphorylation regulated |
| | | | | kinase 1A |
| 1396 | Protein | NP_569120 | DYRK1A | dual-specificity tyrosine-(Y)- |
| | | | | phosphorylation regulated |
| | | | | kinase 1A |
| 1397 | DNA | NM_000182 | HADHA | hydroxyacyl-Coenzyme A |
| | | | | dehydrogenase/3-ketoacyl- |
| | | | | Coenzyme A thiolase/enoyl- |
| | | | | Coenzyme A hydratase |
| | | | | (trifunctional protein), alpha |
| | | | | subunit |
| 1398 | Protein | NP_000173 | HADHA | hydroxyacyl-Coenzyme A |
| | | | | dehydrogenase/3-ketoacyl- |
| | | | | Coenzyme A thiolase/enoyl- |
| | | | | Coenzyme A hydratase |
| | | | | (trifunctional protein), alpha |
| | | | | subunit |
| 1399 | DNA | NM_005359 | MADH4 | MAD, mothers against |
| | | | | decapentaplegic homolog 4 |
| | | | | (Drosophila) |
| 1400 | Protein | NP_005350 | MADH4 | MAD, mothers against |
| | | | | decapentaplegic homolog 4 |
| | | | | (Drosophila) |
| 1401 | DNA | NM_012408 | PRKCBP1 | protein kinase C binding |
| | | | | protein 1 |
| 1402 | Protein | NP_036540 | PRKCBP1 | protein kinase C binding |
| | | | | protein 1 |
| 1403 | DNA | AL050353 | OIP2 | Opa-interacting protein 2 |
| 1404 | DNA | NM_004181 | UCHL1 | ubiquitin carboxyl-terminal |
| | | | | esterase L1 (ubiquitin |
| | | | | thiolesterase) |
| 1405 | Protein | NP_004172 | UCHL1 | ubiquitin carboxyl-terminal |
| | | | | esterase L1 (ubiquitin |
| | | | | thiolesterase) |
| 1406 | DNA | NM_005626 | SFRS4 | splicing factor, arginine/serine- |
| | | | | rich 4 |
| 1407 | Protein | NP_005617 | SFRS4 | splicing factor, arginine/serine- |
| | | | | rich 4 |
| 1408 | DNA | NM_001694 | ATP6V0C | ATPase, H+ transporting, |
| | | | | lysosomal 16 kDa, V0 subunit c |
| 1409 | Protein | NP_001685 | ATP6V0C | ATPase, H+ transporting, |
| | | | | lysosomal 16 kDa, V0 subunit c |
| 1410 | DNA | M88249 | | Cluster Incl. M88249: Human |
| | | | | inter-alpha-trypsin inhibitor |
| | | | | light chain (ITI) gene |
| | | | | /cds = (94.1152) /gb = M88249 |
| | | | | /gi = 186599 /ug = Hs.76177 |
| | | | | /len = 1262 |
| 1411 | Protein | AAA59196 | | Cluster Incl. M88249: Human |
| | | | | inter-alpha-trypsin inhibitor |
| | | | | light chain (ITI) gene |
| | | | | /cds = (94.1152) /gb = M88249 |
| | | | | /gi = 186599 /ug = Hs.76177 |
| | | | | /len = 1262 |
| 1412 | DNA | M80899 | AHNAK | AHNAK nucleoprotein |
| | | | | (desmoyokin) |
| 1413 | Protein | M80899 | AHNAK | AHNAK nucleoprotein |
| | | (Translation) | | (desmoyokin) |
| 1414 | DNA | NM_014611 | MDN1 | MDN1, midasin homolog |
| | | | | (yeast) |
| 1415 | Protein | NP_055426 | MDN1 | MDN1, midasin homolog |
| | | | | (yeast) |
| 1416 | DNA | NM_002167 | ID3 | inhibitor of DNA binding 3, |
| | | | | dominant negative helix-loop- |
| | | | | helix protein |
| 1417 | Protein | NP_002158 | ID3 | inhibitor of DNA binding 3, |
| | | | | dominant negative helix-loop- |
| | | | | helix protein |
| 1418 | DNA | NM_003300 | TRAF3 | TNF receptor-associated factor 3 |
| 1419 | Protein | NP_003291 | TRAF3 | TNF receptor-associated factor 3 |
| 1420 | DNA | NM_145725 | TRAF3 | TNF receptor-associated factor 3 |
| 1421 | DNA | NM_145726 | TRAF3 | TNF receptor-associated factor 3 |
| 1422 | Protein | NP_663778 | TRAF3 | TNF receptor-associated factor 3 |
| 1423 | DNA | NM_001462 | FPRL1 | formyl peptide receptor-like 1 |
| 1424 | Protein | NP_001453 | FPRL1 | formyl peptide receptor-like 1 |
| 1425 | DNA | NM_005649 | ZNF354A | zinc finger protein 354A |
| 1426 | Protein | NP_005640 | ZNF354A | zinc finger protein 354A |
| 1427 | DNA | NM_001399 | ED1 | ectodermal dysplasia 1, |
| | | | | anhidrotic |
| 1428 | Protein | NP_001390 | ED1 | ectodermal dysplasia 1, |
| | | | | anhidrotic |
| 1429 | DNA | NM_014458 | AB026190 | Kelch motif containing protein |
| 1430 | Protein | NP_055273 | AB026190 | Kelch motif containing protein |
| 1431 | DNA | NM_001813 | CENPE | centromere protein E, 312 kDa |
| 1432 | Protein | NP_001804 | CENPE | centromere protein E, 312 kDa |
| 1433 | DNA | NM_002437 | MPV17 | MpV17 transgene, murine |
| | | | | homolog, glomerulosclerosis |
| 1434 | Protein | NP_002428 | MPV17 | MpV17 transgene, murine |
| | | | | homolog, glomerulosclerosis |
| 1435 | DNA | NM_012474 | UMPK | uridine monophosphate kinase |
| 1436 | Protein | NP_036606 | UMPK | uridine monophosphate kinase |
| 1437 | DNA | NM_012304 | FBXL7 | F-box and leucine-rich repeat |
| | | | | protein 7 |
| 1438 | Protein | NP_036436 | FBXL7 | F-box and leucine-rich repeat |
| | | | | protein 7 |
| 1439 | DNA | NM_005030 | PLK | polo-like kinase (Drosophila) |
| 1440 | Protein | NP_005021 | PLK | polo-like kinase (Drosophila) |
| 1441 | DNA | NM_001184 | ATR | ataxia telangiectasia and Rad3 |
| | | | | related |
| 1442 | Protein | NP_001175 | ATR | ataxia telangiectasia and Rad3 |
| | | | | related |
| 1443 | DNA | NM_014851 | KIAA0469 | KIAA0469 gene product |
| 1444 | Protein | NP_055666 | KIAA0469 | KIAA0469 gene product |
| 1445 | DNA | NM_021222 | HTCD37 | TcD37 homolog |
| 1446 | Protein | NP_067045 | HTCD37 | TcD37 homolog |
| 1447 | DNA | NM_005691 | ABCC9 | ATP-binding cassette, sub- |
| | | | | family C (CFTR/MRP), |
| | | | | member 9 |
| 1448 | Protein | NP_005682 | ABCC9 | ATP-binding cassette, sub- |
| | | | | family C (CFTR/MRP), |
| | | | | member 9 |
| 1449 | DNA | NM_020297 | ABCC9 | ATP-binding cassette, sub- |
| | | | | family C (CFTR/MRP), |
| | | | | member 9 |
| 1450 | Protein | NP_064693 | ABCC9 | ATP-binding cassette, sub- |
| | | | | family C (CFTR/MRP), |
| | | | | member 9 |
| 1451 | DNA | NM_020298 | ABCC9 | ATP-binding cassette, sub- |
| | | | | family C (CFTR/MRP), |
| | | | | member 9 |
| 1452 | Protein | NP_064694 | ABCC9 | ATP-binding cassette, sub- |
| | | | | family C (CFTR/MRP), |
| | | | | member 9 |
| 1453 | DNA | NM_003377 | VEGFB | vascular endothelial growth |
| | | | | factor B |
| 1454 | Protein | NP_003368 | VEGFB | vascular endothelial growth |
| | | | | factor B |
| 1455 | DNA | NM_005254 | GABPB1 | GA binding protein |
| | | | | transcription factor, beta |
| | | | | subunit 1, 53 kDa |
| 1456 | Protein | NP_005245 | GABPB1 | GA binding protein |
| | | | | transcription factor, beta |
| | | | | subunit 1, 53 kDa |
| 1457 | DNA | NM_016654 | GABPB1 | GA binding protein |
| | | | | transcription factor, beta |
| | | | | subunit 1, 53 kDa |
| 1458 | Protein | NP_057738 | GABPB1 | GA binding protein |
| | | | | transcription factor, beta |
| | | | | subunit 1, 53 kDa |
| 1459 | DNA | NM_014745 | KIAA0233 | KIAA0233 gene product |
| 1460 | Protein | NP_055560 | KIAA0233 | KIAA0233 gene product |
| 1461 | DNA | NM_014757 | MAML1 | mastermind-like 1 (Drosophila) |
| 1462 | Protein | NP_055572 | MAML1 | mastermind-like 1 (Drosophila) |
| 1463 | DNA | NM_014756 | KIAA0097 | KIAA0097 gene product |
| 1464 | Protein | NP_055571 | KIAA0097 | KIAA0097 gene product |
| 1465 | DNA | NM_002095 | GTF2E2 | general transcription factor IIE, |
| | | | | polypeptide 2, beta 34 kDa |
| 1466 | Protein | NP_002086 | GTF2E2 | general transcription factor IIE, |
| | | | | polypeptide 2, beta 34 kDa |
| 1467 | DNA | Z84718 | | Z84718 /FEATURE = cds#3 |
| | | | | /DEFINITION = HS322B1 |
| | | | | Human DNA sequence from |
| | | | | clone 322B1 on chromosome |
| | | | | 22q11-12, complete sequence |
| | | | | [Homo sapiens] |
| 1468 | DNA | AB002323 | DNCH1 | dynein, cytoplasmic, heavy |
| | | | | polypeptide 1 |
| 1469 | Protein | AB002323 | DNCH1 | dynein, cytoplasmic, heavy |
| | | (Translation) | | polypeptide 1 |
| 1470 | DNA | NM_002070 | GNAI2 | guanine nucleotide binding |
| | | | | protein (G protein), alpha |
| | | | | inhibiting activity polypeptide 2 |
| 1471 | Protein | NP_002061 | GNAI2 | guanine nucleotide binding |
| | | | | protein (G protein), alpha |
| | | | | inhibiting activity polypeptide 2 |
| 1472 | DNA | NM_006755 | TALDO1 | transaldolase 1 |
| 1473 | Protein | NP_006746 | TALDO1 | transaldolase 1 |
| 1474 | DNA | NM_014755 | TRIP-Br2 | transcriptional regulator |
| | | | | interacting with the PHS- |
| | | | | bromodomain 2 |
| 1475 | Protein | NP_055570 | TRIP-Br2 | transcriptional regulator |
| | | | | interacting with the PHS- |
| | | | | bromodomain 2 |
| 1476 | DNA | U80017 | | Cluster Incl. U80017: Homo |
| | | | | sapiens basic transcription |
| | | | | factor 2 p44 (btf2p44) gene, |
| | | | | partial cds, neuronal apoptosis |
| | | | | inhibitory protein (naip) and |
| | | | | survival motor neuron protein |
| | | | | (smn) genes, complete cds |
| | | | | /cds = (33.917) /gb = U80017 |
| | | | | /gi = 1737211 /ug = Hs.77306 /len |
| 1477 | Protein | U80017 (Translation) | | Cluster Incl. U80017: Homo |
| | | | | sapiens basic transcription |
| | | | | factor 2 p44 (btf2p44) gene, |
| | | | | partial cds, neuronal apoptosis |
| | | | | inhibitory protein (naip) and |
| | | | | survival motor neuron protein |
| | | | | (smn) genes, complete cds |
| | | | | /cds = (33.917) /gb = U80017 |
| | | | | /gi = 1737211 /ug = Hs.77306 /len |
| 1478 | DNA | NM_018453 | C14orf11 | chromosome 14 open reading |
| | | | | frame 11 |
| 1479 | Protein | NP_060923 | C14orf11 | chromosome 14 open reading |
| | | | | frame 11 |
| 1480 | DNA | U93305 | | Cluster Incl. U93305: Homo |
| | | | | sapiens A4 differentiation- |
| | | | | dependent protein (A4), triple |
| | | | | LIM domain protein (LMO6), |
| | | | | and synaptophysin (SYP) |
| | | | | genes, complete cds; and |
| | | | | calcium channel alpha-1 |
| | | | | subunit (CACNA1F) gene, |
| | | | | partial cds /cds = (75.533) |
| | | | | /gb = U93305 /gi = 270759 |
| 1481 | Protein | AAB92359 | | Cluster Incl. U93305: Homo |
| | | | | sapiens A4 differentiation- |
| | | | | dependent protein (A4), triple |
| | | | | LIM domain protein (LMO6), |
| | | | | and synaptophysin (SYP) |
| | | | | genes, complete cds; and |
| | | | | calcium channel alpha-1 |
| | | | | subunit (CACNA1F) gene, |
| | | | | partial cds /cds = (75.533) |
| | | | | /gb = U93305 /gi = 270759 |
| 1482 | DNA | NM_007103 | NDUFV1 | NADH dehydrogenase |
| | | | | (ubiquinone) flavoprotein 1, |
| | | | | 51 kDa |
| 1483 | Protein | NP_009034 | NDUFV1 | NADH dehydrogenase |
| | | | | (ubiquinone) flavoprotein 1, |
| | | | | 51 kDa |
| 1484 | DNA | NM_002766 | PRPSAP1 | phosphoribosyl pyrophosphate |
| | | | | synthetase-associated protein 1 |
| 1485 | Protein | NP_002757 | PRPSAP1 | phosphoribosyl pyrophosphate |
| | | | | synthetase-associated protein 1 |
| 1486 | DNA | NM_001662 | ARF5 | ADP-ribosylation factor 5 |
| 1487 | Protein | NP_001653 | ARF5 | ADP-ribosylation factor 5 |
| 1488 | DNA | NM_002346 | LY6E | lymphocyte antigen 6 complex, |
| | | | | locus E |
| 1489 | Protein | NP_002337 | LY6E | lymphocyte antigen 6 complex, |
| | | | | locus E |
| 1490 | DNA | NM_006736 | DNAJB2 | DnaJ (Hsp40) homolog, |
| | | | | subfamily B, member 2 |
| 1491 | Protein | NP_006727 | DNAJB2 | DnaJ (Hsp40) homolog, |
| | | | | subfamily B, member 2 |
| 1492 | DNA | NM_006801 | KDELR1 | KDEL (Lys-Asp-Glu-Leu) |
| | | | | endoplasmic reticulum protein |
| | | | | retention receptor 1 |
| 1493 | Protein | NP_006792 | KDELR1 | KDEL (Lys-Asp-Glu-Leu) |
| | | | | endoplasmic reticulum protein |
| | | | | retention receptor 1 |
| 1494 | DNA | NM_004231 | ATP6V1F | ATPase, H+ transporting, |
| | | | | lysosomal 14 kDa, V1 subunit F |
| 1495 | Protein | NP_004222 | ATP6V1F | ATPase, H+ transporting, |
| | | | | lysosomal 14 kDa, V1 subunit F |
| 1496 | DNA | NM_000516 | GNAS | GNAS complex locus |
| 1497 | Protein | NP_000507 | GNAS | GNAS complex locus |
| 1498 | DNA | NM_016592 | GNAS | GNAS complex locus |
| 1499 | Protein | NP_057676 | GNAS | GNAS complex locus |
| 1500 | DNA | NM_080425 | GNAS | GNAS complex locus |
| 1501 | Protein | NP_536350 | GNAS | GNAS complex locus |
| 1502 | DNA | NM_002618 | PEX13 | peroxisome biogenesis factor |
| | | | | 13 |
| 1503 | Protein | NP_002609 | PEX13 | peroxisome biogenesis factor |
| | | | | 13 |
| 1504 | DNA | NM_006638 | RPP40 | ribonuclease P, 40 kD subunit |
| 1505 | Protein | NP_006629 | RPP40 | ribonuclease P, 40 kD subunit |
| 1506 | DNA | NM_017544 | NRF | transcription factor NRF |
| 1507 | Protein | NP_060014 | NRF | transcription factor NRF |
| 1508 | DNA | AC004893 | | Cluster Incl. AC004893: Homo |
| | | | | sapiens PAC clone DJ0808A01 |
| | | | | from 7q21.1-q31.1 |
| | | | | /cds = (0.2138) /gb = AC004893 |
| | | | | /gi = 3694662 /ug = Hs.119120 |
| | | | | /len = 2139 |
| 1509 | DNA | NM_005063 | SCD | stearoyl-CoA desaturase (delta- |
| | | | | 9-desaturase) |
| 1510 | Protein | NP_005054 | SCD | stearoyl-CoA desaturase (delta- |
| | | | | 9-desaturase) |
| 1511 | DNA | NM_012345 | NUFIP1 | nuclear fragile X mental |
| | | | | retardation protein interacting |
| | | | | protein 1 |
| 1512 | Protein | NP_036477 | NUFIP1 | nuclear fragile X mental |
| | | | | retardation protein interacting |
| | | | | protein 1 |
| 1513 | DNA | NM_004379 | CREB1 | cAMP responsive element |
| | | | | binding protein 1 |
| 1514 | Protein | NP_004370 | CREB1 | cAMP responsive element |
| | | | | binding protein 1 |
| 1515 | DNA | NM_134442 | CREB1 | cAMP responsive element |
| | | | | binding protein 1 |
| 1516 | Protein | NP_604391 | CREB1 | cAMP responsive element |
| | | | | binding protein 1 |
| 1517 | DNA | D86961 | LHFPL2 | lipoma HMGIC fusion partner- |
| | | | | like 2 |
| 1518 | Protein | D86961 (Translation) | LHFPL2 | lipoma HMGIC fusion partner- |
| | | | | like 2 |
| 1519 | DNA | AL031778 | | Cluster Incl. |
| | | | | AL031778: dJ34B21.4.1 |
| | | | | (nuclear transcription factor Y, |
| | | | | alpha (CCAAT-Binding |
| | | | | transcription factor subunit B, |
| | | | | CBF-B, CAAT-Box DNA |
| | | | | binding pr /cds = (175.1218) |
| | | | | /gb = AL031778 /gi = 4153958 |
| | | | | /ug = Hs.797 /len = 3778 |
| 1520 | DNA | NM_015517 | MIZF | MBD2 (methyl-CpG-binding |
| | | | | protein)-interacting zinc finger |
| | | | | protein |
| 1521 | Protein | NP_056332 | MIZF | MBD2 (methyl-CpG-binding |
| | | | | protein)-interacting zinc finger |
| | | | | protein |
| 1522 | DNA | X98834 | SALL2 | sal-like 2 (Drosophila) |
| 1523 | DNA | NM_004425 | ECM1 | extracellular matrix protein 1 |
| 1524 | Protein | NP_004416 | ECM1 | extracellular matrix protein 1 |
| 1525 | DNA | NM_022664 | ECM1 | extracellular matrix protein 1 |
| 1526 | Protein | NP_073155 | ECM1 | extracellular matrix protein 1 |
| 1527 | DNA | NM_000156 | GAMT | guanidinoacetate N- |
| | | | | methyltransferase |
| 1528 | Protein | NP_000147 | GAMT | guanidinoacetate N- |
| | | | | methyltransferase |
| 1529 | DNA | NM_138924 | GAMT | guanidinoacetate N- |
| | | | | methyltransferase |
| 1530 | Protein | NP_620279 | GAMT | guanidinoacetate N- |
| | | | | methyltransferase |
| 1531 | DNA | NM_018224 | FLJ10803 | hypothetical protein FLJ10803 |
| 1532 | Protein | NP_060694 | FLJ10803 | hypothetical protein FLJ10803 |
| 1533 | DNA | NM_018999 | KIAA1128 | KIAA1128 protein |
| 1534 | Protein | NP_061872 | KIAA1128 | KIAA1128 protein |
| 1535 | DNA | NM_004502 | HOXB7 | homeo box B7 |
| 1536 | Protein | NP_004493 | HOXB7 | homeo box B7 |
| 1537 | DNA | NM_017790 | RGS3 | regulator of G-protein |
| | | | | signalling 3 |
| 1538 | Protein | NP_060260 | RGS3 | regulator of G-protein |
| | | | | signalling 3 |
| 1539 | DNA | NM_021106 | RGS3 | regulator of G-protein |
| | | | | signalling 3 |
| 1540 | Protein | NP_066929 | RGS3 | regulator of G-protein |
| | | | | signalling 3 |
| 1541 | DNA | NM_130795 | RGS3 | regulator of G-protein |
| | | | | signalling 3 |
| 1542 | Protein | NP_570613 | RGS3 | regulator of G-protein |
| | | | | signalling 3 |
| 1543 | DNA | NM_134427 | RGS3 | regulator of G-protein |
| | | | | signalling 3 |
| 1544 | Protein | NP_602299 | RGS3 | regulator of G-protein |
| | | | | signalling 3 |
| 1545 | DNA | NM_032182 | KIAA0157 | KIAA0157 protein |
| 1546 | Protein | NP_115558 | KIAA0157 | KIAA0157 protein |
| 1547 | DNA | NM_013446 | MKRN1 | makorin, ring finger protein, 1 |
| 1548 | Protein | NP_038474 | MKRN1 | makorin, ring finger protein, 1 |
| 1549 | DNA | NM_015156 | RCOR | REST corepressor |
| 1550 | Protein | NP_055971 | RCOR | REST corepressor |
| 1551 | DNA | NM_001682 | ATP2B1 | ATPase, Ca++ transporting, |
| | | | | plasma membrane 1 |
| 1552 | Protein | NP_001673 | ATP2B1 | ATPase, Ca++ transporting, |
| | | | | plasma membrane 1 |
| 1553 | DNA | NM_003342 | UBE2G1 | ubiquitin-conjugating enzyme |
| | | | | E2G 1 (UBC7 homolog, C. elegans) |
| 1554 | Protein | NP_003333 | UBE2G1 | ubiquitin-conjugating enzyme |
| | | | | E2G 1 (UBC7 homolog, C. elegans) |
| 1555 | DNA | NM_003470 | USP7 | ubiquitin specific protease 7 |
| | | | | (herpes virus-associated) |
| 1556 | Protein | NP_003461 | USP7 | ubiquitin specific protease 7 |
| | | | | (herpes virus-associated) |
| 1557 | DNA | NM_000688 | ALAS1 | aminolevulinate, delta-, |
| | | | | synthase 1 |
| 1558 | Protein | NP_000679 | ALAS1 | aminolevulinate, delta-, |
| | | | | synthase 1 |
| 1559 | DNA | NM_005153 | USP10 | ubiquitin specific protease 10 |
| 1560 | Protein | NP_005144 | USP10 | ubiquitin specific protease 10 |
| 1561 | DNA | NM_003362 | UNG | uracil-DNA glycosylase |
| 1562 | Protein | NP_003353 | UNG | uracil-DNA glycosylase |
| 1563 | DNA | NM_080911 | UNG | uracil-DNA glycosylase |
| 1564 | Protein | NP_550433 | UNG | uracil-DNA glycosylase |
| 1565 | DNA | NM_015153 | PHF3 | PHD finger protein 3 |
| 1566 | Protein | NP_055968 | PHF3 | PHD finger protein 3 |
| 1567 | DNA | NM_003488 | AKAP1 | A kinase (PRKA) anchor |
| | | | | protein 1 |
| 1568 | Protein | NP_003479 | AKAP1 | A kinase (PRKA) anchor |
| | | | | protein 1 |
| 1569 | DNA | NM_139275 | AKAP1 | A kinase (PRKA) anchor |
| | | | | protein 1 |
| 1570 | Protein | NP_644804 | AKAP1 | A kinase (PRKA) anchor |
| | | | | protein 1 |
| 1571 | DNA | NM_004582 | RABGGTB | Rab geranylgeranyltransferase, |
| | | | | beta subunit |
| 1572 | Protein | NP_004573 | RABGGTB | Rab geranylgeranyltransferase, |
| | | | | beta subunit |
| 1573 | DNA | NM_002713 | PPP1R8 | protein phosphatase 1, |
| | | | | regulatory (inhibitor) subunit 8 |
| 1574 | Protein | NP_002704 | PPP1R8 | protein phosphatase 1, |
| | | | | regulatory (inhibitor) subunit 8 |
| 1575 | DNA | NM_014110 | PPP1R8 | protein phosphatase 1, |
| | | | | regulatory (inhibitor) subunit 8 |
| 1576 | Protein | NP_054829 | PPP1R8 | protein phosphatase 1, |
| | | | | regulatory (inhibitor) subunit 8 |
| 1577 | DNA | NM_138558 | PPP1R8 | protein phosphatase 1, |
| | | | | regulatory (inhibitor) subunit 8 |
| 1578 | Protein | NP_612568 | PPP1R8 | protein phosphatase 1, |
| | | | | regulatory (inhibitor) subunit 8 |
| 1579 | DNA | NM_004354 | | Homo sapiens mRNA; cDNA |
| | | | | DKFZp434B142 (from clone |
| | | | | DKFZp434B142), mRNA |
| | | | | sequence |
| 1580 | Protein | NP_004345 | | Homo sapiens mRNA; cDNA |
| | | | | DKFZp434B142 (from clone |
| | | | | DKFZp434B142), mRNA |
| | | | | sequence |
| 1581 | DNA | NM_012234 | RYBP | RING1 and YY1 binding |
| | | | | protein |
| 1582 | Protein | NP_036366 | RYBP | RING1 and YY1 binding |
| | | | | protein |
| 1583 | DNA | NM_001315 | MAPK14 | mitogen-activated protein |
| | | | | kinase 14 |
| 1584 | Protein | NP_001306 | MAPK14 | mitogen-activated protein |
| | | | | kinase 14 |
| 1585 | DNA | NM_139012 | MAPK14 | mitogen-activated protein |
| | | | | kinase 14 |
| 1586 | Protein | NP_620581 | MAPK14 | mitogen-activated protein |
| | | | | kinase 14 |
| 1587 | DNA | NM_139013 | MAPK14 | mitogen-activated protein |
| | | | | kinase 14 |
| 1588 | Protein | NP_620582 | MAPK14 | mitogen-activated protein |
| | | | | kinase 14 |
| 1589 | DNA | NM_139014 | MAPK14 | mitogen-activated protein |
| | | | | kinase 14 |
| 1590 | Protein | NP_620583 | MAPK14 | mitogen-activated protein |
| | | | | kinase 14 |
| 1591 | DNA | NM_014962 | BTBD3 | BTB (POZ) domain containing 3 |
| 1592 | Protein | NP_055777 | BTBD3 | BTB (POZ) domain containing 3 |
| 1593 | DNA | NM_006340 | BAIAP2 | BAI1-associated protein 2 |
| 1594 | Protein | NP_006331 | BAIAP2 | BAI1-associated protein 2 |
| 1595 | DNA | NM_017450 | BAIAP2 | BAI1-associated protein 2 |
| 1596 | Protein | NP_059344 | BAIAP2 | BAI1-associated protein 2 |
| 1597 | DNA | NM_017451 | BAIAP2 | BAI1-associated protein 2 |
| 1598 | Protein | NP_059345 | BAIAP2 | BAI1-associated protein 2 |
| 1599 | DNA | AL049227 | | Homo sapiens mRNA; cDNA |
| | | | | DKFZp564N1116 (from clone |
| | | | | DKFZp564N1116), mRNA |
| | | | | sequence |
| 1600 | DNA | NM_012066 | 20D7-FC4 | hypothetical protein 20D7-FC4 |
| 1601 | Protein | NP_036198 | 20D7-FC4 | hypothetical protein 20D7-FC4 |
| 1602 | DNA | NM_006675 | NET-5 | transmembrane 4 superfamily |
| | | | | member tetraspan NET-5 |
| 1603 | Protein | NP_006666 | NET-5 | transmembrane 4 superfamily |
| | | | | member tetraspan NET-5 |
| 1604 | DNA | AL080062 | DKFZP564I122 | DKFZP564I122 protein |
| 1605 | Protein | AL080062 | DKFZP564I122 | DKFZP564I122 protein |
| | | (Translation) | | |
| 1606 | DNA | NM_005677 | COLQ | collagen-like tail subunit |
| | | | | (single strand of homotrimer) |
| | | | | of asymmetric |
| | | | | acetylcholinesterase |
| 1607 | Protein | NP_005668 | COLQ | collagen-like tail subunit |
| | | | | (single strand of homotrimer) |
| | | | | of asymmetric |
| | | | | acetylcholinesterase |
| 1608 | DNA | NM_080538 | COLQ | collagen-like tail subunit |
| | | | | (single strand of homotrimer) |
| | | | | of asymmetric |
| | | | | acetylcholinesterase |
| 1609 | Protein | NP_536799 | COLQ | collagen-like tail subunit |
| | | | | (single strand of homotrimer) |
| | | | | of asymmetric |
| | | | | acetylcholinesterase |
| 1610 | DNA | NM_080539 | COLQ | collagen-like tail subunit |
| | | | | (single strand of homotrimer) |
| | | | | of asymmetric |
| | | | | acetylcholinesterase |
| 1611 | Protein | NP_536800 | COLQ | collagen-like tail subunit |
| | | | | (single strand of homotrimer) |
| | | | | of asymmetric |
| | | | | acetylcholinesterase |
| 1612 | DNA | NM_015064 | ELKS | ELKS protein |
| 1613 | Protein | NP_055879 | ELKS | ELKS protein |
| 1614 | DNA | NM_005513 | GTF2E1 | general transcription factor IIE, |
| | | | | polypeptide 1, alpha 56 kDa |
| 1615 | Protein | NP_005504 | GTF2E1 | general transcription factor IIE, |
| | | | | polypeptide 1, alpha 56 kDa |
| 1616 | DNA | NM_013310 | AF038169 | hypothetical protein AF038169 |
| 1617 | Protein | NP_037442 | AF038169 | hypothetical protein AF038169 |
| 1618 | DNA | NM_003846 | PEX11B | peroxisomal biogenesis factor |
| | | | | 11B |
| 1619 | Protein | NP_003837 | PEX11B | peroxisomal biogenesis factor |
| | | | | 11B |
| 1620 | DNA | NM_014602 | PIK3R4 | phosphoinositide-3-kinase, |
| | | | | regulatory subunit 4, p150 |
| 1621 | Protein | NP_055417 | PIK3R4 | phosphoinositide-3-kinase, |
| | | | | regulatory subunit 4, p150 |
| 1622 | DNA | NM_012151 | F8A | coagulation factor VIII- |
| | | | | associated (intronic transcript) |
| 1623 | Protein | NP_036283 | F8A | coagulation factor VIII- |
| | | | | associated (intronic transcript) |
| 1624 | DNA | NM_005334 | HCFC1 | host cell factor C1 (VP16- |
| | | | | accessory protein) |
| 1625 | Protein | NP_005325 | HCFC1 | host cell factor C1 (VP16- |
| | | | | accessory protein) |
| 1626 | DNA | NM_004295 | TRAF4 | TNF receptor-associated factor 4 |
| 1627 | Protein | NP_004286 | TRAF4 | TNF receptor-associated factor 4 |
| 1628 | DNA | NM_145751 | TRAF4 | TNF receptor-associated factor 4 |
| 1629 | Protein | NP_665694 | TRAF4 | TNF receptor-associated factor 4 |
| 1630 | DNA | NM_019005 | FLJ20323 | hypothetical protein FLJ20323 |
| 1631 | Protein | NP_061878 | FLJ20323 | hypothetical protein FLJ20323 |
| 1632 | DNA | NM_002653 | PITX1 | paired-like homeodomain |
| | | | | transcription factor 1 |
| 1633 | Protein | NP_002644 | PITX1 | paired-like homeodomain |
| | | | | transcription factor 1 |
| 1634 | DNA | NM_001810 | CENPB | centromere protein B, 80 kDa |
| 1635 | Protein | NP_001801 | CENPB | centromere protein B, 80 kDa |
| 1636 | DNA | NM_004239 | TRIP11 | thyroid hormone receptor |
| | | | | interactor 11 |
| 1637 | Protein | NP_004230 | TRIP11 | thyroid hormone receptor |
| | | | | interactor 11 |
| 1638 | DNA | NM_006910 | RBBP6 | retinoblastoma binding protein 6 |
| 1639 | Protein | NP_008841 | RBBP6 | retinoblastoma binding protein 6 |
| 1640 | DNA | NM_004697 | PRPF4 | PRP4 pre-mRNA processing |
| | | | | factor 4 homolog (yeast) |
| 1641 | Protein | NP_004688 | PRPF4 | PRP4 pre-mRNA processing |
| | | | | factor 4 homolog (yeast) |
| 1642 | DNA | NM_018096 | FLJ10458 | hypothetical protein similar to |
| | | | | beta-transducin family |
| 1643 | Protein | NP_060566 | FLJ10458 | hypothetical protein similar to |
| | | | | beta-transducin family |
| 1644 | DNA | NM_014255 | TMEM4 | transmembrane protein 4 |
| 1645 | Protein | NP_055070 | TMEM4 | transmembrane protein 4 |
| 1646 | DNA | NM_014001 | GGA3 | golgi associated, gamma |
| | | | | adaptin ear containing, ARF |
| | | | | binding protein 3 |
| 1647 | Protein | NP_054720 | GGA3 | golgi associated, gamma |
| | | | | adaptin ear containing, ARF |
| | | | | binding protein 3 |
| 1648 | DNA | NM_138619 | GGA3 | golgi associated, gamma |
| | | | | adaptin ear containing, ARF |
| | | | | binding protein 3 |
| 1649 | Protein | NP_619525 | GGA3 | golgi associated, gamma |
| | | | | adaptin ear containing, ARF |
| | | | | binding protein 3 |
| 1650 | DNA | NM_003629 | PIK3R3 | phosphoinositide-3-kinase, |
| | | | | regulatory subunit, polypeptide |
| | | | | 3 (p55, gamma) |
| 1651 | Protein | NP_003620 | PIK3R3 | phosphoinositide-3-kinase, |
| | | | | regulatory subunit, polypeptide |
| | | | | 3 (p55, gamma) |
| 1652 | DNA | NM_153250 | MGC40413 | hypothetical protein |
| | | | | MGC40413 |
| 1653 | Protein | NP_694982 | MGC40413 | hypothetical protein |
| | | | | MGC40413 |
| 1654 | DNA | NM_001663 | ARF6 | ADP-ribosylation factor 6 |
| 1655 | Protein | NP_001654 | ARF6 | ADP-ribosylation factor 6 |
| 1656 | DNA | NM_001687 | ATP5D | ATP synthase, H+ transporting, |
| | | | | mitochondrial F1 complex, |
| | | | | delta subunit |
| 1657 | Protein | NP_001678 | ATP5D | ATP synthase, H+ transporting, |
| | | | | mitochondrial F1 complex, |
| | | | | delta subunit |
| 1658 | DNA | NM_001894 | CSNK1E | casein kinase 1, epsilon |
| 1659 | Protein | NP_001885 | CSNK1E | casein kinase 1, epsilon |
| 1660 | DNA | NM_152221 | CSNK1E | casein kinase 1, epsilon |
| 1661 | DNA | NM_005871 | SPF30 | splicing factor 30, survival of |
| | | | | motor neuron-related |
| 1662 | Protein | NP_005862 | SPF30 | splicing factor 30, survival of |
| | | | | motor neuron-related |
| 1663 | DNA | AL080234 | | Homo sapiens clone FBD3 Cri- |
| | | | | du-chat critical region mRNA, |
| | | | | mRNA sequence |
| 1664 | DNA | NM_003799 | RNMT | RNA (guanine-7-) |
| | | | | methyltransferase |
| 1665 | Protein | NP_003790 | RNMT | RNA (guanine-7-) |
| | | | | methyltransferase |
| 1666 | DNA | NM_015144 | BDG-29 | BDG-29 proten |
| 1667 | Protein | NP_055959 | BDG-29 | BDG-29 proten |
| 1668 | DNA | NM_032909 | BDG-29 | BDG-29 proten |
| 1669 | DNA | AB014542 | TNRC15 | trinucleotide repeat containing |
| | | | | 15 |
| 1670 | Protein | AB014542 | TNRC15 | trinucleotide repeat containing |
| | | (Translation) | | 15 |
| 1671 | DNA | NM_001359 | DECR1 | 2,4-dienoyl CoA reductase 1, |
| | | | | mitochondrial |
| 1672 | Protein | NP_001350 | DECR1 | 2,4-dienoyl CoA reductase 1, |
| | | | | mitochondrial |
| 1673 | DNA | NM_023012 | FLJ11021 | hypothetical protein FLJ11021 |
| | | | | similar to splicing factor, |
| | | | | arginine/serine-rich 4 |
| 1674 | Protein | NP_075388 | FLJ11021 | hypothetical protein FLJ11021 |
| | | | | similar to splicing factor, |
| | | | | arginine/serine-rich 4 |
| 1675 | DNA | NM_006265 | RAD21 | RAD21 homolog (S. pombe) |
| 1676 | Protein | NP_006256 | RAD21 | RAD21 homolog (S. pombe) |
| 1677 | DNA | NM_007275 | FUS1 | lung cancer candidate |
| 1678 | Protein | NP_009206 | FUS1 | lung cancer candidate |
| 1679 | DNA | NM_002391 | MDK | midkine (neurite growth- |
| | | | | promoting factor 2) |
| 1680 | Protein | NP_002382 | MDK | midkine (neurite growth- |
| | | | | promoting factor 2) |
| 1681 | DNA | NM_007061 | CDC42EP1 | CDC42 effector protein (Rho |
| | | | | GTPase binding) 1 |
| 1682 | Protein | NP_008992 | CDC42EP1 | CDC42 effector protein (Rho |
| | | | | GTPase binding) 1 |
| 1683 | DNA | NM_152243 | CDC42EP1 | CDC42 effector protein (Rho |
| | | | | GTPase binding) 1 |
| 1684 | Protein | NP_689449 | CDC42EP1 | CDC42 effector protein (Rho |
| | | | | GTPase binding) 1 |
| 1685 | DNA | NM_005620 | S100A11 | S100 calcium binding protein |
| | | | | A11 (calgizzarin) |
| 1686 | Protein | NP_005611 | S100A11 | S100 calcium binding protein |
| | | | | A11 (calgizzarin) |
| 1687 | DNA | NM_004075 | CRY1 | cryptochrome 1 (photolyase- |
| | | | | like) |
| 1688 | Protein | NP_004066 | CRY1 | cryptochrome 1 (photolyase- |
| | | | | like) |
| 1689 | DNA | NM_017503 | SURF2 | surfeit 2 |
| 1690 | Protein | NP_059973 | SURF2 | surfeit 2 |
| 1691 | DNA | NM_001478 | GALGT | UDP-N-acetyl-alpha-D- |
| | | | | galactosamine:(N- |
| | | | | acetylneuraminyl)- |
| | | | | galactosylglucosylceramide N- |
| | | | | acetylgalactosaminyltransferase |
| | | | | (GalNAc-T) |
| 1692 | Protein | NP_001469 | GALGT | UDP-N-acetyl-alpha-D- |
| | | | | galactosamine:(N- |
| | | | | acetylneuraminyl)- |
| | | | | galactosylglucosylceramide N- |
| | | | | acetylgalactosaminyltransferase |
| | | | | (GalNAc-T) |
| 1693 | DNA | NM_000110 | DPYD | dihydropyrimidine |
| | | | | dehydrogenase |
| 1694 | Protein | NP_000101 | DPYD | dihydropyrimidine |
| | | | | dehydrogenase |
| 1695 | DNA | D26488 | KIAA0007 | KIAA0007 protein |
| 1696 | Protein | D26488 (Translation) | KIAA0007 | KIAA0007 protein |
| 1697 | DNA | NM_002475 | MLC1SA | myosin light chain 1 slow a |
| 1698 | Protein | NP_002466 | MLC1SA | myosin light chain 1 slow a |
| 1699 | DNA | W21827 | DKFZP564O092 | DKFZP564O092 protein |
| 1700 | DNA | NM_002496 | NDUFS8 | NADH dehydrogenase |
| | | | | (ubiquinone) Fe—S protein 8, |
| | | | | 23 kDa (NADH-coenzyme Q |
| | | | | reductase) |
| 1701 | Protein | NP_002487 | NDUFS8 | NADH dehydrogenase |
| | | | | (ubiquinone) Fe—S protein 8, |
| | | | | 23 kDa (NADH-coenzyme Q |
| | | | | reductase) |
| 1702 | DNA | NM_031206 | FLJ12525 | hypothetical protein FLJ12525 |
| 1703 | Protein | NP_112483 | FLJ12525 | hypothetical protein FLJ12525 |
| 1704 | DNA | NM_002871 | RABIF | RAB interacting factor |
| 1705 | Protein | NP_002862 | RABIF | RAB interacting factor |
| 1706 | DNA | NM_003631 | PARG | poly (ADP-ribose) |
| | | | | glycohydrolase |
| 1707 | Protein | NP_003622 | PARG | poly (ADP-ribose) |
| | | | | glycohydrolase |
| 1708 | DNA | NM_007026 | DUSP14 | dual specificity phosphatase 14 |
| 1709 | Protein | NP_008957 | DUSP14 | dual specificity phosphatase 14 |
| 1710 | DNA | NM_007024 | PL6 | PL6 protein |
| 1711 | Protein | NP_008955 | PL6 | PL6 protein |
| 1712 | DNA | NM_003815 | ADAM15 | a disintegrin and |
| | | | | metalloproteinase domain 15 |
| | | | | (metargidin) |
| 1713 | Protein | NP_003806 | ADAM15 | a disintegrin and |
| | | | | metalloproteinase domain 15 |
| | | | | (metargidin) |
| 1714 | DNA | NM_004215 | EBAG9 | estrogen receptor binding site |
| | | | | associated, antigen, 9 |
| 1715 | Protein | NP_004206 | EBAG9 | estrogen receptor binding site |
| | | | | associated, antigen, 9 |
| 1716 | DNA | NM_006421 | BIG1 | brefeldin A-inhibited guanine |
| | | | | nucleotide-exchange protein 1 |
| 1717 | Protein | NP_006412 | BIG1 | brefeldin A-inhibited guanine |
| | | | | nucleotide-exchange protein 1 |
| 1718 | DNA | NM_014284 | NCDN | neurochondrin |
| 1719 | Protein | NP_055099 | NCDN | neurochondrin |
| 1720 | DNA | NM_021809 | TGIF2 | TGFB-induced factor 2 (TALE |
| | | | | family homeobox) |
| 1721 | Protein | NP_068581 | TGIF2 | TGFB-induced factor 2 (TALE |
| | | | | family homeobox) |
| 1722 | DNA | NM_015125 | CIC | capicua homolog (Drosophila) |
| 1723 | Protein | NP_055940 | CIC | capicua homolog (Drosophila) |
| 1724 | DNA | NM_004897 | MINPP1 | multiple inositol polyphosphate |
| | | | | histidine phosphatase, 1 |
| 1725 | Protein | NP_004888 | MINPP1 | multiple inositol polyphosphate |
| | | | | histidine phosphatase, 1 |
| 1726 | DNA | NM_006928 | SILV | silver homolog (mouse) |
| 1727 | Protein | NP_008859 | SILV | silver homolog (mouse) |
| 1728 | DNA | NM_015288 | KIAA0239 | KIAA0239 protein |
| 1729 | Protein | NP_056103 | KIAA0239 | KIAA0239 protein |
| 1730 | DNA | NM_006322 | TUBGCP3 | tubulin, gamma complex |
| | | | | associated protein 3 |
| 1731 | Protein | NP_006313 | TUBGCP3 | tubulin, gamma complex |
| | | | | associated protein 3 |
| 1732 | DNA | AL049321 | | Homo sapiens mRNA; cDNA |
| | | | | DKFZp564D156 (from clone |
| | | | | DKFZp564D156), mRNA |
| | | | | sequence |
| 1733 | DNA | NM_007010 | ROK1 | ATP-dependent RNA helicase |
| 1734 | Protein | NP_008941 | ROK1 | ATP-dependent RNA helicase |
| 1735 | DNA | NM_152300 | ROK1 | ATP-dependent RNA helicase |
| 1736 | Protein | NP_689513 | ROK1 | ATP-dependent RNA helicase |
| 1737 | DNA | NM_001155 | ANXA6 | annexin A6 |
| 1738 | Protein | NP_001146 | ANXA6 | annexin A6 |
| 1739 | DNA | NM_004033 | ANXA6 | annexin A6 |
| 1740 | Protein | NP_004024 | ANXA6 | annexin A6 |
| 1741 | DNA | NM_007005 | BCE-1 | BCE-1 protein |
| 1742 | Protein | NP_008936 | BCE-1 | BCE-1 protein |
| 1743 | DNA | NM_000202 | IDS | iduronate 2-sulfatase (Hunter |
| | | | | syndrome) |
| 1744 | Protein | NP_000193 | IDS | iduronate 2-sulfatase (Hunter |
| | | | | syndrome) |
| 1745 | DNA | NM_006123 | IDS | iduronate 2-sulfatase (Hunter |
| | | | | syndrome) |
| 1746 | Protein | NP_006114 | IDS | iduronate 2-sulfatase (Hunter |
| | | | | syndrome) |
| 1747 | DNA | NM_000018 | ACADVL | acyl-Coenzyme A |
| | | | | dehydrogenase, very long chain |
| 1748 | Protein | NP_000009 | ACADVL | acyl-Coenzyme A |
| | | | | dehydrogenase, very long chain |
| 1749 | DNA | NM_006766 | ZNF220 | zinc finger protein 220 |
| 1750 | Protein | NP_006757 | ZNF220 | zinc finger protein 220 |
| 1751 | DNA | NM_003682 | MADD | MAP-kinase activating death |
| | | | | domain |
| 1752 | Protein | NP_003673 | MADD | MAP-kinase activating death |
| | | | | domain |
| 1753 | DNA | NM_130470 | MADD | MAP-kinase activating death |
| | | | | domain |
| 1754 | Protein | NP_569826 | MADD | MAP-kinase activating death |
| | | | | domain |
| 1755 | DNA | NM_130471 | MADD | MAP-kinase activating death |
| | | | | domain |
| 1756 | Protein | NP_569827 | MADD | MAP-kinase activating death |
| | | | | domain |
| 1757 | DNA | NM_130472 | MADD | MAP-kinase activating death |
| | | | | domain |
| 1758 | Protein | NP_569828 | MADD | MAP-kinase activating death |
| | | | | domain |
| 1759 | DNA | AF070546 | DKFZp451J0118 | hypothetical protein |
| | | | | DKFZp451J0118 |
| 1760 | DNA | NM_001450 | FHL2 | four and a half LIM domains 2 |
| 1761 | Protein | NP_001441 | FHL2 | four and a half LIM domains 2 |
| 1762 | DNA | NM_007359 | MLN51 | MLN51 protein |
| 1763 | Protein | NP_031385 | MLN51 | MLN51 protein |
| 1764 | DNA | AA015605 | FLJ20811 | hypothetical protein FLJ20811 |
| 1765 | DNA | NM_006830 | UQCR | ubiquinol-cytochrome c |
| | | | | reductase (6.4 kD) subunit |
| 1766 | Protein | NP_006821 | UQCR | ubiquinol-cytochrome c |
| | | | | reductase (6.4 kD) subunit |
| 1767 | DNA | NM_006302 | GCS1 | glucosidase I |
| 1768 | Protein | NP_006293 | GCS1 | glucosidase I |
| 1769 | DNA | NM_001383 | DPH2L1 | diptheria toxin resistance |
| | | | | protein required for |
| | | | | diphthamide biosynthesis-like 1 |
| | | | | (S. cerevisiae) |
| 1770 | Protein | NP_001374 | DPH2L1 | diptheria toxin resistance |
| | | | | protein required for |
| | | | | diphthamide biosynthesis-like 1 |
| | | | | (S. cerevisiae) |
| 1771 | DNA | NM_004592 | SFRS8 | splicing factor, arginine/serine- |
| | | | | rich 8 (suppressor-of-white- |
| | | | | apricot homolog, Drosophila) |
| 1772 | Protein | NP_004583 | SFRS8 | splicing factor, arginine/serine- |
| | | | | rich 8 (suppressor-of-white- |
| | | | | apricot homolog, Drosophila) |
| 1773 | DNA | NM_152235 | SFRS8 | splicing factor, arginine/serine- |
| | | | | rich 8 (suppressor-of-white- |
| | | | | apricot homolog, Drosophila) |
| 1774 | Protein | NP_689421 | SFRS8 | splicing factor, arginine/serine- |
| | | | | rich 8 (suppressor-of-white- |
| | | | | apricot homolog, Drosophila) |
| 1775 | DNA | NM_015029 | POP1 | processing of precursors 1 |
| 1776 | Protein | NP_055844 | POP1 | processing of precursors 1 |
| 1777 | DNA | NM_014783 | ARHGAP11A | KIAA0013 gene product |
| 1778 | Protein | NP_055598 | ARHGAP11A | KIAA0013 gene product |
| 1779 | DNA | NM_002936 | RNASEH1 | ribonuclease H1 |
| 1780 | Protein | NP_002927 | RNASEH1 | ribonuclease H1 |
| 1781 | DNA | NM_005802 | TP53BPL | tumor protein p53-binding |
| | | | | protein |
| 1782 | Protein | NP_005793 | TP53BPL | tumor protein p53-binding |
| | | | | protein |
| 1783 | DNA | NM_002072 | | Homo sapiens mRNA; cDNA |
| | | | | DKFZp686D0521 (from clone |
| | | | | DKFZp686D0521), mRNA |
| | | | | sequence |
| 1784 | Protein | NP_002063 | | Homo sapiens mRNA; cDNA |
| | | | | DKFZp686D0521 (from clone |
| | | | | DKFZp686D0521), mRNA |
| | | | | sequence |
| 1785 | DNA | NM_000578 | SLC11A1 | solute carrier family 11 |
| | | | | (proton-coupled divalent metal |
| | | | | ion transporters), member 1 |
| 1786 | Protein | NP_000569 | SLC11A1 | solute carrier family 11 |
| | | | | (proton-coupled divalent metal |
| | | | | ion transporters), member 1 |
| 1787 | DNA | NM_000421 | KRT10 | keratin 10 (epidermolytic |
| | | | | hyperkeratosis; keratosis |
| | | | | palmaris et plantaris) |
| 1788 | Protein | NP_000412 | KRT10 | keratin 10 (epidermolytic |
| | | | | hyperkeratosis; keratosis |
| | | | | palmaris et plantaris) |
| 1789 | DNA | NM_006349 | CG1I | putative cyclin G1 interacting |
| | | | | protein |
| 1790 | Protein | NP_006340 | CG1I | putative cyclin G1 interacting |
| | | | | protein |
| 1791 | DNA | AC002073 | | Cluster Incl. AC002073: Human |
| | | | | PAC clone DJ515N1 from |
| | | | | 22q11.2-q22 /cds = (0.2201) |
| | | | | /gb = AC002073 /gi = 2078469 |
| | | | | /ug = Hs.100623 /len = 2202 |
| 1792 | Protein | AAB54054 | | Cluster Incl. AC002073: Human |
| | | | | PAC clone DJ515N1 from |
| | | | | 22q11.2-q22 /cds = (0.2201) |
| | | | | /gb = AC002073 /gi = 2078469 |
| | | | | /ug = Hs.100623 /len = 2202 |
| 1793 | DNA | NM_002126 | HLF | hepatic leukemia factor |
| 1794 | Protein | NP_002117 | HLF | hepatic leukemia factor |
| 1795 | DNA | NM_006280 | SSR4 | signal sequence receptor, delta |
| | | | | (translocon-associated protein |
| | | | | delta) |
| 1796 | Protein | NP_006271 | SSR4 | signal sequence receptor, delta |
| | | | | (translocon-associated protein |
| | | | | delta) |
| 1797 | DNA | NM_007263 | COPE | coatomer protein complex, |
| | | | | subunit epsilon |
| 1798 | Protein | NP_009194 | COPE | coatomer protein complex, |
| | | | | subunit epsilon |
| 1799 | DNA | NM_133476 | ZNF384 | zinc finger protein 384 |
| 1800 | Protein | NP_597733 | ZNF384 | zinc finger protein 384 |
| 1801 | DNA | NM_024056 | MGC5576 | hypothetical protein MGC5576 |
| 1802 | Protein | NP_076961 | MGC5576 | hypothetical protein MGC5576 |
| 1803 | DNA | NM_007373 | SHOC2 | soc-2 suppressor of clear |
| | | | | homolog (C. elegans) |
| 1804 | Protein | NP_031399 | SHOC2 | soc-2 suppressor of clear |
| | | | | homolog (C. elegans) |
| 1805 | DNA | NM_004762 | PSCD1 | pleckstrin homology, Sec7 and |
| | | | | coiled/coil domains 1(cytohesin |
| | | | | 1) |
| 1806 | Protein | NP_004753 | PSCD1 | pleckstrin homology, Sec7 and |
| | | | | coiled/coil domains 1(cytohesin |
| | | | | 1) |
| 1807 | DNA | NM_017456 | PSCD1 | pleckstrin homology, Sec7 and |
| | | | | coiled/coil domains 1(cytohesin |
| | | | | 1) |
| 1808 | Protein | NP_059430 | PSCD1 | pleckstrin homology, Sec7 and |
| | | | | coiled/coil domains 1(cytohesin |
| | | | | 1) |
| 1809 | DNA | NM_018847 | KIAA1354 | KIAA1354 protein |
| 1810 | Protein | NP_061335 | KIAA1354 | KIAA1354 protein |
| 1811 | DNA | NM_003093 | SNRPC | small nuclear ribonucleoprotein |
| | | | | polypeptide C |
| 1812 | Protein | NP_003084 | SNRPC | small nuclear ribonucleoprotein |
| | | | | polypeptide C |
| 1813 | DNA | NM_006948 | STCH | stress 70 protein chaperone, |
| | | | | microsome-associated, 60 kDa |
| 1814 | Protein | NP_008879 | STCH | stress 70 protein chaperone, |
| | | | | microsome-associated, 60 kDa |
| 1815 | DNA | M21259 | | Cluster Incl. M21259: Human |
| | | | | Alu repeats in the region 5 to |
| | | | | the small nuclear |
| | | | | ribonucleoprotein E gene |
| | | | | /cds = (0.278) /gb = M21259 |
| | | | | /gi = 338258 /ug = Hs.1066 |
| | | | | /len = 446 |
| 1816 | DNA | NM_014306 | HSPC117 | hypothetical protein HSPC117 |
| 1817 | Protein | NP_055121 | HSPC117 | hypothetical protein HSPC117 |
| 1818 | DNA | NM_001261 | CDK9 | cyclin-dependent kinase 9 |
| | | | | (CDC2-related kinase) |
| 1819 | Protein | NP_001252 | CDK9 | cyclin-dependent kinase 9 |
| | | | | (CDC2-related kinase) |
| 1820 | DNA | NM_017443 | POLE3 | polymerase (DNA directed), |
| | | | | epsilon 3 (p17 subunit) |
| 1821 | Protein | NP_059139 | POLE3 | polymerase (DNA directed), |
| | | | | epsilon 3 (p17 subunit) |
| 1822 | DNA | AB014527 | CLASP2 | cytoplasmic linker associated |
| | | | | protein 2 |
| 1823 | Protein | AB014527 | CLASP2 | cytoplasmic linker associated |
| | | (Translation) | | protein 2 |
| 1824 | DNA | NM_004599 | | Homo sapiens sterol regulatory |
| | | | | element binding transcription |
| | | | | factor 2 (SREBF2), mRNA |
| 1825 | Protein | NP_004590 | | Sterol regulatory element- |
| | | | | binding transcription factor 2; |
| | | | | sterol regulatory element- |
| | | | | binding protein 2 [Homo |
| | | | | sapiens] |
| 1826 | DNA | NM_013318 | KIAA0515 | KIAA0515 protein |
| 1827 | Protein | NP_037450 | KIAA0515 | KIAA0515 protein |
| 1828 | DNA | D86978 | C7orf14 | chromosome 7 open reading |
| | | | | frame 14 |
| 1829 | Protein | D86978 (Translation) | C7orf14 | chromosome 7 open reading |
| | | | | frame 14 |
| 1830 | DNA | AB020671 | KIAA0864 | KIAA0864 protein |
| 1831 | Protein | AB020671 | KIAA0864 | KIAA0864 protein |
| | | (Translation) | | |
| 1832 | DNA | NM_144586 | MGC29643 | hypothetical protein |
| | | | | MGC29643 |
| 1833 | Protein | NP_653187 | MGC29643 | hypothetical protein |
| | | | | MGC29643 |
| 1834 | DNA | NM_007100 | ATP5I | ATP synthase, H+ transporting, |
| | | | | mitochondrial F0 complex, |
| | | | | subunit e |
| 1835 | Protein | NP_009031 | ATP5I | ATP synthase, H+ transporting, |
| | | | | mitochondrial F0 complex, |
| | | | | subunit e |
| 1836 | DNA | NM_003824 | FADD | Fas (TNFRSF6)-associated via |
| | | | | death domain |
| 1837 | Protein | NP_003815 | FADD | Fas (TNFRSF6)-associated via |
| | | | | death domain |
| 1838 | DNA | NM_014891 | PDAP1 | PDGFA associated protein 1 |
| 1839 | Protein | NP_055706 | PDAP1 | PDGFA associated protein 1 |
| 1840 | DNA | NM_007372 | RNAHP | RNA helicase-related protein |
| 1841 | Protein | NP_031398 | RNAHP | RNA helicase-related protein |
| 1842 | DNA | NM_014928 | | Cluster Incl. AB028969: Homo |
| | | | | sapiens mRNA for KIAA1046 |
| | | | | protein, complete cds |
| | | | | /cds = (577.1782) |
| | | | | /gb = AB028969 /gi = 5689428 |
| | | | | /ug = Hs.89519 /len = 5577 |
| 1843 | Protein | NP_055743 | | Cluster Incl. AB028969: Homo |
| | | | | sapiens mRNA for KIAA1046 |
| | | | | protein, complete cds |
| | | | | /cds = (577.1782) |
| | | | | /gb = AB028969 /gi = 5689428 |
| | | | | /ug = Hs.89519 /len = 5577 |
| 1844 | DNA | NM_005216 | DDOST | dolichyl- |
| | | | | diphosphooligosaccharide- |
| | | | | protein glycosyltransferase |
| 1845 | Protein | NP_005207 | DDOST | dolichyl- |
| | | | | diphosphooligosaccharide- |
| | | | | protein glycosyltransferase |
| 1846 | DNA | NM_014233 | UBTF | upstream binding transcription |
| | | | | factor, RNA polymerase I |
| 1847 | Protein | NP_055048 | UBTF | upstream binding transcription |
| | | | | factor, RNA polymerase I |
| 1848 | DNA | NM_003574 | VAPA | VAMP (vesicle-associated |
| | | | | membrane protein)-associated |
| | | | | protein A, 33 kDa |
| 1849 | Protein | NP_003565 | VAPA | VAMP (vesicle-associated |
| | | | | membrane protein)-associated |
| | | | | protein A, 33 kDa |
| 1850 | DNA | NM_006997 | TACC2 | transforming, acidic coiled-coil |
| | | | | containing protein 2 |
| 1851 | Protein | NP_008928 | TACC2 | transforming, acidic coiled-coil |
| | | | | containing protein 2 |
| 1852 | DNA | NM_018358 | FLJ11198 | hypothetical protein FLJ11198 |
| 1853 | Protein | NP_060828 | FLJ11198 | hypothetical protein FLJ11198 |
| 1854 | DNA | NM_005273 | | Homo sapiens guanine |
| | | | | nucleotide binding protein (G |
| | | | | protein), beta polypeptide 2 |
| | | | | (GNB2), mRNA |
| 1855 | Protein | NP_005264 | | guanine nucleotide-binding |
| | | | | protein, beta-2 subunit; G |
| | | | | protein, beta-2 subunit |
| 1856 | Protein | NP_005264 | GNB2 | guanine nucleotide binding |
| | | | | protein (G protein), beta |
| | | | | polypeptide 2 |
| 1857 | DNA | NM_007027 | TOPBP1 | topoisomerase (DNA) II |
| | | | | binding protein |
| 1858 | Protein | NP_008958 | TOPBP1 | topoisomerase (DNA) II |
| | | | | binding protein |
| 1859 | DNA | NM_005487 | HMG2L1 | high-mobility group protein 2- |
| | | | | like 1 |
| 1860 | Protein | NP_005478 | HMG2L1 | high-mobility group protein 2- |
| | | | | like 1 |
| 1861 | DNA | NM_014791 | MELK | maternal embryonic leucine |
| | | | | zipper kinase |
| 1862 | Protein | NP_055606 | MELK | maternal embryonic leucine |
| | | | | zipper kinase |
| 1863 | DNA | AB028992 | KIAA1069 | KIAA1069 protein |
| 1864 | Protein | AB028992 | KIAA1069 | KIAA1069 protein |
| | | (Translation) | | |
| 1865 | DNA | NM_003921 | BCL10 | B-cell CLL/lymphoma 10 |
| 1866 | Protein | NP_003912 | BCL10 | B-cell CLL/lymphoma 10 |
| 1867 | DNA | NM_004799 | MADHIP | MAD, mothers against |
| | | | | decapentaplegic homolog |
| | | | | (Drosophila) interacting |
| | | | | protein, receptor activation |
| | | | | anchor |
| 1868 | Protein | NP_004790 | MADHIP | MAD, mothers against |
| | | | | decapentaplegic homolog |
| | | | | (Drosophila) interacting |
| | | | | protein, receptor activation |
| | | | | anchor |
| 1869 | DNA | NM_007323 | MADHIP | MAD, mothers against |
| | | | | decapentaplegic homolog |
| | | | | (Drosophila) interacting |
| | | | | protein, receptor activation |
| | | | | anchor |
| 1870 | Protein | NP_015562 | MADHIP | MAD, mothers against |
| | | | | decapentaplegic homolog |
| | | | | (Drosophila) interacting |
| | | | | protein, receptor activation |
| | | | | anchor |
| 1871 | DNA | NM_002912 | REV3L | REV3-like, catalytic subunit of |
| | | | | DNA polymerase zeta (yeast) |
| 1872 | Protein | NP_002903 | REV3L | REV3-like, catalytic subunit of |
| | | | | DNA polymerase zeta (yeast) |
| 1873 | DNA | NM_005470 | SSH3BP1 | spectrin SH3 domain binding |
| | | | | protein 1 |
| 1874 | Protein | NP_005461 | SSH3BP1 | spectrin SH3 domain binding |
| | | | | protein 1 |
| 1875 | DNA | NM_005955 | MTF1 | metal-regulatory transcription |
| | | | | factor 1 |
| 1876 | Protein | NP_005946 | MTF1 | metal-regulatory transcription |
| | | | | factor 1 |
| 1877 | DNA | NM_004868 | GPSN2 | glycoprotein, synaptic 2 |
| 1878 | Protein | NP_004859 | GPSN2 | glycoprotein, synaptic 2 |
| 1879 | DNA | NM_138501 | GPSN2 | glycoprotein, synaptic 2 |
| 1880 | Protein | NP_612510 | GPSN2 | glycoprotein, synaptic 2 |
| 1881 | DNA | NM_007262 | DJ-1 | RNA-binding protein |
| | | | | regulatory subunit |
| 1882 | Protein | NP_009193 | DJ-1 | RNA-binding protein |
| | | | | regulatory subunit |
| 1883 | DNA | NM_006451 | PAIP1 | polyadenylate binding protein- |
| | | | | interacting protein 1 |
| 1884 | Protein | NP_006442 | PAIP1 | polyadenylate binding protein- |
| | | | | interacting protein 1 |
| 1885 | DNA | NM_002491 | NDUFB3 | NADH dehydrogenase |
| | | | | (ubiquinone) 1 beta |
| | | | | subcomplex, 3, 12 kDa |
| 1886 | Protein | NP_002482 | NDUFB3 | NADH dehydrogenase |
| | | | | (ubiquinone) 1 beta |
| | | | | subcomplex, 3, 12 kDa |
| 1887 | DNA | NM_007331 | WHSC1 | Wolf-Hirschhorn syndrome |
| | | | | candidate 1 |
| 1888 | Protein | NP_015627 | WHSC1 | Wolf-Hirschhorn syndrome |
| | | | | candidate 1 |
| 1889 | DNA | NM_014919 | WHSC1 | Wolf-Hirschhorn syndrome |
| | | | | candidate 1 |
| 1890 | Protein | NP_055734 | WHSC1 | Wolf-Hirschhorn syndrome |
| | | | | candidate 1 |
| 1891 | DNA | NM_133330 | WHSC1 | Wolf-Hirschhorn syndrome |
| | | | | candidate 1 |
| 1892 | Protein | NP_579877 | WHSC1 | Wolf-Hirschhorn syndrome |
| | | | | candidate 1 |
| 1893 | DNA | NM_133331 | WHSC1 | Wolf-Hirschhorn syndrome |
| | | | | candidate 1 |
| 1894 | DNA | U55980 | | Homo sapiens cDNA: |
| | | | | FLJ23482 fis, clone |
| | | | | KAIA03142, mRNA sequence |
| 1895 | DNA | AF037989 | | STAT-induced STAT inhibitor- |
| | | | | 2 [Homo sapiens], mRNA |
| | | | | sequence |
| 1896 | Protein | AF037989 | | STAT-induced STAT inhibitor- |
| | | (Translation) | | 2 [Homo sapiens], mRNA |
| | | | | sequence |
| 1897 | DNA | X96924 | | Cluster Incl. X96924: H. sapiens |
| | | | | gene encoding mitochondrial |
| | | | | citrate transport protein |
| | | | | /cds = (0.957) /gb = X96924 |
| | | | | /gi = 1770309 /ug = Hs.111024 |
| | | | | /len = 1522 |
| 1898 | Protein | CAA65633 | | Cluster Incl. X96924: H. sapiens |
| | | | | gene encoding mitochondrial |
| | | | | citrate transport protein |
| | | | | /cds = (0.957) /gb = X96924 |
| | | | | /gi = 1770309 /ug = Hs.111024 |
| | | | | /len = 1522 |
| 1899 | DNA | NM_021079 | NMT1 | N-myristoyltransferase 1 |
| 1900 | Protein | NP_066565 | NMT1 | N-myristoyltransferase 1 |
| 1901 | DNA | AB018257 | ZNF294 | zinc finger protein 294 |
| 1902 | Protein | AB018257 | ZNF294 | zinc finger protein 294 |
| | | (Translation) | | |
| 1903 | DNA | NM_014463 | LSM3 | Lsm3 protein |
| 1904 | Protein | NP_055278 | LSM3 | Lsm3 protein |
| 1905 | DNA | NM_004436 | ENSA | endosulfine alpha |
| 1906 | Protein | NP_004427 | ENSA | endosulfine alpha |
| 1907 | DNA | NM_004528 | MGST3 | microsomal glutathione S- |
| | | | | transferase 3 |
| 1908 | Protein | NP_004519 | MGST3 | microsomal glutathione S- |
| | | | | transferase 3 |
| 1909 | DNA | NM_005387 | NUP98 | nucleoporin 98 kDa |
| 1910 | Protein | NP_005378 | NUP98 | nucleoporin 98 kDa |
| 1911 | DNA | NM_016320 | NUP98 | nucleoporin 98 kDa |
| 1912 | Protein | NP_057404 | NUP98 | nucleoporin 98 kDa |
| 1913 | DNA | NM_139131 | NUP98 | nucleoporin 98 kDa |
| 1914 | Protein | NP_624357 | NUP98 | nucleoporin 98 kDa |
| 1915 | DNA | NM_139132 | NUP98 | nucleoporin 98 kDa |
| 1916 | Protein | NP_624358 | NUP98 | nucleoporin 98 kDa |
| 1917 | DNA | NM_019059 | TOM7 | homolog of Tom7 (S. cerevisiae) |
| 1918 | Protein | NP_061932 | TOM7 | homolog of Tom7 (S. cerevisiae) |
| 1919 | DNA | NM_006423 | RABAC1 | Rab acceptor 1 (prenylated) |
| 1920 | Protein | NP_006414 | RABAC1 | Rab acceptor 1 (prenylated) |
| 1921 | DNA | NM_006022 | TSC22 | transforming growth factor |
| | | | | beta-stimulated protein TSC-22 |
| 1922 | Protein | NP_006013 | TSC22 | transforming growth factor |
| | | | | beta-stimulated protein TSC-22 |
| 1923 | DNA | NM_015902 | DD5 | progestin induced protein |
| 1924 | Protein | NP_056986 | DD5 | progestin induced protein |
| 1925 | DNA | NM_005935 | MLLT2 | myeloid/lymphoid or mixed- |
| | | | | lineage leukemia (trithorax |
| | | | | homolog, Drosophila); |
| | | | | translocated to, 2 |
| 1926 | Protein | NP_005926 | MLLT2 | myeloid/lymphoid or mixed- |
| | | | | lineage leukemia (trithorax |
| | | | | homolog, Drosophila); |
| | | | | translocated to, 2 |
| 1927 | DNA | Y00978 | | PDC-E2 precursor (AA-54 to |
| | | | | 561) [Homo sapiens], mRNA |
| | | | | sequence |
| 1928 | Protein | Y00978 (Translation) | | PDC-E2 precursor (AA-54 to |
| | | | | 561) [Homo sapiens], mRNA |
| | | | | sequence |
| 1929 | DNA | NM_005720 | ARPC1B | actin related protein 2/3 |
| | | | | complex, subunit 1B, 41 kDa |
| 1930 | Protein | NP_005711 | ARPC1B | actin related protein 2/3 |
| | | | | complex, subunit 1B, 41 kDa |
| 1931 | DNA | NM_014706 | SART3 | squamous cell carcinoma |
| | | | | antigen recognised by T cells 3 |
| 1932 | Protein | NP_055521 | SART3 | squamous cell carcinoma |
| | | | | antigen recognised by T cells 3 |
| 1933 | DNA | NM_004698 | HPRP3P | U4/U6-associated RNA |
| | | | | splicing factor |
| 1934 | Protein | NP_004689 | HPRP3P | U4/U6-associated RNA |
| | | | | splicing factor |
| 1935 | DNA | NM_001360 | DHCR7 | 7-dehydrocholesterol reductase |
| 1936 | Protein | NP_001351 | DHCR7 | 7-dehydrocholesterol reductase |
| 1937 | DNA | NM_014623 | MEA | male-enhanced antigen |
| 1938 | Protein | NP_055438 | MEA | male-enhanced antigen |
| 1939 | DNA | U41843 | | Dr1-associated corepressor, |
| | | | | mRNA sequence |
| 1940 | Protein | U41843 (Translation) | | Dr1-associated corepressor, |
| | | | | mRNA sequence |
| 1941 | DNA | NM_014299 | BRD4 | bromodomain containing 4 |
| 1942 | Protein | NP_055114 | BRD4 | bromodomain containing 4 |
| 1943 | DNA | NM_058243 | BRD4 | bromodomain containing 4 |
| 1944 | Protein | NP_490597 | BRD4 | bromodomain containing 4 |
| 1945 | DNA | NM_003103 | SON | SON DNA binding protein |
| 1946 | Protein | NP_003094 | SON | SON DNA binding protein |
| 1947 | DNA | NM_032195 | SON | SON DNA binding protein |
| 1948 | Protein | NP_115571 | SON | SON DNA binding protein |
| 1949 | DNA | NM_058183 | SON | SON DNA binding protein |
| 1950 | Protein | NP_478063 | SON | SON DNA binding protein |
| 1951 | DNA | NM_138925 | SON | SON DNA binding protein |
| 1952 | Protein | NP_620303 | SON | SON DNA binding protein |
| 1953 | DNA | NM_138926 | SON | SON DNA binding protein |
| 1954 | Protein | NP_620304 | SON | SON DNA binding protein |
| 1955 | DNA | NM_005392 | PHF2 | PHD finger protein 2 |
| 1956 | Protein | NP_005383 | PHF2 | PHD finger protein 2 |
| 1957 | DNA | NM_024517 | PHF2 | PHD finger protein 2 |
| 1958 | Protein | NP_078793 | PHF2 | PHD finger protein 2 |
| 1959 | DNA | NM_000175 | GPI | glucose phosphate isomerase |
| 1960 | Protein | NP_000166 | GPI | glucose phosphate isomerase |
| 1961 | DNA | NM_017751 | FLJ20297 | hypothetical protein FLJ20297 |
| 1962 | Protein | NP_060221 | FLJ20297 | hypothetical protein FLJ20297 |
| 1963 | DNA | NM_017951 | FLJ20297 | hypothetical protein FLJ20297 |
| 1964 | Protein | NP_060421 | FLJ20297 | hypothetical protein FLJ20297 |
| 1965 | DNA | AB018310 | KIAA0767 | KIAA0767 protein |
| 1966 | Protein | AB018310 | KIAA0767 | KIAA0767 protein |
| | | (Translation) | | |
| 1967 | DNA | NM_006097 | MYL9 | myosin, light polypeptide 9, |
| | | | | regulatory |
| 1968 | Protein | NP_006088 | MYL9 | myosin, light polypeptide 9, |
| | | | | regulatory |
| 1969 | DNA | NM_005973 | PRCC | papillary renal cell carcinoma |
| | | | | (translocation-associated) |
| 1970 | Protein | NP_005964 | PRCC | papillary renal cell carcinoma |
| | | | | (translocation-associated) |
| 1971 | DNA | NM_014372 | RNF11 | ring finger protein 11 |
| 1972 | Protein | NP_055187 | RNF11 | ring finger protein 11 |
| 1973 | DNA | NM_004645 | COIL | coilin |
| 1974 | Protein | NP_004636 | COIL | coilin |
| 1975 | DNA | NM_001235 | SERPINH2 | serine (or cysteine) proteinase |
| | | | | inhibitor, clade H (heat shock |
| | | | | protein 47), member 2 |
| 1976 | Protein | NP_001226 | SERPINH2 | serine (or cysteine) proteinase |
| | | | | inhibitor, clade H (heat shock |
| | | | | protein 47), member 2 |
| 1977 | DNA | NM_004729 | ALTE | Ac-like transposable element |
| 1978 | Protein | NP_004720 | ALTE | Ac-like transposable element |
| 1979 | DNA | NM_006201 | PCTK1 | PCTAIRE protein kinase 1 |
| 1980 | Protein | NP_006192 | PCTK1 | PCTAIRE protein kinase 1 |
| 1981 | DNA | NM_033018 | PCTK1 | PCTAIRE protein kinase 1 |
| 1982 | DNA | NM_033019 | PCTK1 | PCTAIRE protein kinase 1 |
| 1983 | Protein | NP_148979 | PCTK1 | PCTAIRE protein kinase 1 |
| 1984 | DNA | NM_018074 | FLJ10374 | hypothetical protein FLJ10374 |
| 1985 | Protein | NP_060544 | FLJ10374 | hypothetical protein FLJ10374 |
| 1986 | DNA | NM_001270 | CHD1 | chromodomain helicase DNA |
| | | | | binding protein 1 |
| 1987 | Protein | NP_001261 | CHD1 | chromodomain helicase DNA |
| | | | | binding protein 1 |
| 1988 | DNA | NM_012191 | FUS2 | putative tumor suppressor |
| 1989 | Protein | NP_036323 | FUS2 | putative tumor suppressor |
| 1990 | DNA | NM_005862 | STAG1 | stromal antigen 1 |
| 1991 | Protein | NP_005853 | STAG1 | stromal antigen 1 |
| 1992 | Protein | NP_005393 | RALA | v-ral simian leukemia viral |
| | | | | oncogene homolog A (ras |
| | | | | related) |
| 1993 | DNA | NM_007249 | KLF12 | Kruppel-like factor 12 |
| 1994 | Protein | NP_009180 | KLF12 | Kruppel-like factor 12 |
| 1995 | DNA | NM_016285 | KLF12 | Kruppel-like factor 12 |
| 1996 | Protein | NP_057369 | KLF12 | Kruppel-like factor 12 |
| 1997 | DNA | NM_013299 | HSU79266 | protein predicted by clone |
| | | | | 23627 |
| 1998 | Protein | NP_037431 | HSU79266 | protein predicted by clone |
| | | | | 23627 |
| 1999 | DNA | NM_002915 | RFC3 | replication factor C (activator |
| | | | | 1) 3, 38 kDa |
| 2000 | Protein | NP_002906 | RFC3 | replication factor C (activator |
| | | | | 1) 3, 38 kDa |
| 2001 | DNA | NM_012346 | NUP62 | nucleoporin 62 kDa |
| 2002 | Protein | NP_036478 | NUP62 | nucleoporin 62 kDa |
| 2003 | DNA | NM_016553 | NUP62 | nucleoporin 62 kDa |
| 2004 | Protein | NP_057637 | NUP62 | nucleoporin 62 kDa |
| 2005 | DNA | NM_153718 | NUP62 | nucleoporin 62 kDa |
| 2006 | Protein | NP_714940 | NUP62 | nucleoporin 62 kDa |
| 2007 | DNA | NM_153719 | NUP62 | nucleoporin 62 kDa |
| 2008 | DNA | D64109 | TOB2 | transducer of ERBB2, 2 |
| 2009 | Protein | D64109 (Translation) | TOB2 | transducer of ERBB2, 2 |
| 2010 | DNA | NM_001834 | CLTB | clathrin, light polypeptide (Lcb) |
| 2011 | Protein | NP_001825 | CLTB | clathrin, light polypeptide (Lcb) |
| 2012 | DNA | NM_007097 | CLTB | clathrin, light polypeptide (Lcb) |
| 2013 | Protein | NP_009028 | CLTB | clathrin, light polypeptide (Lcb) |
| 2014 | DNA | NM_018979 | PRKWNK1 | protein kinase, lysine deficient 1 |
| 2015 | Protein | NP_061852 | PRKWNK1 | protein kinase, lysine deficient 1 |
| 2016 | DNA | NM_019892 | PPI5PIV | phosphatidylinositol (4,5) |
| | | | | bisphosphate 5-phosphatase |
| | | | | homolog; phosphatidylinositol |
| | | | | polyphosphate 5-phosphatase |
| | | | | type IV |
| 2017 | Protein | NP_063945 | PPI5PIV | phosphatidylinositol (4,5) |
| | | | | bisphosphate 5-phosphatase |
| | | | | homolog; phosphatidylinositol |
| | | | | polyphosphate 5-phosphatase |
| | | | | type IV |
| 2018 | DNA | NM_004069 | AP2S1 | adaptor-related protein complex |
| | | | | 2, sigma 1 subunit |
| 2019 | Protein | NP_004060 | AP2S1 | adaptor-related protein complex |
| | | | | 2, sigma 1 subunit |
| 2020 | DNA | NM_021575 | AP2S1 | adaptor-related protein complex |
| | | | | 2, sigma 1 subunit |
| 2021 | Protein | NP_067586 | AP2S1 | adaptor-related protein complex |
| | | | | 2, sigma 1 subunit |
| 2022 | DNA | NM_016426 | GTSE1 | G-2 and S-phase expressed 1 |
| 2023 | Protein | NP_057510 | GTSE1 | G-2 and S-phase expressed 1 |
| 2024 | DNA | NM_152696 | Nbak2 | homeodomain interacting |
| | | | | protein kinase 1-like protein |
| 2025 | Protein | NP_689909 | Nbak2 | homeodomain interacting |
| | | | | protein kinase 1-like protein |
| 2026 | DNA | NM_032217 | GTAR | gene trap ankyrin repeat |
| 2027 | Protein | NP_115593 | GTAR | gene trap ankyrin repeat |
| 2028 | DNA | NM_015271 | TRIM2 | tripartite motif-containing 2 |
| 2029 | Protein | NP_056086 | TRIM2 | tripartite motif-containing 2 |
| 2030 | DNA | NM_021005 | NR2F2 | nuclear receptor subfamily 2, |
| | | | | group F, member 2 |
| 2031 | Protein | NP_066285 | NR2F2 | nuclear receptor subfamily 2, |
| | | | | group F, member 2 |
| 2032 | DNA | NM_015079 | KIAA1055 | KIAA1055 protein |
| 2033 | Protein | NP_055894 | KIAA1055 | KIAA1055 protein |
| 2034 | DNA | W28264 | | Unknown (protein for |
| | | | | MGC: 17296) [Homo sapiens], |
| | | | | mRNA sequence |
| 2035 | DNA | NM_021645 | KIAA0266 | KIAA0266 gene product |
| 2036 | Protein | NP_067677 | KIAA0266 | KIAA0266 gene product |
| 2037 | DNA | AL080156 | DKFZP434J214 | DKFZP434J214 protein |
| 2038 | Protein | AL080156 | DKFZP434J214 | DKFZP434J214 protein |
| | | (Translation) | | |
| 2039 | DNA | NM_003449 | TRIM26 | tripartite motif-containing 26 |
| 2040 | Protein | NP_003440 | TRIM26 | tripartite motif-containing 26 |
| 2041 | DNA | NM_014604 | TIP-1 | Tax interaction protein 1 |
| 2042 | Protein | NP_055419 | TIP-1 | Tax interaction protein 1 |
| 2043 | DNA | NM_014570 | ARFGAP3 | ADP-ribosylation factor |
| | | | | GTPase activating protein 3 |
| 2044 | Protein | NP_055385 | ARFGAP3 | ADP-ribosylation factor |
| | | | | GTPase activating protein 3 |
| 2045 | DNA | NM_003605 | OGT | O-linked N-acetylglucosamine |
| | | | | (GlcNAc) transferase (UDP-N- |
| | | | | acetylglucosamine:polypeptide- |
| | | | | N-acetylglucosaminyl |
| | | | | transferase) |
| 2046 | Protein | NP_003596 | OGT | O-linked N-acetylglucosamine |
| | | | | (GlcNAc) transferase (UDP-N- |
| | | | | acetylglucosamine:polypeptide- |
| | | | | N-acetylglucosaminyl |
| | | | | transferase) |
| 2047 | DNA | NM_015898 | FBI1 | HIV-1 inducer of short |
| | | | | transcripts binding protein; |
| | | | | lymphoma related factor |
| 2048 | Protein | NP_056982 | FBI1 | HIV-1 inducer of short |
| | | | | transcripts binding protein; |
| | | | | lymphoma related factor |
| 2049 | DNA | NM_001564 | ING1L | inhibitor of growth family, |
| | | | | member 1-like |
| 2050 | Protein | NP_001555 | ING1L | inhibitor of growth family, |
| | | | | member 1-like |
| 2051 | DNA | NM_014292 | CBX6 | chromobox homolog 6 |
| 2052 | Protein | NP_055107 | CBX6 | chromobox homolog 6 |
| 2053 | DNA | NM_003663 | CGGBP1 | CGG triplet repeat binding |
| | | | | protein 1 |
| 2054 | Protein | NP_003654 | CGGBP1 | CGG triplet repeat binding |
| | | | | protein 1 |
| 2055 | DNA | NM_004329 | BMPR1A | bone morphogenetic protein |
| | | | | receptor, type IA |
| 2056 | Protein | NP_004320 | BMPR1A | bone morphogenetic protein |
| | | | | receptor, type IA |
| 2057 | DNA | NM_015464 | DKFZp564D206 | cystine-knot containing |
| | | | | secreted protein |
| 2058 | Protein | NP_056279 | DKFZp564D206 | cystine-knot containing |
| | | | | secreted protein |
| 2059 | DNA | AI557322 | | Homo sapiens cDNA: |
| | | | | FLJ22256 fis, clone |
| | | | | HRC02860, mRNA sequence |
| 2060 | DNA | AB007928 | KIAA0459 | KIAA0459 protein |
| 2061 | Protein | AB007928 | KIAA0459 | KIAA0459 protein |
| | | (Translation) | | |
| 2062 | DNA | NM_004251 | RAB9A | RAB9A, member RAS |
| | | | | oncogene family |
| 2063 | Protein | NP_004242 | RAB9A | RAB9A, member RAS |
| | | | | oncogene family |
| 2064 | DNA | NM_003223 | TFAP4 | transcription factor AP-4 |
| | | | | (activating enhancer binding |
| | | | | protein 4) |
| 2065 | Protein | NP_003214 | TFAP4 | transcription factor AP-4 |
| | | | | (activating enhancer binding |
| | | | | protein 4) |
| 2066 | DNA | NM_007215 | POLG2 | polymerase (DNA directed), |
| | | | | gamma 2, accessory subunit |
| 2067 | Protein | NP_009146 | POLG2 | polymerase (DNA directed), |
| | | | | gamma 2, accessory subunit |
| 2068 | DNA | NM_004312 | ARR3 | arrestin 3, retinal (X-arrestin) |
| 2069 | Protein | NP_004303 | ARR3 | arrestin 3, retinal (X-arrestin) |
| 2070 | DNA | NM_015569 | KIAA0820 | KIAA0820 protein |
| 2071 | Protein | NP_056384 | KIAA0820 | KIAA0820 protein |
| 2072 | DNA | NM_021140 | UTX | ubiquitously transcribed |
| | | | | tetratricopeptide repeat gene, X |
| | | | | chromosome |
| 2073 | Protein | NP_066963 | UTX | ubiquitously transcribed |
| | | | | tetratricopeptide repeat gene, X |
| | | | | chromosome |
| 2074 | DNA | NM_002131 | HMGA1 | high mobility group AT-hook 1 |
| 2075 | Protein | NP_002122 | HMGA1 | high mobility group AT-hook 1 |
| 2076 | DNA | NM_145899 | HMGA1 | high mobility group AT-hook 1 |
| 2077 | Protein | NP_665906 | HMGA1 | high mobility group AT-hook 1 |
| 2078 | DNA | NM_145901 | HMGA1 | high mobility group AT-hook 1 |
| 2079 | DNA | NM_145902 | HMGA1 | high mobility group AT-hook 1 |
| 2080 | DNA | NM_003009 | SEPW1 | selenoprotein W, 1 |
| 2081 | Protein | NP_003000 | SEPW1 | selenoprotein W, 1 |
| 2082 | DNA | NM_005979 | S100A13 | S100 calcium binding protein |
| | | | | A13 |
| 2083 | Protein | NP_005970 | S100A13 | S100 calcium binding protein |
| | | | | A13 |
| 2084 | DNA | NM_006618 | PLU-1 | putative DNA/chromatin |
| | | | | binding motif |
| 2085 | Protein | NP_006609 | PLU-1 | putative DNA/chromatin |
| | | | | binding motif |
| 2086 | DNA | NM_003592 | CUL1 | cullin 1 |
| 2087 | Protein | NP_003583 | CUL1 | cullin 1 |
| 2088 | DNA | NM_004902 | RNPC2 | RNA-binding region (RNP1, |
| | | | | RRM) containing 2 |
| 2089 | Protein | NP_004893 | RNPC2 | RNA-binding region (RNP1, |
| | | | | RRM) containing 2 |
| 2090 | DNA | NM_003584 | DUSP11 | dual specificity phosphatase 11 |
| | | | | (RNA/RNP complex 1- |
| | | | | interacting) |
| 2091 | Protein | NP_003575 | DUSP11 | dual specificity phosphatase 11 |
| | | | | (RNA/RNP complex 1- |
| | | | | interacting) |
| 2092 | DNA | NM_005809 | PRDX2 | peroxiredoxin 2 |
| 2093 | Protein | NP_005800 | PRDX2 | peroxiredoxin 2 |
| 2094 | DNA | NM_005157 | ABL1 | v-abl Abelson murine leukemia |
| | | | | viral oncogene homolog 1 |
| 2095 | Protein | NP_005148 | ABL1 | v-abl Abelson murine leukemia |
| | | | | viral oncogene homolog 1 |
| 2096 | DNA | NM_007313 | ABL1 | v-abl Abelson murine leukemia |
| | | | | viral oncogene homolog 1 |
| 2097 | Protein | NP_009297 | ABL1 | v-abl Abelson murine leukemia |
| | | | | viral oncogene homolog 1 |
| 2098 | DNA | NM_001356 | DDX3 | DEAD/H (Asp-Glu-Ala- |
| | | | | Asp/His) box polypeptide 3 |
| 2099 | Protein | NP_001347 | DDX3 | DEAD/H (Asp-Glu-Ala- |
| | | | | Asp/His) box polypeptide 3 |
| 2100 | DNA | NM_024005 | DDX3 | DEAD/H (Asp-Glu-Ala- |
| | | | | Asp/His) box polypeptide 3 |
| 2101 | DNA | NM_000938 | POLR2B | polymerase (RNA) II (DNA |
| | | | | directed) polypeptide B, |
| | | | | 140 kDa |
| 2102 | Protein | NP_000929 | POLR2B | polymerase (RNA) II (DNA |
| | | | | directed) polypeptide B, |
| | | | | 140 kDa |
| 2103 | DNA | NM_005080 | XBP1 | X-box binding protein 1 |
| 2104 | Protein | NP_005071 | XBP1 | X-box binding protein 1 |
| 2105 | DNA | AL031781 | QKI | homolog of mouse quaking |
| | | | | QKI (KH domain RNA binding |
| | | | | protein) |
| 2106 | DNA | NM_005095 | ZNF262 | zinc finger protein 262 |
| 2107 | Protein | NP_005086 | ZNF262 | zinc finger protein 262 |
| 2108 | DNA | NM_014837 | C1orf16 | chromosome 1 open reading |
| | | | | frame 16 |
| 2109 | Protein | NP_055652 | C1orf16 | chromosome 1 open reading |
| | | | | frame 16 |
| 2110 | DNA | NM_015057 | KIAA0916 | KIAA0916 protein |
| 2111 | Protein | NP_055872 | KIAA0916 | KIAA0916 protein |
| 2112 | DNA | NM_004094 | EIF2S1 | eukaryotic translation initiation |
| | | | | factor 2, subunit 1 alpha, 35 kDa |
| 2113 | Protein | NP_004085 | EIF2S1 | eukaryotic translation initiation |
| | | | | factor 2, subunit 1 alpha, 35 kDa |
| 2114 | DNA | NM_001681 | ATP2A2 | ATPase, Ca++ transporting, |
| | | | | cardiac muscle, slow twitch 2 |
| 2115 | Protein | NP_001672 | ATP2A2 | ATPase, Ca++ transporting |
| | | | | cardiac muscle, slow twitch 2 |
| 2116 | DNA | NM_170665 | ATP2A2 | ATPase, Ca++ transporting, |
| | | | | cardiac muscle, slow twitch 2 |
| 2117 | Protein | NP_733765 | ATP2A2 | ATPase, Ca++ transporting, |
| | | | | cardiac muscle, slow twitch 2 |
| 2118 | DNA | NM_015255 | KIAA0349 | KIAA0349 protein |
| 2119 | Protein | NP_056070 | KIAA0349 | KIAA0349 protein |
| 2120 | DNA | NM_001031 | RPS28 | ribosomal protein S28 |
| 2121 | Protein | NP_001022 | RPS28 | ribosomal protein S28 |
| 2122 | DNA | NM_006443 | RCL | putative c-Myc-responsive |
| 2123 | Protein | NP_006434 | RCL | putative c-Myc-responsive |
| 2124 | DNA | NM_000988 | RPL27 | ribosomal protein L27 |
| 2125 | Protein | NP_000979 | RPL27 | ribosomal protein L27 |
| 2126 | DNA | U93181 | SBF1 | SET binding factor 1 |
| 2127 | Protein | U93181 (Translation) | SBF1 | SET binding factor 1 |
| 2128 | DNA | AC004877 | | Cluster Incl. AC004877: Homo |
| | | | | sapiens PAC clone DJ0751H13 |
| | | | | from 7q35-qter /cds = (0.1514) |
| | | | | /gb = AC004877 /gi = 3638954 |
| | | | | /ug = Hs.112158 /len = 1515 |
| 2129 | Protein | AC004877 | | Cluster Incl. AC004877: Homo |
| | | (Translation) | | sapiens PAC clone DJ0751H13 |
| | | | | from 7q35-qter /cds = (0.1514) |
| | | | | /gb = AC004877 /gi = 3638954 |
| | | | | /ug = Hs.112158 /len = 1515 |
| 2130 | DNA | NM_003651 | CSDA | cold shock domain protein A |
| 2131 | Protein | NP_003642 | CSDA | cold shock domain protein A |
| 2132 | DNA | NM_004694 | SLC16A6 | solute carrier family 16 |
| | | | | (monocarboxylic acid |
| | | | | transporters), member 6 |
| 2133 | Protein | NP_004685 | SLC16A6 | solute carrier family 16 |
| | | | | (monocarboxylic acid |
| | | | | transporters), member 6 |
| 2134 | DNA | AB028986 | USP22 | ubiquitin specific protease 22 |
| 2135 | Protein | AB028986 | USP22 | ubiquitin specific protease 22 |
| | | (Translation) | | |
| 2136 | DNA | NM_003321 | TUFM | Tu translation elongation |
| | | | | factor, mitochondrial |
| 2137 | Protein | NP_003312 | TUFM | Tu translation elongation |
| | | | | factor, mitochondrial |
| 2138 | DNA | NM_014473 | HSA9761 | putative dimethyladenosine |
| | | | | transferase |
| 2139 | Protein | NP_055288 | HSA9761 | putative dimethyladenosine |
| | | | | transferase |
| 2140 | DNA | NM_014577 | | Cluster Incl. Z98885: Human |
| | | | | DNA sequence from clone |
| | | | | 522J7 on chromosome 22q13.3. |
| | | | | Contains part of a 60S |
| | | | | Ribosomal protein L5 |
| | | | | pseudogene and a Peregrin |
| | | | | (BR140) LIKE gene |
| | | | | downstream of a putative CpG |
| | | | | island. Contains ESTs, STSs |
| | | | | and GSSs /cds = (185.3361) |
| | | | | /gb = Z |
| 2141 | Protein | NP_055392 | | Cluster Incl. Z98885: Human |
| | | | | DNA sequence from clone |
| | | | | 522J7 on chromosome 22q13.3. |
| | | | | Contains part of a 60S |
| | | | | Ribosomal protein L5 |
| | | | | pseudogene and a Peregrin |
| | | | | (BR140) LIKE gene |
| | | | | downstream of a putative CpG |
| | | | | island. Contains ESTs, STSs |
| | | | | and GSSs /cds = (185.3361) |
| | | | | /gb = Z |
| 2142 | DNA | NM_133370 | KIAA1966 | KIAA1966 protein |
| 2143 | Protein | NP_588611 | KIAA1966 | KIAA1966 protein |
| 2144 | DNA | NM_015196 | KIAA0922 | KIAA0922 protein |
| 2145 | Protein | NP_056011 | KIAA0922 | KIAA0922 protein |
| 2146 | DNA | AI655015 | | Homo sapiens mRNA; cDNA |
| | | | | DKFZp586F2224 (from clone |
| | | | | DKFZp586F2224), mRNA |
| | | | | sequence |
| 2147 | DNA | NM_006190 | ORC2L | origin recognition complex, |
| | | | | subunit 2-like (yeast) |
| 2148 | Protein | NP_006181 | ORC2L | origin recognition complex, |
| | | | | subunit 2-like (yeast) |
| 2149 | DNA | NM_005227 | EFNA4 | ephrin-A4 |
| 2150 | Protein | NP_005218 | EFNA4 | ephrin-A4 |
| 2151 | DNA | NM_006714 | ASM3A | acid sphingomyelinase-like |
| | | | | phosphodiesterase |
| 2152 | Protein | NP_006705 | ASM3A | acid sphingomyelinase-like |
| | | | | phosphodiesterase |
| 2153 | DNA | AF150247 | | HSPC060 [Homo sapiens], |
| | | | | mRNA sequence |
| 2154 | DNA | NM_003542 | H4FG | H4 histone family, member G |
| 2155 | Protein | NP_003533 | H4FG | H4 histone family, member G |
| 2156 | DNA | NM_006020 | ALKBH | alkB, alkylation repair homolog |
| | | | | (E. coli) |
| 2157 | Protein | NP_006011 | ALKBH | alkB, alkylation repair homolog |
| | | | | (E. coli) |
| 2158 | DNA | NM_014777 | KIAA0133 | KIAA0133 gene product |
| 2159 | Protein | NP_055592 | KIAA0133 | KIAA0133 gene product |
| 2160 | DNA | NM_006101 | HEC | highly expressed in cancer, rich |
| | | | | in leucine heptad repeats |
| 2161 | Protein | NP_006092 | HEC | highly expressed in cancer, rich |
| | | | | in leucine heptad repeats |
| 2162 | DNA | NM_005785 | SBB103 | hypothetical SBBI03 protein |
| 2163 | Protein | NP_005776 | SBB103 | hypothetical SBBI03 protein |
| 2164 | DNA | NM_014676 | PUM1 | pumilio homolog 1 |
| | | | | (Drosophila) |
| 2165 | Protein | NP_055491 | PUM1 | pumilio homolog 1 |
| | | | | (Drosophila) |
| 2166 | DNA | NM_002657 | PLAGL2 | pleiomorphic adenoma gene- |
| | | | | like 2 |
| 2167 | Protein | NP_002648 | PLAGL2 | pleiomorphic adenoma gene- |
| | | | | like 2 |
| 2168 | DNA | NM_005831 | NDP52 | nuclear domain 10 protein |
| 2169 | Protein | NP_005822 | NDP52 | nuclear domain 10 protein |
| 2170 | DNA | NM_003174 | SVIL | supervillin |
| 2171 | Protein | NP_003165 | SVIL | supeivillin |
| 2172 | DNA | NM_021738 | SVIL | supervillin |
| 2173 | Protein | NP_068506 | SVIL | supervillin |
| 2174 | DNA | NM_005676 | RBM10 | RNA binding motif protein 10 |
| 2175 | Protein | NP_005667 | RBM10 | RNA binding motif protein 10 |
| 2176 | DNA | NM_152856 | RBM10 | RNA binding motif protein 10 |
| 2177 | Protein | NP_690595 | RBM10 | RNA binding motif protein 10 |
| 2178 | DNA | NM_015046 | KIAA0625 | KIAA0625 protein |
| 2179 | Protein | NP_055861 | KIAA0625 | KIAA0625 protein |
| 2180 | DNA | D87450 | KIAA0261 | KIAA0261 protein |
| 2181 | Protein | D87450 (Translation) | KIAA0261 | KIAA0261 protein |
| 2182 | DNA | NM_003489 | NRIP1 | nuclear receptor interacting |
| | | | | protein 1 |
| 2183 | Protein | NP_003480 | NRIP1 | nuclear receptor interacting |
| | | | | protein 1 |
| 2184 | DNA | NM_017528 | WBSCR22 | Williams Beuren syndrome |
| | | | | chromosome region 22 |
| 2185 | Protein | NP_059998 | WBSCR22 | Williams Beuren syndrome |
| | | | | chromosome region 22 |
| 2186 | DNA | NM_006795 | EHD1 | EH-domain containing 1 |
| 2187 | Protein | NP_006786 | EHD1 | EH-domain containing 1 |
| 2188 | DNA | NM_006374 | STK25 | serine/threonine kinase 25 |
| | | | | (STE20 homolog, yeast) |
| 2189 | Protein | NP_006365 | STK25 | serine/threonine kinase 25 |
| | | | | (STE20 homolog, yeast) |
| 2190 | DNA | NM_007040 | E1B-AP5 | E1B-55 kDa-associated protein 5 |
| 2191 | Protein | NP_008971 | E1B-AP5 | E1B-55 kDa-associated protein 5 |
| 2192 | DNA | NM_144732 | E1B-AP5 | E1B-55 kDa-associated protein 5 |
| 2193 | Protein | NP_653333 | E1B-AP5 | E1B-55 kDa-associated protein 5 |
| 2194 | DNA | NM_144733 | E1B-AP5 | E1B-55 kDa-associated protein 5 |
| 2195 | Protein | NP_653334 | E1B-AP5 | E1B-55 kDa-associated protein 5 |
| 2196 | DNA | NM_144734 | E1B-AP5 | E1B-55 kDa-associated protein 5 |
| 2197 | Protein | NP_653335 | E1B-AP5 | E1B-55 kDa-associated protein 5 |
| 2198 | DNA | NM_017715 | ZNF3 | zinc finger protein 3 (A8-51) |
| 2199 | Protein | NP_060185 | ZNF3 | zinc finger protein 3 (A8-51) |
| 2200 | DNA | NM_032924 | ZNF3 | zinc finger protein 3 (A8-51) |
| 2201 | Protein | NP_116313 | ZNF3 | zinc finger protein 3 (A8-51) |
| 2202 | DNA | NM_006371 | CRTAP | cartilage associated protein |
| 2203 | Protein | NP_006362 | CRTAP | cartilage associated protein |
| 2204 | DNA | NM_006372 | NSAP1 | NS1-associated protein 1 |
| 2205 | Protein | NP_006363 | NSAP1 | NS1-associated protein 1 |
| 2206 | DNA | NM_014666 | ENTH | enthoprotin |
| 2207 | Protein | NP_055481 | ENTH | enthoprotin |
| 2208 | DNA | NM_004889 | ATP5J2 | ATP synthase, H+ transporting, |
| | | | | mitochondrial F0 complex, |
| | | | | subunit f, isoform 2 |
| 2209 | Protein | NP_004880 | ATP5J2 | ATP synthase, H+ transporting, |
| | | | | mitochondrial F0 complex, |
| | | | | subunit f, isoform 2 |
| 2210 | DNA | NM_005667 | ZFP103 | zinc finger protein 103 |
| | | | | homolog (mouse) |
| 2211 | Protein | NP_005658 | ZFP103 | zinc finger protein 103 |
| | | | | homolog (mouse) |
| 2212 | DNA | NM_014661 | KIAA0140 | KIAA0140 gene product |
| 2213 | Protein | NP_055476 | KIAA0140 | KIAA0140 gene product |
| 2214 | DNA | NM_015646 | RAP1B | RAP1B, member of RAS |
| | | | | oncogene family |
| 2215 | Protein | NP_056461 | RAP1B | RAP1B, member of RAS |
| | | | | oncogene family |
| 2216 | DNA | NM_172020 | POM121 | POM121 membrane |
| | | | | glycoprotein (rat) |
| 2217 | Protein | NP_742017 | POM121 | POM121 membrane |
| | | | | glycoprotein (rat) |
| 2218 | DNA | NM_012083 | FRAT2 | frequently rearranged in |
| | | | | advanced T-cell lymphomas 2 |
| 2219 | Protein | NP_036215 | FRAT2 | frequently rearranged in |
| | | | | advanced T-cell lymphomas 2 |
| 2220 | DNA | NM_144635 | MGC21688 | hypothetical protein |
| | | | | MGC21688 |
| 2221 | Protein | NP_653236 | MGC21688 | hypothetical protein |
| | | | | MGC21688 |
| 2222 | DNA | NM_006510 | RFP | ret finger protein |
| 2223 | Protein | NP_006501 | RFP | ret finger protein |
| 2224 | DNA | NM_030950 | RFP | ret finger protein |
| 2225 | Protein | NP_112212 | RFP | ret finger protein |
| 2226 | DNA | AI761647 | | Homo sapiens cDNA FLJ36527 |
| | | | | fis, clone TRACH2003941, |
| | | | | mRNA sequence |
| 2227 | DNA | NM_002105 | H2AFX | H2A histone family, member X |
| 2228 | Protein | NP_002096 | H2AFX | H2A histone family, member X |
| 2229 | DNA | NM_005801 | SUI1 | putative translation initiation |
| | | | | factor |
| 2230 | Protein | NP_005792 | SUI1 | putative translation initiation |
| | | | | factor |
| 2231 | DNA | R37702 | | ESTs |
| 2232 | DNA | NM_003358 | UGCG | UDP-glucose ceramide |
| | | | | glucosyltransferase |
| 2233 | Protein | NP_003349 | UGCG | UDP-glucose ceramide |
| | | | | glucosyltransferase |
| 2234 | DNA | NM_006460 | HIS1 | HMBA-inducible |
| 2235 | Protein | NP_006451 | HIS1 | HMBA-inducible |
| 2236 | DNA | NM_018380 | DDX28 | DEAD/H (Asp-Glu-Ala- |
| | | | | Asp/His) box polypeptide 28 |
| 2237 | Protein | NP_060850 | DDX28 | DEAD/H (Asp-Glu-Ala- |
| | | | | Asp/His) box polypeptide 28 |
| 2238 | DNA | NM_001895 | CSNK2A1 | casein kinase 2, alpha 1 |
| | | | | polypeptide |
| 2239 | Protein | NP_001886 | CSNK2A1 | casein kinase 2, alpha 1 |
| | | | | polypeptide |
| 2240 | DNA | NM_003675 | PRPF18 | PRP18 pre-mRNA processing |
| | | | | factor 18 homolog (yeast) |
| 2241 | Protein | NP_003666 | PRPF18 | PRP18 pre-mRNA processing |
| | | | | factor 18 homolog (yeast) |
| 2242 | DNA | NM_001352 | DBP | D site of albumin promoter |
| | | | | (albumin D-box) binding |
| | | | | protein |
| 2243 | Protein | NP_001343 | DBP | D site of albumin promoter |
| | | | | (albumin D-box) binding |
| | | | | protein |
| 2244 | DNA | NM_020126 | DBP | D site of albumin promoter |
| | | | | (albumin D-box) binding |
| | | | | protein |
| 2245 | Protein | NP_064511 | DBP | D site of albumin promoter |
| | | | | (albumin D-box) binding |
| | | | | protein |
| 2246 | DNA | NM_004404 | NEDD5 | neural precursor cell expressed, |
| | | | | developmentally down- |
| | | | | regulated 5 |
| 2247 | Protein | NP_004395 | NEDD5 | neural precursor cell expressed, |
| | | | | developmentally down- |
| | | | | regulated 5 |
| 2248 | DNA | NM_002533 | NVL | nuclear VCP-like |
| 2249 | Protein | NP_002524 | NVL | nuclear VCP-like |
| 2250 | DNA | AI830496 | KIAA1240 | KIAA1240 protein |
| 2251 | DNA | NM_000474 | TWIST | twist homolog |
| | | | | (acrocephalosyndactyly 3; |
| | | | | Saethre-Chotzen syndrome) |
| | | | | (Drosophila) |
| 2252 | Protein | NP_000465 | TWIST | twist homolog |
| | | | | (acrocephalosyndactyly 3; |
| | | | | Saethre-Chotzen syndrome) |
| | | | | (Drosophila) |
| 2253 | DNA | NM_007346 | OGFR | opioid growth factor receptor |
| 2254 | Protein | NP_031372 | OGFR | opioid growth factor receptor |
| 2255 | DNA | NM_001202 | BMP4 | bone morphogenetic protein 4 |
| 2256 | Protein | NP_001193 | BMP4 | bone morphogenetic protein 4 |
| 2257 | DNA | NM_130850 | BMP4 | bone morphogenetic protein 4 |
| 2258 | DNA | NM_130851 | BMP4 | bone morphogenetic protein 4 |
| 2259 | DNA | NM_015421 | DKFZP564K2062 | DKFZP564K2062 protein |
| 2260 | Protein | NP_056236 | DKFZP564K2062 | DKFZP564K2062 protein |
| 2261 | DNA | NM_005924 | MEOX2 | mesenchyme homeo box 2 |
| | | | | (growth arrest-specific homeo |
| | | | | box) |
| 2262 | Protein | NP_005915 | MEOX2 | mesenchyme homeo box 2 |
| | | | | (growth arrest-specific homeo |
| | | | | box) |
| 2263 | DNA | NM_014071 | NCOA6 | nuclear receptor coactivator 6 |
| 2264 | Protein | NP_054790 | NCOA6 | nuclear receptor coactivator 6 |
| 2265 | DNA | NM_015252 | KIAA0903 | KIAA0903 protein |
| 2266 | Protein | NP_056067 | KIAA0903 | KIAA0903 protein |
| 2267 | DNA | NM_001707 | BCL7B | B-cell CLL/lymphoma 7B |
| 2268 | Protein | NP_001698 | BCL7B | B-cell CLL/lymphoma 7B |
| 2269 | DNA | NM_138707 | BCL7B | B-cell CLL/lymphoma 7B |
| 2270 | Protein | NP_619713 | BCL7B | B-cell CLL/lymphoma 7B |
| 2271 | DNA | NM_015251 | KIAA0431 | KIAA0431 protein |
| 2272 | Protein | NP_056066 | KIAA0431 | KIAA0431 protein |
| 2273 | DNA | NM_015497 | DKFZP564G2022 | DKFZP564G2022 protein |
| 2274 | Protein | NP_056312 | DKFZP564G2022 | DKFZP564G2022 protein |
| 2275 | DNA | NM_002480 | PPP1R12A | protein phosphatase 1, |
| | | | | regulatory (inhibitor) subunit |
| | | | | 12A |
| 2276 | Protein | NP_002471 | PPP1R12A | protein phosphatase 1, |
| | | | | regulatory (inhibitor) subunit |
| | | | | 12A |
| 2277 | DNA | NM_004514 | ILF1 | interleukin enhancer binding |
| | | | | factor 1 |
| 2278 | Protein | NP_004505 | ILF1 | interleukin enhancer binding |
| | | | | factor 1 |
| 2279 | DNA | AB020633 | KIAA0826 | KIAA0826 protein |
| 2280 | Protein | AB020633 | KIAA0826 | KIAA0826 protein |
| | | (Translation) | | |
| 2281 | DNA | NM_020465 | NDRG4 | NDRG family member 4 |
| 2282 | Protein | NP_065198 | NDRG4 | NDRG family member 4 |
| 2283 | DNA | NM_022910 | NDRG4 | NDRG family member 4 |
| 2284 | DNA | NM_015966 | SDBCAG84 | serologically defined breast |
| | | | | cancer antigen 84 |
| 2285 | Protein | NP_057050 | SDBCAG84 | serologically defined breast |
| | | | | cancer antigen 84 |
| 2286 | DNA | NM_007198 | PROSC | proline synthetase co- |
| | | | | transcribed homolog (bacterial) |
| 2287 | Protein | NP_009129 | PROSC | proline synthetase co- |
| | | | | transcribed homolog (bacterial) |
| 2288 | DNA | NM_004935 | CDK5 | cyclin-dependent kinase 5 |
| 2289 | Protein | NP_004926 | CDK5 | cyclin-dependent kinase 5 |
| 2290 | DNA | AL049987 | | Homo sapiens mRNA; cDNA |
| | | | | DKFZp564F112 (from clone |
| | | | | DKFZp564F112), mRNA |
| | | | | sequence |
| 2291 | DNA | NM_005994 | TBX2 | T-box 2 |
| 2292 | Protein | NP_005985 | TBX2 | T-box 2 |
| 2293 | DNA | AL050007 | DKFZP564A043 | DKFZP564A043 protein |
| 2294 | Protein | AL050007 | DKFZP564A043 | DKFZP564A043 protein |
| | | (Translation) | | |
| 2295 | DNA | NM_007172 | NUP50 | nucleoporin 50 kDa |
| 2296 | Protein | NP_009103 | NUP50 | nucleoporin 50 kDa |
| 2297 | DNA | NM_153645 | NUP50 | nucleoporin 50 kDa |
| 2298 | Protein | NP_705931 | NUP50 | nucleoporin 50 kDa |
| 2299 | DNA | NM_153684 | NUP50 | nucleoporin 50 kDa |
| 2300 | DNA | NM_002824 | PTMS | parathymosin |
| 2301 | Protein | NP_002815 | PTMS | parathymosin |
| 2302 | DNA | AF052178 | | Homo sapiens clone 24523 |
| | | | | mRNA sequence |
| 2303 | DNA | NM_003583 | DYRK2 | dual-specificity tyrosine-(Y)- |
| | | | | phosphorylation regulated |
| | | | | kinase 2 |
| 2304 | Protein | NP_003574 | DYRK2 | dual-specificity tyrosine-(Y)- |
| | | | | phosphorylation regulated |
| | | | | kinase 2 |
| 2305 | DNA | NM_006482 | DYRK2 | dual-specificity tyrosine-(Y)- |
| | | | | phosphorylation regulated |
| | | | | kinase 2 |
| 2306 | Protein | NP_006473 | DYRK2 | dual-specificity tyrosine-(Y)- |
| | | | | phosphorylation regulated |
| | | | | kinase 2 |
| 2307 | DNA | AI475497 | HELSNF1 | helicase with SNF2 domain 1 |
| 2308 | DNA | NM_016107 | ZFR | zinc finger RNA binding |
| | | | | protein |
| 2309 | Protein | NP_057191 | ZFR | zinc finger RNA binding |
| | | | | protein |
| 2310 | DNA | NM_025137 | FLJ21439 | hypothetical protein FLJ21439 |
| 2311 | Protein | NP_079413 | FLJ21439 | hypothetical protein FLJ21439 |
| 2312 | DNA | NM_017736 | FLJ20274 | hypothetical protein FLJ20274 |
| 2313 | Protein | NP_060206 | FLJ20274 | hypothetical protein FLJ20274 |
| 2314 | DNA | NM_017548 | H41 | hypothetical protein H41 |
| 2315 | Protein | NP_060018 | H41 | hypothetical protein H41 |
| 2316 | DNA | NM_005749 | TOB1 | transducer of ERBB2, 1 |
| 2317 | Protein | NP_005740 | TOB1 | transducer of ERBB2, 1 |
| 2318 | DNA | NM_005803 | FLOT1 | flotillin 1 |
| 2319 | Protein | NP_005794 | FLOT1 | flotillin 1 |
| 2320 | DNA | NM_005138 | SCO2 | SCO cytochrome oxidase |
| | | | | deficient homolog 2 (yeast) |
| 2321 | Protein | NP_005129 | SCO2 | SCO cytochrome oxidase |
| | | | | deficient homolog 2 (yeast) |
| 2322 | DNA | AI312646 | | Homo sapiens mRNA; cDNA |
| | | | | DKFZp564H1916 (from clone |
| | | | | DKFZp564H1916), mRNA |
| | | | | sequence |
| 2323 | DNA | NM_003937 | KYNU | kynureninase (L-kynurenine |
| | | | | hydrolase) |
| 2324 | Protein | NP_003928 | KYNU | kynureninase (L-kynurenine |
| | | | | hydrolase) |
| 2325 | DNA | NM_001827 | CKS2 | CDC28 protein kinase |
| | | | | regulatory subunit 2 |
| 2326 | Protein | NP_001818 | CKS2 | CDC28 protein kinase |
| | | | | regulatory subunit 2 |
| 2327 | DNA | NM_016324 | ZNF274 | zinc finger protein 274 |
| 2328 | Protein | NP_057408 | ZNF274 | zinc finger protein 274 |
| 2329 | DNA | NM_016325 | ZNF274 | zinc finger protein 274 |
| 2330 | Protein | NP_057409 | ZNF274 | zinc finger protein 274 |
| 2331 | DNA | NM_133502 | ZNF274 | zinc finger protein 274 |
| 2332 | Protein | NP_598009 | ZNF274 | zinc finger protein 274 |
| 2333 | DNA | NM_004523 | KNSL1 | kinesin-like 1 |
| 2334 | Protein | NP_004514 | KNSL1 | kinesin-like 1 |
| 2335 | DNA | NM_014885 | APC10 | anaphase-promoting complex |
| | | | | subunit 10 |
| 2336 | Protein | NP_055700 | APC10 | anaphase-promoting complex |
| | | | | subunit 10 |
| 2337 | DNA | NM_002519 | NPAT | nuclear protein, ataxia- |
| | | | | telangiectasia locus |
| 2338 | Protein | NP_002510 | NPAT | nuclear protein, ataxia- |
| | | | | telangiectasia locus |
| 2339 | DNA | NM_002449 | MSX2 | msh homeo box homolog 2 |
| | | | | (Drosophila) |
| 2340 | Protein | NP_002440 | MSX2 | msh homeo box homolog 2 |
| | | | | (Drosophila) |
| 2341 | DNA | NM_002398 | MEIS1 | Meis1, myeloid ecotropic viral |
| | | | | integration site 1 homolog |
| | | | | (mouse) |
| 2342 | Protein | NP_002389 | MEIS1 | Meis1, myeloid ecotropic viral |
| | | | | integration site 1 homolog |
| | | | | (mouse) |
| 2343 | DNA | NM_005085 | NUP214 | nucleoporin 214 kDa |
| 2344 | Protein | NP_005076 | NUP214 | nucleoporin 214 kDa |
| 2345 | DNA | NM_153642 | NUP214 | nucleoporin 214 kDa |
| 2346 | Protein | NP_705906 | NUP214 | nucleoporin 214 kDa |
| 2347 | DNA | NM_004493 | HADH2 | hydroxyacyl-Coenzyme A |
| | | | | dehydrogenase, type II |
| 2348 | Protein | NP_004484 | HADH2 | hydroxyacyl-Coenzyme A |
| | | | | dehydrogenase, type II |
| 2349 | DNA | NM_001329 | CTBP2 | C-terminal binding protein 2 |
| 2350 | Protein | NP_001320 | CTBP2 | C-terminal binding protein 2 |
| 2351 | DNA | NM_022802 | CTBP2 | C-terminal binding protein 2 |
| 2352 | Protein | NP_073713 | CTBP2 | C-terminal binding protein 2 |
| 2353 | DNA | NM_133264 | WIRE | WIRE protein |
| 2354 | Protein | NP_573571 | WIRE | WIRE protein |
| 2355 | DNA | NM_000937 | POLR2A | polymerase (RNA) II (DNA |
| | | | | directed) polypeptide A, |
| | | | | 220 kDa |
| 2356 | Protein | NP_000928 | POLR2A | polymerase (RNA) II (DNA |
| | | | | directed) polypeptide A, |
| | | | | 220 kDa |
| 2357 | DNA | AA643063 | DKFZP434C212 | DKFZP434C212 protein |
| 2358 | DNA | NM_001275 | CHGA | chromogranin A (parathyroid |
| | | | | secretory protein 1) |
| 2359 | Protein | NP_001266 | CHGA | chromogranin A (parathyroid |
| | | | | secretory protein 1) |
| 2360 | DNA | NM_015555 | COASTER | coactivator for steroid receptors |
| 2361 | Protein | NP_056370 | COASTER | coactivator for steroid receptors |
| 2362 | DNA | NM_015874 | KBF2 | H-2K binding factor-2 |
| 2363 | Protein | NP_056958 | KBF2 | H-2K binding factor-2 |
| 2364 | DNA | NM_000687 | AHCY | S-adenosylhomocysteine |
| | | | | hydrolase |
| 2365 | Protein | NP_000678 | AHCY | S-adenosylhomocysteine |
| | | | | hydrolase |
| 2366 | DNA | NM_002376 | MARK3 | MAP/microtubule affinity- |
| | | | | regulating kinase 3 |
| 2367 | Protein | NP_002367 | MARK3 | MAP/microtubule affinity- |
| | | | | regulating kinase 3 |
| 2368 | DNA | NM_003899 | ARHGEF7 | Rho guanine nucleotide |
| | | | | exchange factor (GEF) 7 |
| 2369 | Protein | NP_003890 | ARHGEF7 | Rho guanine nucleotide |
| | | | | exchange factor (GEF) 7 |
| 2370 | DNA | NM_145735 | ARHGEF7 | Rho guanine nucleotide |
| | | | | exchange factor (GEF) 7 |
| 2371 | Protein | NP_663788 | ARHGEF7 | Rho guanine nucleotide |
| | | | | exchange factor (GEF) 7 |
| 2372 | DNA | NM_015634 | DKFZP586B0923 | DKFZP586B0923 protein |
| 2373 | Protein | NP_056449 | DKFZP586B0923 | DKFZP586B0923 protein |
| 2374 | DNA | AB011102 | ZNF292 | zinc finger protein 292 |
| 2375 | Protein | AB011102 | ZNF292 | zinc finger protein 292 |
| | | (Translation) | | |
| 2376 | DNA | NM_024824 | FLJ11806 | hypothetical protein FLJ11806 |
| 2377 | Protein | NP_079100 | FLJ11806 | hypothetical protein FLJ11806 |
| 2378 | DNA | NM_001823 | CKB | creatine kinase, brain |
| 2379 | Protein | NP_001814 | CKB | creatine kinase, brain |
| 2380 | DNA | NM_003211 | TDG | thymine-DNA glycosylase |
| 2381 | Protein | NP_003202 | TDG | thymine-DNA glycosylase |
| 2382 | DNA | NM_003634 | NIPSNAP1 | nipsnap homolog 1 (C. elegans) |
| 2383 | Protein | NP_003625 | NIPSNAP1 | nipsnap homolog 1 (C. elegans) |
| 2384 | DNA | NM_014225 | PPP2R1A | protein phosphatase 2 (formerly |
| | | | | 2A), regulatory subunit A (PR |
| | | | | 65), alpha isoform |
| 2385 | Protein | NP_055040 | PPP2R1A | protein phosphatase 2 (formerly |
| | | | | 2A), regulatory subunit A (PR |
| | | | | 65), alpha isoform |
| 2386 | DNA | T57872 | | EST, Moderately similar to |
| | | | | COXG_HUMAN Cytochrome |
| | | | | c oxidase polypeptide VIb |
| | | | | (AED) [H. sapiens] |
| 2387 | DNA | NM_003792 | EDF1 | endothelial differentiation- |
| | | | | related factor 1 |
| 2388 | Protein | NP_003783 | EDF1 | endothelial differentiation- |
| | | | | related factor 1 |
| 2389 | DNA | NM_153200 | EDF1 | endothelial differentiation- |
| | | | | related factor 1 |
| 2390 | Protein | NP_694880 | EDF1 | endothelial differentiation- |
| | | | | related factor 1 |
| 2391 | DNA | NM_004332 | BPHL | biphenyl hydrolase-like (serine |
| | | | | hydrolase; breast epithelial |
| | | | | mucin-associated antigen) |
| 2392 | Protein | NP_004323 | BPHL | biphenyl hydrolase-like (serine |
| | | | | hydrolase; breast epithelial |
| | | | | mucin-associated antigen) |
| 2393 | DNA | AA290994 | | Homo sapiens cDNA FLJ20722 |
| | | | | fis, clone HEP15411, mRNA |
| | | | | sequence |
| 2394 | DNA | AA554945 | | ESTs, Weakly similar to |
| | | | | hypothetical protein FLJ20378 |
| | | | | [Homo sapiens] [H. sapiens] |
| 2395 | DNA | NM_015626 | WSB1 | SOCS box-containing WD |
| | | | | protein SWiP-1 |
| 2396 | Protein | NP_056441 | WSB1 | SOCS box-containing WD |
| | | | | protein SWiP-1 |
| 2397 | DNA | NM_134264 | WSB1 | SOCS box-containing WD |
| | | | | protein SWiP-1 |
| 2398 | Protein | NP_599026 | WSB1 | SOCS box-containing WD |
| | | | | protein SWiP-1 |
| 2399 | DNA | NM_134265 | WSB1 | SOCS box-containing WD |
| | | | | protein SWiP-1 |
| 2400 | Protein | NP_599027 | WSB1 | SOCS box-containing WD |
| | | | | protein SWiP-1 |
| 2401 | DNA | NM_030980 | FLJ12671 | hypothetical protein FLJ12671 |
| 2402 | Protein | NP_112242 | FLJ12671 | hypothetical protein FLJ12671 |
| 2403 | DNA | NM_017432 | PTOV1 | prostate tumor over expressed |
| | | | | gene 1 |
| 2404 | Protein | NP_059128 | PTOV1 | prostate tumor over expressed |
| | | | | gene 1 |
| 2405 | DNA | W26477 | HELSNF1 | helicase with SNF2 domain 1 |
| 2406 | DNA | NM_003864 | SAP30 | sin3-associated polypeptide, |
| | | | | 30 kDa |
| 2407 | Protein | NP_003855 | SAP30 | sin3-associated polypeptide, |
| | | | | 30 kDa |
| 2408 | DNA | L36531 | ITGA8 | integrin alpha 8 |
| 2409 | Protein | L36531 (Translation) | ITGA8 | integrin, alpha 8 |
| 2410 | DNA | NM_004272 | SYN47 | Homer, neuronal immediate |
| | | | | early gene, 1B |
| 2411 | Protein | NP_004263 | SYN47 | Homer, neuronal immediate |
| | | | | early gene, 1B |
| 2412 | DNA | NM_003213 | TEAD4 | TEA domain family member 4 |
| 2413 | Protein | NP_003204 | TEAD4 | TEA domain family member 4 |
| 2414 | DNA | NM_024112 | C9orf16 | chromosome 9 open reading |
| | | | | frame 16 |
| 2415 | Protein | NP_077017 | C9orf16 | chromosome 9 open reading |
| | | | | frame 16 |
| 2416 | DNA | NM_005544 | IRS1 | insulin receptor substrate 1 |
| 2417 | Protein | NP_005535 | IRS1 | insulin receptor substrate 1 |
| 2418 | DNA | NM_006951 | TAF5 | TAF5 RNA polymerase II, |
| | | | | TATA box binding protein |
| | | | | (TBP)-associated factor, |
| | | | | 100 kDa |
| 2419 | Protein | NP_008882 | TAF5 | TAF5 RNA polymerase II, |
| | | | | TATA box binding protein |
| | | | | (TBP)-associated factor, |
| | | | | 100 kDa |
| 2420 | DNA | NM_139052 | TAF5 | TAF5 RNA polymerase II, |
| | | | | TATA box binding protein |
| | | | | (TBP)-associated factor, |
| | | | | 100 kDa |
| 2421 | Protein | NP_620640 | TAF5 | TAF5 RNA polymerase II, |
| | | | | TATA box binding protein |
| | | | | (TBP)-associated factor, |
| | | | | 100 kDa |
| 2422 | DNA | NM_002692 | POLE2 | polymerase (DNA directed), |
| | | | | epsilon 2 (p59 subunit) |
| 2423 | Protein | NP_002683 | POLE2 | polymerase (DNA directed), |
| | | | | epsilon 2 (p59 subunit) |
| 2424 | DNA | NM_004459 | FALZ | fetal Alzheimer antigen |
| 2425 | Protein | NP_004450 | FALZ | fetal Alzheimer antigen |
| 2426 | DNA | NM_004634 | BRPF1 | bromodomain and PHD finger |
| | | | | containing, 1 |
| 2427 | Protein | NP_004625 | BRPF1 | bromodomain and PHD finger |
| | | | | containing, 1 |
| 2428 | DNA | NM_003624 | RANBP3 | RAN binding protein 3 |
| 2429 | Protein | NP_003615 | RANBP3 | RAN binding protein 3 |
| 2430 | DNA | NM_007320 | RANBP3 | RAN binding protein 3 |
| 2431 | Protein | NP_015559 | RANBP3 | RAN binding protein 3 |
| 2432 | DNA | NM_007321 | RANBP3 | RAN binding protein 3 |
| 2433 | Protein | NP_015560 | RANBP3 | RAN binding protein 3 |
| 2434 | DNA | NM_007322 | RANBP3 | RAN binding protein 3 |
| 2435 | Protein | NP_015561 | RANBP3 | RAN binding protein 3 |
| 2436 | DNA | NM_014902 | KIAA0964 | KIAA0964 protein |
| 2437 | Protein | NP_055717 | KIAA0964 | KIAA0964 protein |
| 2438 | DNA | NM_002414 | MIC2 | antigen identified by |
| | | | | monoclonal antibodies 12E7, |
| | | | | F21 and O13 |
| 2439 | Protein | NP_002405 | MIC2 | antigen identified by |
| | | | | monoclonal antibodies 12E7, |
| | | | | F21 and O13 |
| 2440 | DNA | NM_005534 | IFNGR2 | interferon gamma receptor 2 |
| | | | | (interferon gamma transducer |
| | | | | 1) |
| 2441 | Protein | NP_005525 | IFNGR2 | interferon gamma receptor 2 |
| | | | | (interferon gamma transducer |
| | | | | 1) |
| 2442 | DNA | NM_014827 | KIAA0663 | KIAA0663 gene product |
| 2443 | Protein | NP_055642 | KIAA0663 | KIAA0663 gene product |
| 2444 | DNA | NM_005054 | RANBP2L1 | RAN binding protein 2-like 1 |
| 2445 | Protein | NP_005045 | RANBP2L1 | RAN binding protein 2-like 1 |
| 2446 | DNA | NM_032260 | RANBP2L1 | RAN binding protein 2-like 1 |
| 2447 | Protein | NP_115636 | RANBP2L1 | RAN binding protein 2-like 1 |
| 2448 | DNA | NM_000975 | RPL11 | ribosomal protein L11 |
| 2449 | Protein | NP_000966 | RPL11 | ribosomal protein L11 |
| 2450 | DNA | NM_005730 | OS4 | conserved gene amplified in |
| | | | | osteosarcoma |
| 2451 | Protein | NP_005721 | OS4 | conserved gene amplified in |
| | | | | osteosarcoma |
| 2452 | DNA | NM_000462 | UBE3A | ubiquitin protein ligase E3A |
| | | | | (human papilloma virus E6- |
| | | | | associated protein, Angelman |
| | | | | syndrome) |
| 2453 | Protein | NP_000453 | UBE3A | ubiquitin protein ligase E3A |
| | | | | (human papilloma virus E6- |
| | | | | associated protein, Angelman |
| | | | | syndrome) |
| 2454 | DNA | NM_130838 | UBE3A | ubiquitin protein ligase E3A |
| | | | | (human papilloma virus E6- |
| | | | | associated protein, Angelman |
| | | | | syndrome) |
| 2455 | Protein | NP_570853 | UBE3A | ubiquitin protein ligase E3A |
| | | | | (human papilloma virus E6- |
| | | | | associated protein, Angelman |
| | | | | syndrome) |
| 2456 | DNA | NM_130839 | UBE3A | ubiquitin protein ligase E3A |
| | | | | (human papilloma virus E6- |
| | | | | associated protein, Angelman |
| | | | | syndrome) |
| 2457 | Protein | NP_570854 | UBE3A | ubiquitin protein ligase E3A |
| | | | | (human papilloma virus E6- |
| | | | | associated protein, Angelman |
| | | | | syndrome) |
| 2458 | DNA | NM_004373 | COX6A1 | cytochrome c oxidase subunit |
| | | | | VIa polypeptide 1 |
| 2459 | Protein | NP_004364 | COX6A1 | cytochrome c oxidase subunit |
| | | | | VIa polypeptide 1 |
| 2460 | DNA | NM_022170 | WBSCR1 | Williams-Beuren syndrome |
| | | | | chromosome region 1 |
| 2461 | Protein | NP_071496 | WBSCR1 | Williams-Beuren syndrome |
| | | | | chromosome region 1 |
| 2462 | DNA | NM_031992 | WBSCR1 | Williams-Beuren syndrome |
| | | | | chromosome region 1 |
| 2463 | Protein | NP_114381 | WBSCR1 | Williams-Beuren syndrome |
| | | | | chromosome region 1 |
| 2464 | DNA | NM_002574 | PRDX1 | peroxiredoxin 1 |
| 2465 | Protein | NP_002565 | PRDX1 | peroxiredoxin 1 |
| 2466 | DNA | NM_002166 | ID2 | inhibitor of DNA binding 2, |
| | | | | dominant negative helix-loop- |
| | | | | helix protein |
| 2467 | Protein | NP_002157 | ID2 | inhibitor of DNA binding 2, |
| | | | | dominant negative helix-loop- |
| | | | | helix protein |
| 2468 | DNA | NM_002629 | PGAM1 | phosphoglycerate mutase 1 |
| | | | | (brain) |
| 2469 | Protein | NP_002620 | PGAM1 | phosphoglycerate mutase 1 |
| | | | | (brain) |
| 2470 | DNA | NM_004090 | DUSP3 | dual specificity phosphatase 3 |
| | | | | (vaccinia virus phosphatase |
| | | | | VH1-related) |
| 2471 | Protein | NP_004081 | DUSP3 | dual specificity phosphatase 3 |
| | | | | (vaccinia virus phosphatase |
| | | | | VH1-related) |
| 2472 | DNA | AI222594 | | Homo sapiens mRNA; cDNA |
| | | | | DKFZp564H1916 (from clone |
| | | | | DKFZp564H1916), mRNA |
| | | | | sequence |
| 2473 | DNA | NM_013298 | HSU79252 | hypothetical protein HSU79252 |
| 2474 | Protein | NP_037430 | HSU79252 | hypothetical protein HSU79252 |
| 2475 | DNA | AB007916 | KIAA0447 | KIAA0447 gene product |
| 2476 | Protein | AB007916 | KIAA0447 | KIAA0447 gene product |
| | | (Translation) | | |
| 2477 | DNA | NM_006303 | JTV1 | JTV1 gene |
| 2478 | Protein | NP_006294 | JTV1 | JTV1 gene |
| 2479 | DNA | NM_004773 | TRIP3 | thyroid hormone receptor |
| | | | | interactor 3 |
| 2480 | Protein | NP_004764 | TRIP3 | thyroid hormone receptor |
| | | | | interactor 3 |
| 2481 | DNA | NM_016391 | HSPC111 | hypothetical protein HSPC111 |
| 2482 | Protein | NP_057475 | HSPC111 | hypothetical protein HSPC111 |
| 2483 | DNA | AL046940 | | ESTs, Weakly similar to |
| | | | | hypothetical protein FLJ22184 |
| | | | | [Homo sapiens] [H. sapiens] |
| 2484 | DNA | NM_020151 | STARD7 | START domain containing 7 |
| 2485 | Protein | NP_064536 | STARD7 | START domain containing 7 |
| 2486 | DNA | NM_139267 | STARD7 | START domain containing 7 |
| 2487 | DNA | NM_005234 | NR2F6 | nuclear receptor subfamily 2, |
| | | | | group F, member 6 |
| 2488 | Protein | NP_005225 | NR2F6 | nuclear receptor subfamily 2, |
| | | | | group F, member 6 |
| 2489 | DNA | NM_002967 | SAFB | scaffold attachment factor B |
| 2490 | Protein | NP_002958 | SAFB | scaffold attachment factor B |
| 2491 | DNA | NM_018186 | PACE-1 | ezrin-binding partner PACE-1 |
| 2492 | Protein | NP_060656 | PACE-1 | ezrin-binding partner PACE-1 |
| 2493 | DNA | NM_020423 | PACE-1 | ezrin-binding partner PACE-1 |
| 2494 | Protein | NP_065156 | PACE-1 | ezrin-binding partner PACE-1 |
| 2495 | DNA | NM_001130 | AES | amino-terminal enhancer of |
| | | | | split |
| 2496 | Protein | NP_001121 | AES | amino-terminal enhancer of |
| | | | | split |
| 2497 | DNA | NM_005859 | PURA | purine-rich element binding |
| | | | | protein A |
| 2498 | Protein | NP_005850 | PURA | purine-rich element binding |
| | | | | protein A |
| 2499 | DNA | NM_003032 | SIAT1 | sialyltransferase 1 (beta- |
| | | | | galactoside alpha-2,6- |
| | | | | sialytransferase) |
| 2500 | Protein | NP_003023 | SIAT1 | sialyltransferase 1 (beta- |
| | | | | galactoside alpha-2,6- |
| | | | | sialytransferase) |
| 2501 | DNA | NM_173216 | SIAT1 | sialyltransferase 1 (beta- |
| | | | | galactoside alpha-2,6- |
| | | | | sialytransferase) |
| 2502 | DNA | NM_173217 | SIAT1 | sialyltransferase 1 (beta- |
| | | | | galactoside alpha-2,6- |
| | | | | sialytransferase) |
| 2503 | Protein | NP_775324 | SIAT1 | sialyltransferase 1 (beta- |
| | | | | galactoside alpha-2,6- |
| | | | | sialytransferase) |
| 2504 | DNA | NM_003952 | RPS6KB2 | ribosomal protein S6 kinase, |
| | | | | 70 kDa, polypeptide 2 |
| 2505 | Protein | NP_003943 | RPS6KB2 | ribosomal protein S6 kinase, |
| | | | | 70 kDa, polypeptide 2 |
| 2506 | DNA | NM_015110 | SMC5 | SMC5 protein |
| 2507 | Protein | NP_055925 | SMC5 | SMC5 protein |
| 2508 | DNA | NM_007152 | ZNF195 | zinc finger protein 195 |
| 2509 | Protein | NP_009083 | ZNF195 | zinc finger protein 195 |
| 2510 | DNA | NM_003171 | SUPV3L1 | suppressor of var1, 3-like 1 (S. cerevisiae) |
| 2511 | Protein | NP_003162 | SUPV3L1 | suppressor of var1, 3-like 1 (S. cerevisiae) |
| 2512 | DNA | NM_012265 | C22orf3 | chromosome 22 open reading |
| | | | | frame 3 |
| 2513 | Protein | NP_036397 | C22orf3 | chromosome 22 open reading |
| | | | | frame 3 |
| 2514 | DNA | NM_004053 | BYSL | bystin-like |
| 2515 | Protein | NP_004044 | BYSL | bystin-like |
| 2516 | DNA | NM_014921 | LEC2 | lectomedin-2 |
| 2517 | Protein | NP_055736 | LEC2 | lectomedin-2 |
| 2518 | DNA | NM_015285 | WDR7 | WD repeat domain 7 |
| 2519 | Protein | NP_056100 | WDR7 | WD repeat domain 7 |
| 2520 | DNA | NM_052834 | WDR7 | WD repeat domain 7 |
| 2521 | Protein | NP_443066 | WDR7 | WD repeat domain 7 |
| 2522 | DNA | AB014554 | PPFIA3 | protein tyrosine phosphatase, |
| | | | | receptor type, f polypeptide |
| | | | | (PTPRF), interacting protein |
| | | | | (liprin), alpha 3 |
| 2523 | Protein | AB014554 | PPFIA3 | protein tyrosine phosphatase, |
| | | (Translation) | | receptor type, f polypeptide |
| | | | | (PTPRF), interacting protein |
| | | | | (liprin), alpha 3 |
| 2524 | DNA | NM_003453 | ZNF198 | zinc finger protein 198 |
| 2525 | Protein | NP_003444 | ZNF198 | zinc finger protein 198 |
| 2526 | DNA | NM_005043 | MAP2K7 | mitogen-activated protein |
| | | | | kinase kinase 7 |
| 2527 | Protein | NP_005034 | MAP2K7 | mitogen-activated protein |
| | | | | kinase kinase 7 |
| 2528 | DNA | NM_145185 | MAP2K7 | mitogen-activated protein |
| | | | | kinase kinase 7 |
| 2529 | Protein | NP_660186 | MAP2K7 | mitogen-activated protein |
| | | | | kinase kinase 7 |
| 2530 | DNA | NM_145329 | MAP2K7 | mitogen-activated protein |
| | | | | kinase kinase 7 |
| 2531 | Protein | NP_663302 | MAP2K7 | mitogen-activated protein |
| | | | | kinase kinase 7 |
| 2532 | DNA | NM_014918 | CHSY1 | carbohydrate (chondroitin) |
| | | | | synthase 1 |
| 2533 | Protein | NP_055733 | CHSY1 | carbohydrate (chondroitin) |
| | | | | synthase 1 |
| 2534 | DNA | AB007883 | KIAA0423 | KIAA0423 protein |
| 2535 | Protein | AB007883 | KIAA0423 | KIAA0423 protein |
| | | (Translation) | | |
| 2536 | DNA | NM_004520 | KIF2 | kinesin heavy chain member 2 |
| 2537 | Protein | NP_004511 | KIF2 | kinesin heavy chain member 2 |
| 2538 | DNA | NM_021212 | ZF | HCF-binding transcription |
| | | | | factor Zhangfei |
| 2539 | Protein | NP_067035 | ZF | HCF-binding transcription |
| | | | | factor Zhangfei |
| 2540 | DNA | NM_005360 | MAF | v-maf musculoaponeurotic |
| | | | | fibrosarcoma oncogene |
| | | | | homolog (avian) |
| 2541 | Protein | NP_005351 | MAF | v-maf musculoaponeurotic |
| | | | | fibrosarcoma oncogene |
| | | | | homolog (avian) |
| 2542 | DNA | NM_003668 | MAPKAPK5 | mitogen-activated protein |
| | | | | kinase-activated protein kinase 5 |
| 2543 | Protein | NP_003659 | MAPKAPK5 | mitogen-activated protein |
| | | | | kinase-activated protein kinase 5 |
| 2544 | DNA | NM_139078 | MAPKAPK5 | mitogen-activated protein |
| | | | | kinase-activated protein kinase 5 |
| 2545 | Protein | NP_620777 | MAPKAPK5 | mitogen-activated protein |
| | | | | kinase-activated protein kinase 5 |
| 2546 | DNA | NM_002405 | MFNG | manic fringe homolog |
| | | | | (Drosophila) |
| 2547 | Protein | NP_002396 | MFNG | manic fringe homolog |
| | | | | (Drosophila) |
| 2548 | DNA | NM_006339 | HMG20B | high-mobility group 20B |
| 2549 | Protein | NP_006330 | HMG20B | high-mobility group 20B |
| 2550 | DNA | W72239 | | Homo sapiens mRNA; cDNA |
| | | | | DKFZp434M162 (from clone |
| | | | | DKFZp434M162), mRNA |
| | | | | sequence |
| 2551 | DNA | NM_000835 | GRIN2C | glutamate receptor, ionotropic, |
| | | | | N-methyl D-asparate 2C |
| 2552 | Protein | NP_000826 | GRIN2C | glutamate receptor, ionotropic, |
| | | | | N-methyl D-asparate 2C |
| 2553 | DNA | NM_006007 | ZNF216 | zinc finger protein 216 |
| 2554 | Protein | NP_005998 | ZNF216 | zinc finger protein 216 |
| 2555 | DNA | NM_004725 | BUB3 | BUB3 budding uninhibited by |
| | | | | benzimidazoles 3 homolog |
| | | | | (yeast) |
| 2556 | Protein | NP_004716 | BUB3 | BUB3 budding uninhibited by |
| | | | | benzimidazoles 3 homolog |
| | | | | (yeast) |
| 2557 | DNA | NM_015360 | KIAA0052 | KIAA0052 protein |
| 2558 | Protein | NP_056175 | KIAA0052 | KIAA0052 protein |
| 2559 | DNA | NM_005180 | BMI1 | B lymphoma Mo-MLV |
| | | | | insertion region (mouse) |
| 2560 | Protein | NP_005171 | BMI1 | B lymphoma Mo-MLV |
| | | | | insertion region (mouse) |
| 2561 | DNA | NM_015190 | DNAJC9 | DnaJ (Hsp40) homolog, |
| | | | | subfamily C, member 9 |
| 2562 | Protein | NP_056005 | DNAJC9 | DnaJ (Hsp40) homolog, |
| | | | | subfamily C, member 9 |
| 2563 | DNA | X68560 | SP3 | Sp3 transcription factor |
| 2564 | Protein | X68560 (Translation) | SP3 | Sp3 transcription factor |
| 2565 | DNA | NM_004111 | FEN1 | flap structure-specific |
| | | | | endonuclease 1 |
| 2566 | Protein | NP_004102 | FEN1 | flap structure-specific |
| | | | | endonuclease 1 |
| 2567 | DNA | NM_016030 | CGI-87 | CGI-87 protein |
| 2568 | Protein | NP_057114 | CGI-87 | CGI-87 protein |
| 2569 | DNA | AB023164 | KIAA0947 | KIAA0947 protein |
| 2570 | Protein | AB023164 | KIAA0947 | KIAA0947 protein |
| | | (Translation) | | |
| 2571 | DNA | NM_001949 | E2F3 | E2F transcription factor 3 |
| 2572 | Protein | NP_001940 | E2F3 | E2F transcription factor 3 |
| 2573 | DNA | D87445 | KIAA0256 | KIAA0256 gene product |
| 2574 | Protein | D87445 (Translation) | KIAA0256 | KIAA0256 gene product |
| 2575 | DNA | NM_015342 | KIAA0073 | KIAA0073 protein |
| 2576 | Protein | NP_056157 | KIAA0073 | KIAA0073 protein |
| 2577 | DNA | NM_018416 | FHX | FOXJ2 forkhead factor |
| 2578 | Protein | NP_060886 | FHX | FOXJ2 forkhead factor |
| 2579 | DNA | AB028956 | KIAA1033 | KIAA1033 protein |
| 2580 | Protein | AB028956 | KIAA1033 | KIAA1033 protein |
| | | (Translation) | | |
| 2581 | DNA | NM_004808 | NMT2 | N-myristoyltransferase 2 |
| 2582 | Protein | NP_004799 | NMT2 | N-myristoyltransferase 2 |
| 2583 | DNA | NM_000455 | STK11 | serine/threonine kinase 11 |
| | | | | (Peutz-Jeghers syndrome) |
| 2584 | Protein | NP_000446 | STK11 | serine/threonine kinase 11 |
| | | | | (Peutz-Jeghers syndrome) |
| 2585 | DNA | D83776 | KIAA0191 | KIAA0191 protein |
| 2586 | Protein | D83776 (Translation) | KIAA0191 | KIAA0191 protein |
| 2587 | DNA | AF007128 | | Homo sapiens clone 23870 |
| | | | | mRNA sequence |
| 2588 | DNA | AB018337 | KIAA0794 | KIAA0794 protein |
| 2589 | Protein | AB018337 | KIAA0794 | KIAA0794 protein |
| | | (Translation) | | |
| 2590 | DNA | NM_024051 | MGC3077 | hypothetical protein MGC3077 |
| 2591 | Protein | NP_076956 | MGC3077 | hypothetical protein MGC3077 |
| 2592 | DNA | NM_002646 | PIK3C2B | phosphoinositide-3-kinase, |
| | | | | class 2, beta polypeptide |
| 2593 | Protein | NP_002637 | PIK3C2B | phosphoinositide-3-kinase, |
| | | | | class 2, beta polypeptide |
| 2594 | DNA | NM_005745 | BCAP31 | accessory protein BAP31 |
| 2595 | Protein | NP_005736 | BCAP31 | accessory protein BAP31 |
| 2596 | DNA | NM_001319 | CSNK1G2 | casein kinase 1, gamma 2 |
| 2597 | Protein | NP_001310 | CSNK1G2 | casein kinase 1, gamma 2 |
| 2598 | DNA | NM_005744 | ARIH1 | ariadne homolog, ubiquitin- |
| | | | | conjugating enzyme E2 binding |
| | | | | protein, 1 (Drosophila) |
| 2599 | Protein | NP_005735 | ARIH1 | ariadne homolog, ubiquitin- |
| | | | | conjugating enzyme E2 binding |
| | | | | protein, 1 (Drosophila) |
| 2600 | DNA | NM_005839 | SRRM1 | serine/arginine repetitive matrix 1 |
| 2601 | Protein | NP_005830 | SRRM1 | serine/arginine repetitive matrix 1 |
| 2602 | DNA | NM_004342 | CALD1 | caldesmon 1 |
| 2603 | Protein | NP_004333 | CALD1 | caldesmon 1 |
| 2604 | DNA | NM_033138 | CALD1 | caldesmon 1 |
| 2605 | Protein | NP_149129 | CALD1 | caldesmon 1 |
| 2606 | DNA | NM_033139 | CALD1 | caldesmon 1 |
| 2607 | Protein | NP_149130 | CALD1 | caldesmon 1 |
| 2608 | DNA | NM_033140 | CALD1 | caldesmon 1 |
| 2609 | Protein | NP_149131 | CALD1 | caldesmon 1 |
| 2610 | DNA | NM_033157 | CALD1 | caldesmon 1 |
| 2611 | Protein | NP_149347 | CALD1 | caldesmon 1 |
| 2612 | DNA | NM_021034 | IFITM3 | interferon induced |
| | | | | transmembrane protein 3 (1-8 U) |
| 2613 | Protein | NP_066362 | IFITM3 | interferon induced |
| | | | | transmembrane protein 3 (1-8 U) |
| 2614 | DNA | NM_014900 | KIAA0977 | KIAA0977 protein |
| 2615 | Protein | NP_055715 | KIAA0977 | KIAA0977 protein |
| 2616 | DNA | NM_001865 | | Cluster Incl. |
| | | | | AA978033: oq55e04.s1 Homo |
| | | | | sapiens cDNA, 3′ end |
| | | | | /clone = IMAGE-1590270 |
| | | | | /clone_end = 3′ /gb = AA978033 |
| | | | | /gi = 3155479 /ug = Hs.182684 |
| | | | | /len = 524 |
| 2617 | Protein | NP_001856 | | Cluster Incl. |
| | | | | AA978033: oq55e04.s1 Homo |
| | | | | sapiens cDNA, 3′ end |
| | | | | /clone = IMAGE-1590270 |
| | | | | /clone_end = 3′ /gb = AA978033 |
| | | | | /gi = 3155479 /ug = Hs.182684 |
| | | | | /len = 524 |
| 2618 | DNA | NM_003252 | TIAL1 | TIA1 cytotoxic granule- |
| | | | | associated RNA binding |
| | | | | protein-like 1 |
| 2619 | Protein | NP_003243 | TIAL1 | TIA1 cytotoxic granule- |
| | | | | associated RNA binding |
| | | | | protein-like 1 |
| 2620 | DNA | NM_022333 | TIAL1 | TIA1 cytotoxic granule- |
| | | | | associated RNA binding |
| | | | | protein-like 1 |
| 2621 | Protein | NP_071728 | TIAL1 | TIA1 cytotoxic granule- |
| | | | | associated RNA binding |
| | | | | protein-like 1 |
| 2622 | DNA | NM_007209 | RPL35 | ribosomal protein L35 |
| 2623 | Protein | NP_009140 | RPL35 | ribosomal protein L35 |
| 2624 | DNA | NM_004045 | ATOX1 | ATX1 antioxidant protein 1 |
| | | | | homolog (yeast) |
| 2625 | Protein | NP_004036 | ATOX1 | ATX1 antioxidant protein 1 |
| | | | | homolog (yeast) |
| 2626 | DNA | NM_001418 | EIF4G2 | eukaryotic translation initiation |
| | | | | factor 4 gamma, 2 |
| 2627 | Protein | NP_001409 | EIF4G2 | eukaryotic translation initiation |
| | | | | factor 4 gamma, 2 |
| 2628 | DNA | NM_000352 | ABCC8 | ATP-binding cassette, sub- |
| | | | | family C (CFTR/MRP), |
| | | | | member 8 |
| 2629 | Protein | NP_000343 | ABCC8 | ATP-binding cassette, sub- |
| | | | | family C (CFTR/MRP), |
| | | | | member 8 |
| 2630 | DNA | NM_006153 | NCK1 | NCK adaptor protein 1 |
| 2631 | Protein | NP_006144 | NCK1 | NCK adaptor protein 1 |
| 2632 | DNA | NM_002417 | MKI67 | antigen identified by |
| | | | | monoclonal antibody Ki-67 |
| 2633 | Protein | NP_002408 | MKI67 | antigen identified by |
| | | | | monoclonal antibody Ki-67 |
| 2634 | DNA | AL040137 | | ESTs |
| 2635 | DNA | NM_021145 | DMTF1 | cyclin D binding myb-like |
| | | | | transcription factor 1 |
| 2636 | Protein | NP_066968 | DMTF1 | cyclin D binding myb-like |
| | | | | transcription factor 1 |
| 2637 | DNA | NM_004602 | STAU | staufen, RNA binding protein |
| | | | | (Drosophila) |
| 2638 | Protein | NP_004593 | STAU | staufen, RNA binding protein |
| | | | | (Drosophila) |
| 2639 | DNA | NM_017452 | STAU | staufen, RNA binding protein |
| | | | | (Drosophila) |
| 2640 | DNA | NM_017453 | STAU | staufen, RNA binding protein |
| | | | | (Drosophila) |
| 2641 | Protein | NP_059347 | STAU | staufen, RNA binding protein |
| | | | | (Drosophila) |
| 2642 | DNA | NM_017454 | STAU | staufen, RNA binding protein |
| | | | | (Drosophila) |
| 2643 | DNA | NM_016001 | CGI-48 | CGI-48 protein |
| 2644 | Protein | NP_057085 | CGI-48 | CGI-48 protein |
| 2645 | DNA | AF052138 | | Homo sapiens clone 23718 |
| | | | | mRNA sequence |
| 2646 | DNA | NM_002767 | PRPSAP2 | phosphoribosyl pyrophosphate |
| | | | | synthetase-associated protein 2 |
| 2647 | Protein | NP_002758 | PRPSAP2 | phosphoribosyl pyrophosphate |
| | | | | synthetase-associated protein 2 |
| 2648 | DNA | NM_015658 | DKFZP564C186 | DKFZP564C186 protein |
| 2649 | Protein | NP_056473 | DKFZP564C186 | DKFZP564C186 protein |
| 2650 | DNA | D29954 | KIAA0056 | KIAA0056 protein |
| 2651 | Protein | D29954 (Translation) | KIAA0056 | KIAA0056 protein |
| 2652 | DNA | NM_000529 | MC2R | melanocortin 2 receptor |
| | | | | (adrenocorticotropic hormone) |
| 2653 | Protein | NP_000520 | MC2R | melanocortin 2 receptor |
| | | | | (adrenocorticotropic hormone) |
| 2654 | DNA | NM_002382 | MAX | MAX protein |
| 2655 | Protein | NP_002373 | MAX | MAX protein |
| 2656 | DNA | NM_145112 | MAX | MAX protein |
| 2657 | Protein | NP_660087 | MAX | MAX protein |
| 2658 | DNA | NM_145113 | MAX | MAX protein |
| 2659 | DNA | NM_145114 | MAX | MAX protein |
| 2660 | Protein | NP_660089 | MAX | MAX protein |
| 2661 | DNA | NM_145116 | MAX | MAX protein |
| 2662 | Protein | NP_660092 | MAX | MAX protein |
| 2663 | DNA | NM_005532 | IFI27 | interferon, alpha-inducible |
| | | | | protein 27 |
| 2664 | Protein | NP_005523 | IFI27 | interferon, alpha-inducible |
| | | | | protein 27 |
| 2665 | DNA | NM_000244 | MEN1 | multiple endocrine neoplasia I |
| 2666 | Protein | NP_000235 | MEN1 | multiple endocrine neoplasia I |
| 2667 | DNA | NM_130799 | MEN1 | multiple endocrine neoplasia I |
| 2668 | Protein | NP_570711 | MEN1 | multiple endocrine neoplasia I |
| 2669 | DNA | NM_130800 | MEN1 | multiple endocrine neoplasia I |
| 2670 | DNA | NM_130801 | MEN1 | multiple endocrine neoplasia I |
| 2671 | DNA | NM_130802 | MEN1 | multiple endocrine neoplasia I |
| 2672 | DNA | NM_130803 | MEN1 | multiple endocrine neoplasia I |
| 2673 | DNA | NM_130804 | MEN1 | multiple endocrine neoplasia I |
| 2674 | DNA | NM_004964 | | Histone deacetylase HD1, |
| | | | | mRNA sequence |
| 2675 | Protein | NP_004955 | | Histone deacetylase HD1, |
| | | | | mRNA sequence |
| 2676 | DNA | BA-13885 | | Histone deacetylase HD1, |
| | | | | mRNA sequence |
| 2677 | DNA | NM_003743 | NCOA1 | nuclear receptor coactivator 1 |
| 2678 | Protein | NP_003734 | NCOA1 | nuclear receptor coactivator 1 |
| 2679 | DNA | NM_147223 | NCOA1 | nuclear receptor coactivator 1 |
| 2680 | Protein | NP_671756 | NCOA1 | nuclear receptor coactivator 1 |
| 2681 | DNA | NM_147233 | NCOA1 | nuclear receptor coactivator 1 |
| 2682 | Protein | NP_671766 | NCOA1 | nuclear receptor coactivator 1 |
| 2683 | DNA | NM_001893 | | Casein kinase I delta, mRNA |
| | | | | sequence |
| 2684 | Protein | NP_001884 | | Casein kinase I delta, mRNA |
| | | | | sequence |
| 2685 | DNA | NM_007065 | CDC37 | CDC37 cell division cycle 37 |
| | | | | homolog (S. cerevisiae) |
| 2686 | Protein | NP_008996 | CDC37 | CDC37 cell division cycle 37 |
| | | | | homolog (S. cerevisiae) |
| 2687 | DNA | NM_000534 | PMS1 | PMS1 postmeiotic segregation |
| | | | | increased 1 (S. cerevisiae) |
| 2688 | Protein | NP_000525 | PMS1 | PMS1 postmeiotic segregation |
| | | | | increased 1 (S. cerevisiae) |
| 2689 | DNA | NM_000535 | PMS2 | PMS2 postmeiotic segregation |
| | | | | increased 2 (S. cerevisiae) |
| 2690 | Protein | NP_000526 | PMS2 | PMS2 postmeiotic segregation |
| | | | | increased 2 (S. cerevisiae) |
| 2691 | DNA | NM_001809 | CENPA | centromere protein A, 17 kDa |
| 2692 | Protein | NP_001800 | CENPA | centromere protein A, 17 kDa |
| 2693 | DNA | NM_004419 | DUSP5 | dual specificity phosphatase 5 |
| 2694 | Protein | NP_004410 | DUSP5 | dual specificity phosphatase 5 |
| 2695 | DNA | NM_002887 | RARS | arginyl-tRNA synthetase |
| 2696 | Protein | NP_002878 | RARS | arginyl-tRNA synthetase |
| 2697 | DNA | NM_005521 | TLX1 | T-cell leukemia, homeobox 1 |
| 2698 | Protein | NP_005512 | TLX1 | T-cell leukemia, homeobox 1 |
| 2699 | DNA | NM_003318 | TTK | TTK protein kinase |
| 2700 | Protein | NP_003309 | TTK | TTK protein kinase |
| 2701 | DNA | NM_001291 | CLK2 | CDC-like kinase 2 |
| 2702 | Protein | NP_001282 | CLK2 | CDC-like kinase 2 |
| 2703 | DNA | NM_003993 | CLK2 | CDC-like kinase 2 |
| 2704 | Protein | NP_003984 | CLK2 | CDC-like kinase 2 |
| 2705 | DNA | HG3635-HT3845 | | Zinc Finger Protein, Kruppel- |
| | | | | Like |
| 2706 | DNA | HG1322-HT5143 | | Small Nuclear |
| | | | | Ribonucleoprotein, Polypeptide |
| | | | | C, Alt. Splice 2 |
| 2707 | DNA | HG1751-HT1768 | | Chorionic |
| | | | | Somatomammotropin Hormone |
| | | | | Cs-5 |
| 2708 | DNA | NM_004341 | CAD | carbamoyl-phosphate |
| | | | | synthetase 2, aspartate |
| | | | | transcarbamylase, and |
| | | | | dihydroorotase |
| 2709 | Protein | NP_004332 | CAD | carbamoyl-phosphate |
| | | | | synthetase 2, aspartate |
| | | | | transcarbamylase, and |
| | | | | dihydroorotase |
| 2710 | DNA | NM_006145 | DNAJB1 | DnaJ (Hsp40) homolog, |
| | | | | subfmaily B, member 1 |
| 2711 | Protein | NP_006136 | DNAJB1 | DnaJ (Hsp40) homolog, |
| | | | | subfmaily B, member 1 |
| 2712 | DNA | NM_004039 | ANXA2 | annexin A2 |
| 2713 | Protein | NP_004030 | ANXA2 | annexin A2 |
| 2714 | DNA | NM_002643 | PIGF | phosphatidylinositol glycan, |
| | | | | class F |
| 2715 | Protein | NP_002634 | PIGF | phosphatidylinositol glycan, |
| | | | | class F |
| 2716 | DNA | NM_173074 | PIGF | phosphatidylinositol glycan, |
| | | | | class F |
| 2717 | Protein | NP_775097 | PIGF | phosphatidylinositol glycan, |
| | | | | class F |
| 2718 | DNA | NM_006468 | RPC62 | polymerase (RNA) III (DNA |
| | | | | directed) (62 kD) |
| 2719 | Protein | NP_006459 | RPC62 | polymerase (RNA) III (DNA |
| | | | | directed) (62 kD) |
| 2720 | DNA | NM_003220 | TFAP2A | transcription factor AP-2 alpha |
| | | | | (activating enhancer binding |
| | | | | protein 2 alpha) |
| 2721 | Protein | NP_003211 | TFAP2A | transcription factor AP-2 alpha |
| | | | | (activating enhancer binding |
| | | | | protein 2 alpha) |
| 2722 | DNA | NM_000946 | PRIM1 | primase, polypeptide 1, 49 kDa |
| 2723 | Protein | NP_000937 | PRIM1 | primase, polypeptide 1, 49 kDa |
| 2724 | DNA | NM_003913 | PRPF4B | PRP4 pre-mRNA processing |
| | | | | factor 4 homolog B (yeast) |
| 2725 | Protein | NP_003904 | PRPF4B | PRP4 pre-mRNA processing |
| | | | | factor 4 homolog B (yeast) |
| 2726 | DNA | NM_000956 | PTGER2 | prostaglandin E receptor 2 |
| | | | | (subtype EP2), 53 kDa |
| 2727 | Protein | NP_000947 | PTGER2 | prostaglandin E receptor 2 |
| | | | | (subtype EP2), 53 kDa |
| 2728 | DNA | NM_004398 | DDX10 | DEAD/H (Asp-Glu-Ala- |
| | | | | Asp/His) box polypeptide 10 |
| | | | | (RNA helicase) |
| 2729 | Protein | NP_004389 | DDX10 | DEAD/H (Asp-Glu-Ala- |
| | | | | Asp/His) box polypeptide 10 |
| | | | | (RNA helicase) |
| 2730 | DNA | NM_003345 | UBE2I | ubiquitin-conjugating enzyme |
| | | | | E2I (UBC9 homolog, yeast) |
| 2731 | Protein | NP_003336 | UBE2I | ubiquitin-conjugating enzyme |
| | | | | E2I (UBC9 homolog, yeast) |
| 2732 | DNA | NM_003463 | PTP4A1 | protein tyrosine phosphatase |
| | | | | type IVA, member 1 |
| 2733 | Protein | NP_003454 | PTP4A1 | protein tyrosine phosphatase |
| | | | | type IVA, member 1 |
| 2734 | DNA | NM_006164 | NFE2L2 | nuclear factor (erythroid- |
| | | | | derived 2)-like 2 |
| 2735 | Protein | NP_006155 | NFE2L2 | nuclear factor (erythroid- |
| | | | | derived 2)-like 2 |
| 2736 | DNA | NM_006284 | TAF10 | TAF10 RNA polymerase II, |
| | | | | TATA box binding protein |
| | | | | (TBP)-associated factor, 30 kDa |
| 2737 | Protein | NP_006275 | TAF10 | TAF10 RNA polymerase II, |
| | | | | TATA box binding protein |
| | | | | (TBP)-associated factor, 30 kDa |
| 2738 | DNA | NM_000801 | FKBP1A | FK506 binding protein 1A, |
| | | | | 12 kDa |
| 2739 | Protein | NP_000792 | FKBP1A | FK506 binding protein 1A, |
| | | | | 12 kDa |
| 2740 | DNA | NM_054014 | FKBP1A | FK506 binding protein 1A, |
| | | | | 12 kDa |
| 2741 | DNA | NM_003403 | YY1 | YY1 transcription factor |
| 2742 | Protein | NP_003394 | YY1 | YY1 transcription factor |
| 2743 | DNA | NM_002415 | MIF | macrophage migration |
| | | | | inhibitory factor (glycosylation- |
| | | | | inhibiting factor) |
| 2744 | Protein | NP_002406 | MIF | macrophage migration |
| | | | | inhibitory factor (glycosylation- |
| | | | | inhibiting factor) |
| 2745 | DNA | NM_000296 | PKD1 | polycystic kidney disease 1 |
| | | | | (autosomal dominant) |
| 2746 | Protein | NP_000287 | PKD1 | polycystic kidney disease 1 |
| | | | | (autosomal dominant) |
| 2747 | DNA | NM_006243 | PPP2R5A | protein phosphatase 2, |
| | | | | regulatory subunit B (B56), |
| | | | | alpha isoform |
| 2748 | Protein | NP_006234 | PPP2R5A | protein phosphatase 2, |
| | | | | regulatory subunit B (B56), |
| | | | | alpha isoform |
| 2749 | DNA | NM_014235 | UBL4 | ubiquitin-like 4 |
| 2750 | Protein | NP_055050 | UBL4 | ubiquitin-like 4 |
| 2751 | DNA | NM_004156 | PPP2CB | protein phosphatase 2 (formerly |
| | | | | 2A), catalytic subunit, beta |
| | | | | isoform |
| 2752 | Protein | NP_004147 | PPP2CB | protein phosphatase 2 (formerly |
| | | | | 2A), catalytic subunit, beta |
| | | | | isoform |
| 2753 | DNA | NM_006332 | IFI30 | interferon, gamma-inducible |
| | | | | protein 30 |
| 2754 | Protein | NP_006323 | IFI30 | interferon, gamma-inducible |
| | | | | protein 30 |
| 2755 | DNA | NM_002811 | PSMD7 | proteasome (prosome, |
| | | | | macropain) 26S subunit, non- |
| | | | | ATPase, 7 (Mov34 homolog) |
| 2756 | Protein | NP_002802 | PSMD7 | proteasome (prosome, |
| | | | | macropain) 26S subunit, non- |
| | | | | ATPase, 7 (Mov34 homolog) |
| 2757 | DNA | NM_002806 | PSMC6 | proteasome (prosome, |
| | | | | macropain) 26S subunit, |
| | | | | ATPase, 6 |
| 2758 | Protein | NP_002797 | PSMC6 | proteasome (prosome, |
| | | | | macropain) 26S subunit, |
| | | | | ATPase, 6 |
| 2759 | DNA | NM_003262 | TLOC1 | translocation protein 1 |
| 2760 | Protein | NP_003253 | TLOC1 | translocation protein 1 |
| 2761 | DNA | NM_004954 | MARK2 | MAP/microtubule affinity- |
| | | | | regulating kinase 2 |
| 2762 | Protein | NP_004945 | MARK2 | MAP/microtubule affinity- |
| | | | | regulating kinase 2 |
| 2763 | DNA | NM_017490 | MARK2 | MAP/microtubule affinity- |
| | | | | regulating kinase 2 |
| 2764 | Protein | NP_059672 | MARK2 | MAP/microtubule affinity- |
| | | | | regulating kinase 2 |
| 2765 | DNA | NM_014264 | STK18 | serine/threonine kinase 18 |
| 2766 | Protein | NP_055079 | STK18 | serine/threonine kinase 18 |
| 2767 | DNA | NM_002969 | MAPK12 | mitogen-activated protein |
| | | | | kinase 12 |
| 2768 | Protein | NP_002960 | MAPK12 | mitogen-activated protein |
| | | | | kinase 12 |
| 2769 | DNA | K03022 | U2 small | U2 small nuclear RNA gene |
| | | | nuclear RNA | |
| 2770 | DNA | AK027091 | FLJ23438 fis, | FLJ23438 fis, clone HRC13275 |
| | | | clone | |
| | | | HRC13275 | |
| 2771 | DNA | AL833005 | cDNA | cDNA DKFZp666D074 |
| | | | DKFZp666D074 | |
| 2772 | DNA | BC003629 | clone | clone MGC: 2854 |
| | | | MGC: 2854 | IMAGE: 2987935 |
| | | | IMAGE: 2987935 | |
| 2773 | DNA | L37793 | small nuclear | small nuclear RNA (U2) gene |
| | | | RNA (U2) | |
| | | | gene | |
| 2774 | DNA | U57614 | U2 snRNA | U2 snRNA (RNU2) gene |
| | | | (RNU2) gene | |

Analogs of the biomarkers provided in Table 1 are also within the scope of the invention. Analogs can differ from the naturally occurring biomarker in nucleotide or amino acid sequence or in ways that do not involve sequence, or both. Non-sequence modifications include in vivo or in vitro chemical derivitization. Non-sequence modifications also include changes in acetylation, methylation, phosphorylation, carboxylation, or glycosylation.

Preferred analogs of the biomarkers provided in Table 1 (or biologically active fragments thereof) include those whose sequences differ from the wild-type sequences by one or more conservative amino acid substitutions or by one or more non-conservative amino acid substitutions, deletions, or insertions which do not abolish biological activity. Conservative substitutions typically include, for example, the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine.

The biomarkers of the invention include any biological molecule that can be detected and quantified in a biological sample using standard biochemical assay methods, where the presence and/or quantity of the biomarker in the biological sample: (i) can be used to select an appropriate treatment; or (ii) can be used to monitor the efficacy and progress of treatment with a cdk modulating agent.

In one aspect, the invention includes the biomarker provided in SEQ ID NO:1246 and assigned GenBank Accession No. W28729. It has been discovered that this biomarker has an expression pattern that correlates with inhibition of cdk in cells upon treatment with a cdk modulating agent. The biomarker of SEQ ID NO:1246 was discovered to have the most consistent and robust regulation in response to cdk inhibition.

The invention also includes specialized microarrays, e.g., oligonucleotide microarrays or cDNA microarrays, comprising one or more biomarkers.

The invention also includes kits comprising a suitable container that comprises: one or more microarrays that comprise one or more biomarkers; one or more cdk modulating agents for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like, as further described herein.

The invention also includes antibodies, including polyclonal or monoclonal, directed against one or more of the biomarker polypeptides. Such antibodies can be used in a variety of ways, for example, to purify, detect, and target the biomarker polypeptides of the invention, including both in vitro and in vivo diagnostic, detection, screening, and/or therapeutic methods.

In carrying out any of the methods of the invention, the levels of either a single biomarker or a set of two or more different biomarkers can be assayed. Assay of more than one biomarker may serve to increase the accuracy of monitoring the response of the patient to treatment with the cdk modulating agent, such as the extent of cdk2 inhibition. Measurement of a plurality of biomarkers can be carried out by assaying the different biomarkers in either the same biological sample or in different biological samples taken from the same patient.

In one aspect, the invention provides a method to monitor the response of a patient being treated for a disorder by administration of a cdk modulating agent, comprising: (a) determining the amount of at least one biomarker in a first biological sample taken from the patient prior to an initial treatment with the agent; (b) determining the amount of the biomarker in at least a second biological sample from the patient subsequent to the initial treatment with the agent; and (c) comparing the amount of the biomarker present in the second biological sample with the amount of the biomarker present in the first biological sample; such that a detectable change in the amount of the biomarker in the second biological sample, and/or in any subsequent biological samples, compared to the amount of biomarker present in the first biological sample indicates that the patient is responding positively to the treatment with the agent The detectable change can be a decrease or an increase in the amount of the biomarker in the second biological sample, and/or in any subsequent biological samples.

This method requires that at least two biological samples are taken from the patient at different time points. The first sample is typically obtained prior to an initial treatment with the cdk modulating agent. A second sample is then obtained, and any subsequent samples are also then obtained, after treatment with the cdk modulating agent has begun. In this method, the biomarker is monitored to determine: (i) if the amount of the biomarker is decreasing, (ii) if the rate of decrease in the amount of the biomarker is increasing, (iii) if the amount of the biomarker is increasing, (iv) if the rate of increase in the amount of the biomarker is increasing, or (v) if the amount of biomarker is stabilizing, any one of which may indicate that the patient is responding positively to the treatment depending upon the specific circumstances.

The biomarkers described herein may be upregulated or down-regulated following treatment with one or more cdk modulating agents.

When the biomarker is an upregulated biomarker, it is expected that the amount of the biomarker will increase following treatment with the cdk modulating agent, i.e., that there will be a detectable increase in the amount of the biomarker in the second biological sample (post administration of the cdk modulating agent) compared to the amount of biomarker in the first biological sample (prior to administration of the cdk modulating agent). If the biomarker is an upregulated biomarker and the level of the biomarker has not increased a predetermined or detectable amount, or if the rate of increase of the biomarker level is not sufficiently high, the treatment can be modified, such as by increasing the dosage or the number of treatments, or by changing the cdk modulating agent being administered to a more effective agent, or by combining the cdk modulating agent being used in the treatment with one or more other cdk modulating agents or therapies, or some combination thereof.

When the biomarker is a downregulated biomarker, it is expected that the amount of the biomarker will decrease following treatment with the cdk modulating agent, i.e., that there will be a detectable decrease in the amount of the biomarker in the second biological sample (post administration of the cdk modulating agent) compared to the amount of biomarker in the first biological sample (prior to administration of the cdk modulating agent). If the biomarker is a down-regulated biomarker and the level of the biomarker has not decreased a predetermined or detectable amount, or if the rate of decrease of the biomarker level is not sufficiently high, the treatment can be modified, such as by increasing the dosage or the number of treatments, or by changing the cdk modulating agent being administered to a more effective agent, or by combining the cdk modulating agent being used in the treatment with one or more other cdk modulating agents or therapies, or some combination thereof.

The invention further provides an improvement to a method for treating a patient suffering from a disorder by administration of a cdk modulating agent, wherein the improvement comprises monitoring the level of at least one biomarker in a biological sample taken from the patient at one or more time points during treatment with the agent so as to determine whether an effective amount of the agent is being administered to the patient. An effective amount of the agent is being administered to the patient if the level of a downregulated biomarker in the biological sample detectably decreases, or if a previously observed rate of decrease in the level of the biomarker increases, in response to administration of the agent. In addition, an effective amount of the agent is being administered to the patient if the level of an upregulated biomarker in the biological sample detectably increases, or if a previously observed rate of increase in the level of the biomarker increases, in response to administration of the agent.

The invention further provides an improvement to a method for treating a patient suffering from a disorder by administration of a cdk modulating agent, wherein the improvement comprises monitoring the level of at least one biomarker in a biological sample taken from the patient at one or more time points during treatment with the agent so as to determine when a sufficient time course of treatment with the agent has been completed. In one embodiment, a sufficient time course of treatment with the agent has been completed when the level of a downregulated biomarker detectably decreases below a predetermined level. In another embodiment, a sufficient time course of treatment with the agent has been completed when the level of an upregulated biomarker detectably increases above a predetermined level.

The type of biological sample from which the amount of biomarker is determined will depend on a variety of factors such as the particular biomarker, where and when it is synthesized, where the biomarker may be stored in the tissues, and into what biological tissue or fluid it may be released or otherwise accumulate. Generally, the biological sample will be selected from the group consisting of blood, a blood component such as serum or plasma, cerebrospinal fluid (CSF), saliva, and urine. In one aspect, the biological sample will be blood, serum, plasma, or CSF, and most preferably blood, serum, or plasma. Where more than one biomarker is analyzed, the analysis can be conducted on the same or different biological samples obtained from the patient.

The amount of the biomarker in a biological sample can be determined using standard techniques known in the art. For example, each biomarker can be assayed using biomarker-specific antibodies and immunological methods known in the art. Any appropriate immunoassay method can be used, including radioimmiunoassays, sandwich enzyme-linked immunoassays, competitive binding assays, homogeneous assays, and heterogeneous assays. Alternatively, the amount of biomarker can be determined using other techniques such as magnetic resonance spectroscopy, HPLC, or mass spectrometry. In any case, the assay method selected should be sensitive enough to be able to measure the particular biomarker in a concentration range from normal values found in healthy patients to elevated levels indicating neurological damage. The assay can be carried out in various formats including, e.g., in a microtiter plate format, using automated immunoassay analyzers known in the art.

As used herein, the predetermined level of the biomarker in the biological sample refers to that amount or concentration of the particular biomarker in a biological sample wherein the amount of the biomarker is higher (upregulated biomarkers) or lower (downregulated biomarkers) statistically than that determined to be present in a biological sample obtained from the patient absent the treatment with the cdk modulating agent. The predetermined level depends upon the particular biomarker.

The expression level of the biomarker provides information about the patient's likely response to treatment with a cdk modulating agent. For this purpose, it is often desirable to correct for (normalize away) both differences in the amount of RNA assayed and variability in the quality of the RNA used. Therefore, the assay typically measures and incorporates the expression of certain normalizing genes, including well known housekeeping genes, such as GAPDH and CYPL. Alternatively, or in addition, normalization can be based on the mean or median signal (Ct in the case of RT-PCR) of all of the assayed genes or a large subset thereof (global normalization approach). On a gene-by-gene basis, measured normalized amount of a patient tumor mRNA is compared to the amount found in a reference set of cancer tissue of the same type. The number (N) of cancer tissues in this reference set should be sufficiently high to ensure that different reference sets (as a whole) behave essentially the same way. If this condition is met, the identity of the individual cancer tissues present in a particular set will have no significant impact on the relative amounts of the genes assayed. The cancer tissue reference set can, in one aspect, consist of at least about 30 different cancer tissue specimens.

While the data described herein were generated in cell lines that are routinely used to screen and identify compounds that have potential utility for cancer therapy, the biomarkers may have both diagnostic and prognostic value in other diseases areas in which cdk or pathways in which cdk is involved is of importance, e.g., in immunology, or in cancers or tumors in which cell signaling and/or proliferation controls have gone awry.

Those having skill in the pertinent art will appreciate that cdk and pathways in which cdk is involved are used and functional in cell types other than cell lines of ovarian carcinoma cells and peripheral blood mononuclear cells. Therefore, the biomarkers and biomarker sets of the invention may show utility in cells from other tissues or organs associated with a disease state, or cancers or tumors derived from other tissue types. Non-limiting examples of such cells, tissues and organs include breast, colon, lung, prostate, testes, ovaries, cervix, esophagus, pancreas, spleen, liver, kidney, stomach, lymphocytic and brain, thereby providing a broad and advantageous applicability to the biomarkers described herein Cells for analysis can be obtained by conventional procedures as known in the art, for example, tissue biopsy, aspiration, sloughed cells, e.g., colonocytes, clinical or medical tissue or cell sampling procedures.

### EXAMPLES

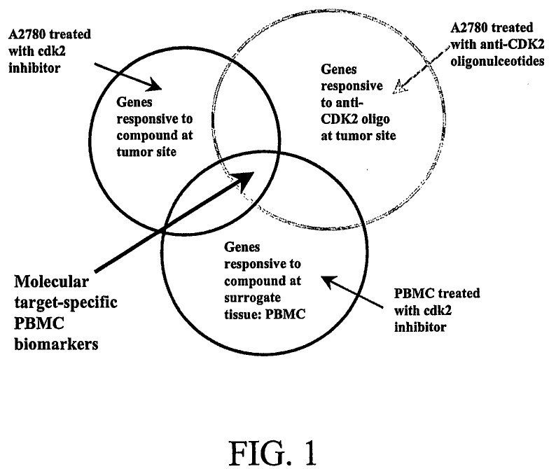

As described below, transcription profiling was used to identify the biomarkers provided above in Table 1. Specifically, transcription profiling of the effect of a certain cdk2 inhibitor on peripheral blood mononuclear cells (PBMCs) was first performed. Next, profiling of a cdk2 inhibitor-treated tumor cell line A2780 at multiple doses and time points was performed to establish a correlation of tumor site response with peripheral blood biomarkers. In order to establish the molecular target-specificity of the potential biomarkers, tumor cell line A2780 treated with anti-cdk2 oligonucleotides was also profiled. Overlapping gene expression changes, as shown in FIG. 1, were selected for further evaluation in human ovarian carcinoma xenograft A2780 that were treated with the cdk2 inhibitor (Example 2). The selected biomarkers were subjected to real-time PCR analysis in order to verify the observed changes from the gene chip analysis. These biomarkers are provided above in Table 1.

In the examples below, the following conditions were employed.

Cdk2 Inhibitor: The cdk2 inhibitor of the examples is N-5-[[5-(1,1-Dimethylethyl)-2-oxazolyl]methyl]thio]-2-thiazolyl-4-piperidinecarboxamide, 0.5-L-tartaric acid salt: 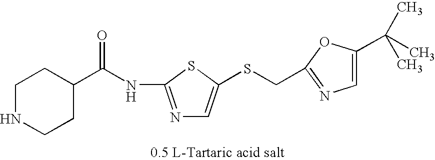
This cdk2 inhibitor was solubilized in 100% DMSO at a concentration of 10 mM. Compound dilutions were made into respective growth media

Cell Culture: The cell lines were maintained in RPMI-1640 plus 10% fetal bovine serum.

Clonogenic Growth Assay: The colony growth inhibition was measured for the A2780 ovarian carcinoma cells using a standard clonogenic assay. In this assay, 200 cells/well were seeded into 6-well tissue culture plates (Falcon™) (Becton, Dickinson and Company, Franklin Lakes, N.J., USA) and allowed to attach for 18 hours. Assay medium consisted of RPMI-1640 plus 10% fetal bovine serum. Cells were then treated in duplicate with a six concentration dose-response curve. The maximum concentration of DMSO never exceeded 0.25%. Cells were exposed to the cdk2 inhibitor for 4, 8, or 24 hours. The cdk2 inhibitor was then removed and the cells were washed with 2 volumes of PBS. The normal growth medium was then replaced. Colonies were fed with fresh media every third day. Colony number was scored on day 10-14 using a Optimax imaging station. The cdk2 inhibitor concentration required to inhibit 50% or 90% of colony formation (IC₅₀ or IC₉₀, respectively) was determined by non-linear regression analysis. The coefficient of variance (standard deviation/mean, n=3)=30%.

Real-Time Quantitative PCR Assays: A Taqman® real-time-PCR fluorogenic assay (Applied Biosystems, Foster City, Calif., USA) was used to quantitate the levels of specific mRNA. The cdk2 inhibitor treated A2780s cells were harvested at approximately 70% confluence and total RNA was prepared using the Qiagen RNeasy 96 Kit.

Taqman® reactions were prepared as follows: 100 ng total RNA; 25 nM-750 nM Forward Primer; 25 nM-750 nM Reverse Primer; 200 nM-400 nM Taqman® Probe (fluorescent dye labeled oligonucleotide primer); 1X Buffer A (Applied Biosystems, Foster City, Calif., USA); 5.5 mM MgCl₂; 300 μM DATP, dGTP, dTTP, dCTP; 1 U Amplitaq Gold; 20 U Superscript 2; 1 U RNase Inhibitor. Real-time PCR was performed using an Applied Biosystems 7700 Sequence Detection System. Conditions were as follows: 48° C. for 20 minutes (reverse transcription), 95° C. for 10 minutes (denaturation and activation of Amplitaq Gold), 40 cycles of PCR (95° C. for 15 seconds, 60° C. for 1 minutes).

The Sequence Detection System generates a Ct (threshold cycle) value that is used to calculate a concentration for each input messenger RNA template. Messenger RNA levels for each gene or fragment thereof of interest were normalized to GAPDH message levels to compensate for variations in total RNA quantity in the input sample. This was done by generating GAPDH Ct values for each cell line. Ct values for the gene or fragments thereof of interest and GAPDH were inserted into the δδCt equation:
Relative Quantity of Nucleic Acid Template=2^{δδCt}=2^{(δCta-δCtb)}(δCta=Ct target−Ct GAPDH, δCtb=Ct reference−Ct GAPDH)
which was used to calculate a normalized relative message level.

Gene Chip Analysis: Gene chips were used to quantitate the levels of gene expression on a large-scale with Affymetrix human gene chips HG-U95A, B, and C (Affymetrix, Inc., Santa Clara, Calif., USA). Gene chip hybridization was performed using an Affymetrix gene chip system including hybridization oven, washing station, scanner, and a computer workstation. Manufacturer's standard protocol was followed. Raw data were generated using Affymetrix Microarray Suite 4.0 software. A threshold of 20 units was assigned to any gene with a calculated expression level below 20, because discrimination of expression below this level could not be performed with confidence.

In Vitro Treatment of PBMC: PBMCs were isolated and incubated with the cdk2 inhibitor in vitro. Specifically, approximately 40 ml of blood were collected for the pilot study and then from 10 volunteers. The 40 ml of blood were then put into five Vacutainer™ CPT™ Mononuclear Cell Preparation Tubes (Product Number: 362753) (Becton, Dickinson and Company, Franklin Lakes, N.J., USA) with Sodium Heparin Anticoagulant 60/cs. Lymphocytes were then removed from the five Vacutainers™ pool and re-suspended in 20 ml of culture medium (RPMI, 10% serum, and glu/Pen/strep). Cells were counted at this step, and then centrifuged gently and then suspended with 4.0 ml of culture medium. Cells were then plated into 6 well plates (0.5 ml/well). Culture medium containing the cdk2 inhibitor or vehicle (3.5 ml) was then added to each well to give a final concentration of 100 nM cdk2 inhibitor in experimental wells, and also a final concentration of 1000 nM cdk2 inhibitor in experimental wells for the 10 subjects.

RNA and protein samples were harvested at 4 and 24 hours after addition of the cdk2 inhibitor. RNA was prepared using the RNeasy-mini RNA kit according to the manufacturer's specifications (Qiagen, Valencia, Calif., USA). For protein samples, cells were washed once with PBS before extracting with 0.5-1.0 ml of modified RIPA buffer [50 mM Tris (pH 8), 150 mM NaCl, 1% NP-40, 0.5% Na-deoxycholate, 0.1% SDS, 0.1% Na3VO4, 0.1 mM NaF, 10 mM β-glycerophosphate, plus Complete® protease inhibitors (Boehringer Mannhiem GmbH, Germany)]. Lysates were frozen at −80° C. Viability of cells at different time points following the cdk2 inhibitor treatment was determined by trypan blue exclusion.

Western Blot Analysis: The cdk2 inhibitor treated A2780s cells were harvested at approximately 70% confluence and total protein was prepared by lysing the cells in RIPA [50 mM Tris (pH 8), 150 mM NaCl, 1% NP-40, 0.5% Na-deoxycholate, 0.1% SDS, 0.1% Na3VO4, 0.1 mM NaF, 10 mM β-glycerophosphate, plus Complete® protease inhibitors (Boehringer Mannhiem GmbH, Germany)] buffer. Cell pellets were resuspended at a density of <2× 10⁷ cells/ml and incubated for 20 minutes on ice followed by a high speed 14,000 rpm centrifugation. The protein supernatant was then removed from the debris and protein content was quantitated using the Micro-BCA assay (Pierce Biotechnology, Inc., Rockford, Ill., USA). Treated extracts (25 μg/lane) were then separated using a 10% SDS-polyacrylamide gel (10.5× 14 cm). Proteins were then transferred from the gel to PVDF-membrane (Millipore Corporation, Billerica, Mass., USA) by exposure to 0.8 Amp/cm² in a semi-dry blotting apparatus (Hoefer Scientific Instruments, San Francisco, Calif., USA). PVDF protein blots were then blocked with 5% non-fat milk in TTBS (0.1% Tween 20 in Tris-buffered saline). Blots were then probed with primary antibody (mouse anti-cdk2 clone D-12, Santa Cruz Biotechnology, Santa Cruz, Calif., USA) in 5% non-fat milk in TTBS for 1-2 hours, followed by three washes with TTBS. An HRP-conjugated secondary antibody (HRP conjugated goat anti-mouse IgG, Promega Corp., Madison, Wis., USA) was then incubated with the blots in TTBS for 30 minutes. The blots were then washed three times with TTBS and developed with ECL-plus western blotting detection system (Amersham Biosciences, Piscataway, N.J., USA).

Cdk2 Antisense Treatment: A mixture of five antisense oligonucleotides targeted against cdk2 mRNA having the following sequences was used:

GCAGUAUACCUCUCGCUCUUGUCAA (SEQ ID NO:2775); UUUGGAAGUUCUCCAUGAAGCGCCA (SEQ ID NO:2776); GUCCAAAGUCUGCUAGCUUGAUGGC (SEQ ID NO:2777); CCCAGGAGGAUUUCAGGAGCUCGGU (SEQ ID NO:2778); UAGAAGUAACUCCUGGCCACACCAC (SEQ ID NO:2779). All gene modulations were based on relative levels of RNA in antisense treated cells versus reverse control oligonucleotide treated cells.

A2780s cells were plated in 6-well tissue culture plates at a density of 1-2×10⁵ cells/well. After an overnight incubation, cells were transfected with the antisense oligonucleotide mixture using Lipofectamine 2000 (Invitrogen Life Technologies, Carlsbad, Calif., USA). Briefly, a 10× lipid solution (10 ug/ml in OptiMEM) and a 10× oligonucleotide mixture (0.5 uM in OptiMEM) were prepared. A 5× solution of lipid/oligonucleotide complex was then prepared by mixing equal volumes of 10× lipid solution and 10× oligonucleotide mixture. The 5× solution of lipid/oligonucleotide complex was allowed to incubate at room temperature for 15 minutes to allow complexes to form. After incubation, the 5× lipid/oligonucleotide complex was diluted in RPMI containing 10% Fetal Bovine Serum to produce a 1× transfection reagent Cells in 6-well culture plates were transfected by replacing the overnight growth media with 1× transfection reagent. Cells were then incubated at various times (0, 12, 16, 20, and 24 hours) prior to harvesting RNA for analysis by Taqman® real-time-PCR fluorogenic assay. In every experiment, an extra well was transfected with a fluoresceinated random oligonucleotide to determine the transfection efficiency using flow cytometry. For all experiments, between 85% and 95% of A2780s cells were transfected.

### Example 1

### Transcription Profiling of Peripheral Blood Mononuclear Cells (PBMCs) Following Treatment with Cdk2 inhibitor, and A2780S Ovarian Carcinoma Cells Following Treatment with Cdk2 inhibitor or Anti-cdk2 Antisense Oligonucleotides

To identify biomarkers, transcriptional profiling was obtained for (i) PBMCs following treatment with cdk2 inhibitor, (ii) A2780S ovarian carcinoma cells following treatment with cdk2 inhibitor, and (iii) A2780S ovarian carcinoma cells following treatment with anti-cdk2 antisense oligonucleotides.

Table 2 lists the doses and time course used for treatment of the A2780 and PBMC cell types.

TABLE 2
Experimental design

| | | Drug Dose | Time course |
|---|---|---|---|
| Cell Type | Treatment | (nM) | (hours) |
| A2780 | cdk2 inhibitor | 0, 20, 100, 200 | 0, 1, 2, 4, 6, 24 |
| PBMC | cdk2 inhibitor | 0, 100 | 0, 4, 24 |
| (pooled 10 | | | |
| subjects) | | | |
| PBMC | cdk2 inhibitor | 0, 100, 1000 | 0, 4, 24 |
| (pilot) | | | |
| A2780 | Anti-cdk2 | Antisense oligo and | 0, 12, 16, 20, 24 |
| | oligonucleotide | control | |

Treatment of A2780 and PBMC was carried out as described above. The doses of the cdk2 inhibitor were derived from an understanding of the kinetics of tumor cell growth inhibition by the cdk2 inhibitor as assessed by proliferation and clonogenic assays (Table 3). This study clearly demonstrated that growth inhibition by the cdk2 inhibitor was time dependent. A minimal exposure of 8 hours was required for effective inhibition of colony formation. The values obtained from the 24 hour clonogenic assay were in good agreement with the 72 hour proliferation assay.

TABLE 3
Inhibition of colony formation by cdk2 inhibitor

| | IC₅₀ (nM) | IC₉₀ (nM) |
|---|---|---|
| A2780s Clonogenic assay, 4 hr. exposure | 302 | >1000 |
| A2780s Clonogenic assay, 8 hr. exposure | 154 | 303 |
| A2780s Clonogenic assay, 24 hr. | 166 | 208 |
| exposure | | |
| A2780s, 72 hr. XTT assay | 95 | 170 |

A pilot experiment of ex vivo treatment of PBMC from one healthy volunteer with the cdk2 inhibitor was first performed. Subsequently, PBMCs from ten healthy human subjects were collected and treated ex vivo with the cdk2 inhibitor. Total RNA was isolated and hybridized to gene chips.

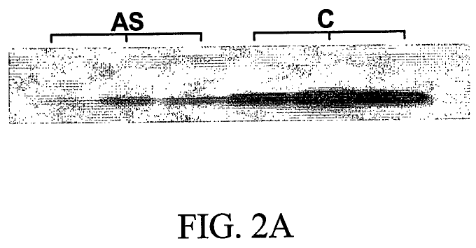

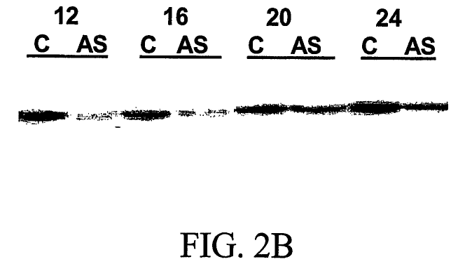

Antisense inhibition of cdk2 expression was optimized for A2780 cells and carried out as described above. Under these conditions, cdk2 protein levels decreased 90% after 24 hours exposure. As shown in FIG. 2A, consistent reduction of cdk2 protein was observed in all three antisense treated wells (AS) relative to the controls wells (C). This resulted in a block in cell cycle progression and apoptosis that is similar to the cdk2 inhibitor treated A2780s cells. The decrease in cdk2 protein in relation to time of exposure was also determined. As shown in FIG. 2B, cdk2 levels were maximally inhibited at 12 hours and protein levels remained reduced through 24 hours.

### Example 2

### Selection of Biomarkers

In order to identify biomarkers for the cdk2 inhibitor that can be used as surrogate endpoints in PBMC and have molecular target-specific response, the expression profiles of the three sets of experiments in Example 1 were compared. Overlapping gene expression changes were selected as shown in FIG. 1.

To allow for the identification of cdk2 specific responses as well as compound specific changes at gene expression level, a statistical method was used to select genes that have gene expression changes associated with dose and time of treatment in the cdk2 inhibitor treated A2780s sample set. The data were analyzed using an analysis of variance (ANOVA) model to study the compound's dose effect and time effect on each gene. First, the data were rescaled to eliminate the chip effects by a linear regression technique. Then, an ANOVA model was fitted for each gene based on two factors—dose and time. The F-test was used to determine if there was significant dose or time effect in terms of the changes in the expression level of a particular gene. Genes with the p-value less than 0.05 in both dose effect test and time effect test were identified. The genes identified with a p-value of less than 0.05 in both dose effect and time effect are provided Table 1.

Overlapping gene expression changes from the three sets of Example 1 were selected for further evaluation in human ovarian carcinoma xenograft A2780 treated with the cdk2 inhibitor.

The human ovarian carcinoma xenograft A2780s were maintained in Balb/c nu/nu nude mice. Tumors were propagated as subcutaneous (sc) transplants using tumor fragments obtained from donor mice. For the cdk2 inhibitor treatment, tumors were allowed to grow to the pre-determined size window of approximately 100-200 mg (tumors outside the range were excluded) and animals were evenly distributed to various treatment and control groups (n=6). Treatment of each animal was based on individual body weight.

The cdk2 inhibitor was first dissolved in a mixture of Cremophor®/ethanol (50:50). One hour prior to administration, the cdk2 inhibitor was diluted with water so that the dosing solutions contained the specified excipient composition, i.e., Cremophor®/ethanol/water (1:1:8, v/v). The volume of all compounds injected was 0.01 ml/gm of mice. The cdk2 inhibitor was administered as a bolus injection intraperitoneal at doses of 36 and 18 mg/kg. Tumor and plasma were sampled at the time points of 4, 7, and 24 hour post treatment. Plasma sample was frozen immediately at −80° C. for pharmacokinetic analysis, and tumor sample was preserved in RNAase free buffer for pharmacogenomic analysis.

Once certain genes were selected as potential biomarkers, real-time PCR assays using fluorescent MGB Taq-man probes were developed as described above.

The selected genes were subjected for real-time PCR analysis as described above in order to verify the observed changes from gene chip analysis.

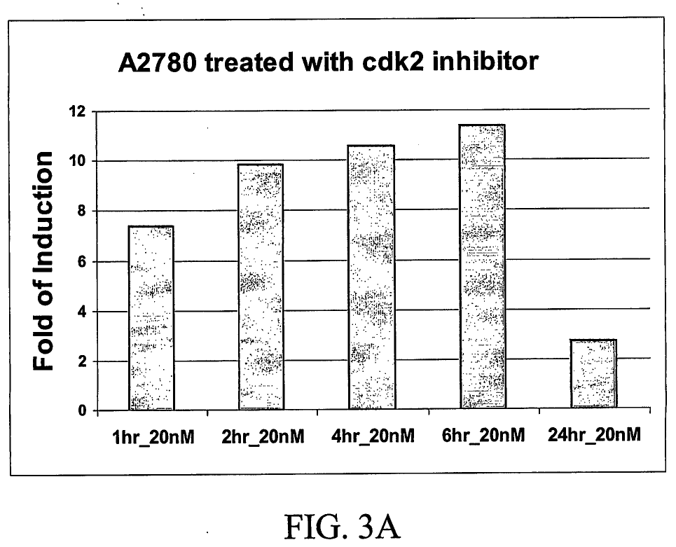

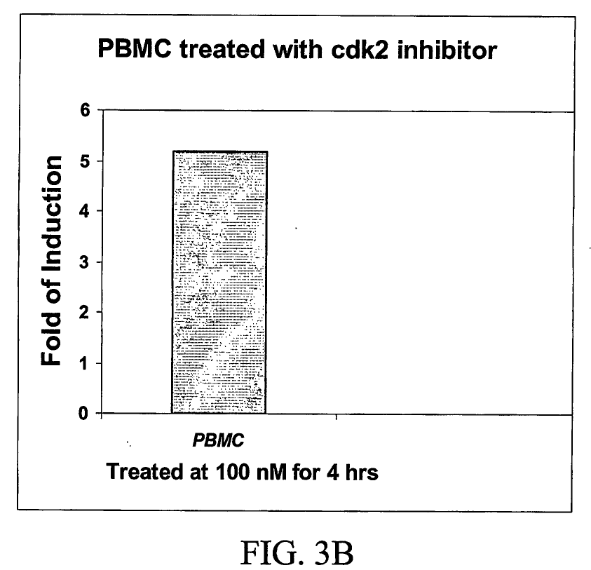

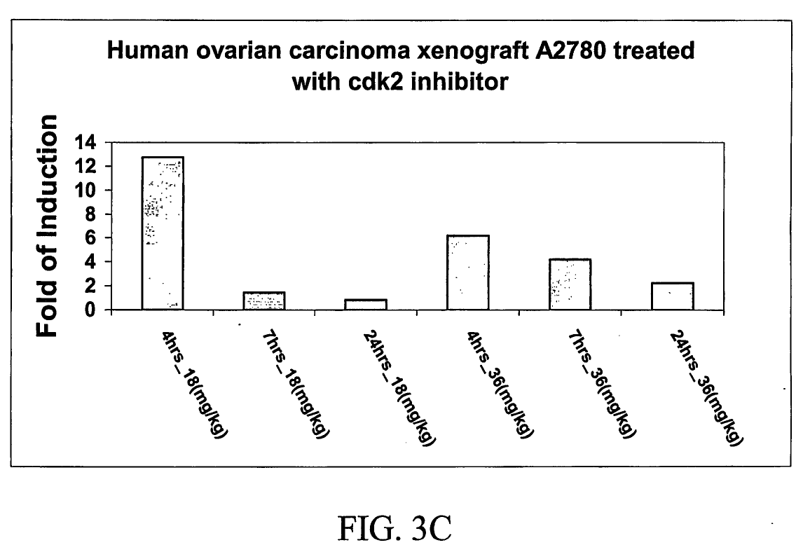

The biomarker W28729 (SEQ ID NO: 1246) was selected as a preferred marker. A same-well multiplex real-time quantitative PCR assay on this biomarker with normalization control, house-keeping gene GAPDH, was developed using Taq-man MGB probes. Gene expression changes for W28729 were measured with real-time quantitative PCR assays in the following sample sets: A2780 human tumor cell line treated with 20 nM of cdk2 inhibitor for different durations (FIG. 3A), PBMC treated with 10n-M cdk2 inhibitor at 4 hours (FIG. 3B); and human ovarian carcinoma xenograft A2780 treated with cdk2 inhibitor at doses of 36 and 18 mg/kg for different durations (FIG. 3C). In cultured A2780 tumor cells, induction of W28729 occurred upon treatment with 20 nM of cdk2 inhibitor, and was detected 1 h after treatment. Upregulation of W28729 expression was also observed upon treatment of human PBMC in vitro with the cdk2 inhibitor. Treatment of nude mice bearing A2780 xenografts with efficacious doses of the cdk2 inhibitor also resulted in induction of W28729, and there was a dose-dependent prolongation of the duration of gene induction.

### Example 3

### W28729 Upregulation

The following experimental methods were used to further study W28729 upregulation.

Patient inclusion criteria: The patient inclusion criteria included: primary solid malignancy refractory to current therapy and adequate bone marrow, hepatic, and renal function.

Treatments: Two different treatments were undertaken: (i) 174-001 Study: 1 hr infusion of BMS-387032 q 3 wks; and (ii) 174-002 Study: 24 hr infusion of BMS-387032 q 3 wks. The sampling times were pre-dose, and 2, 6, 24 hour post-dose.

W28729 Expression Analysis: RT-PCR. Patient blood samples were collected in PAXgene™ Blood Collection Tubes (Qiagen, catalog #762155). Total RNA was isolated following the manufacturer's instructions using a PAXgene™ blood RNA Kit (Qiagen, catalog #762134). W28729 and GAPDH (housekeeping gene) RNA abundance was measured by Taqman assays, using an ABI PRISM 7900 HT Sequence Detection System. W28729 abundance was normalized relative to GAPDH. Primer and probe sequences are as shown below.

| | | |
|---|---|---|
| W28729: | | |
| (5+) | AGTACCGTGAGGTTCCTGATGTG | (SEQ ID NO:2780) |
| (3+) | TGGCAAGCTGAGATCCTAAGG | (SEQ ID NO:2781) |
| Probe | TTATGCGGCAGCCTT | (SEQ ID NO:2782) |
| GAPDH: | | |
| (5+) | CGACAGTCAGCCGCATCTT | (SEQ ID NO:2783) |
| (3+) | AAATCCGTTGACTCCGACCTT | (SEQ ID NO:2784) |
| Probe | CATCGCTCAGACACCA | (SEQ ID NO:2785) |

Results

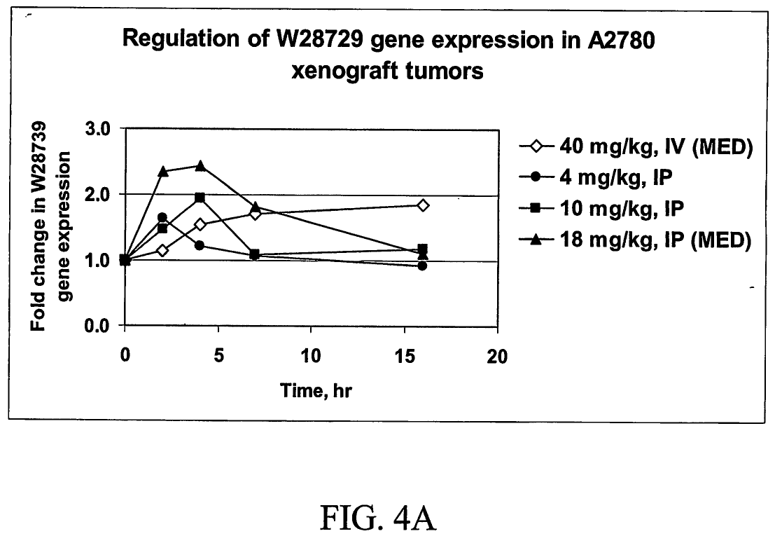

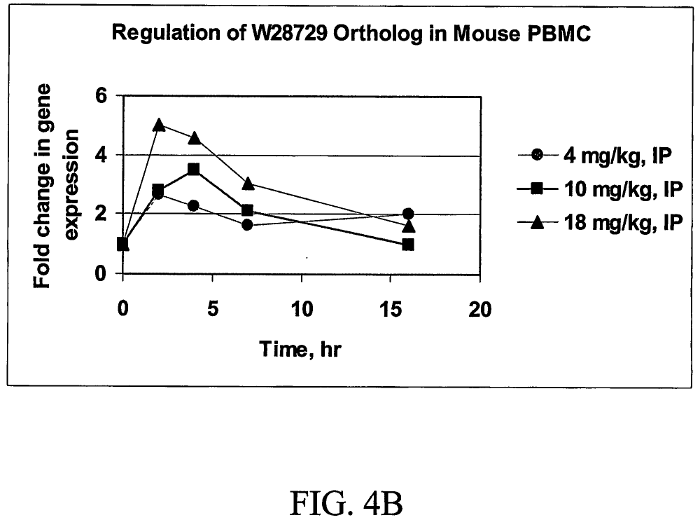

Preclinical Xenografts: In A2780 xenografts given bolus i.p. treatments with BMS-387032, the induction of W28729 in the tumors occurred in a transient, dose-dependent manner (FIG. 4A). At the minimum efficacious dose (MED) of 18 mg/kg/day, the induction was sustained for more than 6 hours. In an infusion regimen using the minimum efficacious dose of 40 mg/kg, gene induction was sustained for at least 16 hours. The gene induction in tumors was accompanied by a strikingly similar pattern of induction of the mouse ortholog sequence (SEQ ID NO:2786; a fragment of mouse genomic DNA sequence locus AL590994), as detected in PBMC isolated from the tumor mice (FIG. 4B). Treatment with an efficacious regimen results in >2 fold induction of the sequence for 6 hours or longer. These data support the use of W28729 gene induction in tumor as a pharmacodynamic biomarker. In addition, these observations support the use of PBMC as a surrogate tissue for monitoring changes in gene expression, that result from treatment with the cdk2 inhibitor.

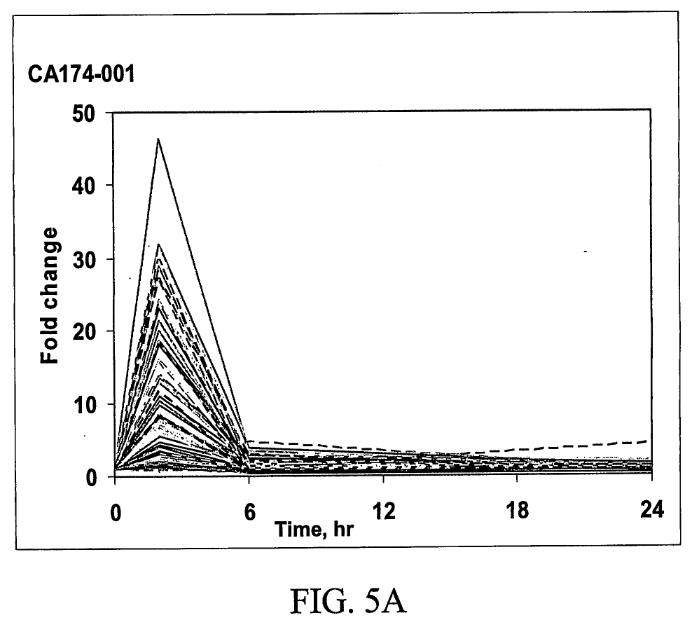

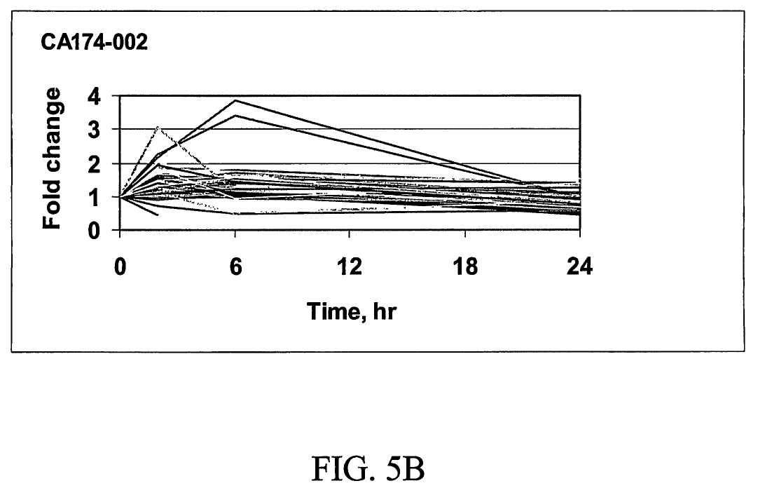

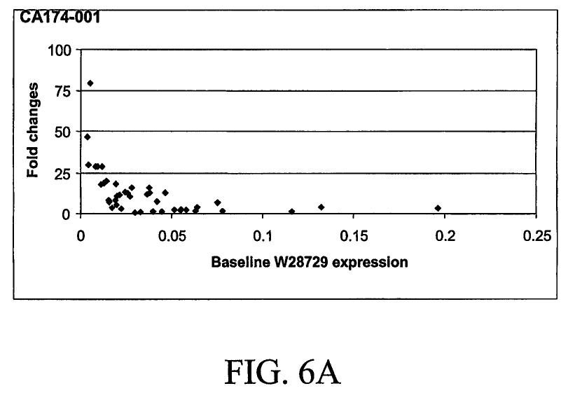

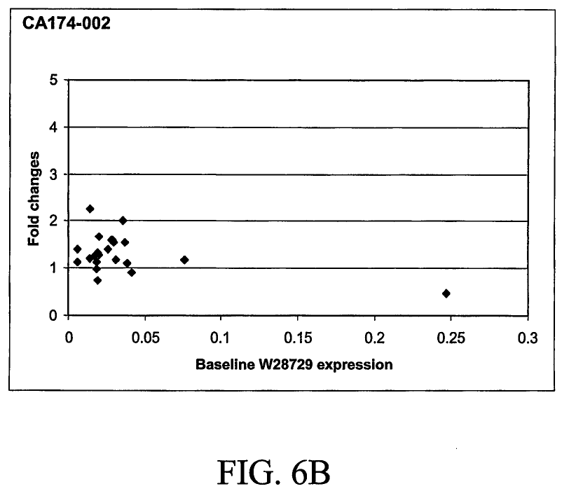

Clinical Trials: In the CA174001 study (1 hour infusion), transient induction of W28729 was detected in PBMC at 2 hours and returned to baseline levels by 6 hours (FIG. 5A). In the CA174-002 study (24 hour infusion), there was modest induction of W28729 expression, which was sustained for 6 hours following end of infusion (FIG. 5B). Each line in FIGS. 5A and 5B represents the extent of gene induction for an individual patient at the specified times after dosing. There was an inverse relationship between baseline expression and the level of maximal gene induction in the CA174-001 study (FIG. 6A). There was no clear relationship between baseline expression and induction magnitude in the CA174-002 study (FIG. 6B). Interpretation of the data from the 24 hour infusion study is difficult because expression data were collected more than 24 hours after the beginning of dosing.

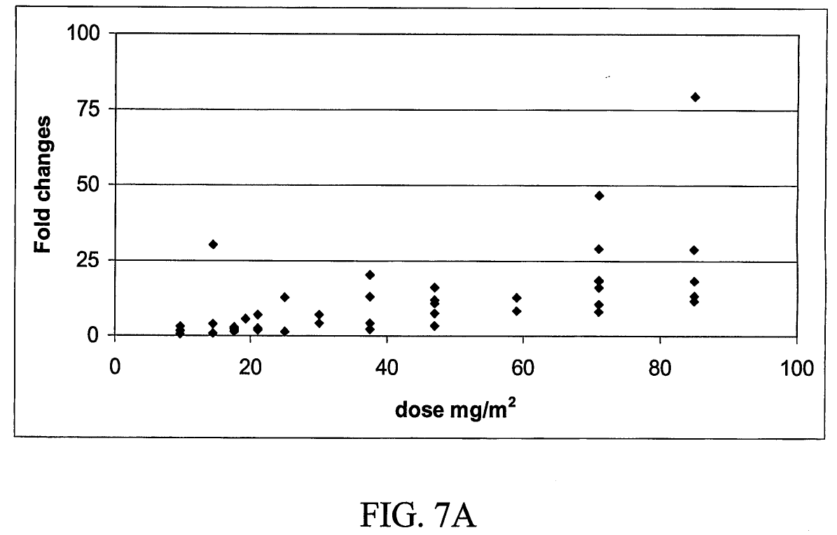

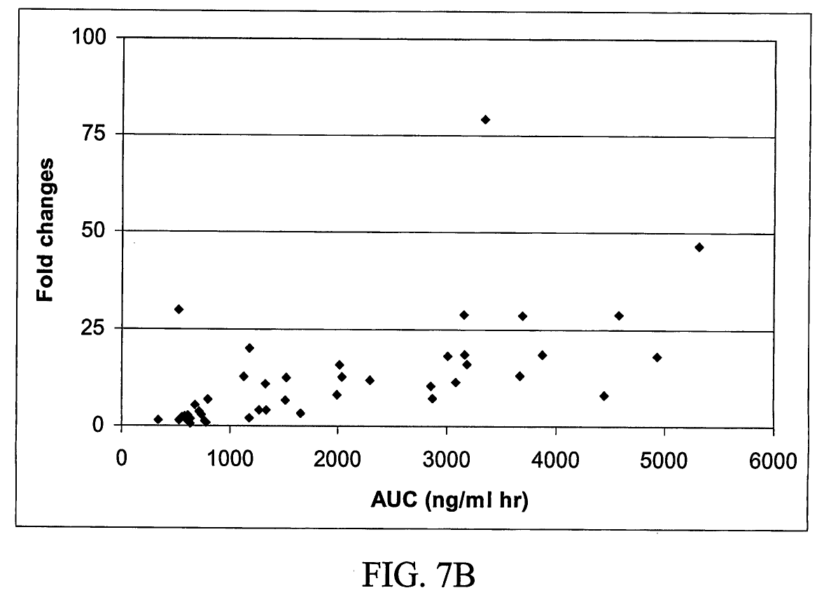

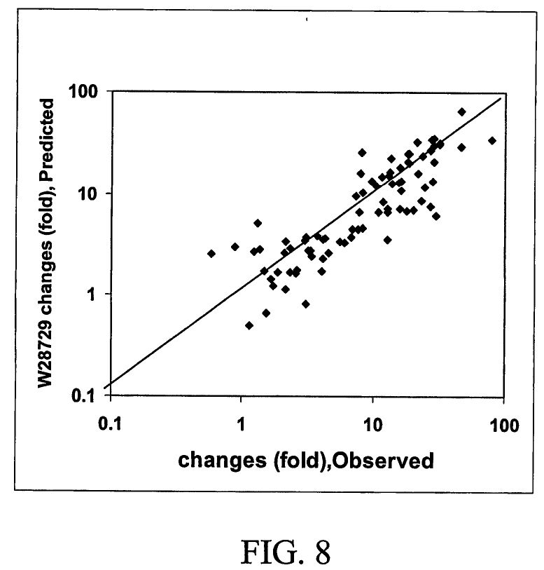

FIGS. 7A and 7B illustrate W28729 induction as a function of dose (FIG. 7A) and AUC (FIG. 7B) from the CA174-001. As shown in FIGS. 7A and 7B, there was a linear relationship between W28729 gene induction and dose or exposure of the cdk2 inhibitor. FIG. 8 provides a prediction of W28729 changes by baseline expression of W28729 and the cdk inhibitor exposure in the CA174-001 study. W28729 gene expression changes can be predicted by the formula: Δ(W28729 expression)=A*AUC*(Baseline expression)^{B}, wherein A=0.000619 and B=−0.537. Induction of W28729 gene can be reliably predicted from drug exposure and baseline W28729 expression.

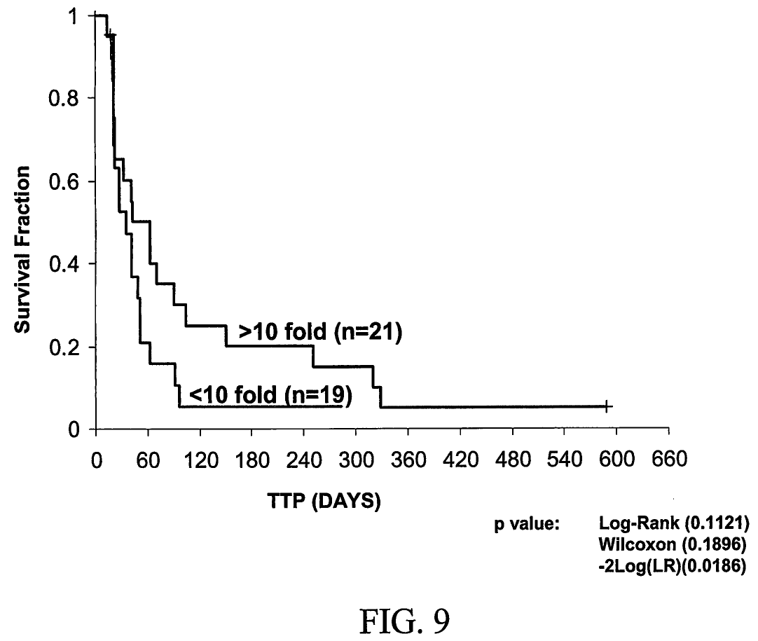

Since the pre-clinical data suggest that the extent and duration of W28729 gene induction correlate with anti-tumor efficacy, the disease outcome of patients who showed different W28729 induction in the CA174-001 study was examined. Interestingly, those patients with high induction appeared to have the most favorable outcome (FIG. 9). These results suggest that W28729 induction is a surrogate marker for prediction of clinical outcome of agents that modulate cdk.

What is claimed is:

1. A method for testing or predicting whether a mammal will respond therapeutically to a method of treating cancer comprising administering an agent that modulates cdk activity, wherein the method comprises:
(a) measuring in the mammal the level of the nucleotide sequence of SEQ ID NO:1246;
(b) exposing the mammal to the agent that modulates cdk activity; and
(c) following the exposing of step (b), measuring in the mammal the level of the nucleotide sequence of SEQ ID NO:1246,
wherein a difference in the level of the nucleotide sequence of SEQ. ID NO:1246 measured in step (c) compared to the level of the nucleotide sequence of SEQ ID NO:1246 measured in step (a) indicates that the mammal will respond therapeutically to said method of treating cancer.

2. The method of claim 1 wherein said agent is N-5-[[5-(1,1-Dimethylethyl)-2-oxazolyl]methyl]thio]-2-thiazolyl-4-piperidinecarboxamide, 0.5-L-tartaric acid salt.

3. A method for determining whether a mammal is responding to an agent that modulates cdk activity, comprising:
(a) obtaining a biological sample from the mammal;
(b) measuring in said biological sample the level of the nucleotide sequence of SEQ ID NO:1246;
(c) correlating said level of the nucleotide sequence of SEQ ID NO:1246 with a baseline level; and
(d) determining whether the mammal is responding to an agent that modulates cdk activity based on said correlation.

4. The method of claim 3 wherein said agent is N-5-[[5-(1,1-Dimethylethyl)-2-oxazolyl]methyl]thio]-2-thiazolyl-4-piperidinecarboxamide, 0.5-L-tartaric acid salt.

5. A method for testing or predicting whether a mammal will respond therapeutically to a method of treating cancer comprising administering an agent that modulates cdk activity, wherein the method comprises:
(a) measuring in the mammal the level of at least one biomarker selected from the biomarkers of Table 1;
(b) exposing the mammal to the agent that modulates cdk activity;
(c) following the exposing of step (b), measuring in the mammal the level of the at least one biomarker,
wherein a difference in the level of the at least one biomarker measured in step (c) compared to the level of the at least one biomarker measured in step (a) indicates that the mammal will respond therapeutically to said method of treating cancer.

6. The method of claim 5 wherein said agent is N-5-[[5-(1,1-Dimethylethyl)-2-oxazolyl]methyl]thio]-2-thiazolyl-4-piperidinecarboxamide, 0.5-L-tartaric acid salt.

7. The method of claim 5 wherein the at least one biomarker is a protein.

8. The method of claim 5 wherein the at least one biomarker is an mRNA transcript.

9. A method for determining whether a mammal is responding to an agent that modulates cdk activity, comprising:
(a) obtaining a biological sample from the mammal;
(b) measuring in said biological sample the level of at least one biomarker selected from the biomarkers of Table 1;
(c) correlating said level of at least one biomarker with a baseline level; and
(d) determining whether the mammal is responding to an agent that modulates cdk activity based on said correlation.

10. A method for determining whether a mammal is responding to an agent that modulates cdk activity, comprising:
(a) exposing the mammal to the agent; and
(b) following the exposing of step (a), measuring in the mammal the level of at least one biomarker selected from the biomarkers of Table 1,
wherein a difference in the level of the at least one biomarker measured in step (b), compared to the level of the at least one biomarker in a mammal that has not been exposed to said agent, indicates that the mammal is responding to the agent that modulates cdk activity.
